(19) United States
(12) Patent Application Publication
(10) Pub. No.: US 2012/0245161 A1
(43) Pub. Date: Sep. 27, 2012

(54) INDOLYL-PIPERIDINYL BENZYLAMINES AS BETA-TRYPTASE INHIBITORS

(75) Inventors: Yong Mi CHOI-SLEDESKI, Belle Mead, NJ (US); Guyan LIANG, Warren, NJ (US); Thaddeus R. NIEDUZAK, Bridgewater, NJ (US); Gregory B. POLI, Bethlehem, PA (US); Patrick Wai-Kwok SHUM, Flemington, NJ (US); Gregory T. STOKLOSA, Hillsborough, NJ (US); Zhicheng ZHAO, Bridgewater, NJ (US)

(73) Assignee: SANOFI, Paris (FR)

(21) Appl. No.: 13/488,539

(22) Filed: Jun. 5, 2012

Related U.S. Application Data
(63) Continuation of application No. PCT/US2010/061461, filed on Dec. 21, 2010.
(60) Provisional application No. 61/289,650, filed on Dec. 23, 2009.

(30) Foreign Application Priority Data
Sep. 21, 2010 (FR) 1057580

(51) Int. Cl. A61K 31/454; C07D 413/14; A61K 31/5377; A61P 11/00; A61K 31/4545; C07D 498/06; A61K 31/5383; C07D 401/06; C07D 401/14

(52) U.S. Cl. 514/230.2; 546/201; 514/323; 544/130; 514/235.2; 546/187; 514/316; 544/101

(57) ABSTRACT

The present invention discloses and claims a series of substituted indolyl-piperidinyl benzylamines of the formula

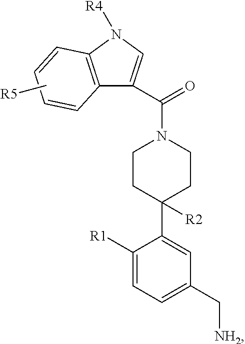

wherein R1, R2, R4 and R5 are as described herein. More specifically, the compounds of this invention are inhibitors of β-tryptase and are, therefore, useful as pharmaceutical agents. Additionally, this invention also discloses methods of preparation of substituted indolyl-piperidinyl benzylamines.

### BACKGROUND OF THE INVENTION

1. Field of the Invention

The present invention relates to a series of substituted indolyl-piperidinyl benzylamines. The compounds of this invention are inhibitors of β-tryptase and are, therefore, useful as pharmaceutical agents. Additionally, this invention also relates to methods of preparation of substituted indolyl-piperidinyl benzylamines and intermediates therefor.

2. Description of the Art

Mast cell mediated inflammatory conditions, in particular asthma, are a growing public health concern. Asthma is frequently characterized by progressive development of hyper-responsiveness of the trachea and bronchi to both immunospecific allergens and generalized chemical or physical stimuli, which lead to the onset of chronic inflammation. Leukocytes containing IgE receptors, notably mast cells and basophils, are present in the epithelium and underlying smooth muscle tissues of bronchi. These leukocytes initially become activated by the binding of specific inhaled antigens to the IgE receptors and then release a number of chemical mediators. For example, degranulation of mast cells leads to the release of proteoglycans, peroxidase, arylsulfatase B, chymase, and tryptase, which results in bronchiole constriction.

Tryptase is stored in the mast cell secretory granules and is the major protease of human mast cells. Tryptase has been implicated in a variety of biological processes, including degradation of vasodilatory and bronchodilatory neuropeptides (Caughey, et al., J. Pharmacol. Exp. Ther., 1988, 244, pages 133-137; Franconi, et al., J. Pharmacol. Exp. Ther., 1988, 248, pages 947-951; and Tam, et al., Am. J. Respir. Cell Mol. Biol., 1990, 3, pages 27-32) and modulation of bronchial responsiveness to histamine (Sekizawa, et al., J. Clin. Invest., 1989, 83, pages 175-179).

As a result, tryptase inhibitors may be useful as anti-inflammatory agents (K Rice, P. A. Sprengler, Current Opinion in Drug Discovery and Development, 1999, 2(5), pages 463-474) particularly in the treatment of chronic asthma (M. Q. Zhang, H. Timmerman, Mediators Inflamm., 1997, 112, pages 311-317), and may also be useful in treating or preventing allergic rhinitis (S. J. Wilson et al, Clin. Exp. Allergy, 1998, 28, pages 220-227), inflammatory bowel disease (S. C. Bischoff et al, Histopathology, 1996, 28, pages 1-13), psoriasis (A. Naukkarinen et al, Arch. Dermatol. Res., 1993, 285, pages 341-346), conjunctivitis (A. A. Irani et al, J. Allergy Clin. Immunol., 1990, 86, pages 34-40), atopic dermatitis (A. Jarvikallio et al, Br. J. Dermatol., 1997, 136, pages 871-877), rheumatoid arthritis (L. C Tetlow et al, Ann. Rheum. Dis., 1998, 54, pages 549-555), osteoarthritis (M. G. Buckley et al, J. Pathol., 1998, 186, pages 67-74), gouty arthritis, rheumatoid spondylitis, and diseases of joint cartilage destruction.

In addition, tryptase has been shown to be a potent mitogen for fibroblasts, suggesting its involvement in the pulmonary fibrosis in asthma and interstitial lung diseases (Ruoss et al., J. Clin. Invest., 1991, 88, pages 493-499).

Therefore, tryptase inhibitors may be useful in treating or preventing fibrotic conditions (J. A. Cairns and A. F. Walls, J. Clin. Invest., 1997, 99, pages 1313-1321) for example, fibrosis, sceleroderma, pulmonary fibrosis, liver cirrhosis, myocardial fibrosis, neurofibromas and hypertrophic scars.

Additionally, tryptase inhibitors may be useful in treating or preventing myocardial infarction, stroke, angina and other consequences of atherosclerotic plaque rupture (M. Jeziorska et al, J. Pathol., 1997, 182, pages 115-122).

Tryptase has also been discovered to activate prostromelysin that in turn activates collagenase, thereby initiating the destruction of cartilage and periodontal connective tissue, respectively.

Therefore, tryptase inhibitors could be useful in the treatment or prevention of arthritis, periodontal disease, diabetic retinopathy, and tumour growth (W. J. Beil et al, Exp. Hematol., (1998) 26, pages 158-169). Also, tryptase inhibitors may be useful in the treatment of anaphylaxis (L. B. Schwarz et al, J. Clin. Invest., 1995, 96, pages 2702-2710), multiple sclerosis (M. Steinhoff et al, Nat. Med. (N.Y.), 2000, 6(2), pages 151-158), peptic ulcers and syncytial viral infections.

Such a compound should readily have utility in treating a patient suffering from conditions that can be ameliorated by the administration of an inhibitor of tryptase, e.g., mast cell mediated inflammatory conditions, inflammation, and diseases or disorders related to the degradation of vasodilatory and bronchodilatory neuropeptides, and have diminished liability for semicarbazide-sensitive amine oxidase (SSAO) metabolism.

β-tryptase is located solely in mast cell granules as the most abundant serine protease and is released following stimulation of the IgE receptor by allergen. In experimental animals, β-tryptase release provokes inflammation and bronchoconstriction characteristic of human asthma. It is also thought to cause fibroblast activation and therefore to have a role in airways remodeling. Levels of β-tryptase are elevated in bronchoalveolar lavage fluid (BALF) from asthmatic patients. Clinical proof-of-concept (bronchial allergen challenge) for asthma has been reported with an inhaled β-tryptase inhibitor (APC-366—since terminated due to bronchial irritation). β-tryptase inhibitors have the potential to impact the symptoms and pathogenesis of a number of proinflammatory indications, in particular, asthma and potentially COPD.

Benzylamine containing tryptase inhibitors, as one popular class of serine protease inhibitors, are also recognized as substrates for amine oxidases, especially SSAO.

All of the references described herein are incorporated herein by reference in their entirety.

Accordingly, it is an object of this invention to provide a series of substituted indolyl-piperidinyl benzylamines that are inhibitors of β-tryptase.

It is also an object of this invention to provide processes for the preparation of the substituted indolyl-piperidinyl benzylamines as disclosed herein.

Other objects and further scope of the applicability of the present invention will become apparent from the detailed description that follows.

### SUMMARY OF THE INVENTION

The present invention provides substituted indolyl-piperidinyl benzylamines of formula (I), and the stereoisomers, enantiomers, racemates and tautomers of said compounds and the pharmaceutically acceptable salts thereof, as inhibitors of β-tryptase, and methods of using the compounds of formula (I) as pharmaceutical agents for the treatment of diseases and disorders.

Thus in accordance with the practice of this invention there is provided a compound of formula (I):

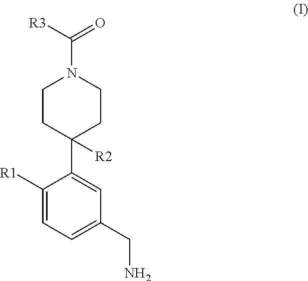

wherein
R1 is F, Cl, Br, OCH₂CO₂CH₃, OCH₂CONW1W2, CH₂OH or optionally substituted alkyl, haloalkyl, alkoxy or haloalkoxy; wherein
W1 and W2 are independently H or alkyl;
R2 is H, F, Cl, Br, OH, CH₂OH, alkyl or alkoxy;
provided R1 and R2 are not H at the same time; and
R3 is aryl or heteroaryl.

This invention further includes various salts of the compounds of formula (I) including various enantiomers or diastereomers of compounds of formula (I).

A further embodiment of the present invention relates to a method for inhibiting β-tryptase activity in a patient comprising administering to said patient a therapeutically effective amount of an inhibitor of β-tryptase. Another embodiment of the present invention relates to a method for inhibiting β-tryptase activity in a patient comprising administering to said patient a therapeutically effective amount of a compound of formula (I).

Another embodiment of the present invention relates to a method for treating a patient suffering from a disease or disorder ameliorated by inhibition of β-tryptase comprising administering to said patient a therapeutically effective amount of a compound of formula (I).

In other aspects of this invention there are also provided various pharmaceutical compositions comprising one or more compounds of formula (I) as well as their therapeutic use in alleviating various diseases which are ameliorated by inhibition of β-tryptase.

### DETAILED DESCRIPTION OF THE INVENTION

The terms as used herein have the following meanings:

As used herein, the expression “(C₁-C₄)alkyl” includes methyl and ethyl groups, and straight-chained or branched propyl, and butyl groups. Particular alkyl groups are methyl, ethyl, n-propyl, isopropyl and tert-butyl. Derived expressions such as “(C₁-C₄)alkoxy”, “(C₁-C₄)alkoxy(C₁-C₄)alkyl”, or “hydroxy(C₁-C₄)alkyl” are to be construed accordingly.

As used herein, the expression “(C₁-C₆)perfluoroalkyl” means that all of the hydrogen atoms in said alkyl group are replaced with fluorine atoms. Illustrative examples include trifluoromethyl and pentafluoroethyl, and straight-chained or branched heptafluoropropyl, nonafluorobutyl, undecafluoropentyl and tridecafluorohexyl groups. Derived expression, “(C₁-C₆)perfluoroalkoxy”, is to be construed accordingly.

“Halogen” or “halo” means chloro, fluoro, bromo, and iodo.

As used herein, “patient” means a warm blooded animal, such as for example rat, mice, dogs, cats, guinea pigs, and primates such as humans.

As used herein, the expression “pharmaceutically acceptable carrier” means a non-toxic solvent, dispersant, excipient, adjuvant, or other material which is mixed with the compound of the present invention in order to permit the formation of a pharmaceutical composition, i.e., a dosage form capable of administration to the patient. One example of such a carrier is pharmaceutically acceptable oil typically used for parenteral administration.

The term “pharmaceutically acceptable salts” as used herein means that the salts of the compounds of the present invention can be used in medicinal preparations. Other salts may, however, be useful in the preparation of the compounds according to the invention or of their pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, hydrobromic acid, nitric acid, sulfamic acid, sulfuric acid, methanesulfonic acid, 2-hydroxyethanesulfonic acid, p-toluenesulfonic acid, fumaric acid, maleic acid, hydroxymaleic acid, malic acid, ascorbic acid, succinic acid, glutaric acid, acetic acid, propionic acid, salicylic acid, cinnamic acid, 2-phenoxybenzoic acid, hydroxybenzoic acid, phenylacetic acid, benzoic acid, oxalic acid, citric acid, tartaric acid, glycolic acid, lactic acid, pyruvic acid, malonic acid, carbonic acid or phosphoric acid. The acid metal salts such as sodium monohydrogen orthophosphate and potassium hydrogen sulfate can also be formed. Also, the salts so formed may present either as mono- or di-acid salts and can exist substantially anhydrous or can be hydrated. Furthermore, where the compounds of the invention carry an acidic moiety, suitable pharmaceutically acceptable salts thereof may include alkali metal salts, e.g. sodium or potassium salts; alkaline earth metal salts, e.g. calcium or magnesium salts, and salts formed with suitable organic ligands, e.g. quaternary ammonium salts.

The expression “stereoisomers” is a general term used for all isomers of the individual molecules that differ only in the orientation of their atoms in space. Typically it includes mirror image isomers that are usually formed due to at least one asymmetric center, (enantiomers). Where the compounds according to the invention possess two or more asymmetric centers, they may additionally exist as diastereoisomers, also certain individual molecules may exist as geometric isomers (cis/trans). Similarly, certain compounds of this invention may exist in a mixture of two or more structurally distinct forms that are in rapid equilibrium, commonly known as tautomers. Representative examples of tautomers include keto-enol tautomers, phenol-keto tautomers, nitroso-oxime tautomers, imine-enamine tautomers, etc. It is to be understood that all such isomers and mixtures thereof in any proportion are encompassed within the scope of the present invention.

As used herein, ‘R’ and ‘S’ are used as commonly used terms in organic chemistry to denote specific configuration of a chiral center. The term ‘R’ (rectus) refers to that configuration of a chiral center with a clockwise relationship of group priorities (highest to second lowest) when viewed along the bond toward the lowest priority group. The term ‘S’ (sinister) refers to that configuration of a chiral center with a counterclockwise relationship of group priorities (highest to second lowest) when viewed along the bond toward the lowest priority group. The priority of groups is based upon sequence rules wherein prioritization is first based on atomic number (in order of decreasing atomic number). A listing and discussion of priorities is contained in Stereochemistry of Organic Compounds, Ernest L. Eliel, Samuel H. Wilen and Lewis N. Mander, editors, Wiley-Interscience, John Wiley & Sons, Inc., New York, 1994.

In addition to the (R)-(S) system, the older D-L system may also be used herein to denote absolute configuration, especially with reference to amino acids. In this system a Fischer projection formula is oriented so that the number 1 carbon of the main chain is at the top. The prefix ‘D’ is used to represent the absolute configuration of the isomer in which the functional (determining) group is on the right side of the carbon at the chiral center and ‘L’, that of the isomer in which it is on the left.

In a broad sense, the term “substituted” is contemplated to include all permissible substituents of organic compounds. In a few of the specific embodiments as disclosed herein, the term “substituted” means substituted with one or more substituents independently selected from the group consisting of (C₁-C₆)alkyl, (C₂-C₆)alkenyl, (C₁-C₆)perfluoroalkyl, phenyl, hydroxy, —CO₂H, an ester, an amide, (C₁-C₆)alkoxy, (C₁-C₆)thioalkyl, (C₁-C₆)perfluoroalkoxy, —NH₂, Cl, Br, I, F, —NH-lower alkyl, and —N(lower alkyl)₂. However, any of the other suitable substituents known to one skilled in the art can also be used in these embodiments.

“Therapeutically effective amount” means an amount of the compound which is effective in treating the named disease, disorder or condition.

The term “treating” refers to:

(i) preventing a disease, disorder or condition from occurring in a patient that may be predisposed to the disease, disorder and/or condition, but has not yet been diagnosed as having it;

(ii) inhibiting the disease, disorder or condition, i.e., arresting its development; and

(iii) relieving the disease, disorder or condition, i.e., causing regression of the disease, disorder and/or condition.

Thus, in accordance with the practice of this invention there is provided a compound of formula (I):

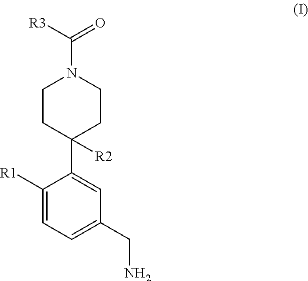

wherein
R1 is F, Cl, Br, OCH₂CO₂CH₃, OCH₂CONW1W2, CH₂OH or optionally substituted alkyl, haloalkyl, alkoxy or haloalkoxy; wherein
W1 and W2 are independently H or alkyl;
R2 is H, F, Cl, Br, OH, CH₂OH, alkyl or alkoxy;
provided R1 and R2 are not H at the same time; and
R3 is aryl or heteroaryl.

This invention further includes various salts of the compounds of formula (I) including various enantiomers or diastereomers of compounds of formula (I). As noted hereinabove and by way of specific examples hereafter all of the salts that can be formed including pharmaceutically acceptable salts are part of this invention. As also noted hereinabove and hereafter all of the conceivable enantiomeric and diastereomeric forms of compounds of formula (I) are part of this invention.

In one of the embodiments, there is provided the compounds of formula (I) wherein R1 is F, Cl, Br, OCH₂CO₂CH₃, OCH₂CONW1W2 or CH₂OH.

In another embodiment of this invention there is also provided a compound of formula (I), wherein R2 is H, F, Cl, Br, OH or CH₂OH.

In yet another embodiment of this invention there is also provided a compound of formula (I), wherein

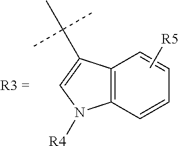

wherein
R4 is alkyl optionally substituted by one or more groups selected from hydroxy, alkoxy, haloalkoxy, cycloalkyl, heterocycloalkyl, aryl, optionally substituted aryl and heteroaryl, or optionally substituted heteroaryl; and
R5 is H, halo, alkoxy, haloalkoxy, alkyl, amido, carboxyl, ureyl, sulfonyl amido, sulfonyl urea, alkyl optionally substituted by one or more groups selected from hydroxy, alkoxy, haloalkoxy, cycloalkyl, heterocycloalkyl, aryl, or heteroaryl.

In yet another embodiment of this invention there is also provided a compound of formula (I), wherein R3 is indolyl that is optionally substituted.

In a further aspect of this invention the compounds encompassed by the scope of this invention without any limitation may be enumerated as shown in the Examples section. All of these compounds may also include corresponding salts wherever possible including the pharmaceutically acceptable salts thereof.

This invention describes a novel alternative scaffold which can be used to generate a series of compounds with beta tryptase inhibitory activity. Based on the structure activity relationship (SAR) of piperidinyl benzylamines several P4 groups were chosen to determine whether this conformationally restricted scaffold would orient the P4 and P1 groups such that the molecules would have utility as beta tryptase inhibitors.

The compounds of this invention can be synthesized by any of the procedures known to one skilled in the art. Specifically, several of the starting materials used in the preparation of the compounds of this invention are known or are themselves commercially available. The compounds of this invention and several of the precursor compounds may also be prepared by methods used to prepare similar compounds as reported in the literature and as further described herein. For instance, see R. C. Larock, “Comprehensive Organic Transformations,” VCH publishers, 1989.

It is also well known that in various organic reactions it may be necessary to protect reactive functional groups, such as for example, amino groups, to avoid their unwanted participation in the reactions. Conventional protecting groups may be used in accordance with standard practice and known to one of skilled in the art, for example, see T. W. Greene and P. G. M. Wuts in “Protective Groups in Organic Chemistry” John Wiley and Sons, Inc., 1991. For example, suitable amine protecting groups include without any limitation sulfonyl (e.g., tosyl), acyl (e.g., benzyloxycarbonyl or t-butoxycarbonyl) and arylalkyl (e.g., benzyl), which may be removed subsequently by hydrolysis or hydrogenation as appropriate. Other suitable amine protecting groups include trifluoroacetyl [—C(═O)CF₃] which may be removed by base catalyzed hydrolysis, or a solid phase resin bound benzyl group, such as a Merrifield resin bound 2,6-dimethoxybenzyl group (Ellman linker) or a 2,6-dimethoxy-4-[2-(polystyrylmethoxy)ethoxy]benzyl, which may be removed by acid catalyzed hydrolysis, for example with TFA.

In another aspect of this embodiment, a specific disease, a disorder or a condition that can be prevented and/or treated with the compound of this invention include, without any limitation the following: joint inflammation, arthritis, rheumatoid arthritis, rheumatoid spondylitis, gouty arthritis, traumatic arthritis, rubella arthritis, psoriatic arthritis, and other chronic inflammatory joint diseases. Other embodiments of physiological conditions that can be treated by the present invention include physiological conditions such as chronic obstructive pulmonary disease (COPD), COPD exacerbations, joint cartilage destruction, ocular conjunctivitis, vernal conjunctivitis, inflammatory bowel disease, asthma, allergic rhinitis, interstitial lung diseases, fibrosis, sceleroderma, pulmonary fibrosis, acute macular degneration, macular degeneration, wet macular degeneration, liver cirrhosis, myocardial fibrosis, neurofibromas, hypertrophic scars, various dermatological conditions, for example, atopic dermatitis and psoriasis,

myocardial infarction, stroke, angina and other consequences of atherosclerotic plaque rupture, as well as periodontal disease, diabetic retinopathy, tumor growth, anaphylaxis, multiple sclerosis, peptic ulcers, and syncytial viral infections.

As described hereinbelow by way of specific examples, the compounds of formula (I) bind to the β-tryptase and demonstrate the ability to inhibit it. The compound of formula I possesses tryptase inhibition activity according to tests described in the literature and described hereinafter, and which test results are believed to correlate to pharmacological activity in humans and other mammals. In addition the compound in formula one is a prodrug of a compound that possesses in-vitro typtase activity according to test described in the literature. Thus, in a further embodiment, the present invention is directed to the use of formula I or a composition comprising it for treating a patient suffering from, or subject to, a condition that can be ameliorated by the administration of an inhibitor of tryptase. For example, the compound of formula I is useful for treating an inflammatory disease, for example, joint inflammation, including arthritis, rheumatoid arthritis and other arthritic condition such as rheumatoid spondylitis, gouty arthritis, traumatic arthritis, rubella arthritis, psoriatic arthritis, osteoarthritis or other chronic inflammatory joint disease, or diseases of joint cartilage destruction, ocular conjunctivitis, vernal conjunctivitis, inflammatory bowel disease, asthma, allergic rhinitis, interstitial lung diseases, fibrosis, sceleroderma, pulmonary fibrosis, liver cirrhosis, myocardial fibrosis, neurofibromas, hypertrophic scars, various dermatological conditions, for example, atopic dermatitis and psoriasis, myocardial infarction, stroke, angina or other consequences of atherosclerotic plaque rupture, as well as periodontal disease, diabetic retinopathy, macular degeneration, acute macular degeneration, wet, macular degeneration, tumor growth, anaphylaxis, multiple sclerosis, peptic ulcers, or a syncytial viral infection.

According to a further feature of the invention there is provided a method for the treatment of a human or animal patient suffering from, or subject to, conditions which can be ameliorated by the administration of an inhibitor of tryptase, for example conditions as hereinbefore described, which comprises the administration to the patient of an effective amount of compound of the invention or a composition containing a compound of the invention. Therefore, the compounds of this invention may have utility in the treatment of diseases or conditions ameliorated by inhibition of β-tryptase. Accordingly, the differential activities of these compounds may allow for utility to ameliorate multiple disease states as specifically enumerated above.

Thus in one aspect of this invention there is provided a method of treating a disease in a patient, said disease selected from the group consisting of joint inflammation, arthritis, rheumatoid arthritis, rheumatoid spondylitis, gouty arthritis, traumatic arthritis, rubella arthritis, psoriatic arthritis, and other chronic inflammatory joint diseases. Other embodiments of physiological conditions that can be treated by the present invention include physiological conditions such as chronic obstructive pulmonary disease (COPD), COPD exacerbations, joint cartilage destruction, ocular conjunctivitis, vernal conjunctivitis, inflammatory bowel disease, asthma, allergic rhinitis, interstitial lung diseases, fibrosis, sceleroderma, pulmonary fibrosis, acute macular degneration, macular degeneration, wet macular degeneration, liver cirrhosis, myocardial fibrosis, neurofibromas, hypertrophic scars, various dermatological conditions, for example, atopic dermatitis and psoriasis,

myocardial infarction, stroke, angina and other consequences of atherosclerotic plaque rupture, as well as periodontal disease, diabetic retinopathy, tumor growth, anaphylaxis, multiple sclerosis, peptic ulcers, and syncytial viral infections; comprising administering to said patient a therapeutically effective amount of a compound of formula (I).

One of skill in the art readily appreciates that the pathologies and disease states expressly stated herein are not intended to be limiting rather to illustrate the efficacy of the compounds of the present invention. Thus it is to be understood that the compounds of this invention may be used to treat any disease caused by the effects of β-tryptase. That is, as noted above, the compounds of the present invention are inhibitors of β-tryptase and may be effectively administered to ameliorate any disease state which is mediated all or in part by β-tryptase.

All of the various embodiments of the compounds of this invention as disclosed herein can be used in the method of treating various disease states as described herein. As stated herein, the compounds used in the method of this invention are capable of inhibiting the effects of β-tryptase and thereby alleviating the effects and/or conditions caused due to the activity of β-tryptase.

In another embodiment of the method of this invention, the compounds of this invention can be administered by any of the methods known in the art. Specifically, the compounds of this invention can be administered by oral, intramuscular, subcutaneous, rectal, intratracheal, intranasal, intraperitoneal or topical route.

Finally, in yet another embodiment of this invention, there is also provided a pharmaceutical composition comprising a pharmaceutically acceptable carrier and a compound of formula (I), including enantiomers, stereoisomers, and tautomers of said compound and pharmaceutically acceptable salts, solvates or derivatives thereof, with said compound having the general structure shown in formula (I) as described herein.

As described herein, the pharmaceutical compositions of this invention feature β-tryptase inhibitory activity and thus are useful in treating any disease, condition or a disorder caused due to the effects of β-tryptase in a patient. Again, as described above, all of the preferred embodiments of the compounds of this invention as disclosed herein can be used in preparing the pharmaceutical compositions as described herein.

Preferably the pharmaceutical compositions of this invention are in unit dosage forms such as tablets, pills, capsules, powders, granules, sterile parenteral solutions or suspensions, metered aerosol or liquid sprays, drops, ampoules, auto-injector devices or suppositories; for oral, parenteral, intranasal, sublingual or rectal administration, or for administration by inhalation or insufflation. Alternatively, the compositions may be presented in a form suitable for once-weekly or once-monthly administration; for example, an insoluble salt of the active compound, such as the decanoate salt, may be adapted to provide a depot preparation for intramuscular injection. An erodible polymer containing the active ingredient may be envisaged. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. Flavored unit dosage forms contain from 1 to 100 mg, for example 1, 2, 5, 10, 25, 50 or 100 mg, of the active ingredient. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the novel compositions of the present invention may be incorporated for administration orally or by injection include aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatin.

The pharmaceutical compositions of this invention can be administered by any of the methods known in the art. In general, the pharmaceutical compositions of this invention can be administered by oral, intramuscular, subcutaneous, rectal, intratracheal, intranasal, intraperitoneal or topical route. The preferred administrations of the pharmaceutical composition of this invention are by oral and intranasal routes. Any of the known methods to administer pharmaceutical compositions by an oral or an intranasal route can be used to administer the composition of this invention.

In the treatment of various disease states as described herein, a suitable dosage level is about 0.01 to 250 mg/kg per day, preferably about 0.05 to 100 mg/kg per day, and especially about 0.05 to 20 mg/kg per day. The compounds may be administered on a regimen of 1 to 4 times per day.

This invention is further illustrated by the following examples which are provided for illustration purposes and in no way limit the scope of the present invention.

### EXAMPLES

### General

As used in the examples and preparations that follow, the terms used therein shall have the meanings indicated: “kg” refers to kilograms, “g” refers to grams, “mg” refers to milligrams, “μg” refers to micrograms, “pg” refers to picograms, “lb” refers to pounds, “oz” refers to ounces, “mol” refers to moles, “mmol” refers to millimoles, “μmole” refers to micromoles, “nmole” refers to nanomoles, “L” refers to liters, “mL” or “ml” refers to milliliters, “μL” refers to microliters, “gal” refers to gallons, “° C.” refers to degrees Celsius, “R_{f}” refers to retention factor, “mp” or “m.p.” refers to melting point, “dec” refers to decomposition, “bp” or “b.p.” refers to boiling point, “mm of Hg” refers to pressure in millimeters of mercury, “cm” refers to centimeters, “nm” refers to nanometers, “abs.” refers to absolute, “conc.” refers to concentrated, “c” refers to concentration in g/mL, “DMSO” refers to dimethyl sulfoxide, “DMF” refers to N,N-dimethylformamide, “CU” refers to 1,1′-carbonyldiimidazole, “DCM” or “CH₂Cl₂” refers to dichloromethane, “DCE” refers to 1,2-dichloroethane, “HCl” refers to hydrochloric acid, “EtOAc” refers to ethyl acetate, “PBS” refers to Phosphate Buffered Saline, “IBMX” refers to 3-isobutyl-1-methylxanthine, “PEG” refers to polyethylene glycol, “MeOH” refers to methanol, “MeNH₂” refers to methyl amine, “N₂” refers to nitrogen gas, “iPrOH” refers to isopropyl alcohol, “Et₂O” refers to ethyl ether, “LAH” refers to lithium aluminum hydride, “heptane” refers to n-heptane, “HMBA-AM” resin refers to 4-hydroxymethylbenzoic acid amino methyl resin, “PdCl₂(dppf)₂” refers to 1,1′-bis(diphenylphosphino)ferrocene-palladium (II) dichloride DCM complex, “HBTU” refers to 2-(1H-benzotriazol-1yl)-1,1,3,3-tetramethyluronium hexafluorophosphate, “DIEA” refers to diisopropylethylamine, “CsF” refers to cesium fluoride, “MeI” refers to methyl iodide, “AcN,” “MeCN” or “CH₃CN” refers to acetonitrile, “TFA” refers to trifluoroacetic acid, “THF” refers to tetrahydrofuran, “NMP” refers to 1-methyl-2-pyrrolidinone, “H₂O” refers to water, “BOC” refers to t-butyloxycarbonyl, “brine” refers to a saturated aqueous sodium chloride solution, “M” refers to molar, “mM” refers to millimolar, “μM” refers to micromolar, “nM” refers to nanomolar, “N” refers to normal, “TLC” refers to thin layer chromatography, “HPLC” refers to high performance liquid chromatography, “HRMS” refers to high resolution mass spectrum, “L.O.D.” refers to loss on drying, “μCi” refers to microcuries, “i.p.” refers to intraperitoneally, “i.v.” refers to intravenously, anhyd=anhydrous; aq=aqueous; min=minute; hr=hour; d=day; sat.=saturated; s=singlet, d=doublet; t=triplet; q=quartet; m=multiplet; dd=doublet of doublets; br=broad; r.t.=room temperature; LC=liquid chromatograph; MS=mass spectrograph; ESI/MS=electrospray ionization/mass spectrograph; RT=retention time; M=molecular ion, “˜”=approximately.

Reactions generally are run under a nitrogen atmosphere. Solvents are dried over magnesium sulfate and are evaporated under vacuum on a rotary evaporator. TLC analyses are performed with EM Science silica gel 60 F254 plates with visualization by UV irradiation. Flash chromatography is performed using Alltech prepacked silica gel cartridges. The ¹H NMR spectra are run at 300 MHz on a Gemin±300 or Varian Mercury 300 spectrometer with an ASW 5 mm probe, and usually recorded at ambient temperature in a deuterated solvent, such as D₂O, DMSO-D₆ or CDCl₃ unless otherwise noted. Chemical shifts values (δ) are indicated in parts per million (ppm) with reference to tetramethylsilane (TMS) as the internal standard.

High Pressure Liquid Chromatography-Mass Spectrometry (LCMS) experiments to determine retention times (R_{T}) and associated mass ions are performed using one of the following methods:

Mass Spectra (MS) are recorded using a Micromass mass spectrometer. Generally, the method used was positive electro-spray ionization, scanning mass m/z from 100 to 1000. Liquid chromatography was performed on a Hewlett Packard 1100 Series Binary Pump & Degasser; Auxiliary detectors used were: Hewlett Packard 1100 Series UV detector, wavelength=220 nm and Sedere SEDEX 75 Evaporative Light Scattering (ELS) detector temperature=46° C., N₂ pressure=4 bar.
LCT: Grad (AcN+0.05% TFA):(H₂O+0.05% TFA)=5:95 (0 min) to 95:5 (2.5 min) to 95:5 (3 min). Column: YMC Jsphere 33×2 4 μM, 1 ml/min
MUX: Column: YMC Jsphere 33×2, 1 ml/min
Grad (AcN+0.05% TFA):(H2O+0.05% TFA)=5:95 (0 min) to 95:5 (3.4 min) to 95:5 (4.4 min).
LCT2: YMC Jsphere 33×2 4 μM, (AcN+0.05% TFA):(H2O+0.05% TFA)=5:95 (0 min) to 95:5 (3.4 min) to 95:5 (4.4 min)
QU: YMC Jsphere 33×2 1 ml/min, (AcN+0.08% formic acid):(H2O+0.1% formic acid)=5:95 (0 min) to 95:5 (2.5 min) to 95:5 (3.0 min)

The following examples describe the procedures used for the preparation of some of the compounds of this invention.

### Example 1

### [4-(5-Aminomethyl-2-fluoro-phenyl)-piperidin-1-yl]-[1-(2-methoxy-ethyl)-7-trifluoromethoxy-1H-indol-3-yl]-methanone hydrochloride

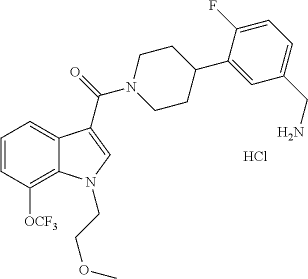

### A. (2-Trifluoromethoxy-phenyl)-carbamic acid ethyl ester

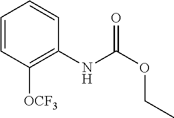

To a solution of 2-(trifluoromethoxy)aniline (15.9 g, 0.09 mol) in DME (300 mL) at −5° C. (ice/salt bath) is added sodium hydride (3.6 g, 60% by weight, 0.09 mol) in portions. The suspension is warmed to r.t. and ethyl chloroformate (7.5 mL, 0.08 mol) is added dropwise. The reaction mixture is stirred for 2 h at r.t. then heated to reflux for 1.5 h. The mixture is then cooled to r.t. and water (150 mL) is slowly added to quench the reaction. The phases are separated and the water layer is extracted with EtOAc (2×100 mL). The combined organic layers are washed with brine, dried over MgSO₄, filtered and concentrated in vacuo. The crude material is purified on silica gel with EtOAc/heptanes (1-5%) as eluent to afford the title product (11.0 g, 49%) as an amber oil.

¹H NMR (300 MHz, CDCl₃) δ 8.20 (d, J=8.1 Hz, 1H), 7.30-7.22 (m, 2H), 7.07 (app t, 1H), 6.90 (br s, 1H), 4.25 (q, J=7.2 Hz, 2H), 1.34 (t, J=7.2 Hz, 3H);

¹⁹F NMR (300 MHz, CDCl₃) δ −57.32 (s, 3F);

LC Rt: 2.96 min; MS 250 (M+1, 94%).

### B. (2-Iodo-6-trifluoromethoxy-phenyl)-carbamic acid ethyl ester

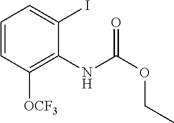

To a solution of (2-trifluoromethoxy-phenyl)-carbamic acid ethyl ester (11.0 g, 44.2 mmol) in THF (200 mL) at −78° C. is added sec-BuLi (1.3 M in cyclohexane, 71.4 mL, 92.8 mmol) dropwise. After 1 h, a solution of 12 (11.22 g, 44.2 mmol) in THF (42 mL) is added dropwise. The resulting orange mixture is stirred at −78° C. for 40 min then saturated NH₄Cl (200 mL) is added, and the cooling bath is removed. The reaction mixture is partitioned between H₂O and diethyl ether. The two layers are separated, and the organic layer is washed with 50% Na₂SO₃, H₂O and brine, dried over MgSO₄, filtered, and concentrated in vacuo to yield a yellow/orange solid as the title product (14.9 g, 90%).

¹H NMR (300 MHz, CDCl₃) δ 7.81 (d, J=8.1 Hz, 1H), 7.29 (m, 1H), 7.05 (app t, 1H), 6.10 (br s, 1H), 4.24 (q, J=7.2 Hz, 2H), 1.33 (t, J=7.2 Hz, 3H);

¹⁹F NMR (300 MHz, CDCl₃) δ −57.28 (s, 3F);

LC Rt 2.93 min; MS 375 (M+1, 97%).

### C. (2-Trifluoromethoxy-6-trimethylsilanylethynyl-phenyl)-carbamic acid ethyl ester

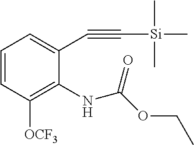

Bis(triphenylphosphine)palladium (II) dichloride (210 mg, 0.30 mmol) and CuI (57 mg, 0.30 mmol) is added to TEA (110 mL) and heated to 80° C. for 20 min. The mixture is cooled to r.t. then (2-iodo-6-trifluoromethoxy-phenyl)-carbamic acid ethyl ester (11.2 g, 29.9 mmol) is added and stirred for 30 min. TMS-acetylene (4.0 mL, 28.4 mmol) is then added dropwise to the reaction mixture and the resulting solution is stirred at r.t. for 1.5 h. Triethylamine is removed in vacuo and the residue is partitioned between water and Et₂O. The organic layer is washed with 1N HCl, brine and dried over MgSO₄, filtered and concentrated in vacuo. The crude material is purified on silica gel with EtOAc/heptane (5-6%) as eluent to give the titled product (8.4 g, 81%) as a yellow solid.

¹H NMR (300 MHz, CDCl₃) δ 7.41 (d, J=7.5 Hz, 1H), 7.29-7.13 (m, 2H), 6.32 (br s, 1H), 4.23 (q, J=7.2 Hz, 2H), 1.30 (t, J=7.1 Hz, 3H), 0.26 (s, 9H);

LC Rt 3.56 min; MS 346 (M+1, 92%).

### D. 7-Trifluoromethoxy-1H-indole

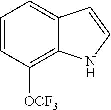

KOH (1.95 g, 34.8 mmol) is heated to 80° C. in t-butanol (55 mL) for 2 hr during which time the solution becomes homogeneous and clear. (2-Trifluoromethoxy-6-trimethylsilanylethynyl-phenyl)-carbamic acid ethyl ester (5.214 g, 15.1 mmol) is added to the solution and heated at 80° C. for 2 h. The solvent is removed in vacuo and the residue partitioned between Et₂O and water. The organic layer is washed with brine, dried over MgSO₄, filtered and concentrated in vacuo. The crude material is purified on silica gel with EtOAc/heptane (1-5%) as eluent to yield the title product (1.82 g, 60%) as a yellow liquid.

¹H NMR (300 MHz, CDCl₃) δ 8.40 (br s, 1H), 7.58-7.55 (m, 1H), 7.25 (m, 1H), 7.09-7.07 (m, 2H), 6.62-6.60 (m, 1H);

¹⁹F NMR (300 MHz, CDCl₃) δ −57.50 (s, 3F);

CHN: Theoretical: C, 53.74%, H, 3.01%, N, 6.96%. Found: C, 53.86%, H, 3.14%, N, 6.97%.

### E. 1-(2-Methoxy-ethyl)-7-trifluoromethoxy-1H-indole

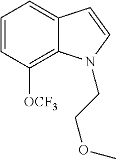

Powder KOH (3.422 g, 61.1 mmol) in DMSO (45 mL) is stirred at r.t. for 5 min under N2 then 7-trifluoromethoxy-1H-indole (3.071 g, 15.3 mmol) in DMSO (5 mL) is added dropwise to the reaction mixture. After 45 min at r.t., 2-methoxyethyl bromide (2.9 mL, 30.6 mmol) is added dropwise and the mixture is stirred at r.t. overnight. LC/MS indicates the reaction is completed. The mixture is partitioned between H₂O and Et₂O. The two layers are separated, and the aqueous layer is extracted with Et₂O (2×). The combined organic layers are washed with H₂O and brine, dried over MgSO₄, filtered, and concentrated in vacuo. The crude material is purified on silica gel with EtOAc/heptane (5-10%) as eluent to afford the title product (3.486 g, 88%) as a yellow oil.

¹H NMR (300 MHz, CDCl₃) δ 7.50 (d, 1H), 7.15 (d, J=3.0 Hz, 1H), 7.05-7.03 (m, 2H), 6.51 (d, J=3.3 Hz, 1H), 4.46 (t, J=5.4 Hz, 2H), 3.70 (t, J=5.4 Hz, 2H), 3.30 (s, 3H);

¹⁹F NMR (300 MHz, CDCl₃) δ −56.45 (s, 3F);

MS 260 (M+1).

### F. 2,2,2-Trifluoro-1-[1-(2-methoxy-ethyl)-7-trifluoromethoxy-1H-indol-3-yl]-ethanone

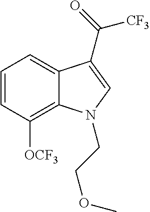

To a mixture of 1-(2-methoxy-ethyl)-7-trifluoromethoxy-1H-indole (1.898 g, 7.32 mmol) in DMF (14 mL) at 0° C. is added TFAA (1.2 mL, 8.63 mmol) dropwise. After addition is completed, the reaction mixture is stirred at 0° C. for 3.5 h and then poured into ice water (50 mL). The solid precipitate is collected and can be used in subsequent steps or taken up in water/EtOAc and further worked up as follows. The two layers are separated, and the organic layer is washed with water, brine, dried over MgSO₄, filtered, and concentrated in vacuo. The crude orange solid (2.6 g, 100%) can be taken on to the next step without further purification.

¹H NMR (300 MHz, CDCl₃) δ 8.35 (d, J=9 Hz, 1H), 8.01 (s, 1H), 7.33 (app t, 1H), 7.26 (m, 1H), 4.55 (t, J=6 Hz, 2H), 3.75 (t, J=6 Hz, 2H), 3.32 (s, 3H);

¹⁹F NMR (300 MHz, CDCl₃) δ −56.41 (s, 3F), −72.16 (s, 3F);

LC Rt 3.59 min; MS 356 (M+1).

### G. 1-(2-Methoxy-ethyl)-7-trifluoromethoxy-1H-indole-3-carboxylic acid

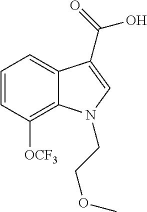

2,2,2-Trifluoro-1-[1-(2-methoxy-ethyl)-7-trifluoromethoxy-1H-indol-3-yl]-ethanone (1.655 g, 4.66 mmol) in 5 N NaOH (20 mL) is heated at 90° C. for two days. The reaction mixture is cooled to room temperature, and then washed with CH₂Cl₂ (3×30 mL). The aqueous layer is slowly acidified to pH ˜4 with conc. HCl and the white powder is collected by suction filtration and air-dried to give the title product (0.923 g, 65%).

¹H NMR (300 MHz, CDCl₃) δ 8.17 (d, J=6 Hz, 1H), 7.97 (s, 1H), 7.27-7.22 (m, 1H), 7.18-7.15 (m, 1H), 4.52 (t, J=6 Hz, 2H), 3.74 (t, J=6 Hz, 2H), 3.32 (s, 3H);

¹⁹F NMR (300 MHz, CDCl₃) δ −56.34 (s, 3F);

LC Rt 3.17 min; MS 345 (M+CH₃CN+1), 304 (M+1, 100%).

### H. 2,2,2-Trifluoro-N-(4-fluoro-3-pyridin-4-yl-benzyl)-acetamide hydrochloride

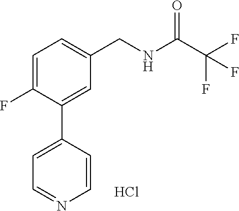

A flask is charged with NaHCO₃ (126 g, 1.5 mol), 3-bromo-4-fluorobenzylamine hydrochloride (120 g, 0.5 mole) and pyridine-4-boronic acid (67.6 g, 0.55 mmol) and isopropyl alcohol (750 mL) and water (375 mL) at r.t. The suspension is degassed with N₂ for 1.0 h at 10° C. Into the mixture is added 1,1′-bis(diphenylphosphino)ferrocene-palladium(II)dichloride dichloromethane complex (PdCl₂dppf-CH₂Cl₂, 16.4 g, 20 mmol). The reaction mixture is ramped to 80° C. while some part is distilled off until the internal temperature reached to 80° C., and stirred for 10 h. After the reaction is completed (HPLC analysis), the mixture is cooled to r.t., and aqueous 2 N HCl (750 mL) is added, and stirred for 0.5 h. The solution is washed with CH₂Cl₂ (750 mL and 500 mL). To the aqueous phase is charged 50% aqueous NaOH (100 mL) to adjust pH>13. After adding n-BuOAc (2.0 L), activated carbon (50 g) is added into the organic layer. This mixture is filtered through a pad of Celite (50 g). Azeotropic distillation is performed. After adding an additional n-BuOAc (1.0 L), the reaction is cooled to 5° C. Trifluoroacetic anhydride (157 g, 0.6 mol) is slowly added into the solution at 5° C. After the reaction is completed (HPLC analysis), the reaction mixture is washed with aqueous 10% Na₂CO₃ (1.0 L). A solution of 5-6 N HCl in isopropanol (120 mL) is introduced into the crude organic layer at 10° C. Additional n-BuOAc (1.0 L) is then added, the suspension is left overnight at r.t. The resultant solid is filtered at 10° C., and dried in oven at 50° C. to give the desired product (124 g, 75%) as a white solid: mp=220° C.

Anal. Calcd for C14H10F4N2O—HCl: C, 50.24; H, 3.31; N, 8.37. Found: C, 50.16; H, 3.08; N, 8.38.

¹H NMR (300 MHz, D₂O) δ 8.70 (d, J=6.9 Hz, 2H), 8.14 (d, J=6.9 Hz, 2H), 7.56-7.20 (m, 3H), 4.51 (s, 2H);

MS (ESI) m/z 299 (M+H).

### I. 2,2,2-trifluoro-N-(4-fluoro-3-piperidin-4-yl-benzyl)-acetamide hydrochloride

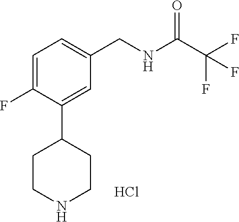

A Parr flask is charged with 2,2,2-trifluoro-N-(4-fluoro-3-pyridin-4-yl-benzyl)-acetamide hydrochloride (123 g, 0.37 mol) and MeOH (740 mL) at room temperature. Then, 5% Pt/C (36.9 g, 30 w/w % is added. The reaction flask is placed in a Parr hydrogenation system and charged with H₂ at 50-60 psi. The mixture is shaken for >48 h while charging H₂ until the pressure reached a steady state (H₂ was refilled to 50-60 psi every 2-3 hours during day time while 10-20 psi is observed without any further refill after overnight). When HPLC analysis shows completion of the reaction, the reaction mixture is filtered through a pad of Celite. The filtrate is distilled at 40-50° C. while adding n-BuOAc (1.25 L). After completion of distillation of MeOH, additional n-BuOAc (1 L) is added. The resultant suspension is allowed to cool to r.t. overnight. The suspension is cooled to 10° C., filtered, and dried in oven at 50° C. to give the desired product (112 g, 89%) as white solid: mp=134° C.

Anal. Calcd for C14H10F4N2O—HCl: C, 50.24; H, 3.31; N, 8.37. Found: C, 50.16; H, 3.08; N, 8.38;

¹H NMR (300 MHz, D₂O) δ 7.16-6.98 (m, 3H), 4.34 (s, 2H), 3.42 (d, J=12.9 Hz, 2H), 3.14-2.99 (m, 3H), 1.98-1.81 (m, 4H);

MS (ESI) m/z 305.4 (M+H).

### J. 2,2,2-Trifluoro-N-(4-fluoro-3-{1-[1-(2-methoxy-ethyl)-7-trifluoromethoxy-1H-indole-3-carbonyl]-piperidin-4-yl}-benzyl)-acetamide

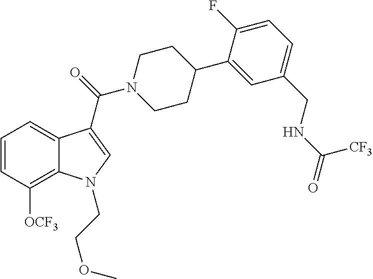

A mixture of 1-(2-methoxy-ethyl)-7-trifluoromethoxy-1H-indole-3-carboxylic acid (0.818 g, 2.70 mmol), Et₃N (0.9 mL, 6.5 mmol), 2,2,2-trifluoro-N-(4-fluoro-3-piperidin-4-yl-benzyl)-acetamide hydrochloride (0.919 g, 2.70 mmol), and EDCI (0.620 g, 3.2 mmol) in CH₂Cl₂ (30 mL) is stirred at r.t. overnight. Both TLC and LC/MS indicate that the reaction is completed. The mixture is diluted with EtOAc (60 mL) and the organic layer is washed with saturated NH₄Cl solution, water and brine. The organic layer is dried over MgSO₄, filtered, and concentrated in vacuo. The crude material is purified on silica gel with MeOH/CH₂Cl₂ (1-5%) as eluent to give the title product (1.368 g, 86%) as a white foamy solid.

¹H NMR (300 MHz, CDCl₃) δ 7.68 (d, J=9 Hz, 1H), 7.46 (s, 1H), 7.15-7.13 (m, 4H), 7.15-7.01 (m, 1H), 6.89 (br s, 1H), 4.52 (br s, 3H), 4.48 (t, J=5.2 Hz, 2H), 3.71 (t, J=5.2 Hz, 2H), 3.30 (s, 3H), 3.20-3.00 (m, 4H), 1.90-1.65 (m, 4H);

¹⁹F NMR (300 MHz, CDCl₃) δ −56.52 (s, 3F), −75.40 (s, 3F), −118.98 (s, 1F);

LC Rt 3.52 min; MS 590 (M+1, 100%).

### K. [4-(5-Aminomethyl-2-fluoro-phenyl)-piperidin-1-yl]-[1-(2-methoxy-ethyl)-7-trifluoromethoxy-1H-indol-3-yl]-methanone hydrochloride

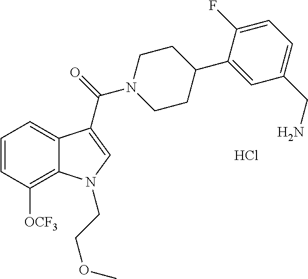

2,2,2-Trifluoro-N-(4-fluoro-3-{1-[1-(2-methoxy-ethyl)-7-trifluoromethoxy-1H-indole-3-carbonyl]-piperidin-4-yl}-benzyl)-acetamide (21.574 g, 36.6 mmol) and K₂OCO₃ (65.5 g, 474 mmol in H₂O/MeOH (290 mL/730 mL)) is stirred at r.t. overnight during which time the suspension becomes homogeneous. The reaction mixture is concentrated in vacuo to remove most of the methanol and the residue is partitioned between H₂O and EtOAc. The two layers are separated, and the organic layer is washed with H₂O and brine, dried over MgSO₄, filtered, and concentrated in vacuo to yield the title product (17.520 g, 97%) as a clear colorless sticky gum.

¹H NMR (300 MHz, CDCl₃) δ 7.70 (d, 1H), 7.48 (s, 1H), 7.20-7.13 (m, 4H), 7.00-6.99 (m, 1H), 4.60 (br s, 2H), 4.49 (t, J=5.1 Hz, 2H), 3.86 (br s, 2H), 3.72 (t, J=5.1 Hz, 2H), 3.30 (s, 3H), 3.20-3.00 (m, 3H), 1.95-1.80 (m, 2H), 1.80-1.63 (m, 4H);

¹⁹F NMR (300 MHz, CDCl₃) δ −56.69 (s, 3F), −121.96 (s, 1F);

LC 2.47 min; MS 494 (M+1, 98%).

To a solution of the above material (2.856 g, 5.59 mmol) in Et₂O (30 mL) is added 2.0 N HCl/Et₂O (3.0 mL, 6.0 mmol) dropwise. A solid precipitate forms and the ethereal solution is decanted off. The solid is collected by vacuum filtration and washed with additional Et₂O. The white product (2.475 g, 4.52 mmol) is dried under high vacuum to removal any residual solvents.

¹H NMR (300 MHz, DMSO-d6) δ 8.35 (br s, 2H), 7.82 (s, 1H), 7.74-7.70 (m, 1H), 7.58 (d, J=5.7 Hz, 1H), 7.39-7.35 (m, 1H), 7.26-7.19 (m, 3H), 4.49 (t, J=5.1 Hz, 2H), 4.44 (br s, 1H), 4.00 (br s, 2H), 3.68 (t, J=5.1 Hz, 2H), 3.32 (s, 3H), 3.14 (m, 2H), 1.83-1.78 (m, 2H), 1.69-1.66 (m, 2H);

¹⁹F NMR (300 MHz, DMSO-d6) δ −55.64 (s, 3F), −119.95 (s, 1F);

LC 2.65 min; MS 494 (M+1, 99%);

CHN: Theoretical: C, 56.14%, H, 5.38%, N, 7.86% (calc'd as 0.27 H₂O). Found: C, 55.91%, H, 5.16%, N, 7.53%.

### Example 2

### [4-(3-Aminomethyl-phenyl)-piperidin-1-yl]-[7-methyl-1-(2-morpholin-4-yl-ethyl)-1H-indol-3-yl]-methanone hydrochloride

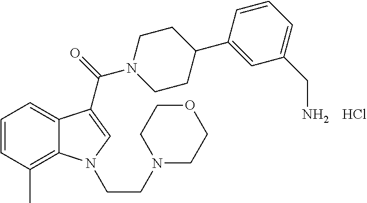

### A. 4-Oxo-piperidine-1-carboxylic acid 2-trimethylsilanyl-ethyl ester

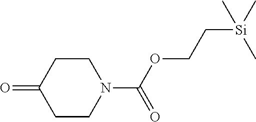

A solution of 4-piperidone monohydrate hydrochloride (25 g, 88.22 mmol), 2-trimethylsilylethyl p-nitrophenylcarbonate (50 mL, 359.7 mmol), triethylamine (50 mL, 0.345 mol) and DMAP (10.78 g, 88.24 mmol) in acetonitrile (300 mL) is warmed under reflux for 2 hours and then allowed to cool to room temperature. The mixture is diluted with dichloromethane (300 mL) and washed with 1 M HCl (3×100 mL) and 1M NaOH (4×100 mL) until all of the yellow color is removed from the organic phase. The organic phase is then washed with brine and dried over MgSO₄. The organic phase is concentrated in vacuo to afford the title compound (19.35 g, 90%) as a colorless oil.

¹H NMR (300 MHz, CDCl₃) δ 4.22 (m, 2H), 3.75 (t, J=6.2 Hz, 4H), 2.44 (t, J=6.2 Hz, 4H), 1.02 (m, 2H), 0.04 (s, 9H).

### B. 4-(3-Cyanophenyl)-3,6-dihydro-2H-pyridine-1-carboxylic acid 2-trimethylsilanyl-ethyl ester

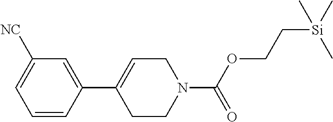

To a flask containing tetrahydrofuran (50 mL) at −70° C. is added 1 M lithium hexamethyldisilazide (60 mL, 60 mmol) dropwise. A solution of 4-oxo-piperidine-1-carboxylic acid 2-trimethylsilanyl-ethyl ester (13.3 g, 55 mmol) is then added via dropping funnel over 20 minutes keeping the internal temperature between −65° C. and −70° C. The solution is stirred at −70° C. for 45 minutes then a solution of phenyltrifluoromethane sulfonamide (19.65 g, 55 mmol) in tetrahydrofuran (75 mL) is added dropwise over 20 minutes. The solution is allowed to warm to 0° C. and stirred for 3 hours. The reaction is then concentrated in vacuo and the residue, 4-trifluoromethanesulfonyloxy-3,6-dihydro-2H-pyridine-1-carboxylic acid 2-trimethyl-silanyl-ethyl ester, is used without further purification.

To a solution of 4-trifluoromethanesulfonyloxy-3,6-dihydro-2H-pyridine-1-carboxylic acid 2-trimethyl-silanyl-ethyl ester (20.65 g, 55 mmol) in acetonitrile (300 mL) is added 3-cyanophenylboronic acid (8.9 g, 60.6 mmol) followed by 2 M sodium carbonate (82.5 mL, 165 mmol), lithium chloride (6.98 g, 165 mmol) and tetrakistriphenylphosphine palladium (0) (3.18 g, 2.8 mmol). The mixture is warmed under reflux for 90 minutes then allowed to cool to room temperature and filtered. The filtrate is concentrated and diluted with 2 M Na₂CO₃ (300 mL) then extracted dichloromethane (3×). The organic phase is washed with brine then separated and dried over MgSO₄. The organic phase is concentrated in vacuo and the crude residue is flash chromatographed over SiO₂ using heptane:EtOAc:DCM (5:1:1) as the eluent to give the title compound (10.46 g, 58%) as a yellow oil.

¹H NMR (300 MHz, CDCl₃) δ 7.65-7.52 (m, 3H), 7.44 (t, J=7.7 Hz, 1H), 6.11 (bs, 1 H), 4.23 (m, 2H), 4.15 (m, 2H), 3.70 (t, J=5.6 Hz, 2H), 2.52 (m, 2H), 1.04 (m, 2H), 0.06 (s, 9H).

### C. 4-(3-Aminomethyl-phenyl)piperidine-1-carboxylic acid 2-trimethylsilanyl-ethyl ester

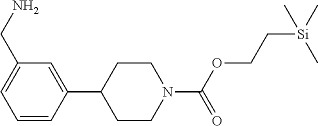

To a slurry of 10% wet Pd/C (5 g) in ethanol (250 mL) is added concentrated HCl (2.9 mL, 34.8 mmol) and 4-(3-cyanophenyl)-3,6-dihydro-2H-pyridine-1-carboxylic acid 2-trimethylsilanyl-ethyl ester (10.4 g, 31.7 mmol). The mixture is hydrogenated at 50 psi for 4 hours. The mixture is then filtered over a cake of Celite and the cake is washed with excess ethanol. The filtrate is then concentrated in vacuo and the residue is triturated with Et₂O/pentane and filtered to give the title compound (7.1 g, 67%) as a white solid.

¹H NMR (300 MHz, CD₃OD) δ 7.41-7.27 (m, 4H), 4.26 (dm, J=13.5 Hz, 2H), 4.20 (m, 2H), 4.09 (s, 2H), 2.92 (bm, 2H), 2.79 (tt, J=12.1, 3.6 Hz, 1H), 1.84 (dm, J=12.9 Hz, 2H), 1.62 (qd, J=12.6, 4.1 Hz, 2H), 1.02 (m, 2H), 0.06 (s, 9H);

MS (APCl, MeOH/H₂O) m/z 336, 335 (M⁺+1, 100), 191.

### D. 4-[3-(tert-Butoxycarbonylamino-methyl)-phenyl]-piperidine-1-carboxylic acid 2-trimethylsilanyl-ethyl ester

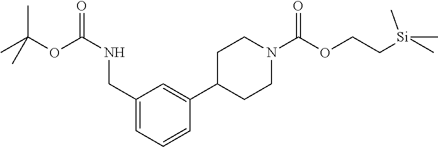

To a solution of 4-(3-aminomethyl-phenyl)-piperidine-1-carboxylic acid 2-trimethylsilanyl-ethyl ester (11.1 g, 29.93 mmol) in dichloromethane (150 mL) and saturated NaHCO₃ (50 mL) is added boc-anhydride (6.54 g, 29.96 mmol). The mixture is stirred overnight at r.t. The organic phase is then separated and washed with water and brine. The organic phase is then separated, dried over MgSO₄ and concentrated in vacuo to give the title compound (13.41 g, 100%) as an oil.

¹H NMR (300 MHz, CDCl₃) δ 7.26 (m, 1H), 7.10 (m, 3H), 4.85 (bs, 1H), 4.29 (d, J=5.8 Hz, 4H), 4.19 (m, 2H), 2.83 (t, J=12.5 Hz, 2H), 2.64 (tt, J=12.0, 3.6 Hz, 1H), 1.81 (m, 2H), 1.60 (m, 2H), 1.45 (s, 9H), 1.01 (t, J=8.4 Hz, 2H), 0.04 (s, 9H).

### E. (3-Piperidin-4-yl-benzyl)-carbamic acid tert-butyl ester

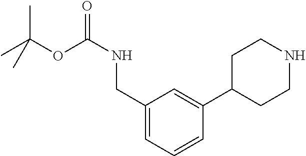

To a solution 4-(3-tert-butoxycarbonylaminomethylphenyl)-piperidine-1-carboxylic acid 2-trimethylsilanyl-ethyl ester (13.41 g, 30.9 mmol) in tetrahydrofuran (200 mL) is added tetra-n-butyl ammonium fluoride (1 M, 34 mL, 34 mmol). The mixture is warmed to 50° C. for 2 hours then allowed to cool to room temperature and stand overnight. To complete the reaction the mixture is heated for an additional 3 h at 50° C. The mixture is then concentrated in vacuo, diluted with 1M HCl and extracted with Et₂O. The aqueous phase is made basic with 1 N NaOH and extracted with EtOAc (3×). The organic phases are combined, washed with brine, separated and dried over MgSO₄. The organic phase is filtered and concentrated in vacuo to afford the title compound (8.3 g, 93%) as a yellow oil which is used without further purification.

¹H NMR (300 MHz, CDCl₃) δ 7.25 (m, 1H), 7.07-7.13 (m, 3H), 4.85 (bs, 1H), 4.29 (d, J=5.1 Hz, 2H), 3.17 (dm, J=12.0 Hz, 2H), 2.72 (td, J=12.0, 2.4 Hz, 2H), 2.60 (tt, J=12.0, 3.6 Hz, 1H), 1.81 (m, 2H), 1.55-1.70 (m, 3H), 1.46 (s, 9H).

### F. 7-Methyl-1-(2-morpholin-4-yl-ethyl)-1H-indole

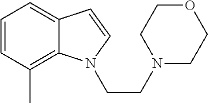

To a solution of 7-methylindole (1.01 g, 7.70 mmol) in N,N-dimethylacetamide at r.t. is added sodium hydride (217 mg, 10.73 mmol). The resulting mixture is stirred for 30 minutes at r.t. then 2-(4-morpholine)ethyl bromide (1.64 g, 8.45 mmol) is added and the resulting mixture is stirred at r.t. overnight. The mixture is diluted with water (100 mL) and ethyl acetate (50 mL). The organic is separated and the aqueous phase is extracted again with ethyl acetate (50 mL). The organic phase is washed with brine then separated and dried over MgSO₄. The organic phase is concentrated in vacuo and the crude residue is flash chromatographed over SiO₂ using 50% EtOAc/heptane as the eluent to afford the title compound (1.60 g, 85%) as an orange oil.

¹H NMR (300 MHz, CDCl₃) δ 7.45 (d, 1H), 7.04 (d, 1H), 7.00-6.90 (m, 2H), 6.45 (d, H), 4.44 (t, 2H), 3.69 (m, 4H), 2.72-2.67 (m, 5H), 2.45 (m, 4H).

### G. 2,2,2-Trifluoro-1-[7-methyl-1-(2-morpholin-4-yl-ethyl)-1H-indol-3-yl]-ethanone

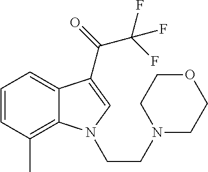

To a solution of 7-methyl-1-(2-morpholin-4-yl-ethyl)-1H-indole (1.60 g, 6.55 mmol) in N,N-dimethylformamide at 0° C. is added trifluoroacetic anhydride (1.1 mL, 7.91 mmol). The resulting mixture is stirred at 0° C. for one hour. The mixture is diluted with 150 mL of water and the aqueous phase is extracted with ethyl acetate (×3). The organic phase is washed with water and brine then separated and dried (MgSO₄). The organic phase is concentrated in vacuo and the crude residue is flash chromatographed over SiO₂ using heptane:EtOAc (35:65) as the eluent to afford the title compound (2.20 g, 99%) as a yellow solid.

¹H NMR (300 MHz, CDCl₃) δ 8.27 (d, 1H), 8.00 (m, 1H), 7.25 (m, 1H), 7.11 (m, 1H), 4.88 (t, 2H), 3.93 (m, 4H), 3.29 (t, 2H), 3.09 (br s, 4H), 2.71 (s, 3H).

### H. 7-Methyl-1-(2-morpholin-4-yl-ethyl)-1H-indole-3-carboxylic acid

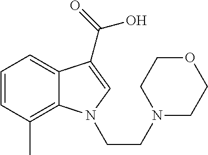

To a solution of 2,2,2-trifluoro-1-[7-methyl-1-(2-morpholin-4-yl-ethyl)-1H-indol-3-yl]-ethanone (2.20 g, 6.46 mmol) in water (10 mL) is added a solution of 10 N sodium hydroxide (16 mL, 160 mmol) and the resulting mixture is refluxed until completion. The mixture is cooled to r.t. and acidified with a solution of 6 N HCl to reach a pH ˜2-3. The aqueous phase is washed with EtOAc (2×), flash freezed, and lyophilized. The resulting solid is triturated with methanol:dichloromethane (1:9). The solid is filtered off, and the filtrate is evaporated in vacuo to afford the title compound (1.80 g, 96%) as a white solid.

¹H NMR (300 MHz, CD₃OD) δ 8.06 (s, 1H), 8.00 (d, 1H), 7.14-7.02 (m, 2H), 4.98 (t, 2H), 4.04-3.94 (br m, 4H), 3.65-3.54 (m, 4H), 3.34 (m, 2H), 2.79 (s, 3H).

### I. (3-{1-[7-Methyl-1-(2-morpholin-4-yl-ethyl)-1H-indole-3-carbonyl]-piperidin-4-yl}-benzyl)-carbamic acid tert-butyl ester

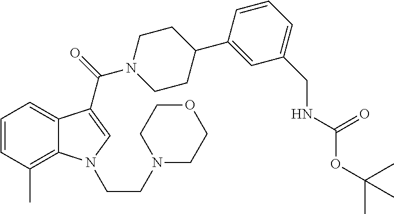

To a solution of 7-methyl-1-(2-morpholin-4-yl-ethyl)-1H-indole-3-carboxylic acid (464 mg, 1.61 mmol), (3-piperidin-4-yl-benzyl)-carbamic acid tert-butyl ester hydrochloride (530 mg, 1.82 mmol) and 1-(3-dimethylaminopropyl)-3-ethylcarbodiimide hydrochloride (426 mg, 2.22 mmol) in dichloromethane is added triethylamine (0.77 mL, 5.50 mmol). The resulting mixture is stirred at r.t. overnight. The mixture is diluted with sat. ammonium chloride (50 mL). The aqueous phase is extracted with ethyl acetate (3×45 mL). The combined organic layers are washed with brine then separated and dried over MgSO₄. The organic phase is concentrated in vacuo and the crude residue is flash chromatographed over SiO₂ using MeOH/CH₂Cl₂ (3:97) as the eluent to afford the title compound (450 mg, 44%) as a white solid.

¹H NMR (300 MHz, CD₃OD) δ 7.58 (s, 1H), 7.53 (d, 1H), 7.28-6.96 (m, 6H), 4.56 (m, 4H), 4.20 (bs, 2H), 3.64 (m, 4H), 3.19-3.07 (m, 3H), 2.87 (m, 1H), 2.74 (m, 5H), 2.47 (m, 4H), 1.89 (m, 2H), 1.80-1.66 (m, 2H), 1.45 (s, 9H).

### J. [4-(3-Aminomethyl-phenyl)-piperidin-1-yl]-[7-methyl-1-(2-morpholin-4-yl-ethyl)-1H-indol-3-yl]-methanone hydrochloride

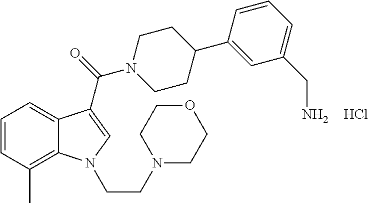

A solution of (3-{1-[7-methyl-1-(2-morpholin-4-yl-ethyl)-1H-indole-3-carbonyl]-piperidin-4-yl}-benzyl)-carbamic acid tert-butyl ester (432 mg, 0.77 mmol) in HCl/dioxane (4 M, 8 mL, 32.0 mmol) is added and stirred for 25 min at r.t. The mixture is vacuum dried and the residue is suspended ether overnight. The suspension is filtered and the cake is rinsed with ether twice. The solid is dried under vacuum to afford the title compound (375 mg, 98%) as a white solid.

¹H NMR (300 MHz, DMSO) δ 8.34 (br s, 3H), 7.80 (s, 1H), 7.55 (d, 1H), 7.46 (s, 1H), 7.38-7.28 (m, 3H), 7.08-6.98 (m, 2H), 4.91 (m, 2H), 4.40 (m, 2H), 4.04-3.96 (m, 1H), 3.89-3.68 (m, 6H), 3.57-3.42 (m, 3H), 3.21-3.02 (m, 4H), 2.86 (m, H), 2.74 (s, 3H), 1.83 (m, 2H), 1.71-1.59 (m, 2H).

### Example 3

### [4-(5-Aminomethyl-2-fluoro-phenyl)-piperidin-1-yl]-[7-methyl-1-(2-morpholin-4-yl-ethyl)-1H-indol-3-yl]-methanone hydrochloride

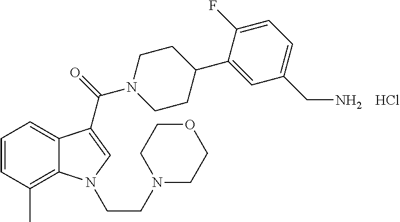

### A. 2,2,2-Trifluoro-N-(4-fluoro-3-{1-[7-methyl-1-(2-morpholin-4-yl-ethyl)-1H-indole-3-carbonyl]-piperidin-4-yl}-benzyl)-acetamide

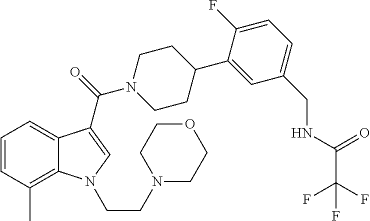

The title compound is prepared in a similar manner as described in Example 2I using 7-methyl-1-(2-morpholin-4-yl-ethyl)-1H-indole-3-carboxylic acid and 2,2,2-trifluoro-N-(4-fluoro-3-piperidin-4-yl-phenyl)-acetamide hydrochloride as the starting materials.

¹H NMR (300 MHz, CD₃OD) δ 7.58 (s, 1H), 7.53 (d, 1H), 7.28 (m, 1H), 7.18 (m, 1H), 7.07-6.96 (m, 3H), 4.56 (m, 4H), 4.41 (s, 2H), 3.64 (m, 4H), 3.17 (m, 3H), 2.73 (m, 6H), 2.47 (m, 4H), 1.90-1.72 (m, 4H).

### B. [4-(5-Aminomethyl-2-fluoro-phenyl)-piperidin-1-yl]-[7-methyl-1-(2-morpholin-4-yl-ethyl)-1H-indol-3-yl]-methanone hydrochloride

To a solution of 2,2,2-trifluoro-N-(4-fluoro-3-{1-[7-methyl-1-(2-morpholin-4-yl-ethyl)-1H-indole-3-carbonyl]-piperidin-4-yl}-benzyl)-acetamide (558 mg, 0.97 mmol) in methanol:water (2:1) is added potassium carbonate (1.33 g, 9.62 mmol). The resulting mixture is stirred at r.t. overnight. The solvent is removed in vacuo and the aqueous residue is partitioned between ethyl acetate and water. The organic layer is separated and the aqueous phase is extracted twice with ethyl acetate. The combined organic layers are washed with brine, dried over MgSO₄, filtered and concentrated in vacuo to yield a solid.

To the solid, HCl in dioxane (4 M, 8 mL, 32.0 mmol) is added and stirred for 25 min. The mixture is vacuum dried and the residue is triturated with ether overnight. The suspension is filtered and the cake is rinsed with ether twice. The solid is dried under vacuum to afford the title compound (490 mg, 98%) as a white solid.

¹H NMR (300 MHz, DMSO-d6) δ 8.29 (br s, 2H), 7.80 (s, H), 7.61-7.54 (m, 2H), 7.37-7.34 (m, 1H), 7.25-7.19 (m, 1H), 7.08-6.98 (m, 2H), 4.90 (m, 2H), 4.40 (m, 1H), 4.02-3.97 (m, 4H), 3.86-3.45 (m, 12H), 2.74 (s, 3H), 1.82-1.62 (m, 4H); MS m/z: [M+H]⁺=461.

### Example 4

### [4-(5-Aminomethyl-2-fluoro-phenyl)-piperidin-1-yl]-[7-methyl-1-(3-phenyl-propyl)-1H-indol-3-yl]-methanone hydrochloride

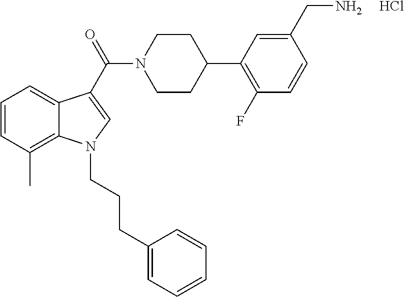

### A. 7-Methyl-1-(3-phenyl-propyl)-1H-indole

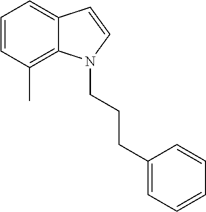

The title compound is prepared in a similar manner as described in Example 1E using 7-methylindole and 1-bromo-3 phenylpropane as the starting materials.

¹H NMR (300 MHz, CDCl₃) δ 7.5 (d, 1H), 7.3 (m, 1H), 7.2 (m, 4H), 7.1-6.9 (m, 3H), 6.5 (d, H), 4.3 (t, 2H), 2.7 (t, 2H), 2.6 (s, 3H), 2.15 (m, 2H); MS m/z: [M+H]⁺=250.

### B. 2,2,2-Trifluoro-1-[7-methyl-1-(3-phenyl-propyl)-1H-indol-3-yl]-ethanone

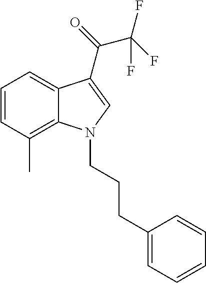

The title compound is prepared in a similar manner as described in Example 1F, using 7-methyl-1-(3-phenyl-propyl)-1H-indole as the starting material.

¹H NMR (300 MHz, CDCl₃) δ 8.3 (d, 1H), 7.8 (s, 1H), 7.3 (m, 3H), 7.2 (m, 3H), 7.15 (d, H), 4.4 (t, 2H), 2.7 (t, 2H), 2.6 (s, 3H), 2.2 (m, 2H);

MS m/z: [M+H]⁺=346.

### C. 7-Methyl-1-(3-phenyl-propyl)-1H-indole-3-carboxylic acid

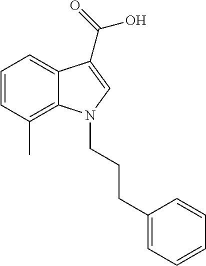

2,2,2-Trifluoro-1-[7-methyl-1-(3-phenyl-propyl)-1H-indol-3-yl]-ethanone (6 g, 17.4 mmol) and 6 N NaOH (75 mL) are heated to reflux overnight. The reaction mixture is cooled to room temperature and acidified to pH=2 with concentrated HCl. The resulting precipitate is collected as the title compound.

¹H NMR (300 MHz, DMSO-d6) δ 12.0 (s, 1H), 8.1 (s, 1H), 7.9 (d, 1H), 7.3 (m, 5H), 7.1 (m, 1H), 6.9 (m, 1H), 4.4 (t, 2H), 3.3 (s, 3H), 2.6 (t, 2H), 2.0 (t, 2H);

MS m/z: [M+H]⁺=294.

### D. 2,2,2-Trifluoro-N-(4-fluoro-3-{1-[7-methyl-1-(3-phenyl-propyl)-1H-indole-3-carbonyl]-piperidin-4-yl}-benzyl)-acetamide

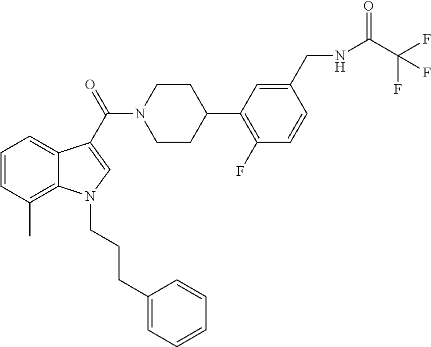

The title compound is prepared in a similar manner as described in Example 2I using 7-methyl-1-(3-phenyl-propyl)-1H-indole-3-carboxylic acid and 2,2,2-trifluoro-N-(4-fluoro-3-piperidin-4-yl-phenyl)-acetamide hydrochloride as the starting materials.

¹H NMR (300 MHz, CDCl₃) δ 7.6 (d, 1H), 7.4 (s, 1H), 7.35-7.0 (m, 10H), 6.6 (bs, 1H), 4.6 (m, 2H), 4.5 (m, 2H), 4.35 (t, 2H), 3.1 (m, 3H), 2.7 (t, 2H), 2.5 (s, 3H), 2.2 (m, 2H), 1.9 (m, 2H), 1.75 (m, 2H);

MS m/z: [M+H]⁺=580.

### E. [4-(5-Aminomethyl-2-fluoro-phenyl)-piperidin-1-yl]-[7-methyl-1-(3-phenyl-propyl)-1H-indol-3-yl]-methanone hydrochloride

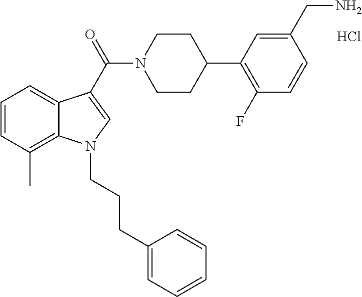

The title compound is prepared in a similar manner as described in Example 1K, using 2,2,2-trifluoro-N-(4-fluoro-3-{1-[7-methyl-1-(3-phenyl-propyl)-1H-indole-3-carbonyl]-piperidin-4-yl}-benzyl)-acetamide as the starting material.

¹H NMR (300 MHz, DMSO-d6) δ 8.3 (bs, 2H), 7.7 (s, H), 7.55 (m, 2H), 7.4-7.2 (m, 7H), 7.0 (m, 1H), 6.9 (m, 1H), 4.4 (m, 4H), 4.0 (t, 2H), 3.1 (m, 3H), 2.7 (t, 2H), 2.6 (s, 3H), 2.1 (m, 2H), 1.8 (m, 2H), 1.75 (m, 2H);

MS m/z: [M+H]⁺=484.

### Example 5

### [4-(5-Aminomethyl-2-fluoro-phenyl)-piperidin-1-yl]-(7-methyl-1-phenethyl-1H-indol-3-yl)-methanone hydrochloride

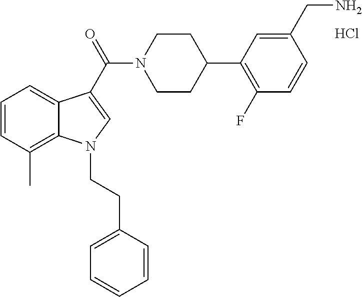

### A. 2,2,2-Trichloro-1-(7-methyl-1H-indol-3-yl)-ethanone

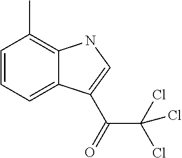

To a solution of 7-methylindole (10 g, 76.3 mmol) in THF (200 mL) at 0° C. is added pyridine (8 mL, 99.2 mmol) followed by dropwise addition of trichloroacetyl chloride (11 mL, 99.2 mmol). The reaction mixture is allowed to warm to r.t. and stir overnight. The reaction mixture is poured into EtOAc and washed with 1N HCl (2×). The organic layers are washed with brine, dried over MgSO₄, filtered, and concentrated in vacuo to give the crude product. Purification by flash chromatography on SiO₂ eluting with 20% ethyl acetate/heptane gives the title compound (19 g, 90% yield).

¹H NMR (300 MHz, DMSO-d6) δ 12.6 (bs, 1H), 8.5 (s, 1H), 8.0 (d, 1H), 7.2 (m, 1H), 7.15 (m, 1H), 3.2 (s, 3H);

MS m/z: [M+H]⁺=276, 278.

### B. 7-Methyl-1H-indole-3-carboxylic acid methyl ester

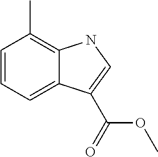

A mixture of 2,2,2-trichloro-1-(7-methyl-1H-indol-3-yl)-ethanone (18.5 g, 66.8 mmol) and 1 N KOH/MeOH (700 mL) is stirred overnight at r.t. The reaction mixture is concentrated in vacuo and the crude product is purified by flash chromatography on SiO₂ eluting with 15% ethyl acetate/heptane to give the titled compound (6 g, 48% yield).

¹H NMR (300 MHz, CDCl₃) δ 8.5 (bs, 1H), 8.1 (d, 1H), 7.9 (d, 1H), 7.2 (m, 1H), 7.1 (m, 1H), 3.9 (s, 3H), 2.5 (s, 3H);

MS m/z: [M+H]⁺=1 90.

### C. 7-Methyl-1-phenethyl-1H-indole-3-carboxylic acid methyl ester

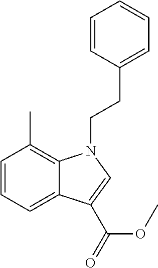

The title compound is prepared in a similar manner as described in Example 2F, using 7-methyl-1H-indole-3-carboxylic acid methyl ester and 2-bromoethylbenzene in DMF as the starting materials.

¹H NMR (300 MHz, CDCl₃) δ 8.1 (d, 1H), 7.6 (s, 1H), 7.3 (m, 2H), 7.2-7.0 (m, 5H), 4.6 (t, 2H), 3.9 (s, 3H), 3.1 (t, 2H), 2.8 (s, 3H);

MS m/z: [M+H]⁺=294.

### D. 7-Methyl-1-phenethyl-1H-indole-3-carboxylic acid

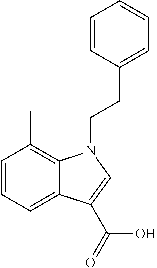

To a solution of 7-methyl-1-phenethyl-1H-indole-3-carboxylic acid methyl ester (0.46 g, 1.57 mmol) in MeOH (20 mL) is added 6 N NaOH (2 mL). The solution is heated to 45° C. for 2 h and then stirred at r.t. overnight. The reaction mixture is acidified to pH=2 with concentrated HCl and the resulting precipitate is collected as the titled compound (0.335 g, 76% yield).

¹H NMR (300 MHz, DMSO-d6) δ 11.9 (bs, 1H), 7.9 (m, 2H), 7.35-7.2 (m, 5H), 7.1 (m, H), 7.0 (m, H), 4.6 (t, 2H), 3.1 (t, 2H), 2.75 (s, 3H);

MS m/z: [M+H]⁺=280.

### E. 2,2,2-Trifluoro-N-{4-fluoro-3-[1-(7-methyl-1-phenethyl-1H-indole-3-carbonyl)-piperidin-4-yl]-benzyl}-acetamide

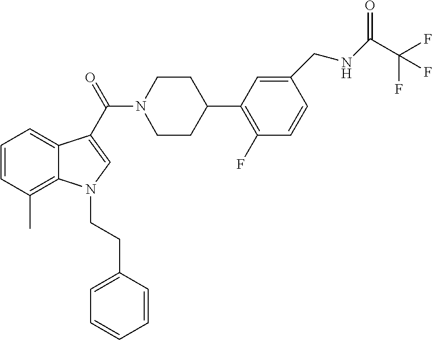

The title compound is prepared in a similar manner as described in Example 2I, using 7-methyl-1-phenethyl-1H-indole-3-carboxylic acid and 2,2,2-trifluoro-N-(4-fluoro-3-piperidin-4-yl-benzyl)-acetamide hydrochloride as the starting materials.

¹H NMR (300 MHz, CDCl₃) δ 7.6 (d, 1H), 7.3 (m, 3H), 7.2-7.0 (m, 8H), 6.6 (bs, H), 4.6 (t, 2H), 4.5 (m, 2H), 4.4 (bs, 1H), 3.15 (m, 3H), 3.0 (m, 2H), 2.8 (s, 3H), 1.85 (m, 2H), 1.7 (m, 2H);

MS m/z: [M+H]⁺=566.

### F. [4-(5-Aminomethyl-2-fluoro-phenyl)-piperidin-1-yl]-(7-methyl-1-phenethyl-1H-indol-3-yl)-methanone hydrochloride

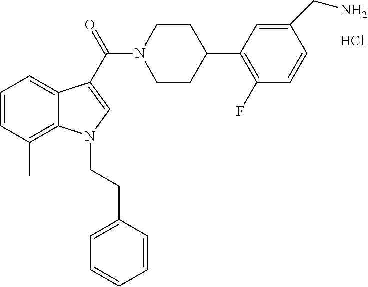

The title compound is prepared in a similar manner as described in Example 1K using 2,2,2-trifluoro-N-{4-fluoro-3-[1-(7-methyl-1-phenethyl-1H-indole-3-carbonyl)-piperidin-4-yl-benzyl}-acetamide as the starting material.

¹H NMR (300 MHz, DMSO-d6) δ 8.2 (bs, 2H), 7.55 (m, 2H), 7.45 (s, 1H), 7.35 (m, 1H), 7.3-7.1 (m, 6H), 7.0 (m, 2H), 4.6 (t, 2H), 4.3 (bs, 2H), 4.0 (t, 2H), 3.0 (m, 5H), 2.8 (s, 3H), 1.8 (m, 2H), 1.6 (m, 2H);

MS m/z: [M+H]⁺=470.

### Example 6

### [4-(5-Aminomethyl-2-fluoro-phenyl)-piperidin-1-yl]-[7-cyclopropyl-1-(2-methoxy-ethyl)-1H-indol-3-yl]-methanone hydrochloride

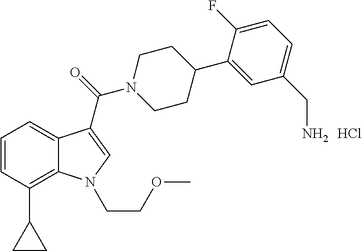

### A. 7-Bromo-1-(2-methoxy-ethyl)-1H-indole

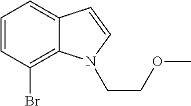

The title compound is prepared in a similar manner as described in Example 1E, using 7-bromo-1H-indole as the starting material.

¹H NMR (300 MHz, CD₃CN) δ 7.55 (d, 1H), 7.33 (d, 1H), 7.20 (d, 1H), 6.91 (t, 1H), 6.47 (d, 1H), 4.68 (t, 2H), 3.69 (t, 2H), 3.22 (s, 3H).

### B. 7-Cyclopropyl-1-(2-methoxy-ethyl)-1H-indole

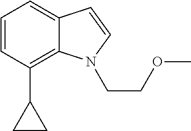

The title compound is prepared according to the procedure by Wallace, D. J. et al. Tetrahedron Letters 2002, 43, 6987-6990 using cyclopropylboronic and 7-bromo-1-(2-methoxy-ethyl)-1H-indole as the starting materials.

¹H NMR (300 MHz, CD₃CN) δ 7.45-7.40 (m, 1H), 7.17 (d, 1H), 6.98-6.91 (m, 2H), 6.45 (d, 1H), 4.88 (t, 2H), 3.69 (t, 2H), 3.25 (s, 3H), 2.41-2.32 (m, 1H), 1.01-0.95 (m, 2H), 0.85-0.80 (m, 2H).

### C. 1-[7-Cyclopropyl-1-(2-methoxy-ethyl)-1H-indol-3-yl]-2,2,2-trifluoro-ethanone

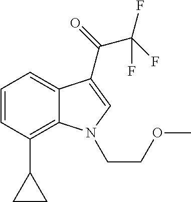

The title compound is prepared in a similar manner as described in Example 2G, using 7-cyclopropyl-1-(2-methoxy-ethyl)-1H-indole as the starting material.

¹H NMR (300 MHz, CD₃CN) δ 8.19-8.17 (m, 2H), 7.24 (t, 1H), 7.15 (m, 1H), 4.91 (t, 2H), 3.77 (t, 2H), 3.26 (s, 3H), 2.41-2.32 (m, 1H), 1.05-0.99 (m, 2H), 0.88-0.83 (m, 2H).

### D. 7-Cyclopropyl-1-(2-methoxy-ethyl)-1H-indole-3-carboxylic acid

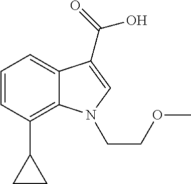

The title compound is prepared in a similar manner as described in Example 2H, using 1-[7-cyclopropyl-1-(2-methoxy-ethyl)-1H-indol-3-yl]-2,2,2-trifluoro-ethanone as the starting material.

¹H NMR (300 MHz, DMSO-d6) δ 11.95 (bs, 1H), 8.01-7.91 (m, 2H), 7.06 (t, 1H), 6.96 (m, 1H), 4.84 (t, 2H), 3.72 (t, 2H), 3.23 (s, 3H), 2.43-2.37 (m, 1H), 1.02-0.95 (m, 2H), 0.85-0.79 (m, 2H).

### E. N-(3-{1-[7-Cyclopropyl-1-(2-methoxy-ethyl)-1H-indole-3-carbonyl]-piperidin-4-yl}-4-fluoro-benzyl)-2,2,2-trifluoro-acetamide

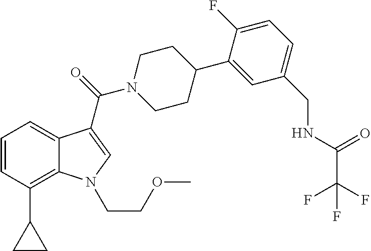

To a solution of 7-cyclopropyl-1-(2-methoxy-ethyl)-1H-indole-3-carboxylic acid (690 mg, 2.66 mmol) in dichloromethane (40 mL) and N,N-dimethylformamide (2 mL) is added 1-(3-dimethylaminopropyl)-3-ethylcarbodiimide hydrochloride (661 mg, 3.45 mmol), 1-hydroxy-benzotriazole (407 mg, 3.01 mmol) and triethylamine (1.15 mL, 8.22 mmol). The resulting mixture is stirred for 20 minutes at r.t. 2,2,2-Trifluoro-N-(4-fluoro-3-piperidin-4-yl-phenyl)-acetamide hydrochloride (1.01 g, 2.96 mmol) is added and heated at 40° C. for 4 hours. The mixture is poured into water and the organic layer separated. The aqueous phase is extracted with ethyl acetate (3×). The combined organic phases are washed with brine, dried over MgSO₄, filtered and concentrated in vacuo. The crude residue is flash chromatographed over SiO₂ using heptane/EtOAc (15:85) as the eluent to afford the title compound (1.34 g, 92%) as a white solid.

¹H NMR (300 MHz, CD₃CN) δ 8.17 (bs, 1H), 7.59-7.56 (m, 1H), 7.45 (s, H), 7.27-7.24 (m, 1H), 7.17-7.12 (m, 1H), 7.05-6.97 (m, 3H), 4.81 (t, 2H), 4.47 (br d, 2H), 3.37 (d, 2H), 3.70 (t, 2H), 3.24 (s, 3H), 3.19-3.00 (m, 3H), 2.39-2.30 (m, 1H), 1.83-1.61 (m, 4H), 1.01-0.93 (m, 2H), 0.84-0.79 (m, 2H).

### F. [4-(5-Aminomethyl-2-fluoro-phenyl)-piperidin-1-yl]-[7-cyclopropyl-1-(2-methoxy-ethyl)-1H-indol-3-yl]-methanone hydrochloride

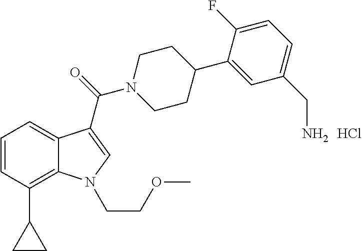

The title compound is prepared in a similar manner as described in Example 3B using N-(3-{1-[7-cyclopropyl-1-(2-methoxy-ethyl)-1H-indole-3-carbonyl]-piperidin-4-yl}-4-fluoro-benzyl)-2,2,2-trifluoro-acetamide as the starting material

¹H NMR (300 MHz, CD₃CN) δ 8.22 (br s, 2H), 7.68-7.65 (m, 1H), 7.57-7.55 (m, 2H), 7.36-7.32 (m, 1H), 7.08-6.96 (m, 3H), 4.79 (t, 2H), 4.40 (br d, 2H), 4.03 (m, 2H), 3.70 (t, 2H), 3.23 (s, 3H), 3.20-2.99 (m, 3H), 2.3-2.29 (m, 1H), 1.79-1.64 (m, 4H), 1.00-0.94 (m, 2H), 0.83-0.78 (m, 2H);

MS m/z: [M+H]⁺=450.

### Example 7

### [4-(5-Aminomethyl-2-fluoro-phenyl)-piperidin-1-yl]-[7-methyl-1-(3-morpholin-4-yl-propyl)-1H-indol-3-yl]-methanone hydrochloride

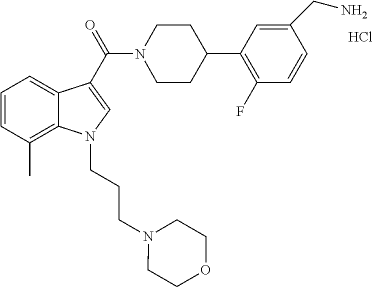

### A. 7-Methyl-1-(3-morpholin-4-yl-propyl)-1H-indole-3-carboxylic acid methyl ester

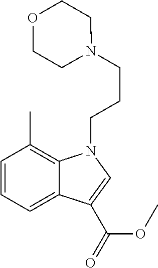

The title compound is prepared in a similar manner as described in Example 2F using 7-methyl-1H-indole-3-carboxylic acid methyl ester and 4-(3-chloro-propyl)-morpholine in DMF as the starting materials.

¹H NMR (300 MHz, CDCl₃) δ 8.1 (d, 1H), 7.8 (s, 1H), 7.2 (m, 1H), 7.0 (m, 1H), 4.45 (t, 2H), 3.9 (m, 3H), 3.7 (m, 4H), 2.8 (s, 3H), 2.4 (m, 4H), 2.3 (m, 2H), 2.0 (m, 2H);

MS m/z: [M+H]⁺=317.

### B. 7-Methyl-1-(3-morpholin-4-yl-propyl)-1H-indole-3-carboxylic acid

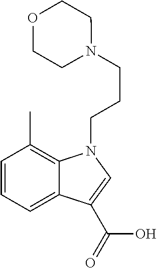

To a solution of 7-methyl-1-(3-morpholin-4-yl-propyl)-1H-indole-3-carboxylic acid methyl ester (1.2 g, 3.8 mmol) in MeOH (30 mL) is added 1 N NaOH (10 mL). The solution is heated to reflux for 2 h and then cooled to room temperature. The reaction mixture is acidified to pH=2 with conc. HCl then concentrated in vacuo to remove MeOH. The aqueous phase is lyophilized to dryness and the solid is triturated with 10% acetonitrile/water and collected to yield the title compound (0.85 g, 74% yield).

¹H NMR (300 MHz, DMSO-d6) δ 9.6 (bs, 1H), 8.1 (s, H), 7.9 (d, 1H), 7.1 (m, 1H), 7.0 (t, 1H), 4.5 (t, 2H), 4.0 (m, 2H), 3.6 (m, 2H), 3.1 (m, 5H), 2.75 (s, 3H), 2.2 (m, 3H);

MS m/z: [M+H]⁺=303.

### C. 2,2,2-Trifluoro-N-(4-fluoro-3-{1-[7-methyl-1-(3-morpholin-4-yl-propyl)-1H-indole-3-carbonyl]-piperidin-4-yl}-benzyl)-acetamide

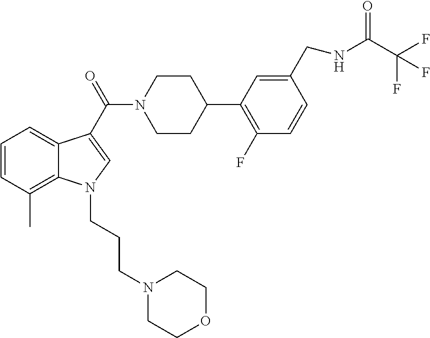

The title compound is prepared in a similar manner as described in Example 2I, using 7-methyl-1-(3-morpholin-4-yl-propyl)-1H-indole-3-carboxylic acid and 2,2,2-trifluoro-N-(4-fluoro-3-piperidin-4-yl-benzyl)-acetamide hydrochloride as the starting materials.

¹H NMR (300 MHz, CDCl₃) δ 7.6 (d, 1H), 7.4 (s, 1H), 7.15 (m, 3H), 7.0 (m, 2H), 6.8 (bs, H), 4.5 (m, 6H), 3.9 (m, 4H), 3.45 (bs, H), 3.15 (m, 2H), 3.0 (m, 2H), 2.8 (m, 1H), 2.65 (s, 3H), 2.35 (m, 2H), 1.9 (m, 2H), 1.7 (m, 2H);

MS m/z: [M+H]⁺=589.

### D. [4-(5-Aminomethyl-2-fluoro-phenyl)-piperidin-1-yl]-[7-methyl-1-(3-morpholin-4-yl-propyl)-1H-indol-3-yl]-methanone hydrochloride

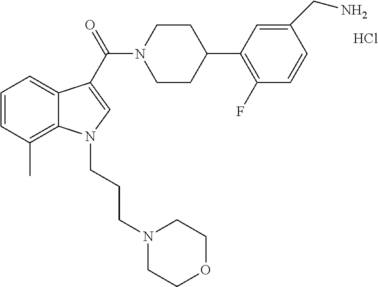

The title compound is prepared in a similar manner as described in Example 1K, using 2,2,2-trifluoro-N-(4-fluoro-3-{1-[7-methyl-1-(3-morpholin-4-yl-propyl)-1H-indole-3-carbonyl]-piperidin-4-yl}-benzyl)-acetamide as the starting material.

¹H NMR (300 MHz, DMSO-d6) δ 8.3 (bs, 2H), 7.8 (s, 1H), 7.55 (m, 2H), 7.4 (m, 1H), 7.2 (m, 1H), 7.0 (m, 2H), 4.45 (m, 4H), 4.0 (m, 4H), 3.75 (t, 2H), 3.15 (m, 8H), 2.75 (s, 3H), 1.8 (m, 2H), 1.7 (m, 2H);

MS m/z: [M+H]⁺=493.

### Example 8

### [4-(5-Aminomethyl-2-fluoro-phenyl)-piperidin-1-yl]-{7-methyl-1-[2-(1-methyl-piperidin-2-yl)-ethyl]-1H-indol-3-yl}-methanone hydrochloride

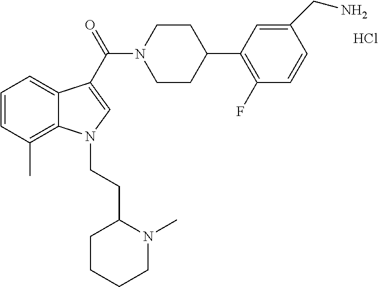

### A. 7-Methyl-1-[2-(1-methyl-piperidin-2-yl)ethyl]-1H-indole

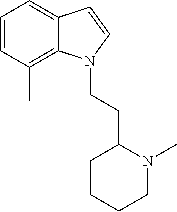

The title compound is prepared in a similar manner as described in Example 1E, using 7-methylindole and 2-(2-chloroethyl)1-methylpiperidine hydrochloride as the starting materials.

¹H NMR (300 MHz, CDCl₃) δ 7.5 (dd, 1H), 7.0 (d, 1H), 6.95 (m, 2H), 6.45 (d, 2H), 4.4 (m, 2H), 2.85 (m, 1H), 2.7 (s, 3H), 2.3 (s, 3H), 2.2-2.0 (m, 4H), 1.8-1.6 (m, 6H);

MS m/z: [M+H]⁺=257.

### B. 2,2,2-Trifluoro-1-{7-methyl-1-[2-(1-methyl-piperidin-2-yl)-ethyl]-1H-indol-3-yl}-ethanone

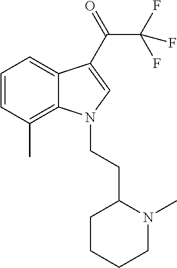

The title compound is prepared in a similar manner as described in Example 1F, using 7-methyl-1-[2-(1-methyl-piperidin-2-yl)ethyl]-1H-indole as the starting material.

¹H NMR (300 MHz, CDCl₃) δ 8.3 (d, 1H), 7.95 (dd, 1H), 7.2 (m, 1H), 7.15 (m, 1H), 4.7 (m, 1H), 4.5 (m, 1H), 3.8 (m, 1H), 3.4 (m, 1H), 3.2 (m, 1H), 2.7 (m, 7H), 2.5 (m, 1H), 2.35 (m, 1H), 2.0 (m, 2H), 1.9 (m, 3H);

MS m/z: [M+H]⁺=353

### C. 7-Methyl-1-[2-(1-methyl-piperidin-2-yl)-ethyl]-1H-indole-3-carboxylic acid

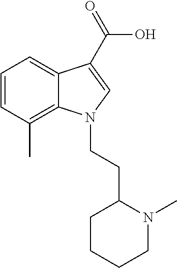

The title compound is prepared in a similar manner as described in Example 4C, using 2,2,2-trifluoro-1-{7-methyl-1-[2-(1-methyl-piperidin-2-yl)-ethyl]-1H-indol-3-yl}-ethanone as the starting material.

¹H NMR (300 MHz, DMSO-d6) δ 12.0 (bs, 1H), 8.2 (s, 1H), 7.95 (d, 1H), 7.0 (m, 2H), 4.5 (m, 2H), 4.1 (m, 1H), 3.2 (s, 3H), 3.15 (m, 1H), 2.7 (m, 6H), 2.1 (m, 2H), 1.8 (m, 4H),

MS m/z: [M+H]⁺=301.

### D. 2,2,2-Trifluoro-N-[4-fluoro-3-(1-{7-methyl-1-[2-(1-methyl-piperidin-2-yl)-ethyl]-1H-indole-3-carbonyl}-piperidin-4-yl)-benzyl]-acetamide

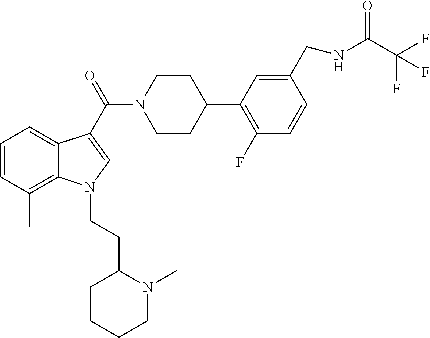

The title compound is prepared in a similar manner as described in Example 2I, using 7-methyl-1-[2-(1-methyl-piperidin-2-yl)-ethyl]-1H-indole-3-carboxylic acid and 2,2,2-trifluoro-N-(4-fluoro-3-piperidin-4-yl-benzyl)acetamide hydrochloride as the starting materials.

MS m/z: [M+H]⁺=587.

### E. [4-(5-Aminomethyl-2-fluoro-phenyl)-piperidin-1-yl]-{7-methyl-1-[2-(1-methyl-piperidin-2-yl)-ethyl]-1H-indol-3-yl}-methanone hydrochloride

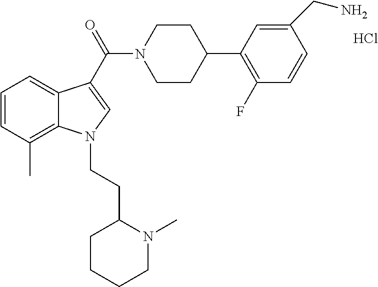

The title compound is prepared in a similar manner as described in Example 1K using 2,2,2-trifluoro-N-[4-fluoro-3-(1-{7-methyl-1-[2-(1-methyl-piperidin-2-yl)-ethyl]-1H-indole-3-carbonyl]-piperidin-4-ylybenzyl}-acetamide as the starting material.

¹H NMR (300 MHz, DMSO-d6) δ 11.2-10.8 (bd, 1H), 8.5 (bs, 2H), 7.8 (d, 1H), 7.65 (m, 1H), 7.5 (m, 1H), 7.4 (m, 1H), 7.3 (m, 1H), 7.0 (m, 2H), 4.4 (m, 4H), 4.0 (m, 2H), 3.4 (m 2H), 3.2 (m, 3H), 2.75 (m, 1H), 2.6 (m, 2H), 2.55 (m, 4H), 2.2-2.0 (m, 2H), 1.8-1.6 (m, 9H);

MS m/z: [M+H]⁺=491.

### Example 9

### [4-(5-Aminomethyl-2-difluoromethyl-phenyl)-piperidin-1-yl]-[1-(2-methoxy-ethyl)-7-methyl-1H-indol-3-yl]-methanone hydrochloride

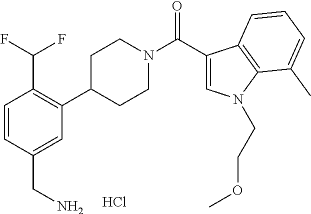

### A. 4-Azidomethyl-2-bromo-benzaldehyde

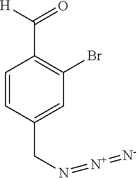

To a solution of 2-bromo-4-bromomethyl-benzaldehyde (US 2003114703 A1) (7.540 g, 27.1 mmol) in DMF (22 mL) is added sodium azide (3.527 g, 54.3 mmol) in portions. The resulting mixture is stirred at r.t. overnight. The mixture is diluted with Et₂O and washed with water then brine. The organic layer is dried over MgSO4, filtered and concentrated in vacuo to give the titled product (6.39 g, 98%) as an orange oil.

¹H NMR (300 MHz, CDCl₃) δ 10.35 (s, 1H), 7.93 (d, J=7.8 Hz, 1H), 7.64 (d, J=1.2 Hz, 1H), 7.40 (dd, J=0.9 Hz, 7.8 Hz, 1H), 4.44 (s, 2H).

### B. 4-Azidomethyl-2-bromo-1-difluoromethyl-benzene

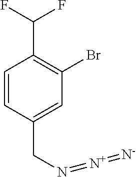

To a solution of 4-azidomethyl-2-bromo-benzaldehyde (6.47 g, 26.9 mmol) in CH₃CN (50 mL) is added 2,2-difluoro-1,3-dimethylimidazolidine (5.4 mL, 43.5 mmol) dropwise. The amber solution is heated at 84° C. overnight. The reaction mixture is cooled to r.t. and diluted with Et₂O. The organic layer is washed with saturated Na₂CO₃ solution, brine and then dried over MgSO₄, filtered and concentrated in vacuo. The crude material is purified on silica gel using EtOAc/heptane (5-30%) as the eluent to give the titled product (5.11 g, 72%) as a pale yellow oil.

¹H NMR (300 MHz, CDCl₃) δ 7.67 (d, J=8.1 Hz, 1H), 7.59 (s, 1H), 7.38 (d, J=7.8 Hz, 1H), 6.90 (t, J=54.6 Hz, 1H), 4.40 (s, 2H);

¹⁹F NMR (300 MHz, CDCl₃) δ −114.4 (2F);

LC 3.21 min; MS 263 (M+1, 88%).

### C. 3-Bromo-4-difluoromethyl-benzylamine

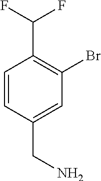

To a solution of 4-azidomethyl-2-bromo-1-difluoromethyl-benzene (4.67 g, 17.8 mmol) in H₂O/THF (7 mL/70 mL) is added triphenylphosphine (9.30 g, 35.5 mmol) in portions. The mixture is stirred at r.t. overnight. The reaction mixture is concentrated in vacuo to remove THF then 1 N HCl (100 mL) is added and the aqueous solution is washed with CH₂Cl₂. The aqueous layer is cooled to 0° C. and 6.25 N NaOH (ca. 30 mL) is added until pH ˜10. The aqueous layer is extracted with CH₂Cl₂ (3×100 mL). The combined organic layers are washed with brine, dried over MgSO₄, filtered and concentrated in vacuo to give the titled product (3.83 g, 91%) as an oil.

¹H NMR (300 MHz, CDCl₃) δ 7.63-7.60 (m, 2H), 7.35 (d, 1H), 6.90 (t, J=54.9 Hz, 1H), 3.91 (s, 2H), 1.42 (br s, 2H);

LC 1.23 min; MS 236, 238 (M+1).

### D. 4-Difluoromethyl-3-pyridin-4-yl-benzylamine

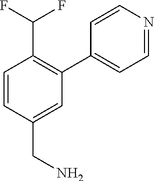

A solution of 3-bromo-4-difluoromethyl-benzylamine (1.023 g, 4.33 mmol), pyridine-4-boronic acid (0.651 g, 4.77 mmol) and NaHCO3 (0.364 g, 4.33 mmol) in H₂O/IPA (3.2 mL/6.6 mL) is degassed with N₂ for 20 min. Pd-dppf Cl₂ is added to the mixture, and the resulting mixture is heated at 85° C. for 22 h. The reaction mixture is cooled to r.t. and 1 N HCl (15 mL) is added. After the resulting mixture is stirred for 20 min, it is washed with CH₂Cl₂ (×3), and the aqueous layer is treated with 50% NaOH (10 mL) to adjust to pH>13. The aqueous layer is extracted with EtOAc (×3) and the combined organic layers are dried over MgSO₄, filtered and concentrated in vacuo to give the titled compound (0.926 g, 91%) as a oil.

¹H NMR (300 MHz, CDCl₃) δ 8.71-8.68 (m, 2H), 7.77 (d, J=8.1 Hz, 1H), 7.52 (d, J=9.9 Hz, 1H), 7.33-7.26 (m, 3H), 6.49 (t, J=54.9 Hz, 1H), 3.99 (s, 2H), 1.5 (br s, 2H).

### E. N-(4-Difluoromethyl-3-pyridin-4-yl-benzyl)-2,2,2-trifluoro-acetamide hydrochloride

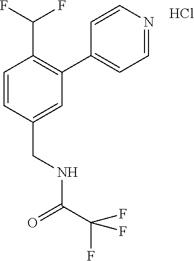

To a solution of 4-difluoromethyl-3-pyridin-4-yl-benzylamine (2.350 g, 10.03 mmol) in n-BuOAc (30 mL) at 0° C. is added trifluoroacetic anhydride dropwise. The resulting solution is stirred for 4 hr then the reaction mixture is washed with 10% Na₂CO₃ (100 mL) and brine. The organic layer is dried over MgSO₄, filtered and concentrated to dryness in vacuo. The residue is taken up in n-BuOAc (5 mL) and 2.0 M HCl in ether is added. The resulting solution is stirred for 20 min then concentrated in vacuo to provide the titled product (3.277 g, 88%) as a foamy solid.

¹H NMR (300 MHz, DMSO-d6) δ 10.15 (br s, 1H), 8.92 (d, J=6 Hz, 1H), 7.83-7.66 (m, 3H), 7.59 (d, J=9 Hz, 1H), 7.42 (s, 1H), 7.16 (t, J=42 Hz, 1H), 4.53 (d, J=6 Hz, 2H),

¹⁹F NMR (300 MHz, DMSO-d6) δ −74.75 (s, 3F), −107.59 (2F).

### F. N-(4-Difluoromethyl-3-piperidin-4-yl-benzyl)-2,2,2-trifluoro-acetamide hydrochloride

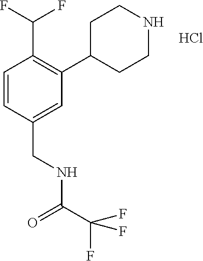

A Parr flask is charged with N-(4-difluoromethyl-3-pyridin-4-yl-benzyl)-2,2,2-trifluoro-acetamide hydrochloride (1.8 g, 4.9 mmol) and MeOH (50 mL) at r.t., then 5% Pt/C (0.53 g, 30 w/w %) is added. The reaction flask is placed in a Parr hydrogenation system and charged with H₂ at 50-60 psi. The mixture is shaken for 24 h while charging H₂ until the pressure reached a steady state. When HPLC analysis shows completion of the reaction, the reaction mixture is filtered through a pad of Celite. The filtrate is concentrated to dryness in vacuo to give the titled product (1.63 g, 88%) as a white solid.

¹H NMR (300 MHz, DMSO-d6) δ 10.09 (br m, 1H), 8.5 (br s, 2H), 7.57 (d, J=7.8 Hz, 1H), 7.31-7.27 (m, 2H), 7.10 (t, 1H), 4.44 (s, 2H), 3.33-3.04 (m, 3H), 3.05-3.00 (m, 2H), 1.83-1.81 (m, 4H);

¹⁹F NMR (300 MHz, DMSO-d6) δ −74.01 (s, 3F), −109.36 (d, 2F);

LC 1.78 min; MS 337 (M+1, 97%).

### G. 1-(2-Methoxy-ethyl)-7-methyl-1H-indole

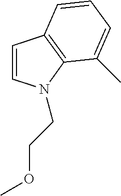

The title product (7.18 g, 99%) is obtained in a similar manner as described in Example 1E using 7-methyl-1H-indole (5 g, 38.2 mmol) as the starting material.

¹H NMR (300 MHz, CDCl₃) δ 7.45 (d, 1H), 7.09 (d, 1H), 7.00-6.90 (m, 2H), 6.45 (d, 1H), 4.50 (t, 2H), 3.71 (t, 2H), 3.30 (s, 3H), 2.70 (s, 3H).

### H. 2,2,2-Trifluoro-1-[1-(2-methoxy-ethyl)-7-methyl-1H-indol-3-yl]-ethanone

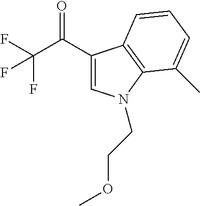

The title product (9 g, 84%) is obtained in a similar manner as described in Example 1F using 1-(2-methoxy-ethyl)-7-methyl-1H-indole (7.1 g, 37.6 mmol) as the starting material. The crude material is purified on silica gel with EtOAc/heptane (15%) as eluent.

¹H NMR (300 MHz, CDCl₃) δ 8.30 (d, 1H), 7.98 (s, 1H), 7.24 (m, 1H), 7.10 (d, 1H), 4.60 (t, 2H), 3.78 (t, 2H), 3.30 (s, 3H), 2.70 (s, 3H).

### I. 1-(2-Methoxy-ethyl)-7-methyl-1H-indole-3-carboxylic acid

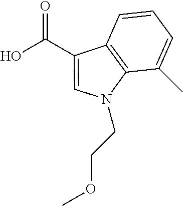

The title product (7.3 g, 100%) is obtained in a similar manner as described in Example 1G using 2,2,2-trifluoro-1-[1-(2-methoxy-ethyl)-7-methyl-1H-indol-3-yl]-ethanone (9 g, 31.6 mmol) as the starting material and heating to reflux then stirring at 110° C. overnight.

¹H NMR (300 MHz, DMSO-d6) δ 11.90 (s, 1H), 7.95 (s, 1H), 7.90 (d, 1H), 7.05 (t, 1H), 6.95 (d, 1H), 4.60 (t, 2H), 3.65 (t, 2H), 3.22 (s, 3H), 2.65 (s, 3H).

### J. N-(4-Difluoromethyl-3-{1-[1-(2-methoxy-ethyl)-7-methyl-1H-indole-3-carbonyl]-piperidin-4-yl}-benzyl)-2,2,2-trifluoro-acetamide

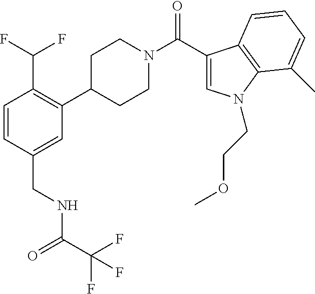

The title product (0.173 g, 68%) is obtained in a similar manner as described in Example 1J using N-(4-difluoromethyl-3-piperidin-4-yl-benzyl)-2,2,2-trifluoro-acetamide hydrochloride (0.172 g, 0.46 mmol) and 1-(2-methoxy-ethyl)-7-methyl-1H-indole-3-carboxylic acid (0.108 g, 0.46 mmol) as the starting materials.

¹H NMR (300 MHz, DMSO-d6) δ 7.58 (d, 1H), 7.50 (d, 1H), 7.45 (s, 1H), 7.31-7.23 (m, 2H), 7.09 (t, J=7.5 Hz, 1H), 6.98 (d, J=7.5 Hz, 1H), 6.81 (t, J=5.2 Hz, 1H), 4.60-4.52 (m, 4H), 3.71 (t, J=5.4 Hz, 2H), 3.50 (br d, 1H), 3.31 (s, 3H), 3.20-3.00 (m, 4H), 2.72 (s, 3H), 1.85-1.72 (m, 4H);

¹⁹F NMR (300 MHz, DMSO-d6) δ −75.39 (s, 3F), −109.00 (d, 2F);

LC 3.29 min; MS 552 (M+1, 94%).

### K. [4-(5-Aminomethyl-2-difluoromethyl-phenyl)-piperidin-1-yl]-[1-(2-methoxy-ethyl)-7-methyl-1H-indol-3-yl]-methanone hydrochloride

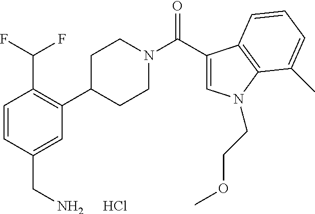

The title product (0.098 g, 64%) is obtained in a similar manner as described in Example 1K using N-(4-difluoromethyl-3-{1-[1-(2-methoxy-ethyl)-7-methyl-1H-indole-3-carbonyl]-piperidin-4-yl}-benzyl)-2,2,2-trifluoro-acetamide (0.173 g, 0.31 mmol) as the starting material.

¹H NMR (300 MHz, DMSO-d6) δ 8.21 (br s, 2H), 7.70-7.51 (m, 4H), 7.41 (d, 1H), 7.39 (t, 1H), 7.05-6.90 (m, 2H), 4.58 (t, 2H), 4.45 (br d, 1H), 4.08 (s, 2H), 3.67 (t, 2H), 3.32 (s, 3H), 3.20-3.00 (br m, 4H), 2.68 (s, 3H), 1.72 (m, 4H);

¹⁹F NMR (300 MHz, DMSO-d6) δ −109.65 (d, J=58.5 Hz, 2F);

LC 2.36 min; MS 456 (M+1, 95%).

### Example 10

### [4-(5-Aminomethyl-2-fluoro-phenyl)-piperidin-1-yl]-[7-methyl-1-(3-pyridin-3-yl-propyl)-1H-indol-3-yl]-methanone dihydrochloride

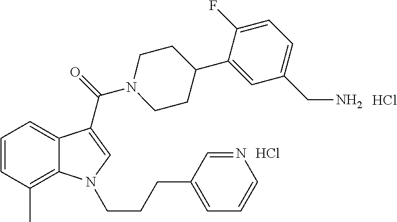

### A. 7-Methyl-1-(3-pyridin-3-yl-propyl)-1H-indole

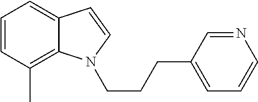

The title compound is prepared in a similar manner as described in Example 1E using 3-(3-bromo-propyl)-pyridine and 7-methylindole as the starting materials.

¹H NMR (300 MHz, CD₃CN) δ 8.37 (m, 2H), 7.49 (m, 1H), 7.36 (m, 1H), 7.20-7.16 (m, 1H), 7.09 (m, 1H), 6.90-6.82 (m, 2H), 6.39 (d, 1H), 4.31 (t, 2H), 2.64-2.50 (m, 5H), 2.07-1.97 (s, 2H).

### B. 2,2,2-Trifluoro-1-[7-methyl-1-(3-pyridin-3-yl-propyl)-1H-indol-3-yl]-ethanone

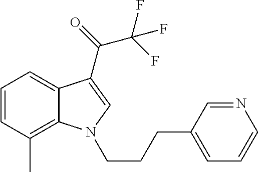

The title compound is prepared in a similar manner as described in Example 2G with 7-methyl-1-(3-pyridin-3-yl-propyl)-1H-indole as the starting material.

¹H NMR (300 MHz, CD₃CN) δ 8.57 (br s, 2H), 8.19-8.09 (m, 3H), 7.69-7.65 (m, 1H), 7.25-7.20 (m, 1H), 7.14-7.12 (m, 1H), 4.51 (t, 2H), 2.86 (t, 2H), 2.69 (s, 3H), 2.29-2.18 (m, 2H).

### C. 7-Methyl-1-(3-pyridin-3-yl-propyl)-1H-indole-3-carboxylic acid

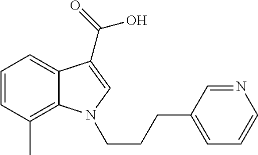

The title compound is prepared in a similar manner as described in Example 2H with 2,2,2-trifluoro-1-[7-methyl-1-(3-pyridin-3-yl-propyl)-1H-indol-3-yl]-ethanone as the starting material.

¹H NMR (300 MHz, DMSO-d6) δ 11.94 (br s, 1H), 8.46-8.40 (m, 2H), 8.03 (s, 1H), 7.91 (d, 1H), 7.66 (m, 1H), 7.32-7.28 (m, 1H), 7.04 (t, 1H), 6.95 (m, 1H), 4.44 (t, 2H), 2.67 (t, 2H), 2.58 (s, 3H), 2.14-2.04 (m, 2H).

### D. 2,2,2-Trifluoro-N-(4-fluoro-3-{1-[7-methyl-1-(3-pyridin-3-yl-propyl)-1H-indole-3-carbonyl]-piperidin-4-yl}-benzyl)-acetamide

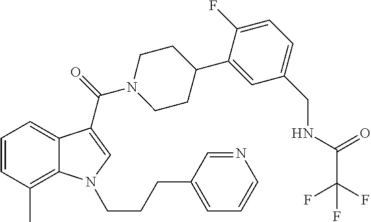

The title compound is prepared in a similar manner as described in Example 6E with 7-methyl-1-(3-pyridin-3-yl-propyl)-1H-indole-3-carboxylic acid and 2,2,2-trifluoro-N-(4-fluoro-3-piperidin-4-yl-benzyl)-acetamide hydrochloride as the starting materials.

¹H NMR (300 MHz, CD₃CN) δ 8.42-8.37 (m, 2H), 8.07 (br s, 1H), 7.57 (d, 2H), 7.43 (s, 1H), 7.29-7.21 (m, 2H), 7.19-7.12 (m, 1H), 7.07-6.99 (m, 2H), 6.95-6.93 (m, 1H), 4.49-4.38 (m, 6H), 3.19-3.01 (m, 3H), 2.69-2.62 (m, 5H), 2.12-2.08 (m, 2H), 1.80-1.63 (m, 4H).

### E. [4-(5-Aminomethyl-2-fluoro-phenyl)-piperidin-1-yl]-[7-methyl-1-(3-pyridin-3-yl-propyl)-1H-indol-3-yl]-methanone dihydrochloride

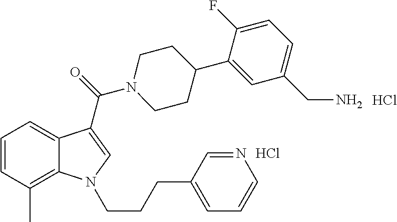

The title compound is prepared in a similar manner as described in Example 3B with 2,2,2-trifluoro-N-(4-fluoro-3-{1-[7-methyl-1-(3-pyridin-3-yl-propyl)-1H-indole-3-carbonyl]-piperidin-4-yl}-benzyl)-acetamide as the starting material.

¹H NMR (300 MHz, DMSO-d6) δ 8.79 (br s, 1H), 8.70 (d, 1H), 8.32 (br s, 3H), 7.87-7.82 (m, 1H), 7.70 (s, 1H), 7.58 (m, 1H), 7.51 (d, 1H), 7.38-7.35 (m, 1H), 7.25-7.19 (m, 1H), 7.03-6.98 (m, 1H), 6.95-6.93 (m, 1H), 4.48-4.38 (m, 4H), 4.00 (m, 2H), 3.19-3.05 (m, 4H), 2.88-2.83 (m, 2H), 2.65 (s, 3H), 2.19-2.14 (m, 2H), 1.18-1.61 (m, 4H);

MS m/z: [M+H]⁺=485.

### Example 11

### [4-(5-Aminomethyl-2-fluoro-phenyl)-piperidin-1-yl]-[1-(2-methoxy-ethyl)-7-trifluoromethyl-1H-indol-3-yl]-methanone hydrochloride

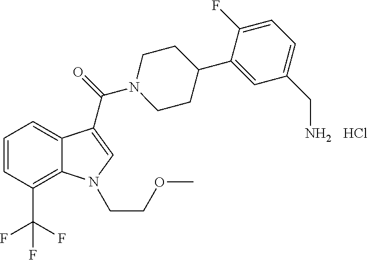

### A. 7-Trifluoromethyl-1H-indole-3-carboxylic acid methyl ester

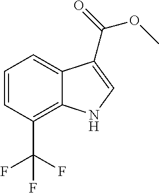

N-(2-Trifluoromethyl-phenyl)-hydroxylamine is prepared according to the procedure by Oxley, P. W. et al. Org. Syn. 1989, 67, 187-190 using 2-nitrobenzotrifluoride as the starting material. The crude mixture is used for the next step without any purification.

The title compound is prepared according to the procedure by Jih Ru Hwn et al. J. Org. Chem. 1994, 59, 1577-1582 using N-(2-trifluoromethyl-phenyl)-hydroxylamine as the starting material.

¹H NMR (300 MHz, CD₃CN) δ 8.37 (d, 1H), 8.02 (m, 1H), 7.57 (d, 1H), 7.36 (t, 1H), 3.86 (s, 3H).

### B. 1-(2-Methoxy-ethyl)-7-trifluoromethyl-1H-indole-3-carboxylic acid methyl ester

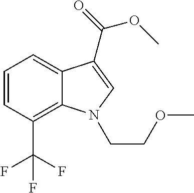

The title compound is prepared in a similar manner as described in Example 2F using 7-trifluoromethyl-1H-indole-3-carboxylic acid methyl ester and 2-methoxyethyl bromide as the starting materials.

¹H NMR (300 MHz, CD₃CN) δ 8.47 (d, 1H), 8.00 (s, 1H), 7.67 (d, 1H), 7.34 (t, 1H), 4.48 (t, 2H), 3.85 (s, 3H), 3.67 (t, 2H), 3.27 (s, 3H).

### C. 1-(2-Methoxy-ethyl)-7-trifluoromethyl-1H-indole-3-carboxylic acid

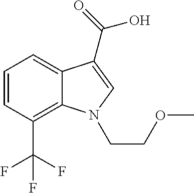

To a solution of 1-(2-methoxy-ethyl)-7-trifluoromethyl-1H-indole-3-carboxylic acid methyl ester (763 mg, 2.53 mmol) in THF:MeOH:H₂O (1:1:1) (15 mL) is added lithium hydroxide hydrate (533 mg, 12.70 mmol) and the mixture is stirred at r.t. overnight. The solvents are removed in vacuo and the aqueous residue is flash frozen and lyophilized. The solid is titrated with EtOAc/DCM/MeOH (8:1:1). The resulting suspension is filtered, and the filtrate is evaporated in vacuo and vacuum dried to give the title compound as a white solid.

¹H NMR (300 MHz, DMSO-d6) δ 8.73 (d, 1H), 7.66 (s, 1H), 7.48 (d, 1H), 7.16 (t, 1H), 4.38 (m, 2H), 3.62 (m, 2H), 3.24 (s, 3H).

### D. 2,2,2-Trifluoro-N-(4-fluoro-3-{1-[1-(2-methoxy-ethyl)-7-trifluoromethyl-1H-indole-3-carbonyl]-piperidin-4-yl}-benzyl)-acetamide

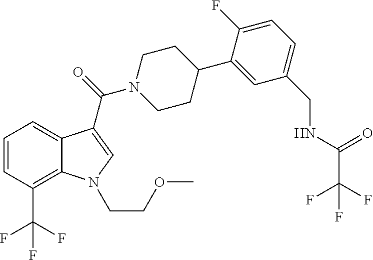

The title compound is prepared in a similar manner as described in Example 6E using 1-(2-methoxy-ethyl)-7-trifluoromethyl-1H-indole-3-carboxylic acid and 2,2,2-trifluoro-N-(4-fluoro-3-piperidin-4-yl-benzyl)-acetamide hydrochloride as the starting materials.

¹H NMR (300 MHz, CD₃CN) δ 8.07 (d, 1H), 7.92 (br s, 1H), 7.65-7.60 (m, 2H), 7.29-7.24 (m, 2H), 7.19-7.14 (m, 1H), 7.07-7.01 (m, 1H), 4.48-4.38 (m, 6H), 3.66 (t, 2H), 3.26 (s, 3H), 3.32-3.04 (m, 3H), 1.87-1.65 (m, 4H).

### E. [4-(5-Aminomethyl-2-fluoro-phenyl)-piperidin-1-yl]-[1-(2-methoxy-ethyl)-7-trifluoromethyl-1H-indol-3-yl]-methanone hydrochloride

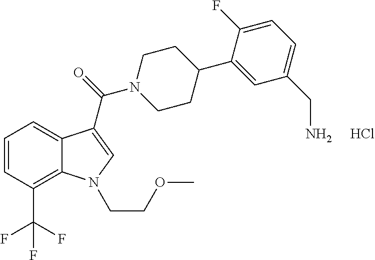

The title compound is prepared in a similar manner as described in Example 3B using 2,2,2-trifluoro-N-(4-fluoro-3-{1-[1-(2-methoxy-ethyl)-7-trifluoromethyl-1H-indole-3-carbonyl]-piperidin-4-yl}-benzyl)-acetamide as the starting material.

¹H NMR (300 MHz, DMSO-d6) δ 8.34 (br s, 3H), 8.08 (d, 1H), 7.82 (s, H), 7.66 (d, 1H), 7.59 (d, 1H), 7.40-7.30 (m, 2H), 7.25-7.19 (m, 1H), 4.49-4.59 (m, 4H), 4.00 (m, 2H), 3.65 (m, 2H), 3.24 (s, 3H), 3.17-3.14 (m, 3H), 1.83-1.63 (m, 4H).

### Example 12

### [4-(5-Aminomethyl-2-fluoro-phenyl)-piperidin-1-yl]-[7-difluoromethyl-1-(2-methoxy-ethyl)-1H-indol-3-yl]-methanone hydrochloride

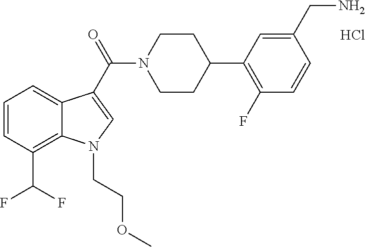

### A. 1-(2-Methoxy-ethyl)-1H-indole-7-carbaldehyde

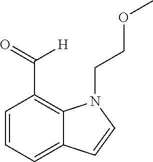

The title compound is prepared in a similar manner as described in Example 1E using 1H-indole-7-carbaldehyde as the starting material.

¹H NMR (300 MHz, CDCl₃) δ 10.0 (s, 1H), 7.9 (d, 1H), 7.7 (d, 1H), 7.2 (m, 2H), 6.6 (d, H), 4.8 (t, 2H), 3.7 (t, 2H), 3.2 (s, 3H);

MS m/z: [M+H]⁺=204

### B. 7-Difluoromethyl-1-(2-methoxy-ethyl)-1H-indole

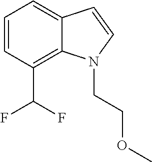

The title compound is prepared according to the procedure by Wong et al Bioorganic & Medicinal Chemistry, 2006, 14, pp 8386-95 using 1-(2-methoxy-ethyl)-1H-indole-7-carbaldehyde as the starting material.

¹H NMR (300 MHz, CDCl₃) δ 7.75 (d, 1H), 7.35 (d, 1H), 7.3 (m, 1H), 7.1 (m, 1H), 7.0 (s, 1H), 6.6 (d, 1H), 4.5 (t, 2H), 3.7 (t, 2H), 3.3 (s, 3H).

MS m/z: [M+H]⁺=226.

### C. 1-[7-Difluoromethyl-1-(2-methoxy-ethyl)-1H-indol-3-yl]-2,2,2-trifluoro-ethanone

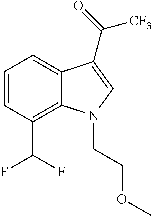

The title compound is prepared in a similar manner as described in Example 1F using 7-difluoromethyl-1-(2-methoxy-ethyl)-1H-indole as the starting material.

¹H NMR (300 MHz, CD₃CN) δ 8.5 (d, 1H), 8.3 (s, 1H), 7.6 (m, 1H), 7.4 (m, 1H), 7.2 (s, 1H), 4.6 (t, 2H), 3.7 (t, 2H), 3.2 (s, 3H).

MS m/z: [M+H]⁺=322.

### D. 7-Difluoromethyl-1-(2-methoxy-ethyl)-1H-indole-3-carboxylic acid

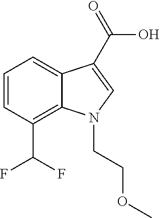

The title compound is prepared in a similar manner as described in Example 4C using 1-[7-difluoromethyl-1-(2-methoxy-ethyl)-1H-indol-3-yl]-2,2,2-trifluoro-ethanone as the starting material.

¹H NMR (300 MHz, CD₃CN) δ 8.5 (d, 1H), 8.3 (s, 1H), 7.6 (m, 1H), 7.4 (m, 1H), 7.2 (s, 1H), 4.6 (t, 2H), 3.7 (t, 2H), 3.2 (s, 3H).

MS m/z: [M+H]⁺=270.

### E. N-(3-{1-[7-Difluoromethyl-1-(2-methoxy-ethyl)-1H-indole-3-carbonyl]-piperidin-4-yl}-4-fluoro-benzyl)-2,2,2-trifluoro-acetamide

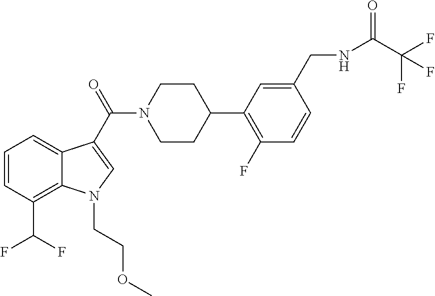

The title compound is prepared in a similar manner as described in Example 2I using 7-difluoromethyl-1-(2-methoxy-ethyl)-1H-indole-3-carboxylic acid and 2,2,2-trifluoro-N-(4-fluoro-3-piperidin-4-yl-benzyl)-acetamide hydrochloride as the starting materials.

¹H NMR (300 MHz, CDCl₃) δ 8.0 (d, 1H), 7.5 (s, 1H), 7.4 (d, 1H), 7.2 (m, 3H), 7.0 (m, 2H), 6.6 (bs, 1H), 4.5 (m, 6H), 3.7 (t, 2H), 3.3 (s, 3H), 3.1 (m, 3H), 1.9 (m, 2H), 1.75 (m, 2H).

MS m/z: [M+H]⁺=556.

### F. [4-(5-Aminomethyl-2-fluoro-phenyl)-piperidin-1-yl]-[7-difluoromethyl-1-(2-methoxy-ethyl)-1H-indol-3-yl]-methanone hydrochloride

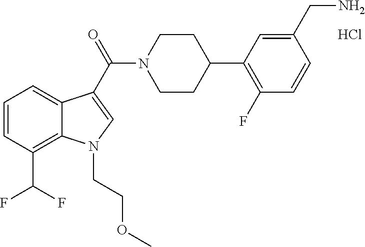

The title compound is prepared in a similar manner as described Example 1K using N-(3-{1-[7-difluoromethyl-1-(2-methoxy-ethyl)-1H-indole-3-carbonyl]-piperidin-4-yl}-4-fluoro-benzyl)-2,2,2-trifluoro-acetamide as the starting material.

¹H NMR (300 MHz, DMSO-d6) δ 8.3 (bs, 2H), 7.9 (d, 1H), 7.8 (s, 1H), 7.5 (m, 2H), 7.3 (m, 1H), 7.2 (m, 2H), 4.6 (t, 2H), 4.5 (m, 2H), 4.0 (t, 2H), 3.7 (m, 2H), 3.6 (m, 2H), 3.3 (s, 3H), 3.2 (m, 2H), 1.9 (m, 2H), 1.7 (m, 2H);

LCMS m/z: [M+H]⁺=460.

### Example 13

### [4-(5-Aminomethyl-2-fluoro-phenyl)-piperidin-1-yl]-[7-methyl-1-(2-pyrrolidin-1-yl-ethyl)-1H-indol-3-yl]-methanone dihydrochloride

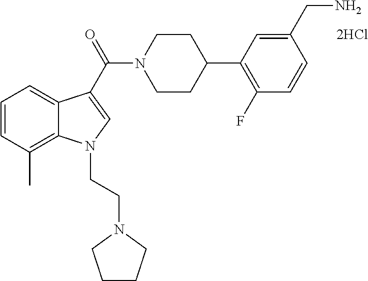

### A. 7-Methyl-1-(2-pyrrolidin-1-yl-ethyl)-1H-indole

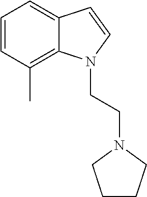

The title compound is prepared in a similar manner as described in Example 1E using 7-methylindole and 1-(2-chloro-ethyl)-pyrrolidine hydrochloride as the starting materials.

¹H NMR (300 MHz, CDCl₃) δ 7.45 (d, 1H), 7.15 (d, 1H), 6.95 (m, 2H), 6.50 (d, 1H), 4.50 (m, 2H), 3.85 (m, 2H), 3.70 (s, 3H), 2.55 (m, 4H), 1.80 (m, 4H).

MS m/z: [M+H]⁺=229.

### B. 2,2,2-Trifluoro-1-[7-methyl-1-(2-pyrrolidin-1-yl-ethyl)-1H-indol-3-yl]-ethanone

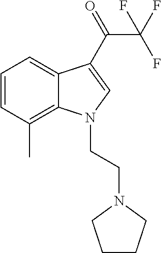

The title compound is prepared in a similar manner as described in Example 1F using 7-methyl-1-(2-pyrrolidin-1-yl-ethyl)-1H-indole as the starting material.

¹H NMR (300 MHz, CDCl₃) δ 8.3 (d, 1H), 8.1 (d, 1H), 7.3 (m, 1H), 7.1 (m, 1H), 5.0 (t, 2H), 3.5 (t, 2H), 2.7 (s, 3H), 1.6 (m, 8H).

MS m/z: [M+H]⁺=229.

### C. 7-Methyl-1-(2-pyrrolidin-1-yl-ethyl)-1H-indole-3-carboxylic acid

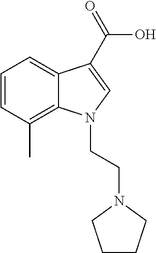

The title compound is prepared in a similar manner as described in Example 4C using 2,2,2-trifluoro-1-[7-methyl-1-(2-pyrrolidin-1-yl-ethyl)-1H-indol-3-yl]-ethanone as the starting material.

¹H NMR (300 MHz, DMSO-d6) δ 12.1 (bs, 1H), 8.2 (d, 1H), 7.95 (d, 1H), 7.2 (m, 1H), 7.0 (m, 1H), 4.8 (t, 2H), 3.5 (m, 4H), 3.0 (t, 2H), 2.7 (s, 3H), 1.9 (m, 4H).

MS m/z: [M+H]⁺=273.

### D. 2,2,2-Trifluoro-N-(4-fluoro-3-{1-[7-methyl-1-(2-pyrrolidin-1-yl-ethyl)-1H-indole-3-carbonyl]-piperidin-4-yl}-benzyl)-acetamide

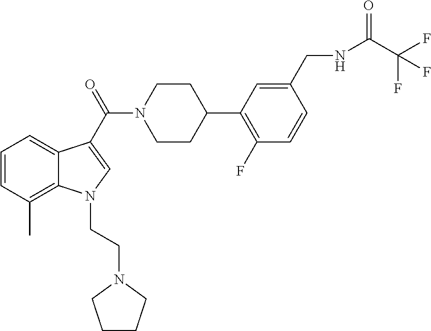

The title compound is prepared in a similar manner as described in Example 2I using 7-methyl-1-(2-pyrrolidin-1-yl-ethyl)-1H-indole-3-carboxylic acid and 2,2,2-trifluoro-N-(4-fluoro-3-piperidin-4-yl-benzyl)-acetamide hydrochloride as the starting materials.

¹H NMR (300 MHz, CDCl₃) δ 7.6 (m, 2H), 7.2-7.0 (m, 5H), 4.9 (t, 2H), 4.6 (bs, 1H), 4.5 (m, 2H), 3.7 (bs, 2H), 3.4 (m, 2H), 3.3-3.0 (m, 5H), 2.8 (s, 3H), 2.6 (bs, 2H), 2.2 (m, 4H), 1.9 (m, 2H), 1.8 (m, 2H);

MS m/z: [M+H]⁺=559.

### E. [4-(5-Aminomethyl-2-fluoro-phenyl)-piperidin-1-yl]-[7-methyl-1-(2-pyrrolidin-1-yl-ethyl)-1H-indol-3-yl]-methanone dihydrochloride

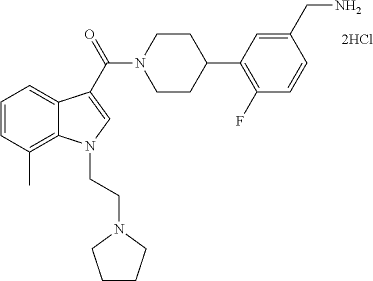

The title compound is prepared in a similar manner as described in Example 1K using 2,2,2-trifluoro-N-(4-fluoro-3-{1-[7-methyl-1-(2-pyrrolidin-1-yl-ethyl)-1H-indole-3-carbonyl]-piperidin-4-yl}-benzyl)-acetamide as the starting material.

¹H NMR (300 MHz, DMSO-d6) δ 11.4 (bs, 1H), 8.4 (bs, 2H), 7.8 (s, 1H), 7.6 (m, 1H), 7.5 (m, 1H), 7.4 (m, 1H), 7.2 (m, 1H), 7.0 (m, 2H), 4.8 (t, 2H), 4.4 (m, 2H), 4.0 (t, 2H), 3.6 (m, 4H), 3.2-3.0 (m, 5H), 2.8 (s, 3H), 2.0-1.8 (m, 8H);

MS m/z: [M+H]⁺=463.

### Example 14

### [4-(5-Aminomethyl-2-fluoro-phenyl)-piperidin-1-yl]-[7-difluoromethoxy-1-(2-methoxy-ethyl)-1H-indol-3-yl]-methanone hydrochloride

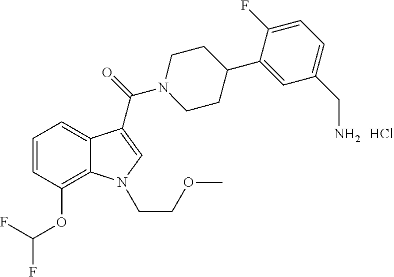

### A. 7-Difluoromethoxy-1H-indole-3-carboxylic acid methyl ester

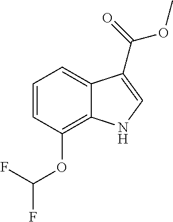

N-(2-Difluoromethoxy-phenyl)-hydroxylamine is prepared according to the procedure by Evans, D. A. et al., Org. Lett., 2006, vol. 8, pp. 3351-3354 using 1-(difluoromethoxy)-2-nitrobenzene as the starting material. The crude product is used for the next step without any purification.

The title compound is prepared in a similar manner as described in Example 11A using N-(2-difluoromethoxy-phenyl)-hydroxylamine as the starting material. The crude mixture is used for the next step without any purification.

### B. 7-Difluoromethoxy-1-(2-methoxy-ethyl)-1H-indole-3-carboxylic acid methyl ester

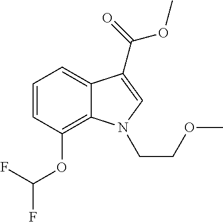

The title compound is prepared in a similar manner as described in Example 2F using 7-difluoromethoxy-1H-indole-3-carboxylic acid methyl ester and 2-methoxyethyl bromide as the starting materials.

¹H NMR (300 MHz, CD₃CN) δ 7.96 (d, 1H), 7.84 (s, 1H), 7.16 (q, 1H), 6.99 (m, 1H), 6.88 (t, 1H), 4.53 (t, 1H), 3.83 (s, 3H), 3.69 (t, 2H), 3.23 (s, 3H).

### C. 7-Difluoromethoxy-1-(2-methoxy-ethyl)-1H-indole-3-carboxylic acid

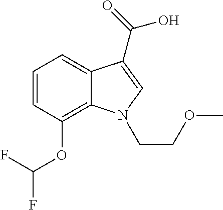

The title compound is prepared in a similar manner as described in Example 11C with 7-difluoromethoxy-1-(2-methoxy-ethyl)-1H-indole-3-carboxylic acid methyl ester as the starting material.

¹H NMR (300 MHz, DMSO-d6) δ 8.17 (d, 1H), 7.58 (s, 1H), 7.30 (t, 1H), 6.99 (t, 1H), 6.86-6.84 (m, 1H), 4.45 (t, 2H), 3.64 (t, 2H), 3.22 (s, 3H).

### D. N-(3-{1-[7-Difluoromethoxy-1-(2-methoxy-ethyl)-1H-indole-3-carbonyl]-piperidin-4-yl}-4-fluoro-benzyl)-2,2,2-trifluoro-acetamide

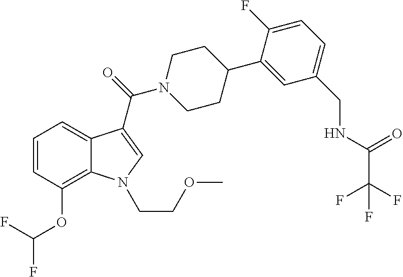

The title compound is prepared in a similar manner as described in Example 6E with 7-difluoromethoxy-1-(2-methoxy-ethyl)-1H-indole-3-carboxylic acid and 2,2,2-trifluoro-N-(4-fluoro-3-piperidin-4-yl-benzyl)-acetamide hydrochloride as the starting materials.

¹H NMR (300 MHz, CD₃CN) δ 8.22 (br s, 1H), 7.61-7.59 (m, 1H), 7.48 (s, 1H), 7.28-7.25 (m, 1H), 7.17-6.95 (m, 4H), 6.89 (t, 1H), 4.54-4.44 (m, 4H), 4.37 (d, 2H), 3.69 (t, 2H), 3.23 (s, 3H), 3.18-3.01 (m, 3H), 1.84-1.62 (m, 4H).

### E. [4-(5-Aminomethyl-2-fluoro-phenyl)-piperidin-1-yl]-[7-difluoromethoxy-1-(2-methoxy-ethyl)-1H-indol-3-yl]-methanone hydrochloride

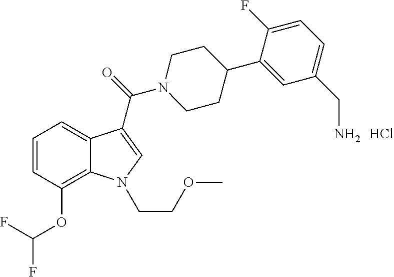

The title compound is prepared in a similar manner as described in Example 3B with N-(3-{1-[7-difluoromethoxy-1-(2-methoxy-ethyl)-1H-indole-3-carbonyl]-piperidin-4-yl}-4-fluoro-benzyl)-2,2,2-trifluoro-acetamide as the starting material.

¹H NMR (300 MHz, DMSO-d6) δ 8.17 (br s, 2H), 7.7 (m, 1H), 7.59-7.53 (m, 2H), 7.35 (m, 2H), 7.26-7.20 (m, 1H), 7.16-7.10 (m, 1H), 6.99 (m, 1H), 4.54-4.51 (m, 2H), 4.44-4.40 (m, 2H), 4.01 (m, 2H), 3.68 (m, 2H), 3.22 (s, 3H), 3.17-3.13 (m, 3H), 1.83-1.60 (m, 4H).

### Example 15

### [4-(5-Aminomethyl-2-fluoro-phenyl)-piperidin-1-yl]-(1-phenethyl-1H-indol-3-yl)-methanone hydrochloride

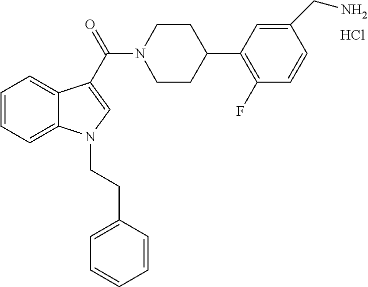

### A. 2,2,2-Trifluoro-N-{4-fluoro-3-[1-(1-phenethyl-1H-indole-3-carbonyl)-piperidin-4-yl]-benzyl}-acetamide

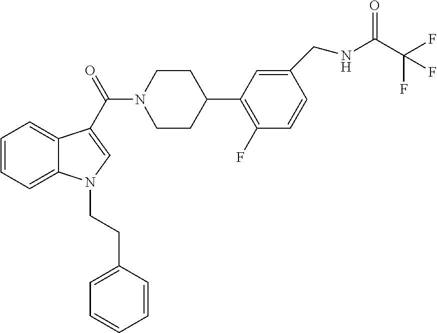

The title compound is prepared in a similar manner as described in Example 2I using 1-phenethyl-1H-indole-3-carboxylic acid and 2,2,2-trifluoro-N-(4-fluoro-3-piperidin-4-yl-phenyl)-acetamide hydrochloride as the starting materials.

¹H NMR (300 MHz, CDCl₃) δ 7.8 (d, 1H), 7.4 (d, 1H), 7.3-7.0 (m, 11H), 6.9 (bs, 1H), 4.5 (t, 2H), 4.4 (m, 4H), 3.2 (t, 2H), 3.0 (m, 3H), 1.9 (m, 2H), 1.75 (m, 2H);

MS m/z: [M+H]⁺=552.

### B. [4-(5-Aminomethyl-2-fluoro-phenyl)-piperidin-1-yl]-(1-phenethyl-1H-indol-3-yl)-methanone hydrochloride

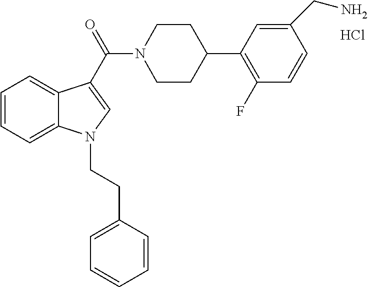

The title compound is prepared in a similar manner as described in Example 1K using 2,2,2-trifluoro-N-{4-fluoro-3-[1-(1-phenethyl-1H-indole-3-carbonyl)-piperidin-4-yl]-benzyl}-acetamide as the starting material.

¹H NMR (300 MHz, DMSO-d6) δ 8.2 (bs, 2H), 7.7 (d, 1H), 7.6 (d, 1H), 7.6 (m, 2H), 7.4 (m, 1H), 7.2 (m, 8H), 4.5 (t, 2H), 4.3 (m, 2H), 4.0 (t, 2H), 3.1 (m, 5H), 1.8 (m, 2H), 1.6 (m, 2H);

MS m/z: [M+H]+=456.

### Example 16

### [4-(5-Aminomethyl-2-fluoro-phenyl)-piperidin-1-yl]-[1-(3-fluoro-propyl)-7-methyl-1H-indol-3-yl]-methanone hydrochloride

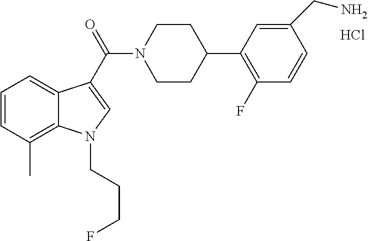

### A. 1-(3-Fluoro-propyl)-7-methyl-1H-indole

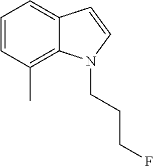

The title compound is prepared in a similar manner as described in Example 1E using 7-methylindole and 1-bromo-3-fluoro-propane as the starting materials.

¹H NMR (300 MHz, CDCl₃) δ 7.5 (d, 1H), 7.1-6.9 (m, 3H), 6.5 (d, 1H), 4.3 (t, 2H), 2.7 (s, 3H), 2.2 (m, H), 2.1 (m, 1H);

MS m/z: [M+H]⁺=192.

### B. 2,2,2-Trifluoro-1-[1-(3-fluoro-propyl)-7-methyl-1H-indol-3-yl]-ethanone

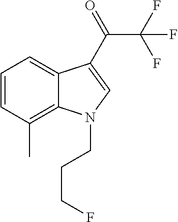

The title compound is prepared in a similar manner as described in Example 1F using 1-(3-fluoro-propyl)-7-methyl-1H-indole as the starting material.

¹H NMR (300 MHz, CDCl₃) δ 8.3 (d, 1H), 7.9 (d, 1H), 7.3 (m, 1H), 7.1 (m, 1H), 4.6 (m, 3H), 4.4 (t, 1H), 2.7 (s, 3H), 2.3 (m, 1H), 2.2 (m, 1H).

MS m/z: [M+H]⁺=288.

C-1. 1-(3-Fluoro-propyl)-7-methyl-1H-indole-3-carboxylic acid and C-2. 1-(3-Hydroxy-propyl)-7-methyl-1H-indole-3-carboxylic acid

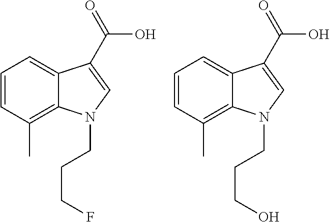

The title compounds are prepared in a similar manner as described in Example 4C using 2,2,2-trifluoro-1-[1-(3-fluoro-propyl)-7-methyl-1H-indol-3-yl]-ethanone as the starting material.

MS m/z: [M+H]⁺=234, 236 The crude 1:1 mixture is used in the next step.

### D-1. 2,2,2-Trifluoro-N-(4-fluoro-3-{1-[1-(3-fluoro-propyl)-7-methyl-1H-indole-3-carbonyl]-piperidin-4-yl}-benzyl)-acetamide and D-2. 2,2,2-Trifluoro-N-(4-fluoro-3-{1-[1-(3-hydroxy-propyl)-7-methyl-1H-indole-3-carbonyl]-piperidin-4-yl}-benzyl)-acetamide

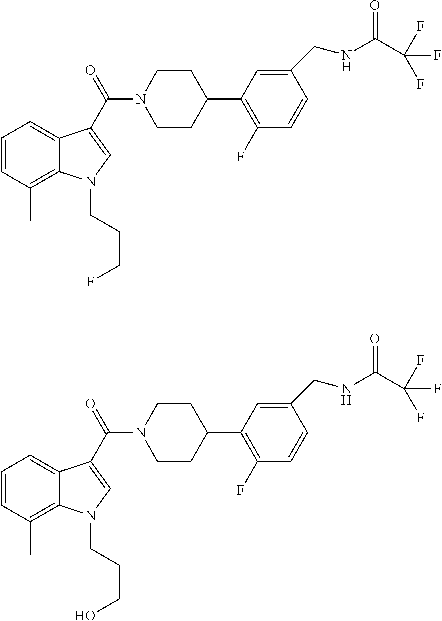

The title compounds are prepared in a similar manner as described in Example 2I using a mixture of 1-(3-fluoro-propyl)-7-methyl-1H-indole-3-carboxylic acid and 1-(3-hydroxy-propyl)-7-methyl-1H-indole-3-carboxylic acid and 2,2,2-trifluoro-N-(4-fluoro-3-piperidin-4-yl-phenyl)-acetamide hydrochloride as the starting materials.

D-1 ¹H NMR (300 MHz, CDCl₃) δ 7.6 (d, 1H), 7.4 (s, 1H), 7.2-7.0 (m, 5H), 6.8 (bs, 1H), 4.5 (m, 7H), 4.4 (t, 1H), 3.1 (m, 3H), 2.7 (s, 3H), 2.3 (m, 1H), 2.2 (m, 1H), 1.9 (m, 2H), 1.8 (m, 2H). MS m/z: [M+H]⁺=522.

D-2 ¹H NMR (300 MHz, CDCl₃) δ 7.6 (d, 1H), 7.5 (s, 1H), 7.2-7.0 (m, 5H), 6.9 (bs, 1H), 4.5 (m, 6H), 3.6 (t, 2H), 3.1 (m, 3H), 2.7 (s, 3H), 2.0 (m, 3H), 1.9 (m, 2H), 1.8 (m, 2H). LCMS m/z: [M+H]⁺=520.

### E. [4-(5-Aminomethyl-2-fluoro-phenyl)-piperidin-1-yl]-[1-(3-fluoro-propyl)-7-methyl-1H-indol-3-yl]-methanone hydrochloride

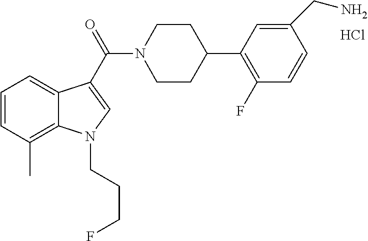

The title compound is prepared in a similar manner as described in Example 1K using 2,2,2-trifluoro-N-(4-fluoro-3-{1-[1-(3-fluoro-propyl)-7-methyl-1H-indole-3-carbonyl]-piperidin-4-yl}-benzyl)-acetamide as the starting material.

¹H NMR (300 MHz, DMSO-d6) δ 8.3 (bs, 2H), 7.7 (s, 1H), 7.6 (m, 2H), 7.4 (m, 1H), 7.2 (m, 1H), 7.0 (m, 2H), 4.6 (m, 4H), 4.4 (m, 2H), 4.0 (t, 2H), 3.1 (m, 3H), 2.7 (s, 3H), 2.2 (m, 1H), 2.1 (m, 1H), 1.8 (m, 2H), 1.75 (m, 2H);

MS m/z: [M+H]⁺=426.

### Example 17

### [4-(5-Aminomethyl-2-fluoro-phenyl)-piperidin-1-yl]-(1-phenethyl-1H-indol-3-yl)-methanone hydrochloride

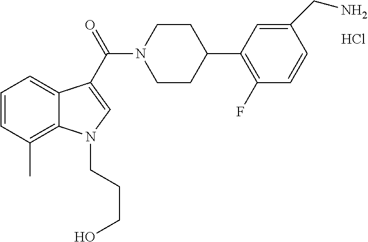

The title compound is prepared in a similar manner as described Example 1K using 2,2,2-trifluoro-N-(4-fluoro-3-{1-[1-(3-hydroxy-propyl)-7-methyl-1H-indole-3-carbonyl]-piperidin-4-yl}-benzyl)-acetamide as the starting material.

¹H NMR (300 MHz, DMSO-d6) δ 8.2 (bs, 2H), 7.6 (s, 1H), 7.6 (m, 2H), 7.4 (m, 1H), 7.2 (m, 1H), 7.0 (m, 2H), 4.4 (m, 4H), 4.0 (t, 2H), 3.4 (m, 3H), 3.2 (m, 3H), 2.7 (s, 3H), 1.9-1.6 (m, 6H);

MS m/z: [M+H]⁺=424.

### Example 18

### 3-[4-(5-Aminomethyl-2-fluoro-phenyl)-piperidine-1-carbonyl]-1-(2-methoxy-ethyl)-1H-indole-7-carbonitrile

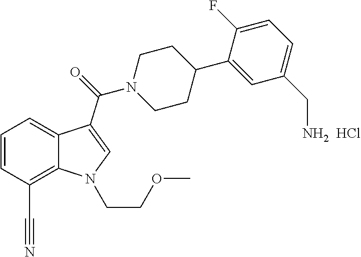

### A. 7-Cyano-1-(2-methoxy-ethyl)-1H-indole-3-carboxylic acid

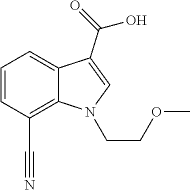

The title compound is prepared according to the procedure of WO2005/040133 (pp. 152) using 7-bromo-1-(2-methoxy-ethyl)-1H-indole-3-carboxylic acid (example 6A) and copper cyanide (I) as the starting materials. The crude mixture is used for the next step without any purification.

### B. N-(3-{1-[7-Cyano-1-(2-methoxy-ethyl)-1H-indole-3-carbonyl]-piperidin-4-yl}-4-fluoro-benzyl)-2,2,2-trifluoro-acetamide

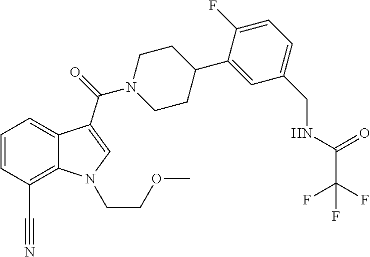

The title compound is prepared in a similar manner as described in Example 6E using 7-cyano-1-(2-methoxy-ethyl)-1H-indole-3-carboxylic acid and 2,2,2-trifluoro-N-(4-fluoro-3-piperidin-4-yl-benzyl)-acetamide hydrochloride as the starting materials.

¹H NMR (300 MHz, CD₃CN) δ 8.32 (br s, 1H), 8.06 (d, 1H), 7.62-7.59 (m, 2H), 7.27-7.23 (m, 2H), 7.16-7.12 (m, 1H), 7.05-6.98 (m, 1H), 4.66 (t, 2H), 4.46 (br d, 2H), 4.37 (m, 2H), 3.76 (t, 2H), 3.24 (s, 3H), 3.18-2.98 (m, 3H), 1.84-1.62 (m, 4H).

### C. 3-[4-(5-Aminomethyl-2-fluoro-phenyl)-piperidine-1-carbonyl]-1-(2-methoxy-ethyl)-1H-indole-7-carbonitrile hydrochloride

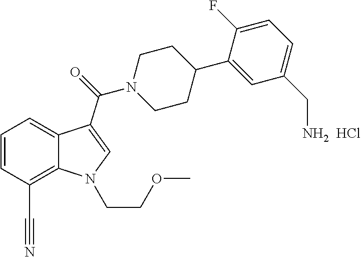

The title compound is prepared in a similar manner as described in Example 3B using N-(3-{1-[7-cyano-1-(2-methoxy-ethyl)-1H-indole-3-carbonyl]-piperidin-4-yl}-4-fluoro-benzyl)-2,2,2-trifluoro-acetamide as the starting material.

¹H NMR (300 MHz, DMSO-d6) δ 8.32 (br s, 3H), 8.09 (d, 1H), 7.90 (s, 1H), 7.73 (d, 1H), 7.58 (m, 1H), 7.40-7.19 (m, 3H), 4.70 (t, 2H), 4.41 (br d, 2H), 4.00 (m, 2H), 3.76 (t, 2H), 3.23 (s, 3H), 3.17-3.01 (m, 4H), 1.81-1.63 (m, 4H).

### Example 19

### [4-(5-Aminomethyl-2-fluoro-phenyl)-piperidin-1-yl]-[7-cyclobutyl-1-(2-methoxy-ethyl)-1H-indol-3-yl]-methanone hydrochloride

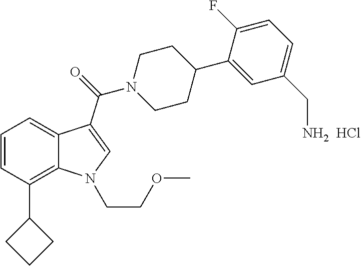

### A. 7-Cyclobutyl-1-(2-methoxy-ethyl)-1H-indole

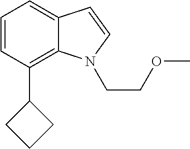

The title compound is prepared in a similar manner as described in Example 6B using cyclobutylboronic acid as the starting material.

¹H NMR (300 MHz, CD₃CN) δ 7.41-7.38 (m, 1H), 7.18-7.12 (m, 2H), 7.01 (t, 1H), 6.42 (m, 1H), 4.45 (t, 2H), 4.15-4.01 (m, 1H), 3.62 (t, 2H), 3.22 (s, 3H), 2.41-2.20 (m, 4H), 2.08-1.95 (m, 2H).

### B. 1-[7-Cyclobutyl-1-(2-methoxy-ethyl)-1H-indol-3-yl]-2,2,2-trifluoro-ethanone

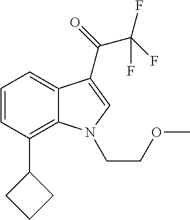

The title compound is prepared in a similar manner as described in Example 2G using 7-cyclobutyl-1-(2-methoxy-ethyl)-1H-indole as the starting material.

¹H NMR (300 MHz, CD₃CN) δ 8.19 (d, 1H), 8.13 (m, 1H), 7.41 (m, 1H), 7.35-7.30 (m, 1H), 4.58 (t, 2H), 4.10 (quin, 1H), 3.71 (t, 2H), 3.24 (s, 3H), 2.42-2.21 (m, 4H), 2.12-1.96 (m, 1H), 1.94-1.82 (m, 1H).

### C. 7-Cyclobutyl-1-(2-methoxy-ethyl)-1H-indole-3-carboxylic acid

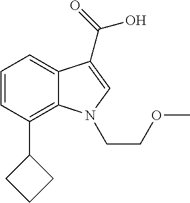

The title compound is prepared in a similar manner as described in Example 2H using 1-[7-cyclobutyl-1-(2-methoxy-ethyl)-1H-indol-3-yl]-2,2,2-trifluoro-ethanone as the starting material.

¹H NMR (300 MHz, DMSO-d6) δ 11.95 (br s, 1H), 7.96-7.93 (m, 2H), 7.24 (m, 1H), 7.16 (m, 1H), 4.53 (t, 2H), 4.07 (quin, 1H), 3.66 (t, 2H), 3.22 (s, 3H), 2.38-2.29 (m, 2H), 2.26-2.17 (m, 2H), 2.08-1.96 (m, 1H), 1.89-1.80 (m, 1H).

### D. N-(3-{1-[7-Cyclobutyl-1-(2-methoxy-ethyl)-1H-indole-3-carbonyl]-piperidin-4-yl}-4-fluoro-benzyl)-2,2,2-trifluoro-acetamide

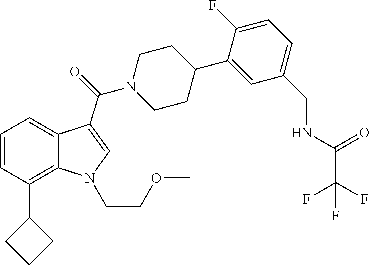

The title compound is prepared in a similar manner as described in Example 6E using 7-cyclobutyl-1-(2-methoxy-ethyl)-1H-indole-3-carboxylic acid and 2,2,2-trifluoro-N-(4-fluoro-3-piperidin-4-yl-benzyl)-acetamide hydrochloride as the starting materials.

¹H NMR (300 MHz, CD₃CN) δ 7.96 (br s, 1H), 7.58 (d, 1H), 7.41 (m, 1H), 7.28-7.23 (m, 2H), 7.18-7.10 (m, 2H), 7.06-7.00 (m, 1H), 4.50-4.40 (m, 4H), 4.38 (m, 2H), 4.08 (quin, 1H), 3.65 (m, 2H), 3.22 (s, 3H), 3.18-3.00 (m, 3H), 2.40-2.20 (m, 4H), 2.08-1.99 (m, 1H), 1.84-1.62 (m, 5H).

### E. [4-(5-Aminomethyl-2-fluoro-phenyl)-piperidin-1-yl]-[7-cyclobutyl-1-(2-methoxy-ethyl)-1H-indol-3-yl]-methanone hydrochloride

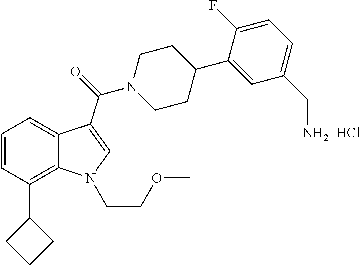

The title compound is prepared in a similar manner as described in Example 3B using N-(3-{1-[7-cyclobutyl-1-(2-methoxy-ethyl)-1H-indole-3-carbonyl]-piperidin-4-yl}-4-fluoro-benzyl)-2,2,2-trifluoro-acetamide as the starting material.

¹H NMR (300 MHz, DMSO-d6) δ 8.31 (br s, 2H), 7.62 (s, 1H), 7.57 (d, 2H), 7.40-7.35 (m, 1H), 7.25-7.19 (m, 2H), 7.15-7.10 (m, 1H), 4.51 (t, 2H), 4.42 (br d, 2H), 4.13-3.98 (m, 3H), 3.65 (t, 2H), 3.21 (s, 3H), 3.15-2.98 (m, 3H), 2.39-2.30 (m, 2H), 2.27-2.17 (m, 2H), 2.09-1.97 (m, 1H), 1.90-1.61 (m, 5H).

### Example 20

### [4-(5-Aminomethyl-2-fluoro-phenyl)-piperidin-1-yl]-[1-(2-hydroxy-ethyl)-7-methyl-1H-indol-3-yl]-methanone hydrochloride

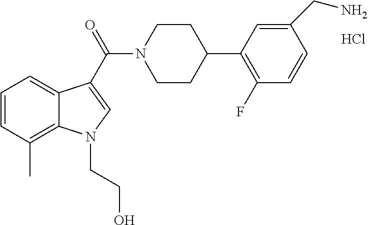

### A-1. 2-(7-methyl-indol-1-yl)ethanol and A-2. 2-[2-(7-methyl-indol-1-yl)-ethoxy]-ethanol

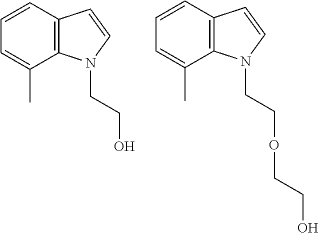

The title compounds are prepared in a similar manner as described in Example 1E using 7-methylindole and 2-bromoethanol as the starting materials.

A-1 ¹H NMR (300 MHz, CDCl₃) δ 7.5 (d, 1H), 7.2 (d, 1H), 7.0 (m, 2H), 6.5 (d, 1H), 4.5 (t, 2H), 3.9 (t, 2H), 2.7 (s, 3H). MS m/z: [M+H]⁺=176.

A-2 ¹H NMR (300 MHz, CDCl₃) δ 7.5 (d, 1H), 7.2 (d, 1H), 7.0 (m, 2H), 6.5 (d, 1H), 4.5 (t, 2H), 3.8 (t, 2H), 3.6 (m, 2H), 3.4 (m, 2H), 2.7 (s, 3H). MS m/z: [M+H]⁺=220.

### B. Trifluoro-acetic acid 2-[7-methyl-3-(2,2,2-trifluoro-acetyl)indol-1-yl]-ethyl ester

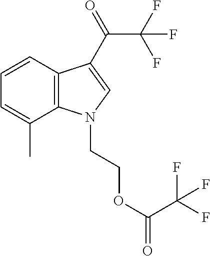

The title compound is prepared in a similar manner as described in Example 1F using 2-(7-methyl-indol-1-yl)ethanol as the starting material.

¹H NMR (300 MHz, CDCl₃) δ 8.3 (d, 1H), 8.0 (s, 1H), 7.3 (m, 1H), 7.1 (m, 1H), 4.6 (t, 2H), 4.0 (t, 2H), 2.7 (s, 3H);

MS m/z: [M+H]⁺=367.

### C. 1-(2-Hydroxy-ethyl)-7-methyl-1H-indole-3-carboxylic acid

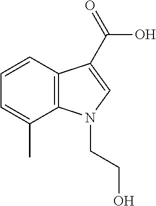

The title compound is prepared in a similar manner as described in Example 4C using trifluoro-acetic acid 2-[7-methyl-3-(2,2,2-trifluoro-acetyl)-indol-1-yl]-ethyl ester as the starting material.

¹H NMR (300 MHz, DMSO-d6) δ 11.9 (s, 1H), 8.0 (s, 1H), 7.9 (d, 1H), 7.1 (m, 1H), 6.9 (m, 1H), 5.0 (t, 1H), 4.5 (t, 2H), 3.7 (t, 2H), 2.7 (s, 3H).

MS m/z: [M+H]⁺=220.

### D. 2,2,2-Trifluoro-N-(4-fluoro-3-{1-[1-(2-hydroxy-ethyl)-7-methyl-1H-indole-3-carbonyl]-piperidin-4-yl}-benzyl)-acetamide

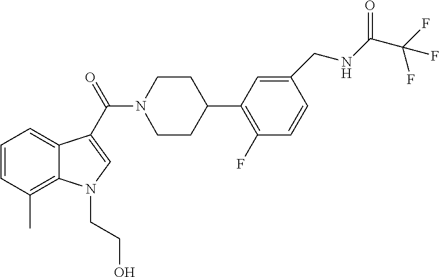

The title compound is prepared in a similar manner as described in Example 2I using 1-(2-hydroxy-ethyl)-7-methyl-1H-indole-3-carboxylic acid and 2,2,2-trifluoro-N-(4-fluoro-3-piperidin-4-yl-phenyl)-acetamide hydrochloride as the starting materials.

¹H NMR (300 MHz, DMSO-d6) δ 10.0 (s, 1H), 7.6 (s, 1H), 7.5 (d, 1H), 7.2 (d, 2H), 7.0 (m, 2H), 5.0 (t, 1H), 4.4 (m, 6H), 3.7 (m, 2H), 3.1 (m, 3H), 2.7 (s, 3H), 1.8 (m, 2H), 1.65 (m, 2H).

MS m/z: [M+H]⁺=506.

### E. [4-(5-Aminomethyl-2-fluoro-phenyl)-piperidin-1-yl]-[1-(2-hydroxy-ethyl)-7-methyl-1H-indol-3-yl]-methanone hydrochloride

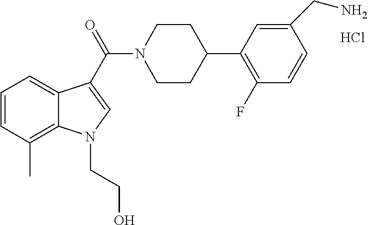

The title compound is prepared in a similar manner as described in Example 1K using 2,2,2-trifluoro-N-(4-fluoro-3-{1-[1-(2-hydroxy-ethyl)-7-methyl-1H-indole-3-carbonyl]-piperidin-4-yl}-benzyl)-acetamide as the starting material.

¹H NMR (300 MHz, DMSO-d6) δ 8.2 (bs, 2H), 7.6 (s, H), 7.6 (m, 2H), 7.4 (m, H), 7.2 (m, H), 7.0 (m, 2H), 4.4 (m, 5H), 4.0 (m, 2H), 3.7 (m, 2H), 3.1 (m, 3H), 2.7 (s, 3H), 1.8 (m, 2H), 1.7 (m, 2H).

MS m/z: [M+H]⁺=410.

### Example 21

### [4-(5-Aminomethyl-2-fluoro-phenyl)-piperidin-1-yl]-{1-[2-(2-hydroxy-ethoxy)-ethyl]-7-methyl-1H-indol-3-yl}-methanone hydrochloride

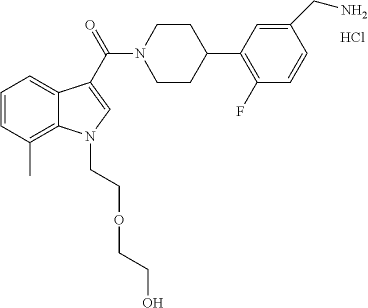

### A. Trifluoro-acetic acid 2-{2-[7-methyl-3-(2,2,2-trifluoro-acetyl)-indol-1-yl]-ethoxy}-ethyl ester

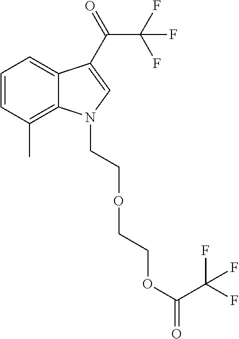

The title compound is prepared in a similar manner as described in Example 1F using 2-[2-(7-methyl-indol-1-yl)-ethoxy]-ethanol as the starting material.

¹H NMR (300 MHz, CDCl₃) δ 8.3 (d, 1H), 7.9 (s, 1H), 7.3 (m, 1H), 7.1 (m, 1H), 4.6 (t, 2H), 4.4 (t, 2H), 3.9 (t, 2H), 3.7 (t, 2H), 2.7 (s, 3H).

MS m/z: [M+H]⁺=412.

### B. 1-[2-(2-Hydroxy-ethoxy)-ethyl]-7-methyl-1H-indole-3-carboxylic acid

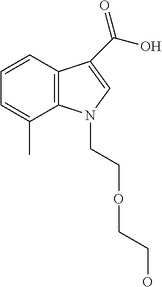

The title compound is prepared in a similar manner as described in Example 4C using trifluoro-acetic acid 2-{2-[7-methyl-3-(2,2,2-trifluoro-acetyl)-indol-1-yl]-ethoxy}-ethyl ester as the starting material.

¹H NMR (300 MHz, DMSO-d6) δ 11.9 (s, 1H), 8.0 (s, 1H), 7.9 (d, 1H), 7.1 (m, 1H), 6.9 (m, 1H), 4.6 (m, 3H), 3.8 (m, 2H), 3.4 (m, 4H), 2.7 (s, 3H).

MS m/z: [M+H]⁺=264.

### C. 2,2,2-Trifluoro-N-[4-fluoro-3-(1-{1-[2-(2-hydroxy-ethoxy)-ethyl]-7-methyl-1H-indole-3-carbonyl]-piperidin-4-yl)-benzyl}-acetamide

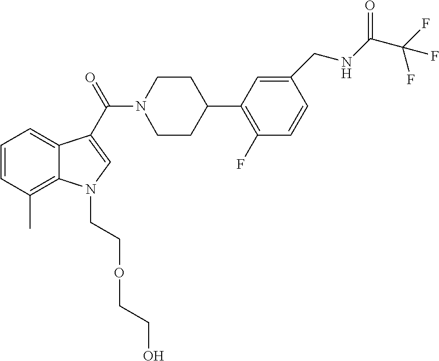

The title compound is prepared in a similar manner as described in Example 2I using 1-[2-(2-hydroxy-ethoxy)-ethyl]-7-methyl-1H-indole-3-carboxylic acid and 2,2,2-trifluoro-N-(4-fluoro-3-piperidin-4-yl-phenyl)-acetamide hydrochloride as the starting materials.

¹H NMR (300 MHz, CDCl₃) δ 7.6 (m, 2H), 7.2-7.0 (m, 5H), 6.8 (bs, 1H), 4.6 (m, 4H), 4.5 (d, 2H) 3.8 (t, 2H), 3.6 (m, 2H), 3.4 (m, 2H), 3.1 (m, 3H), 2.7 (s, 3H), 2.4 (m, H), 1.9 (m, 2H), 1.8 (m, 2H).

MS m/z: [M+H]⁺=550.

### D. [4-(5-Aminomethyl-2-fluoro-phenyl)-piperidin-1-yl]-{1-[2-(2-hydroxy-ethoxy)-ethyl]-7-methyl-1H-indol-3-yl}-methanone hydrochloride

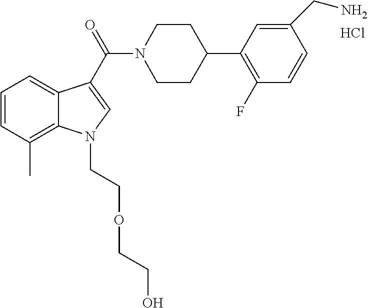

The title compound is prepared in a similar manner as described in Example 1K using 2,2,2-trifluoro-N-[4-fluoro-3-(1-{1-[2-(2-hydroxy-ethoxy)-ethyl]-7-methyl-1H-indole-3-carbonyl}-piperidin-4-yl)-benzyl]-acetamide as the starting material.

¹H NMR (300 MHz, DMSO-d6) δ 8.5 (bs, 2H), 7.7 (s, 1H), 7.6 (m, 2H), 7.4 (m, 1H), 7.2 (m, 1H), 7.0 (m, 2H), 4.6 (m, 2H), 4.4 (d, 2H), 4.0 (m, 2H), 3.7 (m, 3H), 3.1 (m, 3H), 2.7 (s, 3H), 1.8-1.6 (m, 4H).

MS m/z: [M+H]⁺=454.

### Example 22

### [4-(5-Aminomethyl-2-fluoro-phenyl)-piperidin-1-yl]-[1-(2-hydroxy-ethyl)-7-trifluoromethoxy-1H-indol-3-yl]-methanone hydrochloride

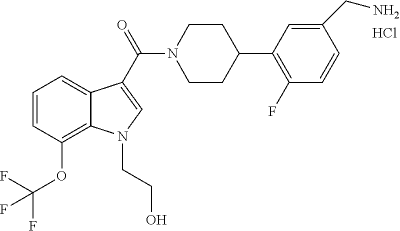

### A-1. 2-(7-Trifluoromethoxy-indol-1-yl)ethanol and A-2. 2-[2-(7-Trifluoromethoxy-indol-1-yl)-ethoxy]-ethanol

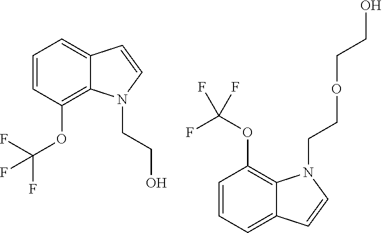

The title compounds are prepared in a similar manner as described in Example 1E using 7-trifluoromethoxy-1H-indole and 2-bromoethanol as the starting materials.

A-1 ¹H NMR (300 MHz, CDCl₃) δ 7.5 (d, 1H), 7.2 (d, 1H), 7.1 (m, 2H), 6.55 (d, 1H), 4.5 (t, 2H), 4.0 (m, 2H).

A-2 ¹H NMR (300 MHz, CDCl₃) δ 7.5 (d, 1H), 7.2 (d, 1H), 7.1 (m, 2H), 6.55 (d, 1H), 4.5 (t, 2H), 3.8 (t, 2H), 3.6 (m, 2H), 3.4 (m, 2H).

### B. 2,2,2-Trifluoro-1-[1-(2-hydroxy-ethyl)-7-trifluoromethoxy-1H-indol-3-yl]-ethanone

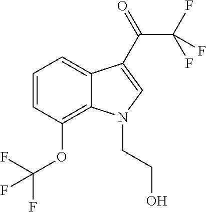

The title compound is prepared in a similar manner as described in Example 1F using 2-(7-trifluoromethoxy-indol-1-yl)-ethanol as the starting material.

¹H NMR (300 MHz, CDCl₃) δ 8.4 (d, 1H), 8.0 (d, 1H), 7.4 (m, 2H), 4.6 (t, 2H), 4.0 (t, 2H).

### C. 1-(2-Hydroxy-ethyl)-7-trifluoromethoxy-1H-indole-3-carboxylic acid

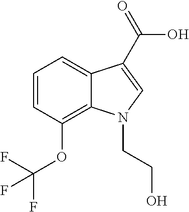

The title compound is prepared in a similar manner as described in Example 4C using 2,2,2-trifluoro-1-[1-(2-hydroxy-ethyl)-7-trifluoromethoxy-1H-indol-3-yl]-ethanone as the starting material.

¹H NMR (300 MHz, DMSO-d6) δ12.2 (s, 1H), 8.1 (m, 2H), 7.2 (m, 2H), 5.0 (m, H), 4.4 (t, 2H), 3.7 (t, 2H).

### D. 2,2,2-Trifluoro-N-(4-fluoro-3-{1-[1-(2-hydroxy-ethyl)-7-trifluoromethoxy-1H-indole-3-carbonyl]-piperidin-4-yl}-benzyl)-acetamide

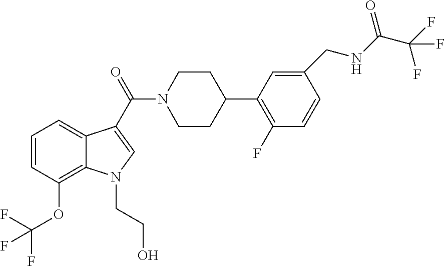

The title compound is prepared in a similar manner as described Example 2I using 1-(2-hydroxy-ethyl)-7-trifluoromethoxy-1H-indole-3-carboxylic acid and 2,2,2-trifluoro-N-(4-fluoro-3-piperidin-4-yl-phenyl)-acetamide hydrochloride as the starting materials.

¹H NMR (300 MHz, DMSO-d6) δ 10.0 (bs, 1H), 7.8 (s, 1H), 7.7 (m, 1H), 7.3 (m, 1H), 7.1 (m, 4H), 5.0 (t, 1H), 4.4 (m, 6H), 3.7 (m, 2H), 3.1 (m, 3H), 1.8 (m, 2H), 1.7 (m, 2H).

### E. [4-(5-Aminomethyl-2-fluoro-phenyl)-piperidin-1-yl]-[1-(2-hydroxy-ethyl)-7-trifluoromethoxy-1H-indol-3-yl]-methanone hydrochloride

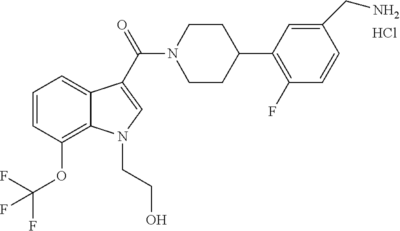

The title compound is prepared in a similar manner as described in Example 1K using 2,2,2-trifluoro-N-(4-fluoro-3-{1-[1-(2-hydroxy-ethyl)-7-trifluoromethoxy-1H-indole-3-carbonyl]-piperidin-4-yl}-benzyl)-acetamide as the starting material.

¹H NMR (300 MHz, DMSO-d6) δ 7.8 (s, 1H), 7.7 (m, 1H), 7.6 (m, 1H), 7.4 (m, 1H), 7.2 (m, 3H), 4.4 (m, 4H), 4.0 (m, 2H), 3.75 (m, 3H), 3.1 (m, 3H), 1.8-1.7 (m, 4H).

MS m/z: [M+H]⁺=480.

### Example 23

### (4-Aminomethyl-2-{1-[1-(2-methoxy-ethyl)-7-methyl-1H-indole-3-carbonyl]-piperidin-4-yl}-phenoxy)-acetic acid methyl ester hydrochloride

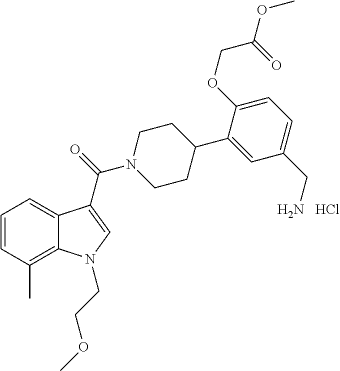

### A. 4-Aminomethyl-2-bromo-phenol hydrobromide

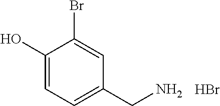

To a mixture of 4-hydroxybenzylamine (6.0 g, 48.7 mmol) in acetic acid (30 mL) is added HBr in acetic acid (33% wt, 24 mL) dropwise. After the addition is completed, bromine (25 mL, 48.7 mmol) in acetic acid (25 mL) is added dropwise. The resulting mixture is stirred at room temperature overnight. The precipitate is collected by suction filtration, washed with acetic acid and Et₂O, dried in vacuo to yield the product (8.05 g, 58%) as a white powder.

¹H NMR (300 MHz, DMSO-d6) δ 8.00 (br s, 3H), 7.65 (s, 1H), 7.30-7.20 (m, 1H), 7.05-6.85 (m, 1H), 4.05-3.75 (m, 2H).

### B (3-Bromo-4-hydroxy-benzyl)-carbamic acid tert-butyl ester

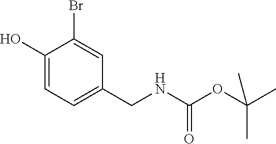

A mixture of 4-aminomethyl-2-bromo-phenol hydrobromide (5.0 g, 17.6 mmol), DIEA (6.1 mL, 35.3 mmol), and di-t-butyl dicarbonate (4.34 g, 19.4 mmol) in CH₂Cl₂ (50 mL) is stirred at r.t. overnight. The solution is washed with sat. NaHCO3, 10% aqueous citric acid, H₂O, and brine, dried over Na₂SO₄, filtered, and concentrated in vacuo. The crude material is purified on silica gel with CH₂Cl₂/MeOH (100/0 to 96/4) as eluent to give the product (3.54 g, 66%) as a brown oil.

¹H NMR (300 MHz, CDCl₃) δ 7.40 (s, 1H), 7.20-7.05 (m, 1H), 7.00-6.85 (m, 1H), 6.55 (s, 1H), 4.80 (br s, 1H), 4.30-4.05 (m, 2H);

LC Rt 0.85 min; MS 248 (M-54, 100%).

### C. [2-Bromo-4-(tert-butoxycarbonylamino-methyl)-phenoxy]-acetic acid methyl ester

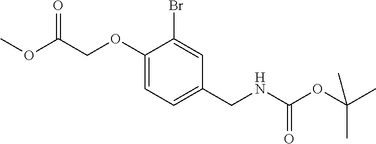

A mixture of (3-bromo-4-hydroxy-benzyl)-carbamic acid tert-butyl ester (2.46 g, 8.14 mmol), methyl bromoacetate (3.85 mL, 40.7 mmol), and Cs₂CO₃ (6.6 g, 20.35 mmol) in THF (20 mL) is stirred at r.t. overnight. The reaction mixture is filtered, and the filtrate is concentrated in vacuo. The crude material is purified on silica gel with heptanes/EtOAc (95/5 to 75/25) as eluent to give the crude product (3.54 g) as a white solid. This crude material is used in the next step without further purification.

### D. [4-(tert-Butoxycarbonylamino-methyl)-2-pyridin-4-yl-phenoxy]-acetic acid methyl ester

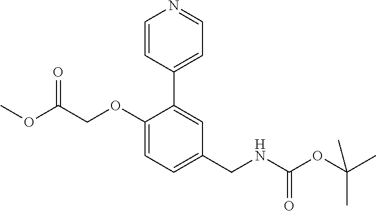

A mixture of [2-bromo-4-(tert-butoxycarbonylamino-methyl)-phenoxy]-acetic acid methyl ester (2.0 g, 5.34 mmol), pyridine-4-boronic acid (0.78 g, 6.41 mmol), Cs₂CO₃ (3.48 g, 10.68 mol), Pd(dppf)Cl₂.CH₂Cl₂ (0.29 g, 10% mol) in dioxane/H₂O (29 mL, 10/1) is heated at 80° C. for 4 h. The reaction mixture is cooled to r.t., and then concentrated in vacuo. The residue is partitioned between CH₂Cl₂ and H₂O. The two layers are separated, and the organic layer is washed with H₂O, and brine, dried over Na₂SO₄, filtered, and concentrated in vacuo. The crude material is purified on silica gel with CH₂Cl₂/MeOH (100/0 to 96/4) as eluent to give the product (1.17 g, 58%) as a light brown foam.

¹H NMR (300 MHz, CDCl₃) δ 8.64 (d, J=6.0 Hz, 2H), 7.52 (d, J=6 Hz, 2H), 7.26 (m, 2H), 6.84 (d, J=8.6 Hz), 4.86 (br s, 1H), 4.65 (s, 1H), 4.30 (d, J=5.7 Hz, 1H), 3.78 (s, 3H), 1.46 (s, 9H);

LC Rt 0.74 min; MS 373 (M+H, 100%).

### E. [4-(tert-Butoxycarbonylamino-methyl)-2-piperidin-4-yl-phenoxy]-acetic acid methyl ester

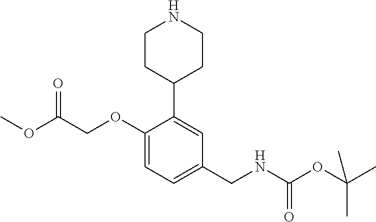

A mixture of [4-(tert-butoxycarbonylamino-methyl)-2-pyridin-4-yl-phenoxy]-acetic acid methyl ester and PtO₂ (30 mg) in MeOH (10 mL) and acetic acid (1 mL) is hydrogenated at 50-60 psi at r.t. for 3 h. The reaction mixture is filtered through Celite, and the filtrate is concentrated in vacuo. The residue is partitioned between CH₂Cl₂ and sat'd NaHCO₃. The two layers are separated and the organic layer is washed with H2O and brine, dried over Na₂SO₄, filtered, and concentrated in vacuo to give the crude material (299 mg) as a yellow foam. This crude material is used in the next step without further purification.

LC Rt 0.67 min; MS 379 (M+H, 100%).

### F. (4-(tert-Butoxycarbonylamino-methyl)-2-{1-[1-(2-methoxy-ethyl)-7-methyl-1H-indole-3-carbonyl]-piperidin-4-yl}-phenoxy)-acetic acid methyl ester

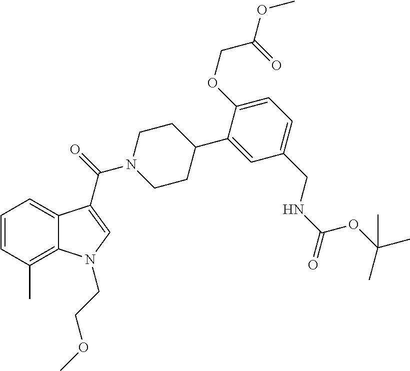

A mixture of 1-(2-methoxy-ethyl)-7-methyl-1H-indole-3-carboxylic acid (90 mg, 0.387 mmol), DIEA (134 μL, 0.77 mmol), [4-(tert-butoxycarbonylamino-methyl)-2-piperidin-4-yl-phenoxy]-acetic acid methyl ester (146 mg, 0.38 mmol), and EDCI (88 mg, 0.46 mmol) in CH₂Cl₂ (5 mL) is stirred at r.t. overnight. The mixture is partitioned between H₂O and CH₂Cl₂. The two layers are separated, and the organic layer is washed with 10% citric acid, sat. NaHCO₃, and brine, dried over Na₂SO₄, filtered, and concentrated in vacuo. The crude material is purified on silica gel with CH₂Cl₂/MeOH (100/0 to 96/4) as eluent to give the product (199 mg, 87%) as a light yellow foam.

¹H NMR (300 MHz, CDCl₃) δ 7.65-7.50 (m, 1H), 7.40 (s, 1H), 7.20-6.85 (m, 5H), 6.75-6.65 (m, 1H), 4.80 (br s, 1H), 4.75-4.35 (m, 6H), 4.30-4.10 (m, 2H), 3.75 (s, 3H), 3.70-3.60 (m, 2H), 3.40-3.20 (m, 4H), 3.15-2.85 (m, 2H), 2.70 (s, 3H), 2.00-1.50 (m, 4H), 1.40 (s, 9H);

LC Rt 1.02 min; MS 594 (M+H, 100%).

### G. (4-Aminomethyl-2-{1-[1-(2-methoxy-ethyl)-7-methyl-1H-indole-3-carbonyl]-piperidin-4-yl}-phenoxy)-acetic acid methyl ester hydrochloride

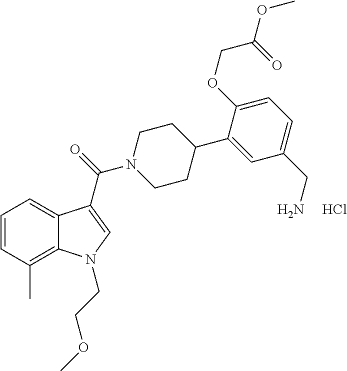

A mixture of (4-(tert-butoxycarbonylamino-methyl)-2-{1-[1-(2-methoxy-ethyl)-7-methyl-1H-indole-3-carbonyl]-piperidin-4-yl}-phenoxy)-acetic acid methyl ester (190 mg, 0.2 mmol) in 4 M HCl in dioxane (3 mL) is stirred at r.t. for 1 h. The mixture is concentrated in vacuo, and the residue is triturated with Et₂O (4×) to give the product (160 mg, 94%) as a beige powder.

¹H NMR (300 MHz, DMSO-d6) δ 8.13 (br s, 3H), 7.61 (s, 1H), 7.55 (d, J=7.5 Hz, 1H), 7.45-7.35 (m, 1H), 7.30-7.20 (m, 1H), 7.10-6.85 (m, 3H), 4.89 (s, 2H), 4.65-4.30 (m, 4H), 4.00-3.85 (m, 2H), 3.69 (s, 2H), 3.57 (s, 3H), 3.22 (s, 3H), 3.15-2.90 (m, 1H), 2.51 (s, 3H), 2.60-2.30 (m, 2H), 1.90-1.50 (m, 4H);

LC 0.72 min; MS 494 (M+H, 100%).

### Example 24

### (4-Aminomethyl-2-{1-[1-(2-methoxy-ethyl)-7-trifluoromethoxy-1H-indole-3-carbonyl]-piperidin-4-yl}-phenoxy)-acetic acid methyl ester hydrochloride

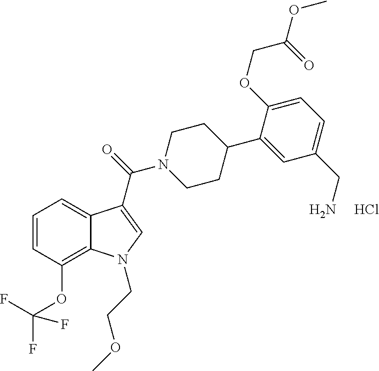

### A. (4-(tert-Butoxycarbonylamino-methyl)-2-{1-[1-(2-methoxy-ethyl)-7-trifluoromethoxy-1H-indole-3-carbonyl]-piperidin-4-yl}-phenoxy)-acetic acid methyl ester

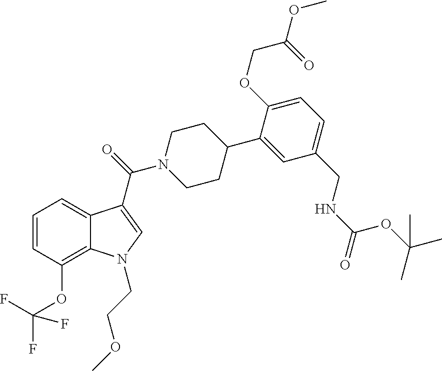

A mixture of 1-(2-methoxy-ethyl)-7-trifluoromethoxy-1H-indole-3-carboxylic acid (117 mg, 0.387 mmol), DIEA (134 μL, 0.77 mmol), [4-(tert-butoxycarbonylamino-methyl)-2-piperidin-4-yl-phenoxy]-acetic acid methyl ester (146 mg, 0.38 mmol), and EDCI (88 mg, 0.46 mmol) in CH₂Cl₂ (5 mL) is stirred at r.t. for 4 h. The mixture is partitioned between H₂O and CH₂Cl₂. The two layers are separated, and the organic layer is washed with 10% citric acid, sat. NaHCO₃, and brine, dried over Na₂SO₄, filtered, and concentrated in vacuo. The crude material is purified on silica gel with CH₂Cl₂/MeOH (100/0 to 96/4) as eluent to give the product (140 mg, 54%) as a light yellow foam.

¹H NMR (300 MHz, CDCl₃) δ 7.75-7.65 (m, 1H), 7.47 (s, 1H), 7.20-7.00 (m, 4H), 6.68 (d, J=8.2 Hz, 1H), 4.90-4.30 (m, 4H), 4.24 (d, J=5.5 Hz, 1H), 3.79 (s, 3H), 3.75-3.65 (m, 3H), 3.30 (s, 3H), 3.20-2.85 (m, 2H), 2.00-1.60 (m, 4H), 1.46 (s, 9H); LC Rt 1.09 min; MS 664 (M+H, 100%).

### B. (4-Aminomethyl-2-{1-[1-(2-methoxy-ethyl)-7-trifluoromethoxy-1H-indole-3-carbonyl]-piperidin-4-yl}-phenoxy)-acetic acid methyl ester hydrochloride

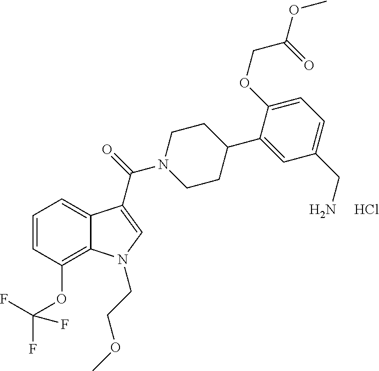

A mixture (4-(tert-butoxycarbonylamino-methyl)-2-{1-[1-(2-methoxy-ethyl)-7-trifluoromethoxy-1H-indole-3-carbonyl]-piperidin-4-yl}-phenoxy)-acetic acid methyl ester (140 mg, 0.21 mmol) in 4 M HCl in dioxane (3 mL) is stirred at r.t. for 1 h. The mixture is concentrated in vacuo, and the residue is triturated with Et₂O (4×) to give the product (114 mg, 90%) as a slightly yellow powder.

¹H NMR (300 MHz, DMSO-d6) δ 8.13 (br s, 3H), 7.79 (s, 1H), 7.75-7.65 (m, 1H), 7.41 (s, 1H), 7.30-7.10 (m, 3H), 7.00-6.90 (m, 1H), 4.90 (s, 2H), 4.60-4.30 (m, 3H), 4.05-3.85 (m, 2H), 3.69 (s, 2H), 3.60 (m, 1H), 3.22 (s, 3H), 3.20-2.95 (m, 2H), 2.00-1.50 (m, 4H);

LC 0.80 min; MS 564 (M+H, 100%).

### Example 25

### 2-(4-Aminomethyl-2-{1-[1-(2-methoxy-ethyl)-7-methyl-1H-indole-3-carbonyl]-piperidin-4-yl}-phenoxy)-N,N-dimethyl-acetamide hydrochloride

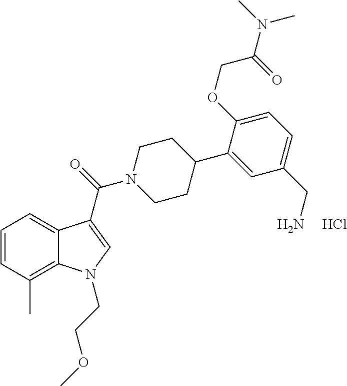

### A. [4-(tert-Butoxycarbonylamino-methyl)-2-pyridin-4-yl-phenoxy]-acetic acid

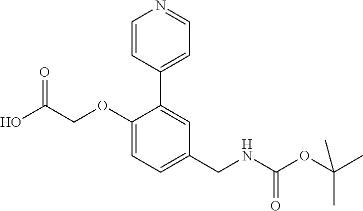

A mixture of [2-bromo-4-(tert-butoxycarbonylamino-methyl)-phenoxy]-acetic acid methyl ester (0.5 g, 1.34 mmol) in MeOH (5 mL) and 1.0 M NaOH (5 mL) is stirred at r.t. for 1 h. The reaction is concentrated in vacuo, and then partitioned between H₂O and Et₂O. The two layers are separated, and the aqueous is acidified to pH˜4 with 10% citric acid. The acidified aqueous layer is extracted with EtOAc (3×). The combined organic layers are washed with brine, dried ouver Na₂SO₄, filtered, and concentrated in vacuo to yield the product (420 mg, 87%) as a beige solid. This material is used in the next step without further purification.

LC 0.60 min; MS 359 (M+H, 100%).

### B. (4-Dimethylcarbamoylmethoxy-3-pyridin-4-yl-benzyl)-carbamic acid tert-butyl ester

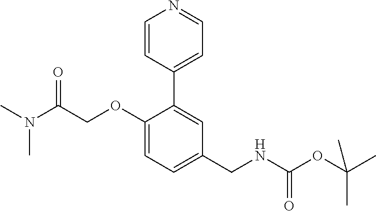

A mixture of [4-(tert-butoxycarbonylamino-methyl)-2-pyridin-4-yl-phenoxy]-acetic acid (420 mg, 1.17 mmol), DIEA (449 μL, 2.58 mmol), dimethylamine (2.0M in THF 649 μL, 1.29 mmol), and EDCI (270 mg, 1.40 mmol) in CH₂Cl₂ (10 mL) is stirred at r.t. overnight. The mixture is partitioned between sat. NaHCO3 and CH₂Cl₂. The two layers are separated, and the organic layer is washed with H₂O, and brine, dried over Na₂SO₄, filtered, and concentrated in vacuo. The crude material is purified on silica gel with CH₂Cl₂/MeOH (99/1 to 95/5) as eluent to give the product (140 mg, 30%) as a light yellow foam.

¹H NMR (300 MHz, CDCl₃) δ 8.60 (s, 2H), 7.60-7.40 (m, 3H), 7.05-6.90 (m, 2H), 4.90 (br s, 1H), 4.70 (s, 2H), 4.60-4.40 (m, 2H), 2.90 (s, 6H), 1.40 (s, 9H); LC Rt 0.62 min;

MS 386 (M+H, 100%).

### C. (4-Dimethylcarbamoylmethoxy-3-piperidin-4-yl-benzyl)-carbamic acid tert-butyl ester

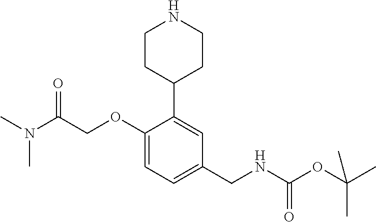

A mixture of (4-dimethylcarbamoylmethoxy-3-pyridin-4-yl-benzyl)-carbamic acid tert-butyl ester (135 mg, 0.35 mmol) and PtO₂ (50 mg) in MeOH (5 mL) and acetic acid (1 mL) is hydrogenated at 50-60 psi at r.t. for 3 h. The reaction mixture is filtered through Celite, and the filtrate is concentrated in vacuo. The residue is partitioned between CH₂Cl₂ and sat NaHCO₃. The two layers are separated, and the organic layer is washed with H₂O and brine, dried over Na₂SO₄, filtered, and concentrated in vacuo to give the crude material (54 mg) as a white foam. This crude material is used in the next step without further purification.

### D. (4-Dimethylcarbamoylmethoxy-3-{1-[1-(2-methoxy-ethyl)-7-methyl-1H-indole-3-carbonyl]-piperidin-4-yl}-benzyl)-carbamic acid tert-butyl ester

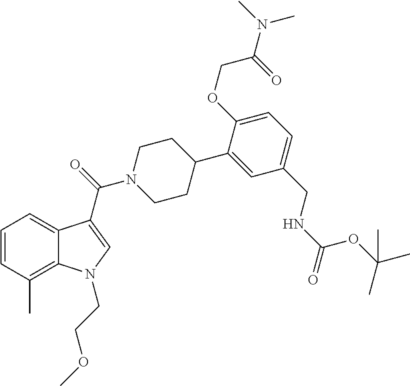

A mixture of 1-(2-methoxy-ethyl)-7-methyl-1H-indole-3-carboxylic acid (32 mg, 0.13 mmol), DIEA (48 μL, 0.27 mmol), (4-dimethylcarbamoylmethoxy-3-piperidin-4-yl-benzyl)-carbamic acid tert-butyl ester (54 mg, 0.13 mmol), and EDCI (32 mg, 0.16 mmol) in CH₂Cl₂ (5 mL) is stirred at r.t. overnight. The mixture is partitioned between H₂O and CH₂Cl₂. The two layers are separated, and the organic layer is washed with 10% citric acid, sat. NaHCO₃, and brine, dried over Na₂SO₄, filtered, and concentrated in vacuo to give a white foam. This crude material is used in the next step without further purification.

LC Rt 0.96 min; MS 607 (M+H, 100%).

### E. 2-(4-Aminomethyl-2-{1-[1-(2-methoxy-ethyl)-7-methyl-1H-indole-3-carbonyl]-piperidin-4-yl}-phenoxy)-N,N-dimethyl-acetamide hydrochloride

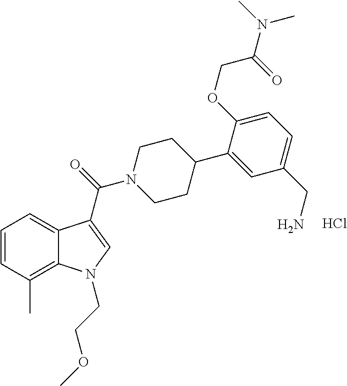

A mixture (4-dimethylcarbamoylmethoxy-3-{1-[1-(2-methoxy-ethyl)-7-methyl-1H-indole-3-carbonyl]-piperidin-4-yl}-benzyl)-carbamic acid tert-butyl ester (from the previous step) in 4 M HCl in dioxane (3 mL) is stirred at r.t. for 1 h. The mixture is concentrated in vacuo, and crude material is purified by RP-HPLC to yield the product (13 mg) as a slightly white powder.

¹H NMR (300 MHz, DMSO-d6) δ 8.02 (br s, 3H), 7.60 (s, 1H), 7.75 (d, J=6.6 Hz, 1H), 7.35 (d, J=1.8 Hz, 1H), 7.30-7.15 (m, 1H), 7.10-6.85 (m, 3H), 4.91 (s, 2H), 4.57 (t, J=7.0 Hz, 1H), 4.44 (br d, J=12.1 Hz, 1H), 3.94 (s, 2H), 3.67 (t, J=5.4 Hz, 2H), 3.15 (s, 3H), 3.05-2.90 (m, 4H), 2.83 (s, 3H), 2.67 (s, 3H), 2.60-2.40 (m, 1H), 1.95-1.50 (m, 4H);

LC 2.11 min; MS 507 (M+H, 100%).

### Example 26

### 2-(4-Aminomethyl-2-{1-[1-(2-methoxy-ethyl)-7-methyl-1H-indole-3-carbonyl]-piperidin-4-yl}-phenoxy)-N-methyl-acetamide hydrochloride

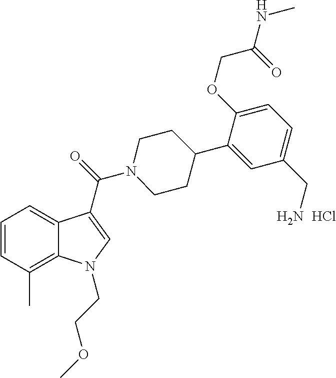

### A. (4-(tert-Butoxycarbonylamino-methyl)-2-{1-[1-(2-methoxy-ethyl)-7-methyl-1H-indole-3-carbonyl]-piperidin-4-yl}-phenoxy)-acetic acid

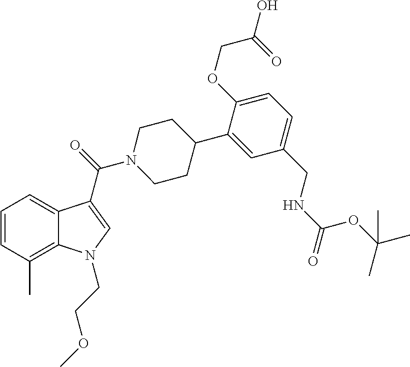

A mixture of (4-(tert-butoxycarbonylamino-methyl)-2-{1-[1-(2-methoxy-ethyl)-7-methyl-1H-indole-3-carbonyl]-piperidin-4-yl}-phenoxy)-acetic acid methyl ester (250 mg, 0.42 mmol, Example 23F) in MeOH (2.5 mL) and 1.0 M NaOH (2.5 mL) is stirred at r.t. for 30 min. The reaction mixture is concentrated in vacuo, and the residue is partitioned between H₂O and Et₂O. The two layers are separated, and the aqueous is acidified to pH˜4 with 10% citric acid. The acidified aqueous layers is extracted with CH₂Cl₂ (3×), and the combined organic extracts are washed with H₂O and brine, dried over Na₂SO₄, filtered, and concentrated in vacuo to yield the product (257 mg, 100%) as a yellow foam.

¹H NMR (300 MHz, CDCl₃) δ 7.75-7.55 (m, 1H), 7.45 (s, 1H), 7.20-6.85 (m, 4H), 6.80-6.60 (m, 1H), 5.00-4.30 (m, 5H), 4.35-4.10 (m, 2H), 4.00-3.50 (m, 3H), 3.40-2.80 (m, 6H), 2.70 (s, 3H), 2.00-1.65 (m, 4H), 1.45 (s, 9H);

LC Rt 0.94 min; MS 580 (M+H, 100%).

### B. (3-{1-[1-(2-Methoxy-ethyl)-7-methyl-1H-indole-3-carbonyl]-piperidin-4-yl}-4-methylcarbamoylmethoxy-benzyl)-carbamic acid tert-butyl ester

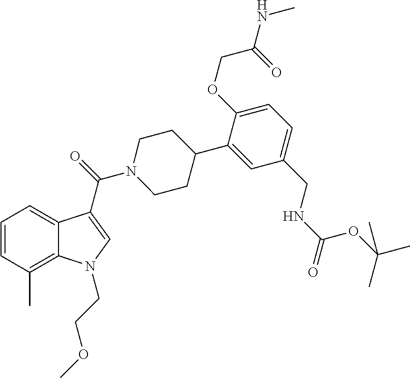

A mixture of (4-(tert-butoxycarbonylamino-methyl)-2-{1-[1-(2-methoxy-ethyl)-7-methyl-1H-indole-3-carbonyl]-piperidin-4-yl}-phenoxy)-acetic acid (257 mg, 0.44 mmol), DIEA (102 μL, 0.53 mmol), methylamine (2.0 M in THF, 4 mL), and EDCI (270 mg, 1.40 mmol) in CH₂Cl₂ (5 mL) is stirred at r.t. for 6 h. The two layers are separated, and the organic layer is washed with 10% citric acid, sat. NaHCO₃, and brine, dried over Na₂SO₄, filtered, and concentrated in vacuo. The crude material is purified on silica gel with CH₂Cl₂/MeOH (100/0 to 94/6) as eluent to give the product (58 mg, 22%) as a light yellow foam.

¹H NMR (300 MHz, CDCl₃) δ 7.65-7.55 (m, 1H), 7.45 (s, 1H), 7.20-6.90 (m, 4H), 6.80-6.65 (m, 1H), 4.80 (br s, 1H), 4.60-4.35 (m, 6H), 4.30-4.10 (m, 2H), 3.80-3.60 (t, 2H), 3.30 (s, 3H), 3.30-2.95 (m, 3H), 2.90 (s, 3H), 2.70 (s, 3H), 1.95-1.60 (m, 4H), 1.45 (s, 9H);

LC Rt 0.93 min; MS 593 (M+H, 100%).

### C. 2-(4-Aminomethyl-2-{1-[1-(2-methoxy-ethyl)-7-methyl-1H-indole-3-carbonyl]-piperidin-4-yl}-phenoxy)-N-methyl-acetamide hydrochloride

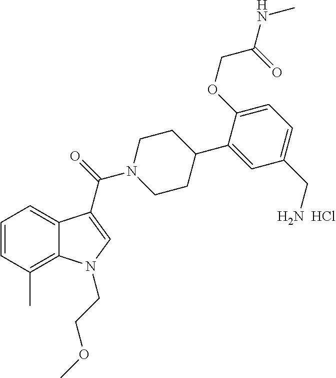

A mixture (3-{1-[1-(2-methoxy-ethyl)-7-methyl-1H-indole-3-carbonyl]-piperidin-4-yl}-4-methylcarbamoylmethoxy-benzyl)-carbamic acid tert-butyl ester (58 mg, 0.098 mmol) in 4 M HCl in dioxane (2 mL) is stirred at r.t. for 1 h. The mixture is concentrated in vacuo, and the residue is washed with Et₂O several times to give the product (40 mg, 77%) as a white powder.

¹H NMR (300 MHz, DMSO-d6) δ 8.11 (br s, 3H), 8.00-7.85 (m, 1H), 7.61 (s, 1H), 7.54 (d, J=7.1 Hz, 1H), 7.39 (s, 1H), 7.30-7.20 (m, 1H), 7.10-6.80 (m, 3H), 4.65-4.30 (m, 5H), 4.05-3.80 (m, 2H), 3.80-3.20 (m, 4H), 3.22 (s, 3H), 3.20-2.80 (m, 2H), 2.67 (s, 3H), 2.40 (s, 3H), 1.90-1.40 (m, 4H);

LC 0.65 min; MS 493 (M+H, 100%).

### Example 27

### [4-(5-Aminomethyl-2-fluoro-phenyl)-piperidin-1-yl]-{1-[2-(2-hydroxy-ethoxy)-ethyl]-7-trifluoromethoxy-1H-indol-3-yl}-methanone hydrochloride

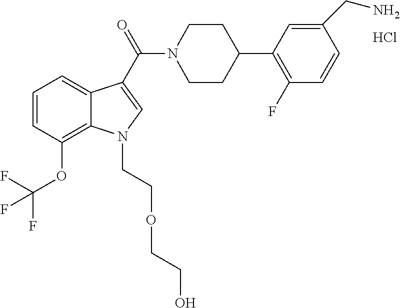

### A-1. 2,2,2-Trifluoro-1-{1-[2-(2-hydroxy-ethoxy)-ethyl]-7-trifluoromethoxy-1H-indol-3-yl}-ethanone and A-2. Trifluoro-acetic acid 2-{2-[3-(2,2,2-trifluoro-acetyl)-7-trifluoromethoxy-indol-1-yl]-ethoxy}-ethyl ester

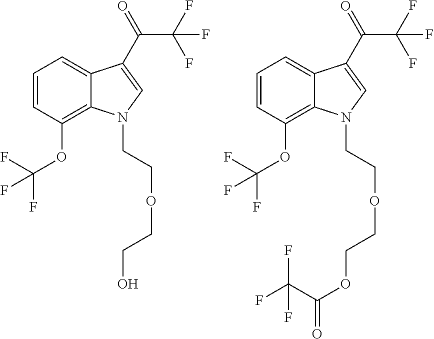

The title compounds are prepared in a similar manner as described in Example 1F using 2-[2-(7-trifluoromethoxy-indol-1-yl)ethoxy]-ethanol as the starting material. The purified 1:1 mixtures of products are used in the next step.

### B. 1-[2-(2-Hydroxy-ethoxy)-ethyl]-7-trifluoromethoxy-1H-indole-3-carboxylic acid

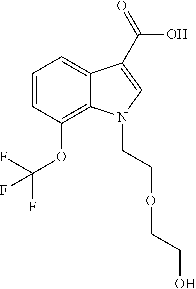

The title compound is prepared in a similar manner as described in Example 4C using the 1:1 mixture of 2,2,2-trifluoro-1-{1-[2-(2-hydroxy-ethoxy)-ethyl]-7-trifluoromethoxy-1H-indol-3-yl}-ethanone and trifluoro-acetic acid 2-{2-[3-(2,2,2-trifluoro-acetyl)-7-trifluoromethoxy-indol-1-yl]-ethoxy}-ethyl ester as the starting material.

¹H NMR (300 MHz, DMSO-d6) δ 12.2 (bs, 1H), 8.2 (s, 1H), 8.1 (m, 1H), 7.2 (m, 2H), 4.5 (m, 4H), 3.8 (m, 3H), 3.4 (t, 2H).

### C. 2,2,2-Trifluoro-N-[4-fluoro-3-(1-{1-[2-(2-hydroxy-ethoxy)-ethyl]-7-trifluoromethoxy-1H-indole-3-carbonyl}-piperidin-4-yl)-benzyl]-acetamide

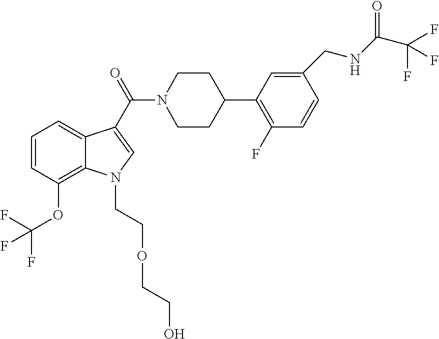

The title compound is prepared in a similar manner as described Example 2I using 1-[2-(2-hydroxy-ethoxy)-ethyl]-7-trifluoromethoxy-1H-indole-3-carboxylic acid and 2,2,2-trifluoro-N-(4-fluoro-3-piperidin-4-yl-phenyl)-acetamide hydrochloride as the starting materials.

¹H NMR (300 MHz, CDCl₃) δ 7.7 (d, 1H), 7.6 (s, 1H), 7.2 (m, 5H), 7.1 (m, 1H), 6.8 (bs, 1H), 4.6 (m, 6H), 3.8 (m, 2H), 3.6 (m, 2H), 3.5 (m, 2H), 3.1 (m, 2H), 1.9 (m, 2H), 1.8 (m, 2H).

LCMS m/z: [M+H]⁺=620.

### D. [4-(5-Aminomethyl-2-fluoro-phenyl)-piperidin-1-yl]-{1-[2-(2-hydroxy-ethoxy)-ethyl]-7-trifluoromethoxy-1H-indol-3-yl}-methanone hydrochloride

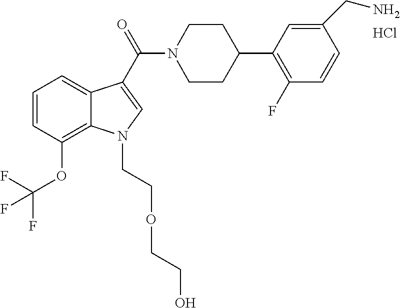

The title compound is prepared in a similar manner as described in Example 1K using 2,2,2-trifluoro-N-[4-fluoro-3-(1-{1-[2-(2-hydroxy-ethoxy)-ethyl]-7-trifluoromethoxy-1H-indole-3-carbonyl}-piperidin-4-yl)-benzyl]-acetamide as the starting material.

¹H NMR (300 MHz, DMSO-d6) δ 8.4 (bs, 2H), 7.8 (s, 1H), 7.7 (m, 1H), 7.6 (m, 1H), 7.4 (m, 1H), 7.2 (m, 3H), 4.4 (m, 4H), 4.0 (m, 2H), 3.8 (m, 3H), 3.4 (m, 4H), 3.1 (m, 3H), 1.8-1.7 (m, 4H).

LCMS m/z: [M+H]⁺=524.

### Example 28

### 2-(4-Aminomethyl-2-{1-[1-(2-methoxy-ethyl)-7-trifluoromethoxy-1H-indole-3-carbonyl]-piperidin-4-yl}-phenoxy)-N-methyl-acetamide hydrochloride

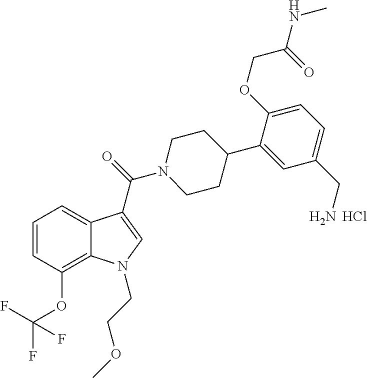

### A. (4-(tert-Butoxycarbonylamino-methyl)-2-{1-[1-(2-methoxy-ethyl)-7-trifluoromethoxy-1H-indole-3-carbonyl]-piperidin-4-yl}-phenoxy)-acetic acid

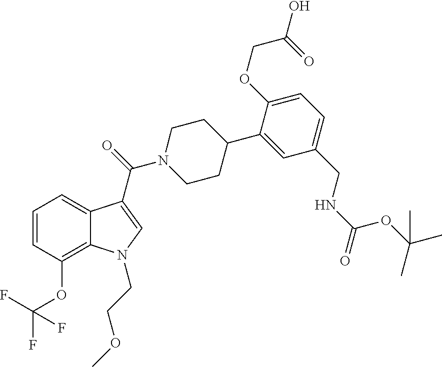

A mixture of (4-(tert-butoxycarbonylamino-methyl)-2-{1-[1-(2-methoxy-ethyl)-7-trifluoromethoxy-1H-indole-3-carbonyl]-piperidin-4-yl}-phenoxy)-acetic acid methyl ester (100 mg, 0.15 mmol, Example 24A) in MeOH (2.0 mL) and 1.0 M NaOH (2.0 mL) is stirred at r.t. for 30 min. The reaction mixture is concentrated in vacuo, and the residue is partitioned between H₂O and Et₂O. The two layers are separated, and the aqueous is acidified to pH˜4 with 10% citric acid. The acidified aqueous layers are extracted with CH₂Cl₂ (3×), and the combined organic extracts are washed with H₂O and brine, dried over Na₂SO₄, filtered, and concentrated in vacuo to yield the product (98 mg, 100%) as a yellow foam.

¹H NMR (300 MHz, CDCl₃) δ 7.75-7.65 (m, 1H), 7.55-7.45 (m, 1H), 7.20-6.95 (m, 5H), 6.80-6.60 (m, 1H), 4.70-4.35 (m, 7H), 4.30-4.10 (m, 2H), 3.80-3.60 (m, 2H), 3.30 (s, 3H), 3.30-2.80 (m, 3H), 2.10-1.65 (m, 4H), 1.45 (s, 9H);

LC Rt 1.01 min; MS 650 (M+H, 100%).

### B. (3-{1-[1-(2-Methoxy-ethyl)-7-trifluoromethoxy-1H-indole-3-carbonyl]-piperidin-4-yl}-4-methylcarbamoylmethoxy-benzyl)-carbamic acid tert-butyl ester

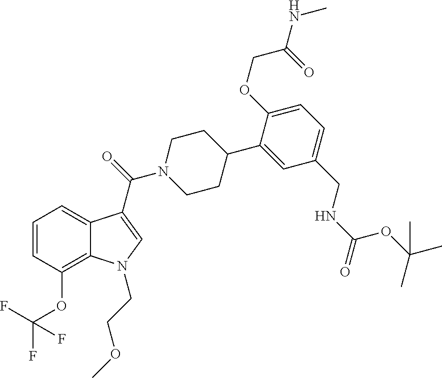

To a mixture of (4-(tert-butoxycarbonylamino-methyl)-2-{1-[1-(2-methoxy-ethyl)-7-trifluoromethoxy-1H-indole-3-carbonyl]-piperidin-4-yl}-phenoxy)-acetic acid (196 mg, 0.30 mmol) in acetone (3 mL) at 0° C. is added TEA (42 μL, 0.30 mmol) dropwise. After 30 min, iso-butyl chloroformate (39 μL, 0.30 mmol) is added. After 30 min, the reaction mixture is filtered, and the filtrate is concentrated in vacuo. The residue is redissolved in acetone (5 mL), and methylamine (4 mL, 40% in H₂O) is added. After this mixture is stirred at r.t. for 30 min, it is concentrated in vacuo. The residue is partitioned between CH₂Cl₂ and 10% citric acid. The two layers are separated, and the organic layer is washed with sat. NaHCO₃, H₂O, and brine, dried over Na₂SO₄, filtered, and concentrated in vacuo. The crude material is purified on silica gel using CH₂Cl₂/MeOH (100/0 to 96/4) as eluent to give the product (45 mg, 22%) as a white solid.

¹H NMR (300 MHz, CDCl₃) δ 7.75-7.65 (m, 1H), 7.50 (s, 1H), 7.25-7.00 (m, 4H), 6.85-6.75 (m, 1H), 6.30 (br s, 1H), 4.90-4.40 (m, 7H), 4.35-4.15 (m, 2H), 3.80-3.60 (t, 2H), 3.35 (s, 3H), 3.30-2.95 (m, 3H), 2.90 (s, 3H), 2.70 (s, 3H), 2.00-1.60 (m, 4H), 1.40 (s, 9H),

LC Rt 1.01 min; MS 663 (M+H, 100%).

### C. 2-(4-Aminomethyl-2-{1-[1-(2-methoxy-ethyl)-7-trifluoromethoxy-1H-indole-3-carbonyl]-piperidin-4-yl}-phenoxy)-N-methyl-acetamide hydrochloride

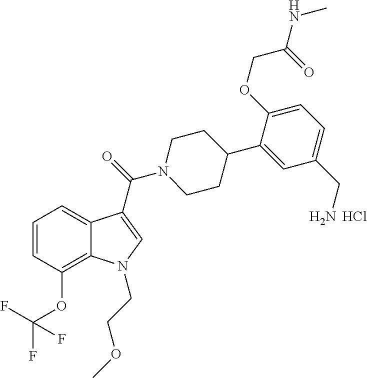

A mixture of (3-{1-[1-(2-methoxy-ethyl)-7-trifluoromethoxy-1H-indole-3-carbonyl]-piperidin-4-yl}-4-methylcarbamoylmethoxy-benzyl)-carbamic acid tert-butyl ester (40 mg, 0.06 mmol) in 4 M HCl in dioxane (2 mL) is stirred at r.t. for 30 min. The mixture is concentrated in vacuo, and the residue is washed with Et₂O several times to give the product (28 mg, 73%) as a slightly pink powder.

¹H NMR (300 MHz, DMSO-d6) δ 8.11 (br s, 3H), 8.00-7.85 (m, 1H), 7.79 (s, 1H), 7.75-7.65 (m, 1H), 7.39 (s, 1H), 7.35-7.15 (m, 2H), 6.90 (d, J=8.5 Hz, 1H), 4.65-4.30 (m, 6H), 4.05-3.90 (m, 2H), 3.68 (t, J=5.3 Hz, 3H), 3.21 (s, 3H), 3.20-3.30 (m, 3H), 2.67 (d, J=4.6 Hz, 3H), 1.90-1.50 (m, 4H);

LC 0.73 min; MS 563 (M+H, 100%).

### Example 29

### 2-(4-Aminomethyl-2-{1-[1-(2-methoxy-ethyl)-7-trifluoromethoxy-1H-indole-3-carbonyl]-piperidin-4-yl}-phenoxy)-N,N-dimethyl-acetamide hydrochloride

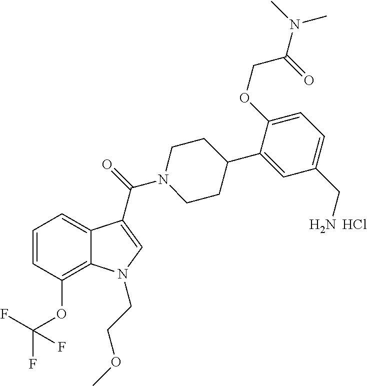

### A (4-Dimethylcarbamoylmethoxy-3-{1-[1-(2-methoxy-ethyl)-7-trifluoromethoxy-1H-indole-3-carbonyl]-piperidin-4-yl}-benzyl)-carbamic acid tert-butyl ester

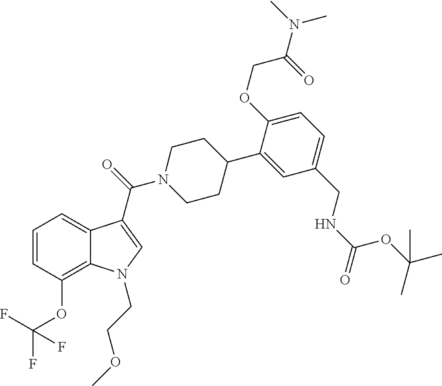

To a mixture of (4-(tert-butoxycarbonylamino-methyl)-2-{1-[1-(2-methoxy-ethyl)-7-trifluoromethoxy-1H-indole-3-carbonyl]-piperidin-4-yl}-phenoxy)-acetic acid (160 mg, 0.24 mmol, example 24A) in acetone (1 mL) at 0° C. is added TEA (41 μL, 0.27 mmol) dropwise. After 30 min, iso-butyl chloroformate (35 μL, 0.27 mmol) in acetone (1 mL) is added. After 30 min, the reaction mixture is filtered, and the filtrate is concentrated in vacuo. The residue is redissolved in THF (3 mL), and methylamine (4 mL, 2.0 M in THF) is added. After this mixture is stirred at r.t. for 30 min, it is concentrated in vacuo. The residue is partitioned between CH₂Cl₂ and 10% citric acid. The two layers are separated, and the organic layer is washed with sat. NaHCO₃, H₂O, and brine, dried over Na₂SO₄, filtered, and concentrated in vacuo. The crude material is purified on silica gel with CH₂Cl₂/MeOH (100/0 to 96/4) as eluent to give the product (110 mg, 67%) as a white solid.

¹H NMR (300 MHz, CDCl₃) δ 7.75-7.60 (m, 1H), 7.45 (s, 1H), 7.25-7.00 (m, 4H), 6.85-6.65 (m, 1H), 4.80-4.40 (m, 7H), 4.30-4.10 (m, 2H), 3.80-3.60 (m, 2H), 3.30 (s, 3H), 3.30-2.70 (m, 9H), 2.00-1.60 (m, 4H), 1.40 (s, 9H);

LC Rt 1.04 min; MS 677 (M+H, 100%).

### B. 2-(4-Aminomethyl-2-{1-[1-(2-methoxy-ethyl)-7-trifluoromethoxy-1H-indole-3-carbonyl]-piperidin-4-yl}-phenoxy)-N,N-dimethyl-acetamide hydrochloride

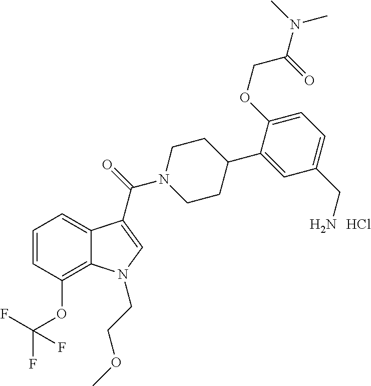

A mixture of (4-dimethylcarbamoylmethoxy-3-{1-[1-(2-methoxy-ethyl)-7-trifluoromethoxy-1H-indole-3-carbonyl]-piperidin-4-yl}-benzyl)-carbamic acid tert-butyl ester (108 mg, 0.16 mmol) in 4 M HCl in dioxane (2 mL) is stirred at r.t. for 30 min. The mixture is concentrated in vacuo, and the residue is washed with Et₂O several times to give the product (82 mg, 83%) as a white powder.

¹H NMR (300 MHz, DMSO-d6) δ 8.11 (br s, 3H), 7.79 (s, 1H), 7.75-7.70 (m, 1H), 7.37 (d, J=2 Hz, 1H), 7.30-7.10 (m, 3H), 6.91 (d, J=8.6 Hz, 1H), 4.91 (s, 2H), 4.60-4.30 (m, 3H), 4.05-3.85 (m, 2H), 3.68 (t, J=5.3 Hz, 3H), 3.25 (m, 1H), 3.21 (s, 3H), 3.15 (m, 2H), 3.01 (s, 3H), 2.83 (s, 3H), 1.95-1.50 (m, 4H);

LC 0.75 min; MS 577 (M+H, 100%).

### Example 30

### [4-(5-Aminomethyl-2-fluoro-phenyl)-piperidin-1-yl]-{1-[2-(1-methyl-piperidin-2-yl)-ethyl]-1H-indol-3-yl}-methanone dihydrochloride

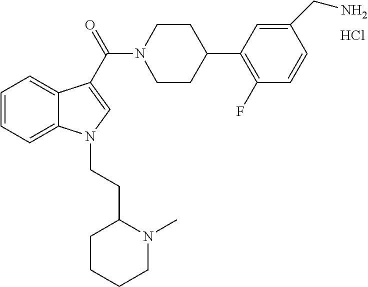

### A. 1-[2-(1-Methyl-piperidin-2-yl)ethyl]-1H-indole-3-carboxylic acid methyl ester

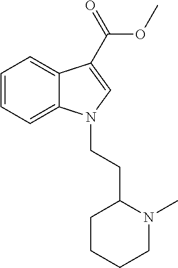

The title compound is prepared in a similar manner as described in Example 2F using 1H-indole-3-carboxylic acid methyl ester and 2-(2-chloroethyl)1-methylpiperidine hydrochloride in DMF as the starting materials.

¹H NMR (300 MHz, CDCl₃) δ 8.2 (m, 1H), 7.8 (s, 1H), 7.4 (m, 1H), 7.3 (m, 2H), 4.2 (m, 2H), 3.9 (s, 3H), 3.6 (m, 1H), 2.3 (s, 3H), 2.2-2.0 (m, 4H), 1.8-1.6 (m, 6H).

### B. 1-[2-(1-Methyl-piperidin-2-yl)ethyl]-1H-indole-3-carboxylic acid

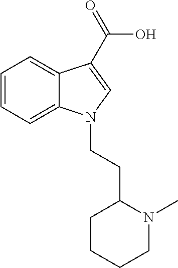

The title compound is prepared in a similar manner as described in Example 5D using 1-[2-(1-methyl-piperidin-2-yl)-ethyl]-1H-indole-3-carboxylic acid methyl ester as the starting material.

¹H NMR (300 MHz, DMSO-d6) δ 12.4 (bs, 1H), 8.2 (s, 1H), 8.0 (d, 1H), 7.6 (d, 1H), 7.2 (m, 2H), 4.4 (m, 2H), 3.1 (m, 2H), 2.8 (m, 2H), 2.6 (m, 1H), 2.5 (s, 3H), 2.1 (m, 2H), 1.8 (m, 4H).

### C. 2,2,2-Trifluoro-N-[4-fluoro-3-(1-{1-[2-(1-methyl-piperidin-2-yl)-ethyl]-1H-indole-3-carbonyl}-piperidin-4-yl)-benzyl]-acetamide

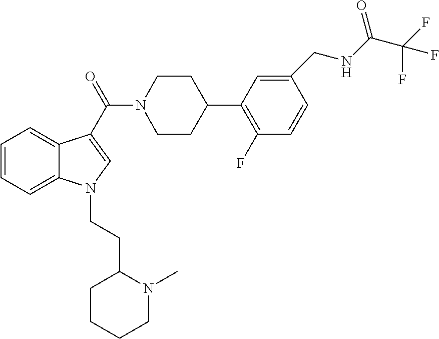

The title compound is prepared in a similar manner as described in Example 2I using 1-[2-(1-methyl-piperidin-2-yl)-ethyl]-1H-indole-3-carboxylic acid and 2,2,2-trifluoro-N-(4-fluoro-3-piperidin-4-yl-phenyl)acetamide hydrochloride as the starting materials.

¹H NMR (300 MHz, CDCl₃) δ 7.7 (d, 1H), 7.5 (s, 1H), 7.4 (d, 1H), 7.3-7.1 (m, 4H), 7.0 (m, 1H), 6.7 (bs, 1H), 4.6 (m, 2H), 4.45 (m, 2H), 4.2 (m, 2H), 3.1 (m, 3H), 2.9 (m, 1H), 2.3 (s, 3H), 2.1 (m, 4H), 1.8-1.5 (m, 10H).

LCMS m/z: [M+H]+=573

### D. [4-(5-Aminomethyl-2-fluoro-phenyl)-piperidin-1-yl]-{1-[2-(1-methyl-piperidin-2-yl)-ethyl]-1H-indol-3-yl}-methanone dihydrochloride

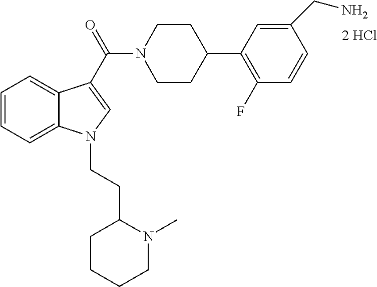

The title compound is prepared in a similar manner as described in Example 1K using 2,2,2-trifluoro-N-[4-fluoro-3-(1-{1-[2-(1-methyl-piperidin-2-yl)-ethyl]-1H-indole-3-carbonyl}-piperidin-4-yl)-benzyl]-acetamide as the starting material.

¹H NMR (300 MHz, DMSO-d6) δ 11.0 (bd, 1H), 8.5 (bs, 2H), 7.9 (s, 1H), 7.7 (m, 1H), 7.6 (m, 1H), 7.4 (m, 1H), 7.2 (m, 4H), 4.4 (m, 4H), 4.0 (m, 2H), 3.4 (m, 2H), 3.1 (m, 4H), 2.3 (s, 3H), 2.0 (m, 2H), 1.8-1.7 (m, 10H).

MS m/z: [M+H]⁺=477.

### Example 31

### 2-(4-Aminomethyl-2-{1-[1-(2-methoxy-ethyl)-7-methyl-1H-indole-3-carbonyl]-piperidin-4-yl}-phenoxy)-acetamide hydrochloride

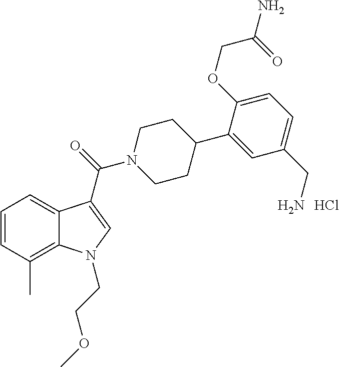

### A. (4-Carbamoylmethoxy-3-{1-[1-(2-methoxy-ethyl)-7-methyl-1H-indole-3-carbonyl]-piperidin-4-yl}-benzyl)-carbamic acid tert-butyl ester

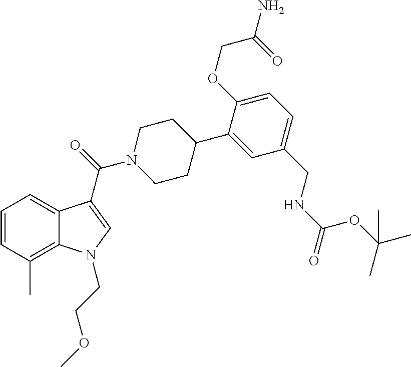

A mixture of 1-(2-methoxy-ethyl)-7-methyl-1H-indole-3-carboxylic acid (165 mg, 0.70 mmol), DIEA (245 μL, 1.40 mmol), (4-carbamoylmethoxy-3-piperidin-4-yl-benzyl)-carbamic acid tert-butyl ester (249 mg, 0.68 mmol, Example 32C), HOBT (114 mg, 0.84 mmol), and EDCI (162 mg, 0.84 mmol) in CH₂Cl₂ (10 mL) is stirred at r.t. for 6 h. The mixture is partitioned between H₂O and CH₂Cl₂. The two layers are separated, and the organic layer is washed with 10% citric acid, sat. NaHCO₃, and brine, dried over Na₂SO₄, filtered, and concentrated in vacuo. The crude material is purified on silica gel with CH₂Cl₂/MeOH (100/0 to 96/4) as eluent to give the product (97 mg, 24%) as a beige foam.

¹H NMR (300 MHz, CDCl₃) δ 7.70-7.50 (m, 1H), 7.45 (s, 1H), 7.20-7.00 (m, 3H), 7.00-6.90 (m, 1H), 6.85-6.70 (m, 1H), 6.35 (br s, 1H), 5.65 (br s, 1H), 4.80 (br s, 1H), 4.70-4.40 (m, 6H), 4.35-4.15 (m, 2H), 3.80-3.60 (m, 2H), 3.30 (s, 3H), 3.20-2.95 (m, 3H), 2.00-1.60 (m, 4H), 1.45 (s, 9H);

LC Rt 0.90 min; MS 579 (M+H, 100%).

### B. 2-(4-Aminomethyl-2-{1-[1-(2-methoxy-ethyl)-7-methyl-1H-indole-3-carbonyl]-piperidin-4-yl}-phenoxy)-acetamide hydrochloride

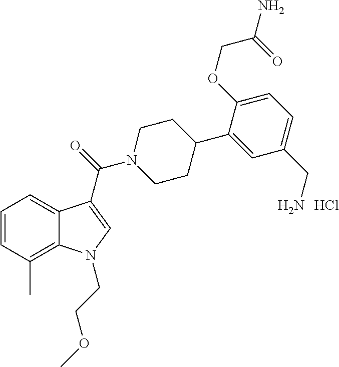

A mixture of (4-carbamoylmethoxy-3-{1-[1-(2-methoxy-ethyl)-7-methyl-1H-indole-3-carbonyl]-piperidin-4-yl}-benzyl)-carbamic acid tert-butyl ester (95 mg, 0.16 mmol) in 4 M HCl in dioxane (3 mL) is stirred at r.t. for 1 h. The mixture is concentrated in vacuo, and the residue is triturated with Et₂O (4×). The solid is dissolved in H₂O, and the resulting solution is lyophilized to give the product (66 mg, 80%) as a white fluffy powder.

¹H NMR (300 MHz, DMSO-d6) δ 8.10 (br s, 3H), 7.61 (s, 1H), 7.60-7.50 (m, 1H), 7.50-7.05 (m, 3H), 7.50-6.80 (m, 4H), 4.60-4.30 (m, 6H), 4.00-3.80 (m, 2H), 3.75-3.60 (m, 2H), 3.22 (s, 3H), 3.20-2.90 (m, 2H), 2.67 (s, 3H), 1.90-1.45 (m, 4H);

LC 0.60 min; MS 479 (M+H, 100%).

### Example 32

### 2-(4-Aminomethyl-2-{1-[1-(2-methoxy-ethyl)-7-trifluoromethoxy-1H-indole-3-carbonyl]-piperidin-4-yl}-phenoxy)-acetamide hydrochloride

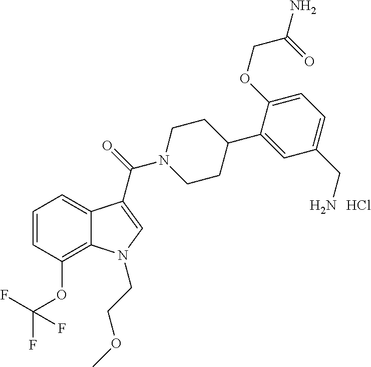

### A. (3-Bromo-4-carbamoylmethoxy-benzyl)-carbamic acid tert-butyl ester

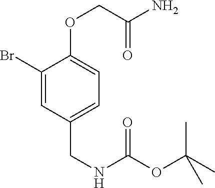

A mixture of (3-bromo-4-hydroxy-benzyl)-carbamic acid tert-butyl ester (1.30 g, 4.30 mmol), 2-bromoacetamide (0.59 g, 4.3 mmol), and Cs₂CO₃ (3.50 g, 10.8 mmol) in THF (15 mL) is stirred at 60° C. for 1 h. The reaction mixture is filtered, and the filtrate is concentrated in vacuo. The crude material is crystallized from EtOAc give the product (1.15 g, 74%) as a white power.

¹H NMR (300 MHz, CDCl₃) δ 7.50 (s, 1H), 7.35-7.10 (m, 1H), 7.00-6.70 (m, 3H), 5.75 (br s, 1H), 4.85 (m, 1H), 4.50 (s, 2H), 4.35-4.10 (m, 2H), 1.40 (s, 9H);

LC Rt 0.79 min.

### B. (4-Carbamoylmethoxy-3-pyridin-4-yl-benzyl)-carbamic acid tert-butyl ester

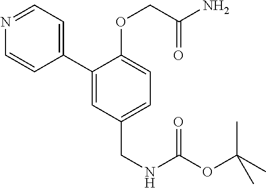

A mixture of (3-bromo-4-carbamoylmethoxy-benzyl)-carbamic acid tert-butyl ester (1.00 g, 2.78 mmol), pyridine-4-boronic acid (0.41 g, 3.34 mmol), Cs₂CO₃ (1.81 g, 5.56 mol), Pd(dppf)Cl₂.CH₂Cl₂ (0.20, 10% mol) in dioxane/H₂O (16 mL, 10/1) is heated at 80° C. for 2 h and then at r.t. overnight. The reaction mixture is cooled to r.t., and then concentrated in vacuo. The residue is partitioned between EtOAc and H₂O. The two layers are separated, and the organic layer is washed with H₂O, and brine, dried over Na₂SO₄, filtered, and concentrated in vacuo. The crude material is purified on silica gel with CH₂Cl₂/MeOH (100/0 to 95/5) as eluent to give the product (0.44 g, 44%) as a brown foam.

¹H NMR (300 MHz, CDCl₃) 8.80-8.60 (m, 2H), 7.55-7.10 (m, 4H), 7.00-6.80 (m, 2H), 4.86 (br s, 1H), 6.10 (br s, 1H), 5.60 (br s, 1H), 4.90 (br s, 1H), 4.70 (s, 2), 4.40-4.20 (m, 2H), 1.45 (s, 9H);

LC Rt 0.56 min; MS 359 (M+H, 100%).

### C. (4-Carbamoylmethoxy-3-piperidin-4-yl-benzyl)-carbamic acid tert-butyl ester

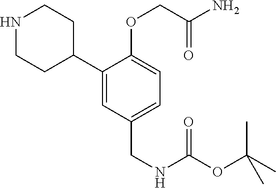

A mixture of (4-carbamoylmethoxy-3-pyridin-4-yl-benzyl)-carbamic acid tert-butyl ester (440 mg, 1.23 mmol) and PtO₂ (50 mg) in MeOH (15 mL) and acetic acid (1 mL) is hydrogenated at 50-60 psi at r.t. for 4 h. The reaction mixture is filtered through Celite, and the filtrate is concentrated in vacuo. The residue is partitioned between CH₂Cl₂ and sat NaHCO₃. The two layers are separated and the organic layer is washed with H₂O and brine, dried over Na₂SO₄, filtered, and concentrated in vacuo to give the crude material (501 mg) as a white foam. This crude material is used in the next step without further purification.

¹H NMR (300 MHz, CDCl₃) δ 7.20-7.05 (m, 2H), 6.85-6.70 (m, 1H), 6.50 (br s, 1H), 5.60 (br s, 1H), 4.75 (br s, 1H), 4.50 (s, 4H), 4.40-4.10 (m, 3H), 3.30-3.10 (m, 1H), 3.10-2.90 (m, 1H), 2.90-2.65 (m, 1H), 2.30 (s, 1H), 2.20-1.50 (m, 4H) 1.40 (s, 9H);

LC Rt 0.56 min; MS 364 (M+H, 100%).

### D. (4-Carbamoylmethoxy-3-{1-[1-(2-methoxy-ethyl)-7-trifluoromethoxy-1H-indole-3-carbonyl]-piperidin-4-yl}-benzyl)-carbamic acid tert-butyl ester

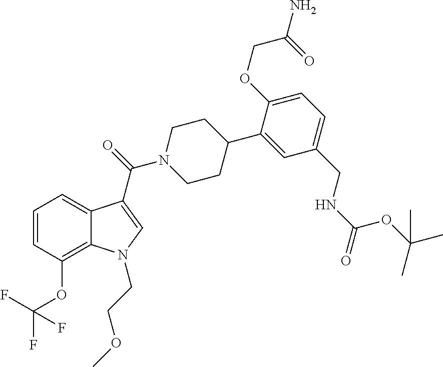

A mixture of 1-(2-methoxy-ethyl)-7-trifluoromethoxy-1H-indole-3-carboxylic acid (219 mg, 0.73 mmol), DIEA (251 μL, 1.44 mmol), (4-carbamoylmethoxy-3-piperidin-4-yl-benzyl)-carbamic acid tert-butyl ester (256 mg, 0.72 mmol), HOBT (117 mg, 0.86 mmol), and EDCI (166 mg, 0.86 mmol) in CH₂Cl₂ (10 mL) is stirred at r.t. for 6 h. The mixture is partitioned between H₂O and CH₂Cl₂. The two layers are separated, and the organic layer is washed with 10% citric acid, sat. NaHCO₃, and brine, dried over Na₂SO₄, filtered, and concentrated in vacuo. The crude material is purified on silica gel with CH₂Cl₂/MeOH (100/0 to 90/10) as eluent to give the product (141 mg, 31%) as a light yellow foam.

¹H NMR (300 MHz, CDCl₃) δ 7.80-7.60 (m, 1H), 7.45 (s, 1H), 7.20-7.00 (m, 4H), 6.85-6.70 (m, 1H), 6.35 (br s, 1H), 5.70 (br s, 1H), 4.80 (br s, 1H), 4.70-4.35 (m, 6H), 4.30-4.10 (m, 2H), 3.80-3.60 (m, 2H), 3.30 (s, 3H), 3.25-3.00 (m, 3H), 2.00-1.50 (m, 4H), 1.40 (s, 9H);

LC Rt 0.99 min; MS 649 (M+H, 100%).

### E. 2-(4-Aminomethyl-2-{1-[1-(2-methoxy-ethyl)-7-trifluoromethoxy-1H-indole-3-carbonyl]-piperidin-4-yl}-phenoxy)-acetamide hydrochloride

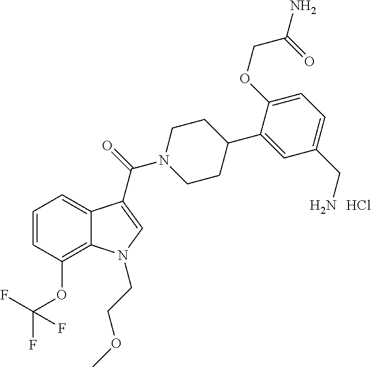

A mixture of (4-carbamoylmethoxy-3-{1-[1-(2-methoxy-ethyl)-7-trifluoromethoxy-1H-indole-3-carbonyl]-piperidin-4-yl}-benzyl)-carbamic acid tert-butyl ester (130 mg, 0.2 mmol) in 4 M HCl in dioxane (3 mL) is stirred at r.t. for 1 h. The mixture is concentrated in vacuo, and the residue is triturated with Et₂O (4×) to give the product (96 mg, 82%) as a beige powder.

¹H NMR (300 MHz, DMSO-d6) δ 8.19 (br s, 3H), 7.79 (s, 1H), 7.75-7.65 (m, 1H), 7.40 (s, 1H), 7.30-7.10 (m, 2H), 6.95-6.85 (m, 1H), 4.60-4.25 (m, 4H), 4.00-3.85 (m, 2H), 3.80-3.60 (m, 2H), 3.40-3.25 (m, 2H), 3.21 (s, 3H), 3.20-3.00 (m, 2H), 2.51 (s, 3H), 1.95-1.50 (m, 4H);

LC 0.70 min; MS 549 (M+H, 100%).

### Example 33

### (4-Aminomethyl-2-{1-[1-(2-methoxy-ethyl)-7-methyl-1H-indole-3-carbonyl]-piperidin-4-yl}-phenoxy)-acetic acid hydrochloride

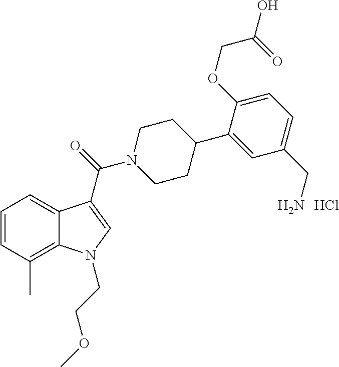

A mixture of (4-aminomethyl-2-{1-[1-(2-methoxy-ethyl)-7-methyl-1H-indole-3-carbonyl]-piperidin-4-yl}-phenoxy)-acetic acid methyl ester hydrochloride (136 mg, 0.25 mmol, example 23G) in MeOH (1.35 mL) and 1 M NaOH (1.35 mL) is stirred at r.t. for 15 min. The reaction is concentrated in vacuo. The residue is acidified to pH ˜3 with 1M HCl. The mixture is purified by RP-HPLC to give the product (63 mg, 48%) as a white solid.

¹H NMR (300 MHz, DMSO-d6) δ 13.0 (br s, 1H), 8.10 (br s, 3H), 7.61 (s, 1H), 7.54 (d, J=7.7 Hz, 1H), 7.45-7.35 (m, 1H), 7.30-7.15 (m, 1H), 7.10-6.85 (m, 3H), 4.76 (s, 2H), 4.65-4.30 (m, 4H), 3.94 (s, 2H), 3.67 (t, J=5.3 Hz, 2H), 3.22 (s, 3H), 3.15-2.90 (m, 2H), 2.67 (s, 3H), 1.90-1.45 (m, 4H);

LC 0.65 min; MS 480 (M+H, 100%).

### Example 34

### [4-(5-Aminomethyl-2-fluoro-phenyl)-piperidin-1-yl]-[1-(3-fluoro-propyl)-7-trifluoromethoxy-1H-indol-3-yl]-methanone hydrochloride

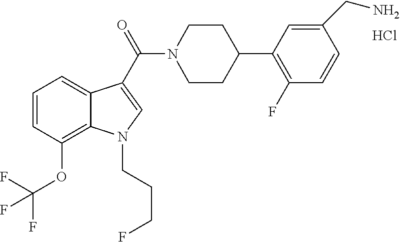

### A. 1-(3-Fluoro-propyl)-7-trifluoromethoxy-1H-indole

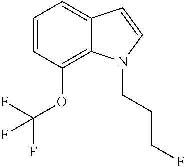

The title compound is prepared in a similar manner as described in Example 1E using 7-trifluoromethoxy-1H-indole and 1-bromo-3-fluoro-propane as the starting materials.

¹H NMR (300 MHz, CDCl₃) δ 7.5 (d, 1H), 7.1 (m, 3H), 6.5 (m, 1H), 4.5 (m, 3H), 4.3 (m, H), 2.3 (m, H), 2,2 (m, H).

MS m/z: [M+H]⁺=262.

### B. 2,2,2-Trifluoro-1-[1-(3-fluoro-propyl)-7-trifluoromethoxy-1H-indol-3-yl]-ethanone

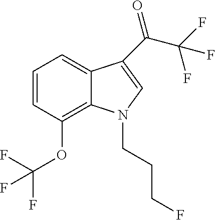

The title compound is prepared in a similar manner as described in Example 1F using 1-(3-fluoro-propyl)-7-trifluoromethoxy-1H-indole as the starting material.

¹H NMR (300 MHz, CDCl₃) δ 8.4 (d, 1H), 7.9 (s, 1H), 7.4 (m, 1H), 7.3 (m, 1H), 4.6 (m, 3H), 4.4 (m, 1H), 2.4 (m, 1H), 2.3 (m, 1H).

MS m/z: [M+H]⁺=358.

### C. 1-(3-Fluoro-propyl)-7-trifluoromethoxy-1H-indole-3-carboxylic acid

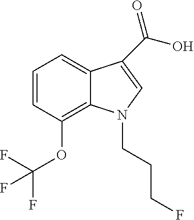

The title compound is prepared in a similar manner as described in Example 4C using 2,2,2-trifluoro-1-[1-(3-fluoro-propyl)-7-trifluoromethoxy-1H-indol-3-yl]-ethanone as the starting material.

¹H NMR (300 MHz, DMSO-d6) δ 12.3 (bs, 1H), 8.2 (s, 1H), 8.1 (m, 1H), 7.2 (m, 2H), 4.6-4.3 (m, 4H), 2.2 (m, 2H).

MS m/z: [M+H]⁺=306.

### D. 2,2,2-Trifluoro-N-(4-fluoro-3-{1-[1-(3-fluoro-propyl)-7-trifluoromethoxy-1H-indole-3-carbonyl]-piperidin-4-yl}-benzyl)-acetamide

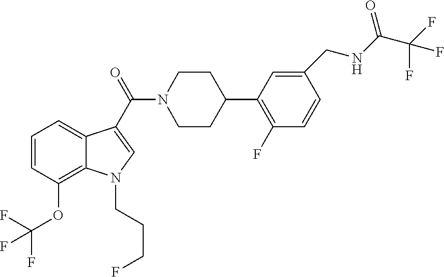

The title compound is prepared in a similar manner as described in Example 2I using 1-(3-fluoro-propyl)-7-trifluoromethoxy-1H-indole-3-carboxylic acid and 2,2,2-trifluoro-N-(4-fluoro-3-piperidin-4-yl-phenyl)-acetamide hydrochloride as the starting materials.

¹H NMR (300 MHz, DMSO-d6) δ 10.0 (m, 1H), 7.8 (s, 1H), 7.7 (d, 1H), 7.3 (d, 1H), 7.2 (m, 4H), 4.6 (t, 1H), 4.4 (m, 7H), 3.1 (m, 3H), 2.2 (m, 2H), 1.8 (m, 2H), 1.7 (m, 2H)

MS m/z: [M+H]⁺=592.

### E. [4-(5-Aminomethyl-2-fluoro-phenyl)-piperidin-1-yl]-[1-(3-fluoro-propyl)-7-trifluoromethoxy-1H-indol-3-yl]-methanone hydrochloride

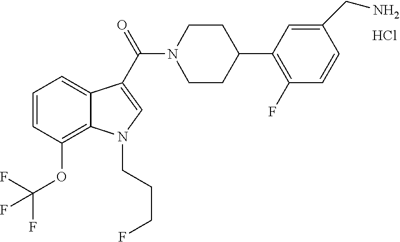

The title compound is prepared in a similar manner as described in Example 1K using 2,2,2-trifluoro-N-(4-fluoro-3-{1-[1-(3-fluoro-propyl)-7-trifluoromethoxy-1H-indole-3-carbonyl]-piperidin-4-yl}-benzyl)-acetamide as the starting material.

¹H NMR (300 MHz, DMSO-d6) δ 8.4 (bs, 2H), 7.9 (s, 1H), 7.8 (m, 1H), 7.6 (m, 1H), 7.4 (m, 1H), 7.2 (m, 3H), 4.6-4.35 (m, 6H), 4.0 (m, 2H), 3.1 (m, 3H), 2.2 (m, 2H), 1.9-1.6 (m, 4H).

LCMS m/z: [M+H]+=496.

### Example 35

### [4-(5-Aminomethyl-2-fluoro-phenyl)-piperidin-1-yl]-[1-(2-pyridin-4-yl-ethyl)-1H-indol-3-yl]-methanone dihydrochloride

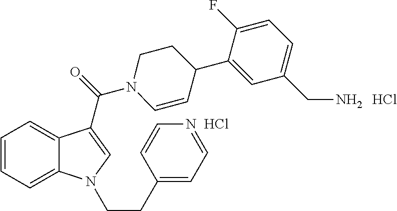

### A. 1-(2-Pyridin-4-yl-ethyl)-1H-indole

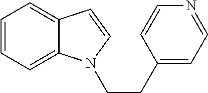

The title compound is prepared according to the procedure by Gill, A. L. et al. J. Med. Chem., 2005, vol. 48, pp. 414-426 using 4-vinylpyridine and indole as the starting materials.

¹H NMR (300 MHz, CDCl₃) δ 8.49-8.47 (m, 2H), 7.66-7.63 (m, 1H), 7.33-7.30 (m, 1H), 7.25-7.19 (m, 1H), 7.15-7.10 (m, 1H), 6.97-6.95 (m, 2H), 6.87 (d, 1H), 6.45-6.43 (m, 1H), 4.38 (t, 2H), 3.11 (t, 2H).

### B. 2,2,2-Trifluoro-1-[1-(2-pyridin-4-yl-ethyl)-1H-indol-3-yl]-ethanone

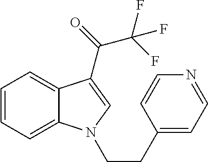

The title compound is prepared in a similar manner as described in Example 2G using 1-(2-pyridin-4-yl-ethyl)-1H-indole as the starting material.

¹H NMR (300 MHz, CDCl₃) δ 8.53-8.51 (m, 2H), 8.44-8.40 (m, 1H), 7.64 (m, 1H), 7.41-7.39 (m, 3H), 6.97-6.95 (m, 2H), 4.49 (t, 2H), 3.19 (t, 2H).

### C. 1-(2-Pyridin-4-yl-ethyl)-1H-indole-3-carboxylic acid

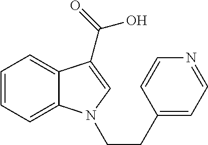

The title compound is prepared in a similar manner as described in Example 2H using 2,2,2-trifluoro-1-[1-(2-pyridin-4-yl-ethyl)-1H-indol-3-yl]-ethanone as the starting material.

¹H NMR (300 MHz, DMSO-d6) δ 8.78-8.76 (m, 2H), 8.04 (s, 1H), 8.01-7.98 (m, 1H), 7.87-7.85 (m, 2H), 7.65-7.63 (m, 1H), 7.26-7.16 (m, 2H), 4.64 (t, 2H), 3.42 (t, 2H).

### D. 2,2,2-Trifluoro-N-(4-fluoro-3-{1-[1-(2-pyridin-4-yl-ethyl)-1H-indole-3-carbonyl]-piperidin-4-yl}-benzyl)-acetamide

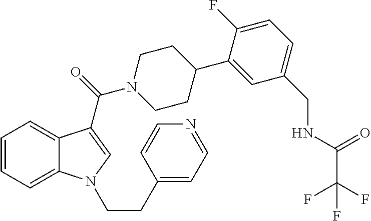

The title compound is prepared in a similar manner as described in Example 6E using 1-(2-pyridin-4-yl-ethyl)-1H-indole-3-carboxylic acid and 2,2,2-trifluoro-N-(4-fluoro-3-piperidin-4-yl-phenyl)-acetamide hydrochloride as the starting materials.

¹H NMR (300 MHz, CDCl₃) δ 8.58 (br d, 2H), 7.72-7.69 (m, 1H), 7.61 (br s, 1H), 7.34-7.28 (m, 5H), 7.17-7.07 (m, 3H), 7.04-6.98 (m, 1H), 4.54 (t, 2H), 4.49-4.47 (m, 4H), 3.38 (t, 2H), 3.19-3.04 (m, 3H), 1.88-1.84 (m, 2H), 1.68-1.56 (m, 2H).

### E. [4-(5-Aminomethyl-2-fluoro-phenyl)-piperidin-1-yl]-[1-(2-pyridin-4-yl-ethyl)-1H-indol-3-yl]-methanone dihydrochloride

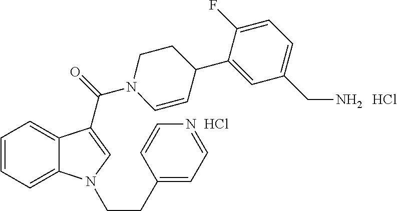

The title compound is prepared in a similar manner as described in Example 3B using 2,2,2-trifluoro-N-(4-fluoro-3-{1-[1-(2-pyridin-4-yl-ethyl)-1H-indole-3-carbonyl]-piperidin-4-yl}-benzyl)-acetamide as the starting material.

¹H NMR (300 MHz, DMSO-d6) δ 8.81 (br d, 2H), 8.52 (br s, 3H), 7.92 (d, 2H), 7.70 (s, 1H), 7.68-7.63 (m, 3H), 7.42-7.36 (m, 1H), 7.24-7.13 (m, 3H), 4.66 (t, 2H), 4.31 (br d, 2H), 4.03-3.98 (m, 2H), 3.46 (t, 2H), 3.18-3.02 (m, 3H), 1.81-1.77 (m, 2H), 1.72-1.61 (m, 2H).

### Example 36

### [4-(5-Aminomethyl-2-chloro-phenyl)-piperidin-1-yl]-[1-(2-methoxy-ethyl)-7-trifluoromethoxy-1H-indol-2-yl]-methanone hydrochloride

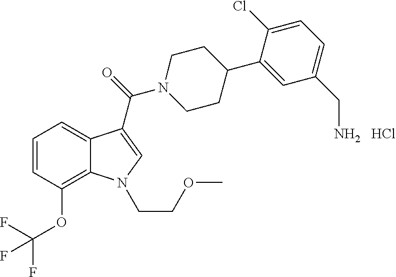

### A. (3-Bromo-4-chloro-phenyl)-methanol

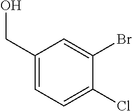

To a mixture of 3-bromo-4-chloro-benzoic acid (10.0 g, 42.5 mmol) in THF (50 mL) under nitrogen at 0° C. is added 1.0 M solution of borane. THF (55.3 mL, 55.3 mmol). After stirring at ambient temperature overnight the reaction is poured into a mixture of NaHCO₃/H₂O/ice and extracted with EtOAc. The organic layer is washed with saturated aqueous NaCl, dried over MgSO₄, filtered and concentrated in vacuo to provide the desired product (9.4 g, 100%) as a clear, colorless oil.

¹H NMR (300 MHz, CDCl₃) δ 7.61 (s, 1H), 7.41 (d, J=5.1 Hz, 1H), 7.21 (d, J=5.1 Hz, 1H), 4.63 (s, 2H), 2.12 (br s, 1H).

### B. Methanesulfonic acid 3-bromo-4-chloro-benzyl ester

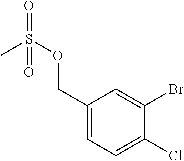

To a mixture of 3-bromo-4-chloro-phenyl)-methanol (6.0 g, 27.1 mmol) in THF (50 mL) under nitrogen at 0° C. is added triethyl amine (3.6 g, 35.3 mmol) followed by methanesulfonyl chloride (4.0 g, 35.3 mmol). After stirring at ambient temperature for 1 h the reaction is poured into a mixture of NaHCO₃/H₂O/ice and extracted with EtOAc. The organic layer is washed with saturated aqueous NaCl, dried over MgSO₄, filtered and concentrated in vacuo to provide the desired product (8.0 g, 99%) as a white solid.

¹H NMR (300 MHz, CDCl₃) δ 7.49 (m, 1H), 7.49 (m, 1H), 7.28 (m, 1H), 5.17 (s, 2H), 3.00 (s, 3H).

### C. 2-(3-Bromo-4-chloro-benzyl)-isoindole-1,3-dione

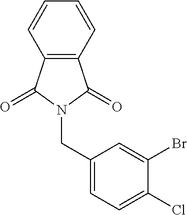

To a mixture of methanesulfonic acid 3-bromo-4-chloro-benzyl ester (7.5 g, 25.1 mmol) in DMF (60 mL) under nitrogen is added potassium phthalimide (5.6 g, 30.1 mmol). After heating on a steam bath for 2 h the reaction is poured into a mixture of H₂O/ice. The white solid is dried in vacuo and recrystallized from CH₂Cl₂/heptanes to deliver the desired product (6.1 g, 69%) as a white powder.

¹H NMR (300 MHz, CDCl₃) δ 7.88 (m, 2H), 7.73 (m, 2H), 7.68 (m, 1H), 7.39 (m, 1H), 7.31 (m, 1H), 4.79 (s, 2H).

### D. 4-[2-Chloro-5-(1,3-dioxo-1,3-dihydro-isoindol-2-ylmethyl)-phenyl]-piperidine-1-carboxylic acid tert-butyl ester

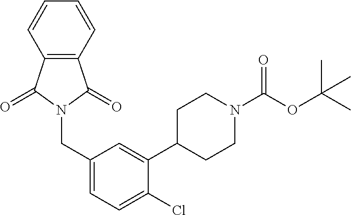

The compound is prepared utilizing the procedure described in J. Org. Chem. 2004, 69, 5120. From 2-(3-bromo-4-chloro-benzyl)-isoindole-1,3-dione (3.5 g, 10.0 mmol) is obtained the titled compound (2.9 g, 64%) as an off white foam. 1.1 g (31%) of the starting material was recovered.

¹H NMR (300 MHz, CDCl₃) δ 7.95 (m, 2H), 7.72 (m, 2H), 7.24 (m, 3H), 4.79 (s, 2H), 4.23 (m, 2H), 3.10 (m, 1H), 2.85 (m, 2H), 1.83 (m, 2H), 1.49 (s, 9H).

MS m/z: [M+H]⁺=455.

### E. 2-(4-Chloro-3-piperidin-4-yl-benzyl)isoindole-1,3-dione; hydrochloride

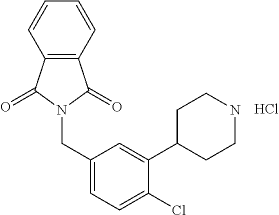

4-[2-Chloro-5-(1,3-dioxo-1,3-dihydro-isoindol-2-ylmethyl)-phenyl]-piperidine-1-carboxylic acid tert-butyl ester (1.00 g, 2.20 mmol) is stirred in methanolic HCl (20 mL) at 50° C. for 1 h. The reaction is cooled to 0° C. The resulting precipitate is filtered off and dried to deliver the titled compound (0.77 g, 89.5%) as a white solid (mp 285-287° C.).

¹H NMR (300 MHz, DMSO-d6) δ 8.75 (m, 1H), 8.44 (m, 1H), 7.91 (m, 4H), 7.43 (m, 1H), 7.29 (m, 1H), 7.20 (m, 1H), 4.77 (s, 2H), 3.40-2.90 (m, 5H), 1.97-1.72 (m, 4H).

LCMS m/z: [M+H]⁺=355.

### F. 2-(4-Chloro-3-{1-[1-(2-methoxy-ethyl)-7-trifluoromethoxy-1H-indole-2-carbonyl]-piperidin-4-yl}-benzyl)-isoindole-1,3-dione

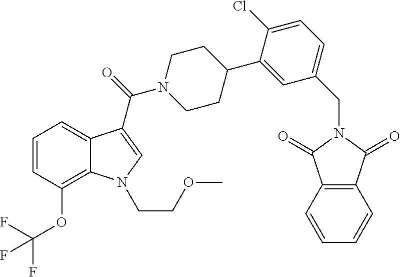

To a mixture of 1-(2-methoxy-ethyl)-7-trifluoromethoxy-1H-indole-2-carboxylic acid (0.25 g, 0.82 mmol) in THF (5 mL) under nitrogen is added 1-[3-(dimethylamino)propyl]-3-ethylcarbodiimide hydrochloride (0.17 g, 0.91 mmol) followed by triethylamine (0.32 g, 3.2 mmol) and DMF (3 mL). After stirring 1 h at ambient temperature, 2-(4-chloro-3-piperidin-4-yl-benzyl)-isoindole-1,3-dione hydrochloride (0.35 g, 0.91 mmol) is added and the reaction is stirred overnight. The reaction is quenched with aqueous 10% HCl solution and extracted with EtOAc. The combined organic layers are washed with aqueous saturated NaHCO₃ solution, dried over MgSO₄, filtered and concentrated in vacuo. The crude material is purified on silica gel with 50% EtOAc/heptanes as eluent to deliver the titled compound (0.2 g, 34%) as a white foam.

¹H NMR (300 MHz, CDCl₃) δ 7.86 (m, 2H), 7.73 (m, 2H), 7.25 (m, 7H), 4.80 (s, 2H), 4.57 (m, 2H), 4.45 (m, 2H), 3.75 (m, 2H), 3.32 (s, 3H), 3.25 (m, 1H), 3.05 (m, 2H), 1.93 (m, 2H), 1.68 (m, 2H).

MS m/z: [M+H]+=641.

### G. [4-(5-Aminomethyl-2-chloro-phenyl)-piperidin-1-yl]-[1-(2-methoxy-ethyl)-7-trifluoromethoxy-1H-indol-2-yl]-methanone hydrochloride

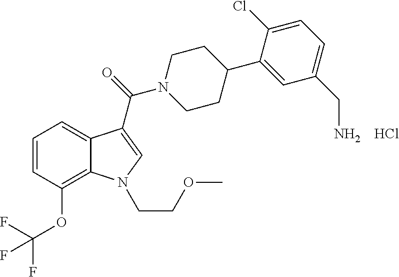

To a solution of 2-(4-chloro-3-{1-[1-(2-methoxy-ethyl)-7-trifluoromethoxy-1H-indole-2-carbonyl]-piperidin-4-yl}-benzyl)-isoindole-1,3-dione (0.46 g, 0.72 mmol) in THF (8 mL) is added hydrazine (0.51 g, 15.9 mmol) and the reaction heated to reflux. After 2 h the reaction was concentrated in vacuo to deliver a slurry which was triturated with EtOAc. The organic layer is treated with methanolic HCl and concentrated in vacuo. Recrystallization from EtOAc/MeOH delivers the titled compound (0.35 g, 89%) as an off-white solid.

¹H NMR (300 MHz, DMSO-d6) δ 8.21 (br s, 3H), 7.80 (m, 1H), 7.74 (m, 1H), 7.59 (m, 1H), 7.49 (m, 1H), 7.31 (m, 1H), 7.21 (m, 1H), 4.49 (m, 4H), 4.03 (m, 2H), 3.68 (m, 2H), 3.40-3.30 (m, 6H), 2.0-1.6 (m, 4H).

MS m/z: [M+H]⁺=510.

### Example 37

### [4-(5-Aminomethyl-2-fluoro-phenyl)-piperidin-1-yl]-[1-(2-diethylamino-ethyl)-1H-indol-3-yl]-methanone dihydrochloride

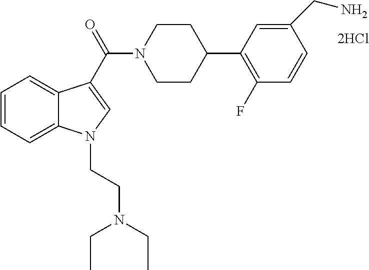

### A. Diethyl-(2-indol-1-yl-ethyl)-amine

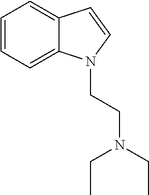

The title compound is prepared in a similar manner as described in Example 1E using 1H-indole and (2-bromo-ethyl)-diethyl-amine hydrobromide as the starting materials.

¹H NMR (300 MHz, CDCl₃) δ 7.6 (d, 1H), 7.4 (d, 1H), 7.3-7.0 (m, 3H), 6.5 (d, 1H), 4.2 (t, 2H), 2.8 (t, 2H), 2.6 (m, 4H), 1.0 (m, 6H).

MS m/z: [M+H]⁺=217.

### B. 1-[1-(2-Diethylamino-ethyl)-1H-indol-3-yl]-2,2,2-trifluoro-ethanone

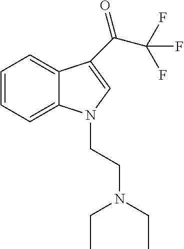

The title compound is prepared in a similar manner as described in Example 1F using diethyl-(2-indol-1-yl-ethyl)-amine as the starting material.

¹H NMR (300 MHz, CDCl₃) δ 8.4 (d, 1H), 8.1 (s, 1H), 7.5 (m, 3H), 4.8 (t, 2H), 3.5 (t, 2H), 3.2 (m, 4H), 1.5 (m, 6H).

MS m/z: [M+H]+=313.

### C. 1-(2-Diethylamino-ethyl)-1H-indole-3-carboxylic acid hydrochloride

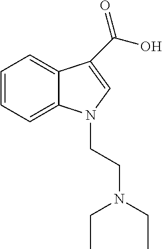

The title compound is prepared in a similar manner as described in Example 2H using 1-[1-(2-diethylamino-ethyl)-1H-indol-3-yl]-2,2,2-trifluoro-ethanone as the starting material.

¹H NMR (300 MHz, DMSO-d6) δ 12.0 (s, 1H), 10.6 (bs, 1H), 8.2 (s, 1H), 8.0 (d, 1H), 7.75 (d, 1H), 7.3 (m, 2H), 4.7 (t, 2H), 3.5 (t, 2H), 3.2 (m, 4H), 1.2 (m, 6H).

MS m/z: [M+H]⁺=261.

### D. N-(3-{1-[1-(2-Diethylamino-ethyl)-1H-indole-3-carbonyl]-piperidin-4-yl}-4-fluoro-benzyl)-2,2,2-trifluoro-acetamide trifluorocetate

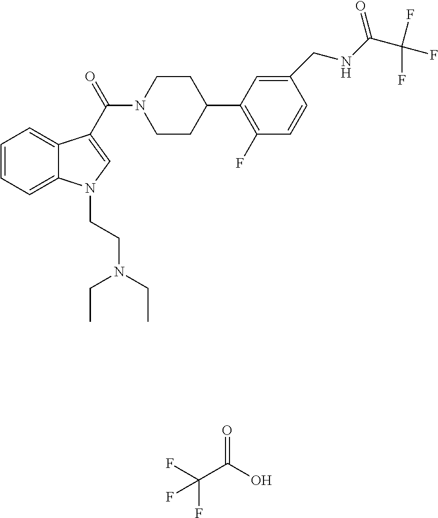

The title compound is prepared in a similar manner as described in Example 2I using 1-(2-diethylamino-ethyl)-1H-indole-3-carboxylic acid hydrochloride and 2,2,2-trifluoro-N-(4-fluoro-3-piperidin-4-yl-phenyl)-acetamide hydrochloride as the starting materials and is purified by RP-HPLC.

¹H NMR (300 MHz, CDCl₃) δ 7.8 (d, 1H), 7.7 (s, 1H), 7.4 (m, 2H), 7.2 (m, 2H), 7.0 (m, 3H), 4.8 (t, 2H), 4.6 (m, 2H), 4.5 (m, 2H), 3.4 (t, 2H), 3.1 (m, 7H), 1.9-1.8 (m, 4H), 1.3 (m, 6H).

LCMS m/z: [M+H]⁺=547.

### E. [4-(5-Aminomethyl-2-fluoro-phenyl)-piperidin-1-yl]-[1-(2-diethylamino-ethyl)-1H-indol-3-yl]-methanone dihydrochloride

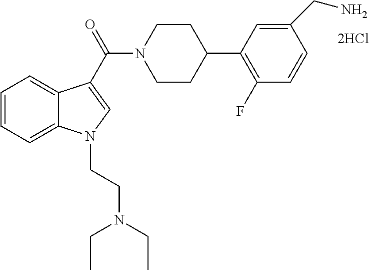

The title compound is prepared in a similar manner as described in Example 1K using N-(3-{1-[1-(2-diethylamino-ethyl)-1H-indole-3-carbonyl]-piperidin-4-yl}-4-fluoro-benzyl)-2,2,2-trifluoro-acetamide trifluorocetate as the starting material.

¹H NMR (300 MHz, DMSO-d6) δ 10.5 (bs, 1H), 8.3 (bs, 2H), 8.0 (s, 1H), 7.7 (m, 2H), 7.6 (d, 1H), 7.4-7.1 (m, 4H), 4.7 (t, 2H), 4.4 (m, 2H), 4.0 (m, 2H), 3.5 (m, 2H), 3.2 (m, 7H), 1.9-1.6 (m, 4H), 1.2 (m, 6H).

MS m/z: [M+H]⁺=451.

### Example 38

### [4-(5-Aminomethyl-2-fluoro-phenyl)-piperidin-1-yl]-[1-(2-pyrrolidin-1-yl-ethyl)-1H-indol-3-yl]-methanone dihydrochloride

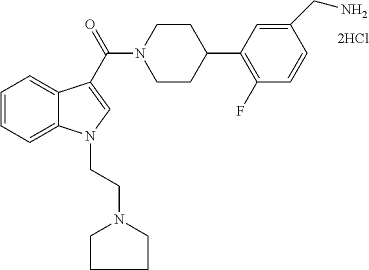

### A. 1-(2-Pyrrolidin-1-yl-ethyl)-1H-indole

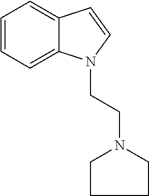

The title compound is prepared in a similar manner as described in Example 1E using 1H-indole and 1-(2-Chloro-ethyl)-pyrrolidine hydrochloride as the starting materials.

¹H NMR (300 MHz, CD₃OD) δ 7.7 (d, 1H), 7.4 (d, 1H), 7.3-7.1 (m, 3H), 6.5 (d, 1H), 4.3 (t, 2H), 2.9 (t, 2H), 2.6 (m, 4H), 1.8 (m, 4H).

MS m/z: [M+H]⁺=215.

### B. 2,2,2-Trifluoro-1-[1-(2-pyrrolidin-1-yl-ethyl)-1H-indol-3-yl]-ethanone

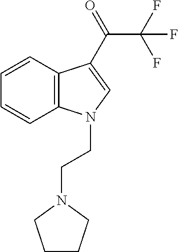

The title compound is prepared in a similar manner as described in Example 1F using 1-(2-pyrrolidin-1-yl-ethyl)-1H-indole as the starting material.

¹H NMR (300 MHz, CDCl₃) δ 8.4 (d, 1H), 8.1 (s, 1H), 7.4 (m, 3H), 4.8 (t, 2H), 3.8 (bs, H), 3.5 (t, 2H), 2.7 (bs, 1H), 2.1 (bs, 4H), 1.6 (bs, 2H).

MS m/z: [M+H]⁺=311.

### C. 1-(2-Pyrrolidin-1-yl-ethyl)-1H-indole-3-carboxylic acid hydrochloride

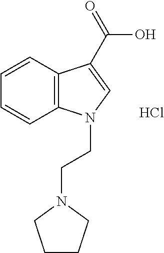

The title compound is prepared in a similar manner as described in Example 2H using 2,2,2-trifluoro-1-[1-(2-pyrrolidin-1-yl-ethyl)-1H-indol-3-yl]-ethanone as the starting material.

¹H NMR (300 MHz, DMSO-d6) δ 12.1 (s, 1H), 10.8 (bs, 1H), 8.2 (s, 1H), 8.1 (d, 1H), 7.75 (d, 1H), 7.3 (m, 2H), 4.7 (t, 2H), 3.6 (t, 2H), 3.5 (m, 2H), 3.0 (m, 2H), 1.9 (m, 2H), 1.8 (m, 2H).

MS m/z: [M+H]⁺=259.

### D. 2,2,2-Trifluoro-N-(4-fluoro-3-{1-[1-(2-pyrrolidin-1-yl-ethyl)-1H-indole-3-carbonyl]-piperidin-4-yl}-benzyl)-acetamide trifluorocetate

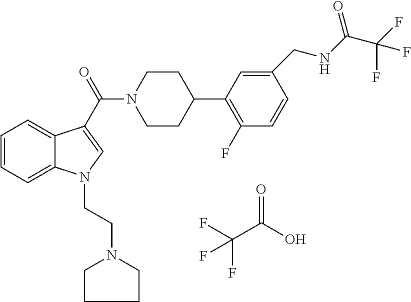

The title compound is prepared in a similar manner as described in Example 2I using 1-(2-pyrrolidin-1-yl-ethyl)-1H-indole-3-carboxylic acid hydrochloride and 2,2,2-trifluoro-N-(4-fluoro-3-piperidin-4-yl-phenyl)-acetamide hydrochloride as the starting materials and is purified by RP-HPLC.

¹H NMR (300 MHz, CDCl₃) δ 7.8 (d, 1H), 7.6 (s, 1H), 7.4-7.1 (m, 6H), 7.0 (m, 1H), 4.8 (t, 2H), 4.6 (m, 2H), 4.4 (m, 2H), 3.7 (m, 2H), 3.6 (m, 2H), 3.2 (m, 3H), 2.6 (m, 3H), 2.4 (m, 3 h), 1.9 (m, 2H), 1.8 (m, 2H).

MS m/z: [M+H]⁺=545.

### E. [4-(5-Aminomethyl-2-fluoro-phenyl)-piperidin-1-yl]-[1-(2-pyrrolidin-1-yl-ethyl)-1H-indol-3-yl]-methanone dihydrochloride

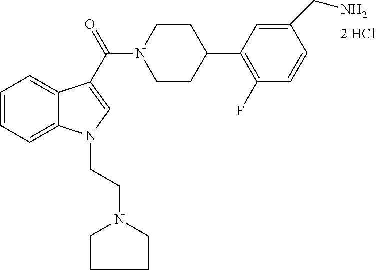

The title compound is prepared in a similar manner as described in Example 1K using 2,2,2-trifluoro-N-(4-fluoro-3-{1-[1-(2-pyrrolidin-1-yl-ethyl)-1H-indole-3-carbonyl]-piperidin-4-yl}-benzyl)-acetamide trifluorocetate as the starting material.

¹H NMR (300 MHz, DMSO-d6) δ11.0 (bs, 1H), 8.4 (bs, 2H), 7.9 (s, H), 7.8 (m, 2H), 7.6 (d, 1H), 7.4-7.2 (m, 4H), 4.7 (t, 2H), 4.5 (d, 2H), 4.0 (t, 2H), 3.6 (t, 2H), 3.2 (m, 3H), 2.9 (m, 3H), 2.0-1.6 (m, 9H).

MS m/z: [M+H]⁺=449.

### Example 39

### [4-(5-Aminomethyl-2-fluoro-phenyl)-piperidin-1-yl]-[7-fluoro-1-(2-methoxy-ethyl)-1H-indol-3-yl]-methanone hydrochloride

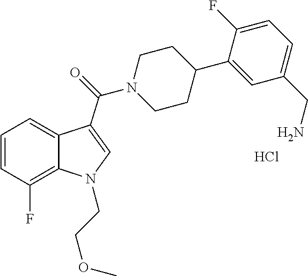

### A. 7-Fluoro-1-(2-methoxy-ethyl)-1H-indole

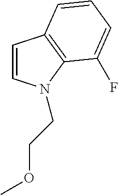

The title product (1.43 g, 100%) is obtained in a similar manner as described in Example 1E using 7-fluoro-1H-indole (1.00 g, 7.4 mmol) as the starting material. ¹H NMR (300 MHz, CDCl₃) δ 7.36 (d, J=8.1 Hz, 1H), 7.11 (d, J=3 Hz, 1H), 7.00-6.95 (m, 1H), 6.86 (dd, 1H), 6.48 (m, 1H), 4.45 (t, J=5.7 Hz, 2H), 3.73 (t, J=5.7 Hz, 2H), 3.30 (s, 3H);

¹⁹F NMR (300 MHz, CDCl₃) δ −135.93 (d, 3F).

### B. 2,2,2-Trifluoro-1-[7-fluoro-1-(2-methoxy-ethyl)-1H-indol-3-yl]-ethanone

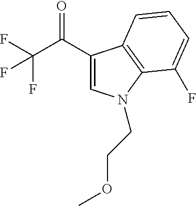

The title product (1.76 g, 82%) is obtained in a similar manner as described in Example 1F using 7-fluoro-1-(2-methoxy-ethyl)-1H-indole (1.43 g, 7.4 mmol) as the starting material.

¹H NMR (300 MHz, CDCl₃) δ 8.00 (d, 1H), 7.96 (s, 1H), 7.30-7.25 (m, 1H), 7.05 (dd, 1H), 4.53 (t, J=5.1 Hz, 2H), 3.77 (t, J=5.1 Hz, 2H), 3.32 (s, 3H);

¹⁹F NMR (300 MHz, CDCl₃) δ −72.25 (s, 3F), −134.23 (s, 1F).

### C. 7-Fluoro-1-(2-methoxy-ethyl)-1H-indole-3-carboxylic acid

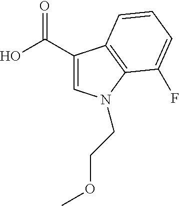

The title product (1.44 g, 100%) is obtained in a similar manner as described in Example 1G using 2,2,2-trifluoro-1-[7-fluoro-1-(2-methoxy-ethyl)-1H-indol-3-yl]-ethanone (1.76 g, 6.1 mmol) as the starting material.

¹H NMR (300 MHz, DMSO-d6) δ 12.17 (s, 1H), 8.04 (s, 1H), 7.83 (d, J=7.5 Hz, 1H), 7.18-7.11 (m, 1H), 7.08-7.01 (m, 1H), 4.50 (t, J=5.1 Hz, 2H), 3.69 (t, J=5.1 Hz, 2H), 3.22 (s, 3H);

¹⁹F NMR (300 MHz, DMSO-d6) δ −134.07 (d, 1F).

### D. 2,2,2-Trifluoro-N-(4-fluoro-3-{1-[7-fluoro-1-(2-methoxy-ethyl)-1H-indole-3-carbonyl]-piperidin-4-yl}-benzyl)-acetamide

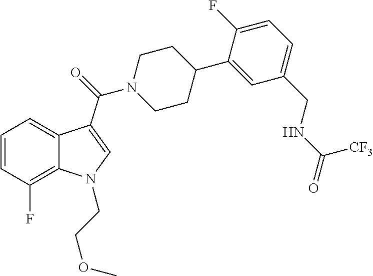

The title product (1.51 g, 85%) is obtained in a similar manner as described in Example 1J using 7-fluoro-1-(2-methoxy-ethyl)-1H-indole-3-carboxylic acid (0.81 g, 3.4 mmol) as the starting material.

¹H NMR (300 MHz, CDCl₃) δ 7.57 (d, 1H), 7.45 (s, 1H), 7.20-7.00 (m, 4H), 6.98-6.8 (dd, 1H), 6.70 (br s, 1H), 4.6 (br d, 1H), 4.50-4.60 (m, 4H), 3.74 (t, 2H), 3.31 (s, 3H), 3.20-3.00 (m, 4H), 1.95-1.90 (m, 2H), 1.80-1.70 (m, 2H);

¹⁹F NMR (300 MHz, CDCl₃) δ −74.57 (s, 3F), −119.11 (s, 1F), −135.10 (s, 1F).

### E. [4-(5-Aminomethyl-2-fluoro-phenyl)-piperidin-1-yl]-[7-fluoro-1-(2-methoxy-ethyl)-1H-indol-3-yl]-methanone hydrochloride

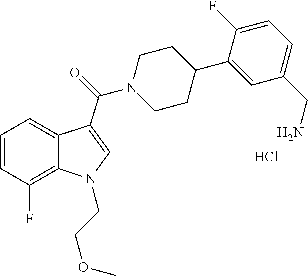

The title product (0.81 g, 60%) is obtained in a similar manner as described in Example 1K using 2,2,2-trifluoro-N-(4-fluoro-3-{1-[7-fluoro-1-(2-methoxy-ethyl)-1H-indole-3-carbonyl]-piperidin-4-yl}-benzyl)-acetamide (1.51 g, 2.9 mmol) as the starting material.

¹H NMR (300 MHz, DMSO-d6) δ 8.27 (br s, 2H), 7.75 (s, 1H), 7.57-55 (m, 1H), 7.52 (d, J=7.5 Hz, 1H), 7.36 (m, 1H), 7.23 (dd, J=8.7, 10.5 Hz, 1H), 7.11-7.01 (m, 2H), 4.50 (m, 2H), 4.40 (br d, 1H), 4.00 (t, 2H), 3.70 (t, 2H), 3.22 (s, 3H), 3.17-3.09 (m, 4H), 1.82-1.79 (m, 2H), 1.70-1.60 (m, 2H);

¹⁹F NMR (300 MHz, DMSO-d6) δ −119.89 (s, 1F), −134.37 (s, 1F)

LC 2.34 min; MS 428 (M+1, 100%).

### Example 40

### [4-(5-Aminomethyl-2-fluoro-phenyl)-piperidin-1-yl]-[1-(2-pyridin-2-yl-ethyl)-1H-indol-3-yl]-methanone dihydrochloride

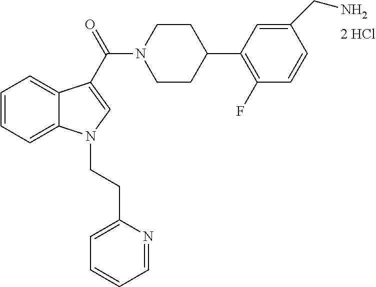

### A. 1-(2-Pyridin-2-yl-ethyl)-1H-indole

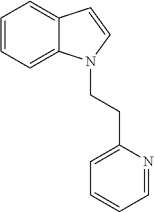

The title compound is prepared in a similar manner as described in Example 1E using 1H-Indole and 2-(2-bromo-ethyl)-pyridine hydrobromide as the starting materials.

¹H NMR (300 MHz, CDCl₃) δ 8.6 (d, 1H), 7.6 (d, 1H), 7.5 (m, 1H), 7.4 (d, 1H), 7.2 (m, 3H), 7.0 (d, H), 6.9 (d, 1H), 6.4 (d, 1H), 4.6 (t, 2H), 3.3 (t, 2H).

MS m/z: [M+H]⁺=223.

### B. 2,2,2-Trifluoro-1-[1-(2-pyridin-2-yl-ethyl)-1H-indol-3-yl]-ethanone

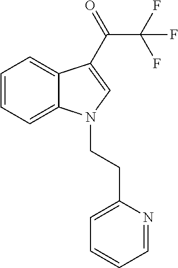

The title compound is prepared in a similar manner as described in Example 1F using 1-(2-pyridin-2-yl-ethyl)-1H-indole as the starting material.

¹H NMR (300 MHz, CDCl₃) δ 8.8 (bs, 1H), 8.4 (d, 1H), 7.9 (m, 1H), 7.7 (s, 1H), 7.6-7.2 (m, 4H), 7.0 (m, 1H), 4.8 (t, 2H), 3.6 (t, 2H).

MS m/z: [M+H]⁺=319.

### C. 1-(2-Pyridin-2-yl-ethyl)-1H-indole-3-carboxylic acid hydrochloride

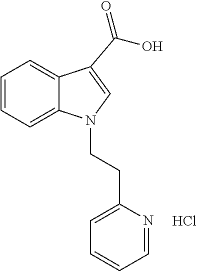

The title compound is prepared in a similar manner as described in Example 4C using 2,2,2-trifluoro-1-[1-(2-pyridin-2-yl-ethyl)-1H-indol-3-yl]-ethanone as the starting material.

¹H NMR (300 MHz, DMSO-d6) δ 8.6 (d, 1H), 8.0 (m, 3H), 7.6 (d, 4H), 7.5 (m, 2H), 4.7 (t, 2H), 3.4 (t, 2H).

MS m/z: [M+H]⁺=267.

### D. 2,2,2-Trifluoro-N-(4-fluoro-3-{1-[1-(2-pyridin-2-yl-ethyl)-1H-indole-3-carbonyl]-piperidin-4-yl}-benzyl)-acetamide

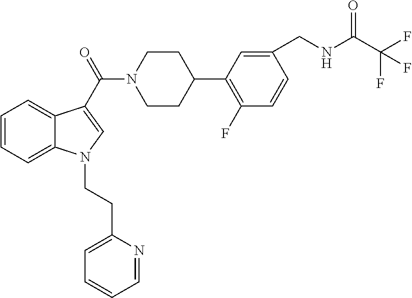

The title compound is prepared in a similar manner as in Example 2I using 1-(2-pyridin-2-yl-ethyl)-1H-indole-3-carboxylic acid hydrochloride and 2,2,2-trifluoro-N-(4-fluoro-3-piperidin-4-yl-phenyl)-acetamide hydrochloride as the starting materials.

¹H NMR (300 MHz, CDCl₃) δ 8.6 (d, 1H), 7.8 (d, 1H), 7.5 (m, 1H), 7.4 (d, 1H), 7.2 (m, 6H), 7.0 (m, 1H), 6.9 (d, 1H), 6.6 (bs, 1H), 4.6 (t, 2H), 4.5 (m, 4H), 3.3 (t, 2H), 3.1 (m, 3H), 1.9 (m, 2H), 1.8 (m, 2H).

MS m/z: [M+H]⁺=553.

### E. [4-(5-Aminomethyl-2-fluoro-phenyl)-piperidin-1-yl]-[1-(2-pyridin-2-yl-ethyl)-1H-indol-3-yl]-methanone dihydrochloride

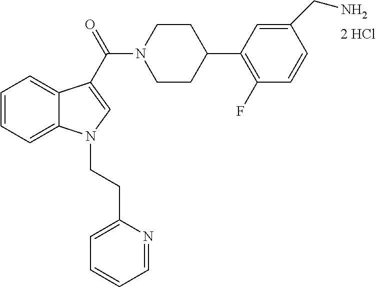

The title compound is prepared in a similar manner as described in Example 1K using 2,2,2-trifluoro-N-(4-fluoro-3-{1-[1-(2-pyridin-2-yl-ethyl)-1H-indole-3-carbonyl]-piperidin-4-yl}-benzyl)-acetamide as the starting material.

¹H NMR (300 MHz, DMSO-d6) δ 8.7 (d, 1H), 8.4 (bs, 2H), 8.2 (m, 1H), 7.6 (m, 1H), 7.4 (m, 1H), 7.2 (m, 3H), 4.7 (t, 2H), 4.4 (m, 2H), 4.0 (m, 2H), 3.4 (t, 2H), 3.1 (m, 3H), 1.8 (m, 2H), 1.6 (m, 2H).

MS m/z: [M+H]⁺=449.

### Example 41

### [4-(5-Aminomethyl-2-fluoro-phenyl)-piperidin-1-yl]-[1-(2-piperidin-1-yl-ethyl)-1H-indol-3-yl]-methanone dihydrochloride

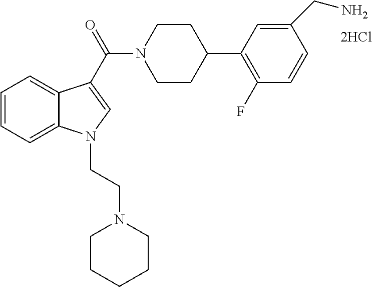

### A. 1-(2-Piperidin-1-yl-ethyl)-1H-indole

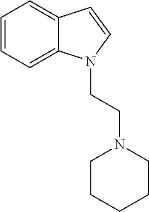

The title compound is prepared in a similar manner as described in Example 1E using 1H-indole and 1-(2-chloro-ethyl)-piperidine hydrochloride as the starting materials.

¹H NMR (300 MHz, CDCl₃) δ 7.6 (d, 1H), 7.4 (d, 1H), 7.2 (m, 4H), 6.5 (d, 1H), 4.3 (t, 2H), 2.7 (t, 2H), 2.4 (m, 4H), 1.6 (m, 3H), 1.4 (m, 2H).

MS m/z: [M+H]⁺=229.

### B. 2,2,2-Trifluoro-1-[1-(2-piperidin-1-yl-ethyl)-1H-indol-3-yl]-ethanone

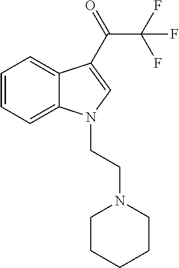

The title compound is prepared in a similar manner as described in Example 1F using 1-(2-piperidin-1-yl-ethyl)-1H-indole as the starting material.

¹H NMR (300 MHz, CDCl₃) δ 8.4 (d, 1H), 8.0 (m, 1H), 7.4 (m, 3H), 4.8 (t, 2H), 3.6 (m, 2H), 3.4 (t, 2H), 2.6 (m, 2H), 1.9 (m, 6H).

MS m/z: [M+H]⁺=325.

### C. 1-(2-Piperidin-1-yl-ethyl)-1H-indole-3-carboxylic acid hydrochloride

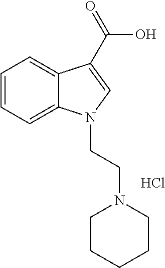

The title compound is prepared in a similar manner as described in Example 2H using 2,2,2-trifluoro-1-[1-(2-piperidin-1-yl-ethyl)-1H-indol-3-yl]-ethanone as the starting material.

¹H NMR (300 MHz, DMSO-d6) δ 12.0 (bs, 1H), 11.0 (bs, 1H), 8.2 (s, 1H), 8.0 (d, 1H), 7.8 (d, 1H), 7.3 (m, 2H), 4.8 (t, 2H), 3.5 (m, 4H), 3.0 (m, 2H), 1.7 (m, 5H), 1.4 (m, 1H)

LCMS m/z: [M+H]⁺=273.

### D. 2,2,2-Trifluoro-N-(4-fluoro-3-{1-[1-(2-piperidin-1-yl-ethyl)-1H-indole-3-carbonyl]-piperidin-4-yl}-benzyl)-acetamide trifluorocetate

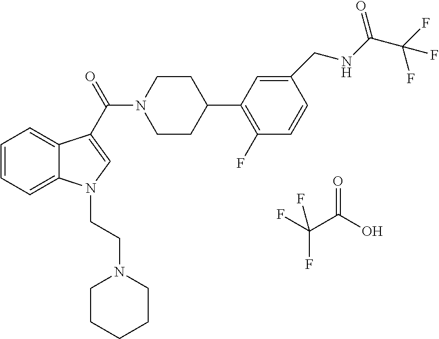

The title compound is prepared in a similar manner as described in Example 2I using 1-(2-piperidin-1-yl-ethyl)-1H-indole-3-carboxylic acid hydrochloride and 2,2,2-trifluoro-N-(4-fluoro-3-piperidin-4-yl-phenyl)-acetamide hydrochloride as the starting materials and is purified by RP-HPLC.

¹H NMR (300 MHz, CDCl₃) δ 7.8 (d, 1H), 7.6 (s, 1H), 7.4-7.1 (m, 5H), 7.0-6.8 (m, 2H), 4.8 (t, 2H), 4.6 (m, 2H), 4.4 (d, 2H), 3.5 (m, 2H), 3.4 (t, 2H), 3.1 (m, 3H), 2.6 (m, 2H), 2.0-1.6 (m, 9H), 1.4 (m, 1H).

MS m/z: [M+H]⁺=559.

### E. [4-(5-Aminomethyl-2-fluoro-phenyl)-piperidin-1-yl]-[1-(2-piperidin-1-yl-ethyl)-1H-indol-3-yl]-methanone dihydrochloride

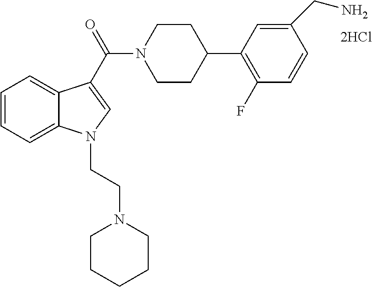

The title compound is prepared in a similar manner as described Example 1K using 2,2,2-trifluoro-N-(4-fluoro-3-{1-[1-(2-piperidin-1-yl-ethyl)-1H-indole-3-carbonyl]-piperidin-4-yl}-benzyl)-acetamide trifluorocetate as the starting material.

¹H NMR (300 MHz, DMSO-d6) δ 11.0 (bs, 1H), 8.4 (bs, 2H), 7.9 (s, 1H), 7.8 (d, 2H), 7.6 (d, 1H), 7.4 (m, 1H), 7.2 (m, 3H), 4.8 (t, 2H), 4.5 (m, 2H), 4.0 (m, 4H), 3.4 (m, 3H), 3.2 (m, 3H), 2.9 (m, 2H), 2.0-1.6 (m, 8H) 1.4 (m, H).

MS m/z: [M+H]⁺=463.

### Example 42

### [4-(5-Aminomethyl-2-fluoro-phenyl)-piperidin-1-yl]-[4-chloro-1-(2-methoxy-ethyl)-1H-indol-3-yl]-methanone

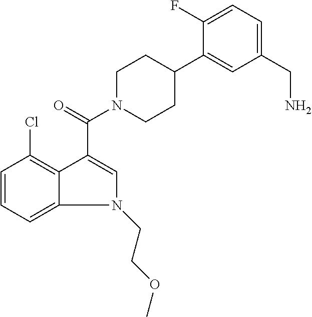

### A. 4-Chloro-1-(2-methoxy-ethyl)-1H-indole

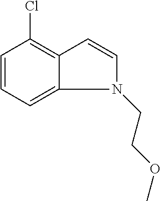

To a 0° C. solution of 4-chloroindole (2.0 g, 13.2 mmol) in DMF (40 mL) under nitrogen is added sodium hydride (0.5 g, 19.8 mmol, 60% suspension in oil). After stirring for 10 min at 0° C., 1-bromo-2-methoxy-ethane (2.7 g, 19.8 mmol) is added followed by a catalytic amount of NaI. After stirring an additional 2 h at 0° C. the reaction is quenched with aqueous sat NaHCO₃ solution and extracted with EtOAc. The combined organic layers are dried over MgSO₄, filtered and concentrated in vacuo. The crude material is purified on silica gel with 30% EtOAc/heptane as eluent to deliver the titled compound (2.8 g, 100%) as an orange oil.

¹H NMR (300 MHz, CDCl₃) δ 7.25 (m, 2H), 7.11 (m, 2H), 6.63 (m, 1H), 4.29 (t, J=5.3 Hz, 2H), 3.70 (t, J=5.3 Hz, 2H), 3.32 (s, 3H).

### B. 1-[4-Chloro-1-(2-methoxy-ethyl)-1H-indol-3-yl]-2,2,2-trifluoro-ethanone

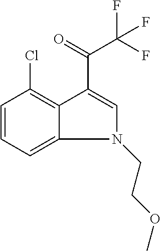

To a 0° C. solution of 4-chloro-1-(2-methoxy-ethyl)-1H-indole (2.8 g, 13.2 mmol) in DMF (20 mL) under nitrogen is added trifluoracetic anhydride (11.1 g, 52.8 mmol). The reaction is allowed to warm to r.t. over 9 h. At 0° C. the reaction is quenched with aqueous sat NaHCO₃ solution and extracted with EtOAc. The combined organic layers are dried over MgSO₄, filtered and concentrated in vacuo to deliver the titled compound (3.2 g, 79%) as white needles (recrystallized from EtOAc/heptane). mp 57-59° C.

¹H NMR (300 MHz, CDCl₃) δ 8.06 (m, 1H), 7.30 (m, 3H), 4.36 (t, J=4.9 Hz, 2H), 3.74 (t, J=4.9 Hz, 2H), 3.33 (s, 3H);

MS 306 (M+1).

### C. 4-Chloro-1-(2-methoxy-ethyl)-1H-indole-3-carboxylic acid

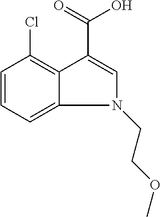

To a solution of 1-[4-chloro-1-(2-methoxy-ethyl)-1H-indol-3-yl]-2,2,2-trifluoro-ethanone (2.0 g, 6.54 mmol) in EtOH/H₂O (40 mL/20 mL) is added KOH (3.7 g, 65.4 mmol) and the reaction is heated on a steam bath for 30 min. The resulting clear solution is concentrated in vacuo, cooled to 0° C., acidified with 10% aqueous HCl and extracted with EtOAc. The organic layer is dried over MgSO₄, filtered and concentrated in vacuo. The resulting solid is recrystallized from CH₂Cl₂/heptane to deliver the desired product (1.51 g, 91%) as a light orange solid: mp 142-145° C.

¹H NMR (300 MHz, CDCl₃) δ 8.06 (m, 1H), 7.30 (m, 3H), 4.31 (t, J=4.9 Hz, 2H), 3.73 (t, J=4.9 Hz, 2H), 3.32 (s, 3H);

MS 254 (M+1).

### D. N-(3-{1-[4-Chloro-1-(2-methoxy-ethyl)-1H-indole-3-carbonyl]-piperidin-4-yl}-4-fluoro-benzyl)-2,2,2-trifluoro-acetamide

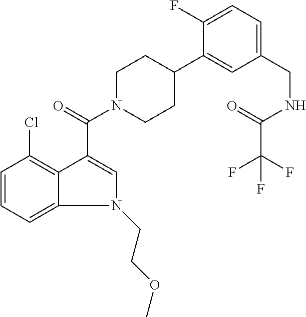

To a mixture of 4-chloro-1-(2-methoxy-ethyl)-1H-indole-3-carboxylic acid (0.25 g, 1.0 mmol) in THF (6 mL) under nitrogen is added carbonyl diimidazole (0.19 g, 1.2 mmol). After heating to boil and then stirring 1 h at ambient temperature 2,2,2-trifluoro-N-(4-fluoro-3-piperidin-4-yl-benzyl)-acetamide (0.33 g, 1.1 mmol) is added and the reaction is heated to reflux overnight. The reaction is quenched with 10% aqueous HCl and extracted with EtOAc. The combined organic layers are washed with aqueous saturated NaHCO₃ solution, dried over MgSO₄, filtered and concentrated in vacuo to deliver the titled compound (0.41 g, 76%) as a white foam.

¹H NMR (300 MHz, CDCl₃) δ 7.2-7.0 (m, 7H), 6.6 (br s, 1H), 5.0 (m, 1H), 4.5 (m, 2H), 4.3 (t, J=5.3 Hz, 2H), 3.8 (m, 1H), 3.7 (t, J=5.3 Hz, 2H) 3.3 (s, 3H), 3.1 (m, 2H), 2.9 (m, 1H), 1.9-1.6 (m, 4H);

MS 540 (M+1).

### E. [4-(5-Aminomethyl-2-fluoro-phenyl)-piperidin-1-yl]-[4-chloro-1-(2-methoxy-ethyl)-1H-indol-3-yl]-methanone

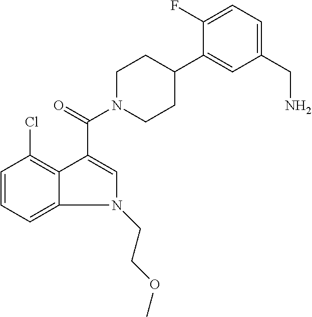

To a solution of N-(3-{1-[4-chloro-1-(2-methoxy-ethyl)-1H-indole-3-carbonyl]-piperidin-4-yl}-4-fluoro-benzyl)-2,2,2-trifluoro-acetamide (0.40 g, 0.74 mmol) in MeOH (6 mL) and H₂O (2 mL) is added aqueous 50% NaOH solution (1.0 mL). After stirring at ambient temperature overnight the mixture is concentrated in vacuo. To the resulting material is added aqueous sat NaHCO₃ solution and the mixture extracted with EtOAc. The combined organic layers are dried over MgSO₄, filtered and concentrated in vacuo diluted with MeOH and adsorbed onto silica gel. This material is purified on silica gel using 5% MeOH/CH₂Cl₂ as the eluent. Concentration of appropriate fractions delivers the titled compound (0.19 g, 58%) as a white foam.

¹H NMR (300 MHz, CDCl₃) δ 7.34 (m, 2H), 7.19 (m, 5H), 6.98 (m, 1H), 5.01 (m, 1H), 4.28 (t, J=5.3 Hz, 2H), 3.84 (m, 2H), 3.71 (t, J=5.3 Hz, 2H), 3.32 (s, 3H), 3.11 (m, 3H), 2.90 (m, 1H), 1.94-1.52 (m, 5H);

MS 444 (M+1).

### Example 43

### [4-(5-Aminomethyl-2-fluoro-phenyl)-piperidin-1-yl]-(1-butyl-7-trifluoromethoxy-1H-indol-3-yl)-methanone hydrochloride

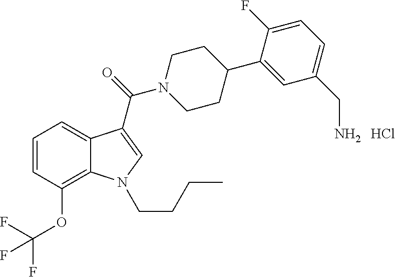

### A. 1-Butyl-7-trifluoromethoxy-1H-indole

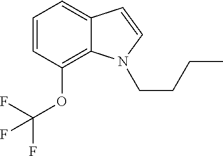

The title compound is prepared in a similar manner as described in Example 1E using 7-trifluoromethoxyindole and 1-bromobutane as the starting materials.

¹H NMR (300 MHz, CDCl₃) δ 7.53-7.50 (dd, 1H), 7.10-7.00 (m, 3H), 6.50 (d, 1H), 4.28 (t, 2H), 1.80 (quin, 2H), 1.35 (sext, 2H), 0.95 (t, 3H).

### B. 1-(1-Butyl-7-trifluoromethoxy-1H-indol-3-yl)-2,2,2-trifluoro-ethanone

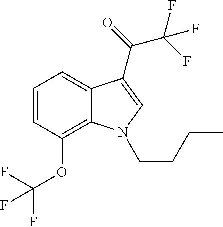

The title compound is prepared in a similar manner as described in Example 2G using 1-butyl-7-trifluoromethoxy-1H-indole as the starting material.

¹H NMR (300 MHz, CDCl₃) δ 8.35 (d, 1H), 7.90 (m, 1H), 7.36-7.31 (m, 1H), 7.24 (br s, 1H), 4.37 (t, 2H), 1.89 (quin, 2H), 1.40 (sext, 2H), 0.98 (t, 3H).

### C. 1-Butyl-7-trifluoromethoxy-1H-indole-3-carboxylic acid

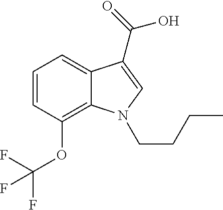

The title compound is prepared in a similar manner as described in Example 2H using 1-(1-butyl-7-trifluoromethoxy-1H-indol-3-yl)-2,2,2-trifluoro-ethanone as the starting material.

¹H NMR (300 MHz, DMSO-d6) δ 8.22-8.18 (m, 1H), 7.94 (s, 1H), 7.18-7.11 (m, 2H), 4.28 (t, 2H), 1.72 (quin, 2H), 1.25 (sext, 2H), 0.87 (t, 3H).

### D. N-{3-[1-(1-Butyl-7-trifluoromethoxy-1H-indole-3-carbonyl)-piperidin-4-yl]-4-fluoro-benzyl}-2,2,2-trifluoro-acetamide

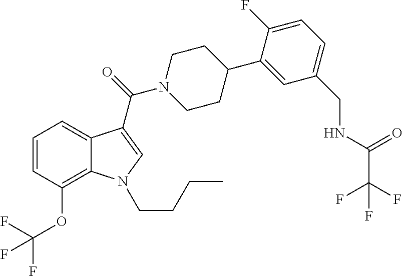

The title compound is prepared in a similar manner as described in Example 6E using 1-butyl-7-trifluoromethoxy-1H-indole-3-carboxylic acid and 2,2,2-trifluoro-N-(4-fluoro-3-piperidin-4-yl-benzyl)-acetamide hydrochloride as the starting materials.

¹H NMR (300 MHz, CD₃OD) δ 7.69-7.65 (m, 2H), 7.30-7.27 (m, 1H), 7.20-7.15 (m, 3H), 7.07-7.01 (m, 1H), 4.52 (br s, 2H), 4.41-4.34 (m, 4H), 3.20 (br s, 4H), 1.91-1.72 (m, 6H), 1.35 (sext, 2H), 0.95 (t, 3H).

### E. [4-(5-Aminomethyl-2-fluoro-phenyl)-piperidin-1-yl]-(1-butyl-7-trifluoromethoxy-1H-indol-3-yl)-methanone hydrochloride

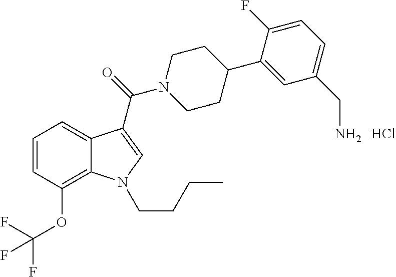

The title compound is prepared in a similar manner as described in Example 3B using N-{3-[1-(1-butyl-7-trifluoromethoxy-1H-indole-3-carbonyl)-piperidin-4-yl]-4-fluoro-benzyl}-2,2,2-trifluoro-acetamide as the starting material.

¹H NMR (300 MHz, DMSO-d6) δ 8.57 (br s, 3H), 7.91 (s, 1H), 7.76-7.70 (m, 1H), 7.67-7.64 (m, 1H), 7.43-7.38 (m, 1H), 7.24-7.17 (m, 3H), 4.41 (br d, 2H), 4.33 (t, 2H), 4.02-3.96 (m, 2H), 3.20-3.07 (m, 3H), 1.83-1.66 (m, 6H), 1.28 (sext, 2H), 0.90 (t, 3H).

### Example 44

### [4-(5-Aminomethyl-2-fluoro-phenyl)-piperidin-1-yl]-[1-(2-methoxy-ethyl)-4-methyl-1H-indol-3-yl]-methanone hydrochloride

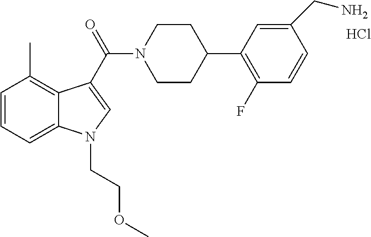

### A. 1-(2-Methoxy-ethyl)-4-methyl-1H-indole

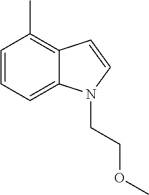

The title compound is prepared in a similar manner as described in Example 1E using 4-methylindole as the starting material.

¹H NMR (300 MHz, CDCl₃) δ 7.2 (m, 3H), 6.9 (m, 1H), 6.5 (d, 1H), 4.3 (t, 2H), 3.7 (t, 2H), 3.3 (s, 3H), 2.6 (s, 3H).

MS m/z: [M+H]⁺=190.

### B. 2,2,2-Trifluoro-1-[1-(2-methoxy-ethyl)-4-methyl-1H-indol-3-yl]-ethanone

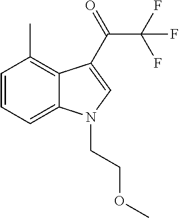

The title compound is prepared in a similar manner as described in Example 1F using 1-(2-methoxy-ethyl)-4-methyl-1H-indole as the starting material.

¹H NMR (300 MHz, CDCl₃) δ 8.0 (s, 1H), 7.3 (m, 2H), 7.1 (m, 1H), 4.4 (t, 2H), 3.8 (t, 2H), 3.4 (s, 3H), 2.8 (s, 3H).

MS m/z: [M+H]⁺=234.

### C. 1-(2-Methoxy-ethyl)-4-methyl-1H-indole-3-carboxylic acid

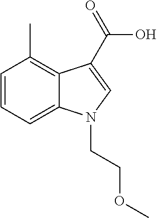

The title compound is prepared in a similar manner as described in Example 4C using 2,2,2-trifluoro-1-[1-(2-methoxy-ethyl)-4-methyl-1H-indol-3-yl]-ethanone as the starting material.

¹H NMR (300 MHz, DMSO-d6) δ 11.7 (bs, 1H), 8.0 (s, 1H), 7.4 (d, 1H), 7.2 (m, 1H), 6.9 (m, 1H), 4.4 (t, 2H), 3.8 (t, 2H), 3.2 (s, 3H), 2.8 (s, 3H).

MS m/z: [M+H]⁺=234.

### D. 2,2,2-Trifluoro-N-(4-fluoro-3-{1-[1-(2-methoxy-ethyl)-4-methyl-1H-indole-3-carbonyl]-piperidin-4-yl}-benzyl)acetamide

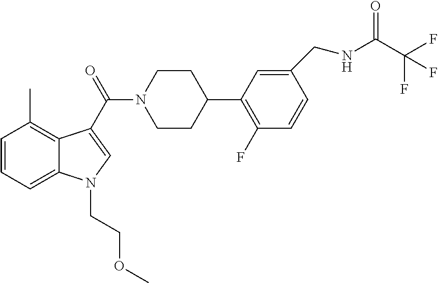

The title compound is prepared in a similar manner as described in Example 2I using 1-(2-methoxy-ethyl)-4-methyl-1H-indole-3-carboxylic acid and 2,2,2-trifluoro-N-(4-fluoro-3-piperidin-4-yl-benzyl)-acetamide hydrochloride as the starting materials.

¹H NMR (300 MHz, CDCl₃) δ 7.3-7.1 (m, 5H), 7.0 (m, 2H), 6.8 (bs, 1H), 4.5 (m, 2H), 4.3 (m, 2H), 3.7 (m, 2H), 3.3 (s, 3H), 2.6 (s, 3H), 1.9-1.6 (m, 6H).

MS m/z: [M+H]⁺=520.

### E [4-(5-Aminomethyl-2-fluoro-phenyl)-piperidin-1-yl]-[1-(2-methoxy-ethyl)-4-methyl-1H-indol-3-yl]-methanone hydrochloride

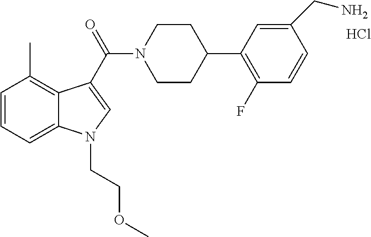

The title compound is prepared in a similar manner as described in Example 1K using 2,2,2-trifluoro-N-(4-fluoro-3-{1-[1-(2-methoxy-ethyl)-4-methyl-1H-indole-3-carbonyl]-piperidin-4-yl}-benzyl)-acetamide as the starting material.

¹H NMR (300 MHz, DMSO-d6) δ 8.4 (bs, 2H), 7.5 (m, 2H), 7.4 (m, 2H), 7.2 (m, 1H), 7.1 (m, 1H), 6.9 (m, 1H), 4.4 (t, 2H), 4.0 (m, 2H), 3.7 (t, 2H), 3.6 (m, 2H), 3.2 (s, 3H), 3.1 (m, 3H), 2.4 (s, 3H), 1.8 (m, 2H), 1.6 (m, 2H).

MS m/z: [M+H]⁺=424.

### Example 45

### [4-(5-Aminomethyl-2-fluoro-phenyl)-piperidin-1-yl]-[1-(2,2-difluoro-2-phenyl-ethyl)-1H-indol-3-yl]-methanone hydrochloride

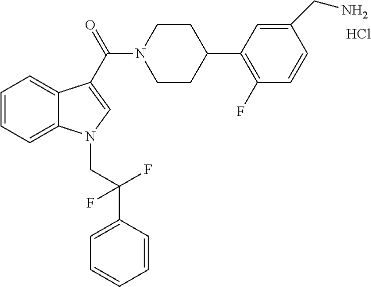

### A. 1-(2-Oxo-2-phenyl-ethyl)-1H-indole-3-carboxylic acid methyl ester

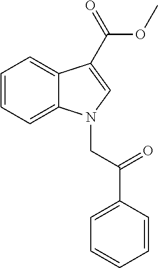

A mixture of 1H-indole-3-carboxylic acid methyl ester (2.00 g, 11.43 mmol), 2-bromo-1-phenyl-ethanone (2.27 g, 11.43 mmol) and K₂CO₃ (3.15 g, 22.86 mmol) in acetonitrile (70 mL) is heated to reflux overnight. The reaction mixture is poured into ice/water and the resulting precipitate is collected. The crude product is triterated with CH₂Cl₂ to yield the titled compound (1.25 g, 37%).

¹H NMR (300 MHz, CDCl₃) δ 8.2 (m, 1H), 8.0 (m, 2H), 7.8 (m, 1H), 7.7 (m, 1H), 7.5 (m, 2H), 7.2 (m, 3H), 5.6 (s, 2H), 3.9 (s, 3H).

MS m/z: [M+H]⁺=294.

### B. 1-(2,2-Difluoro-2-phenyl-ethyl)-1H-indole-3-carboxylic acid methyl ester

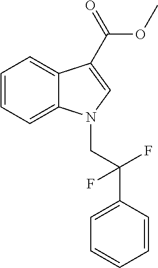

A solution of 1-(2-oxo-2-phenyl-ethyl)-1H-indole-3-carboxylic acid methyl ester (0.7 g, 2.4 mmol) and diethylaminosulfur trifluoride (0.8 g, 4.8 mmol) in benzene (15 mL) is heated at 65° C. overnight. The reaction mixture is poured into EtOAc and the organic layer washed with H₂O (2×) and brine, dried with MgSO₄, filtered and concentrated in vacuo to give the crude product. Purification by flash chromatography on SiO₂ eluting with 10% ethyl acetate/heptane gives 0.2 g, (26%) of the title compound.

¹H NMR (300 MHz, CDCl₃) δ 8.2 (m, 1H), 7.7 (m, 1H), 7.4-7.25 (m, 8H), 4.7 (m, 2H), 3.9 (s, 3H).

MS m/z: [M+H]⁺=316.

### C. 1-(2,2-Difluoro-2-phenyl-ethyl)-1H-indole-3-carboxylic acid

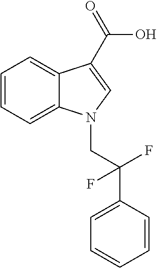

The title compound is prepared in a similar manner as described in Example 5D using 1-(2,2-difluoro-2-phenyl-ethyl)-1H-indole-3-carboxylic acid methyl ester as the starting material.

¹H NMR (300 MHz, DMSO-d6) δ12.1 (bs, 1H), 8.0 (m, 2H), 7.5 (m, 6H), 7.2 (m, 2H), 5.1 (t, 2H).

MS m/z: [M+H]⁺=302.

### D. N-(3-{1-[1-(2,2-Difluoro-2-phenyl-ethyl)-1H-indole-3-carbonyl]-piperidin-4-yl}-4-fluoro-benzyl)-2,2,2-trifluoro-acetamide

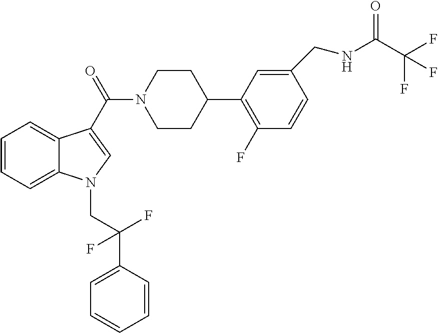

The title compound is prepared in a similar manner as described in Example 2I using 1-(2,2-difluoro-2-phenyl-ethyl)-1H-indole-3-carboxylic acid and 2,2,2-trifluoro-N-(4-fluoro-3-piperidin-4-yl-benzyl)-acetamide hydrochloride as the starting materials.

¹H NMR (300 MHz, CDCl₃) δ 7.7 (m, 1H), 7.4-7.2 (m, 11H), 7.1 (m, 1H), 6.6 (bs, H), 4.6 (m, 2H), 4.5 (m, 4H), 3.1 (m, 3H), 1.9 (m, 2H), 1.7 (m, 2H).

MS m/z: [M+H]⁺=588.

### E. [4-(5-Aminomethyl-2-fluoro-phenyl)-piperidin-1-yl]-[1-(2,2-difluoro-2-phenyl-ethyl)-1H-indol-3-yl]-methanone hydrochloride

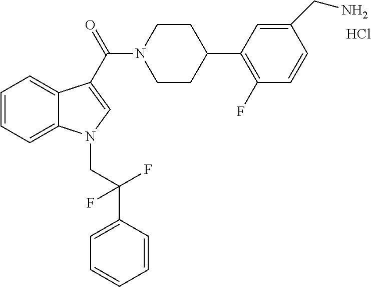

The title compound is prepared in a similar manner as described Example 1K using N-(3-{1-[1-(2,2-difluoro-2-phenyl-ethyl)-1H-indole-3-carbonyl]-piperidin-4-yl}-4-fluoro-benzyl)-2,2,2-trifluoro-acetamide as the starting material.

¹H NMR (300 MHz, DMSO-d6) δ 8.5 (bs, 2H), 7.8-7.0 (m, 13H), 5.2 (m, 2H), 4.4 (m, 2H), 4.0 (m, 2H), 3.2 (m, 3H), 1.9-1.6 (m, 4H).

LCMS m/z: [M+H]⁺=492.

### Example 46

### [4-(5-Aminomethyl-2-fluoro-phenyl)-piperidin-1-yl]-(1-butyl-4-methoxy-1H-indol-3-yl)-methanone hydrochloride

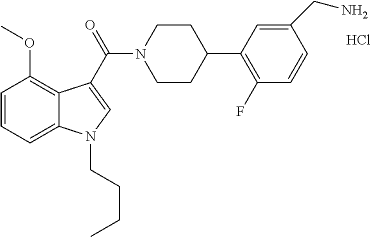

### A. 1-Butyl-4-methoxy-1H-indole

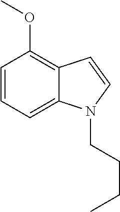

The title compound is prepared in a similar manner as described in Example 1E using 4-methoxyindole and 1-bromo-butane as the starting materials.

¹H NMR (300 MHz, CDCl₃) δ 7.1 (m, 1H), 7.0 (m, 2H), 6.6 (d, H), 6.5 (d, 1H), 4.1 (t, 2H), 4.0 (s, 3H), 1.8 (m, 2H), 1.5 (m, 2H), 0.9 (m, 3H).

MS m/z: [M+H]⁺=204.

### B. 1-(1-Butyl-4-methoxy-1H-indol-3-yl)-2,2,2-trifluoro-ethanone

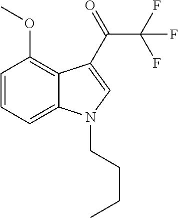

The title compound is prepared in a similar manner as described in Example 1F using 1-butyl-4-methoxy-1H-indole as the starting material.

¹H NMR (300 MHz, CDCl₃) δ 7.8 (d, 1H), 7.3 (m, 1H), 7.0 (d, 1H), 6.8 (d, 1H), 4.2 (t, 2H), 4.0 (s, 3H), 1.9 (m, 2H), 1.4 (m, 2H), 1.0 (m, 3H).

MS m/z: [M+H]⁺=300.

### C. 1-Butyl-4-methoxy-1H-indole-3-carboxylic acid

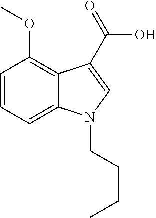

1-(1-Butyl-4-methoxy-1H-indol-3-yl)-2,2,2-trifluoro-ethanone (2.6 g, 8.7 mmol) in 6 N NaOH (35 mL) is heated to reflux until no starting material is present. The reaction mixture is cooled to room temperature, diluted with H₂O (100 mL) and acidified to pH=2 with concentrated HCl. The reaction mixture is extracted with EtOAc (2×) and the organic layers are combined and dried over Na₂SO₄, filtered and concentrated in vacuo to yield the title compound (2.0 g, 93%).

¹H NMR (300 MHz, DMSO-d6) δ 11.6 (s, 1H), 8.0 (s, 1H), 7.2 (d, 1H), 6.8 (m, 1H), 4.2 (t, 2H), 4.0 (s, 3H), 1.8 (m, 2H), 1.3 (m, 2H), 0.8 (m, 3H).

LCMS m/z: [M+H]⁺=248.

### D. N-{3-[1-(1-Butyl-4-methoxy-1H-indole-3-carbonyl)-piperidin-4-yl]-4-fluoro-benzyl}-2,2,2-trifluoro-acetamide

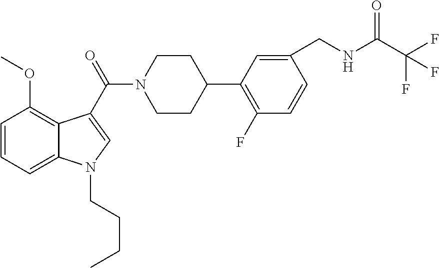

The title compound is prepared in a similar manner as described in Example 2I using 1-butyl-4-methoxy-1H-indole-3-carboxylic acid and 2,2,2-trifluoro-N-(4-fluoro-3-piperidin-4-yl-benzyl)-acetamide hydrochloride as the starting materials.

¹H NMR (300 MHz, CDCl₃) δ 7.2 (m, 4H), 7.0 (m, 2H), 6.7 (bs, 1H), 6.5 (d, 1H), 4.5 (m, 2H), 4.2 (m, 4H), 3.9 (s, 3H), 3.1-2.8 (m, 3H), 1.9-1.8 (m, 6H), 1.4 (m, 2H), 0.9 (m, 3H).

MS m/z: [M+H]=534.

### E. [4-(5-Aminomethyl-2-fluoro-phenyl)-piperidin-1-yl]-(1-butyl-4-methoxy-1H-indol-3-yl)-methanone hydrochloride

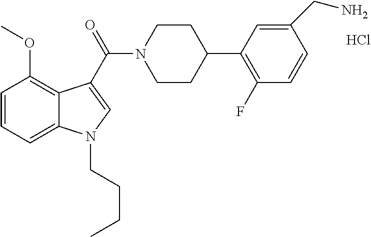

The title compound is prepared in a similar manner as described in Example 1K using N-{3-[1-(1-butyl-4-methoxy-1H-indole-3-carbonyl)-piperidin-4-yl]-4-fluoro-benzyl}-2,2,2-trifluoro-acetamide as the starting material.

¹H NMR (300 MHz, DMSO-d6) δ 8.4 (bs, 2H), 7.6 (m, 1H), 7.4 (m, 2H), 7.2 (m, 1H), 7.1 (m, 2H), 6.6 (m, 1H), 4.2 (m, 2H), 4.0 (m, 2H), 3.8 (s, 3H), 3.5 (m, 2H), 3.1-2.8 (m, 3H), 1.9-1.6 (m, 6H), 1.2 (m, 2H), 0.9 (m, 3H).

MS m/z: [M+H]⁺=438.

### Example 47

### [4-(5-Aminomethyl-2-fluoro-phenyl)-piperidin-1-yl]-[4-methoxy-1-(2-methoxy-ethyl)-1H-indol-3-yl]-methanone hydrochloride

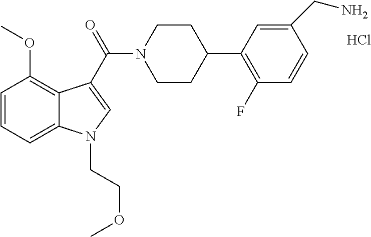

### A. 4-Methoxy-1-(2-methoxy-ethyl)-1H-indole

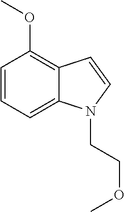

The title compound is prepared in a similar manner as described in Example 1E using 4-methoxyindole as the starting material.

¹H NMR (300 MHz, CDCl₃) δ 7.2-7.1 (m, 2H), 7.0 (d, 1H), 6.6 (d, 1H), 6.5 (d, 1H), 4.3 (t, 2H), 4.0 (s, 3H), 3.7 (t, 2H), 3.3 (s, 3H).

MS m/z: [M+H]⁺=206.

### B. 2,2,2-Trifluoro-1-[4-methoxy-1-(2-methoxy-ethyl)-1H-indol-3-yl]-ethanone

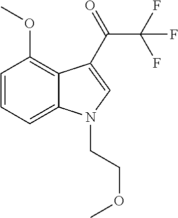

The title compound is prepared in a similar manner as described in Example 1F using 4-methoxy-1-(2-methoxy-ethyl)-1H-indole as the starting material.

¹H NMR (300 MHz, CDCl₃) δ 8.0 (m, 1H), 7.3 (m, 1H), 7.0 (d, 1H), 6.8 (d, 1H), 4.4 (t, 2H), 4.0 (s, 3H), 3.8 (t, 2H), 3.3 (s, 3H).

MS m/z: [M+H]⁺=302.

### C. 4-Methoxy-1-(2-methoxy-ethyl)-1H-indole-3-carboxylic acid

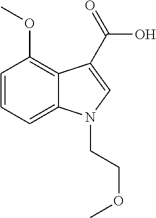

The title compound is prepared in a similar manner as described in Example 4C using 2,2,2-trifluoro-1-[4-methoxy-1-(2-methoxy-ethyl)-1H-indol-3-yl]-ethanone as the starting material.

¹H NMR (300 MHz, DMSO-d6) δ 11.6 (s, 1H), 8.0 (s, 1H), 7.2 (m, 2H), 6.8 (d, 1H), 4.4 (t, 2H), 3.9 (s, 3H), 3.6 (t, 2H), 3.2 (s, 3H).

MS m/z: [M+H]⁺=250.

### D. 2,2,2-Trifluoro-N-(4-fluoro-3-{1-[4-methoxy-1-(2-methoxy-ethyl)-1H-indole-3-carbonyl]-piperidin-4-yl}-benzyl)-acetamide

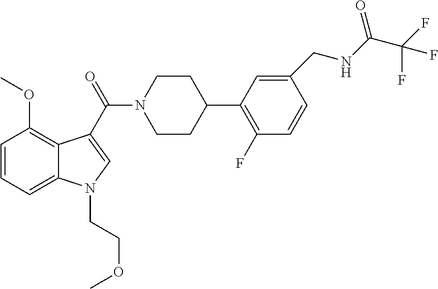

The title compound is prepared in a similar manner as described in Example 2I using 4-methoxy-1-(2-methoxy-ethyl)-1H-indole-3-carboxylic acid and 2,2,2-trifluoro-N-(4-fluoro-3-piperidin-4-yl-benzyl)-acetamide hydrochloride as the starting materials.

¹H NMR (300 MHz, CDCl₃) δ 7.2 (m, 3H), 7.0 (m, 2H), 6.8 (bs, 1H), 6.6 (d, 1H), 5.0 (bs, 1H), 4.5 (m, 2H), 4.3 (t, 2H), 3.9 (s, 3H), 3.7 (t, 2H), 3.3 (s, 3H), 3.1 (m, 3H), 1.9-1.6 (m, 6H).

MS m/z: [M+H]⁺=535.

### E. [4-(5-Aminomethyl-2-fluoro-phenyl)-piperidin-1-yl]-[4-methoxy-1-(2-methoxy-ethyl)-1 H-indol-3-yl]-methanone hydrochloride

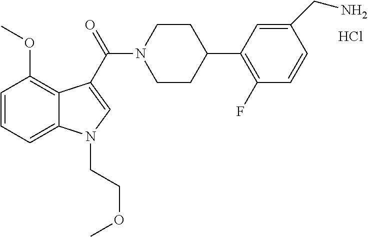

The title compound is prepared in a similar manner as described in Example 1K using 2,2,2-trifluoro-N-(4-fluoro-3-{1-[4-methoxy-1-(2-methoxy-ethyl)-1H-indole-3-carbonyl]-piperidin-4-yl}-benzyl)-acetamide as the starting material.

¹H NMR (300 MHz, DMSO-d6) δ 8.3 (bs, 2H), 7.5 (m, 1H), 7.4 (m, 2H), 7.2 (m, 3H), 6.6 (m, 1H), 4.4 (m, 2H), 4.0 (m, 2H), 3.9 (s, 3H), 3.8-3.5 (m, 5H), 3.3 (s, 3H), 3.1 (m, 2H), 1.8 (m, 2H), 1.6 (m, 2H).

MS m/z: [M+H]⁺=440.

### Example 48

### [4-(5-Aminomethyl-2-fluoro-phenyl)-piperidin-1-yl]-[4-bromo-1-(2-methoxy-ethyl)-1H-indol-3-yl]-methanone hydrochloride

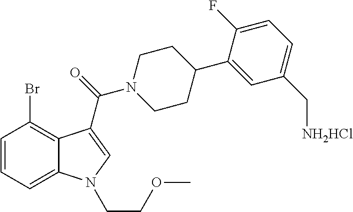

A 4-Bromo-1-(2-methoxy-ethyl)-1H-indole

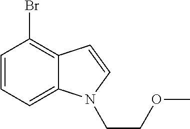

The title compound is prepared in a similar manner as described in Example 1E using 2-bromoethylmethylether as the starting material.

¹H NMR (300 MHz, CDCl₃) δ 7.32-7.28 (m, 2H), 7.22 (d, 1H), 7.10-7.04 (m, 1H), 6.56 (m, 1H), 4.28 (t, 2H), 3.70 (t, 2H), 3.31 (s, 3H).

### B 1-[4-Bromo-1-(2-methoxy-ethyl)-1H-indol-3-yl]-2,2,2-trifluoro-ethanone

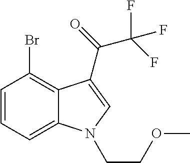

The title compound is prepared in a similar manner as described in Example 2G using 4-bromo-1-(2-methoxy-ethyl)-1H-indole as the starting material.

¹H NMR (300 MHz, CDCl₃) δ 7.99 (m, 1H), 7.49 (m, 1H), 7.32-7.29 (m, 1H), 7.13 (t, 1H), 4.28 (t, 2H), 3.67 (t, 2H), 3.25 (s, 3H).

### C 4-Bromo-1-(2-methoxy-ethyl)-1H-indole-3-carboxylic acid

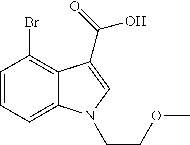

The title compound is prepared in a similar manner as described in Example 2H using 1-[4-bromo-1-(2-methoxy-ethyl)-1H-indol-3-yl]-2,2,2-trifluoro-ethanone as the starting material.

¹H NMR (300 MHz, CD₃OD) δ 8.00 (s, 1H), 7.51-7.48 (m, 1H), 7.42-7.40 (m, 1H), 7.10 (t, 1H), 4.36 (t, 2H), 3.70 (t, 2H), 3.26 (s, 3H).

### D N-(3-{1-[4-Bromo-1-(2-methoxy-ethyl)-1H-indole-3-carbonyl]-piperidin-4-yl}-4-fluoro-benzyl)-2,2,2-trifluoro-acetamide

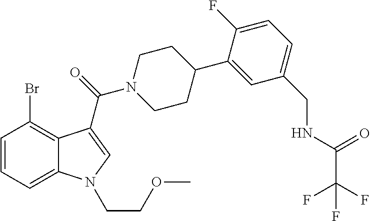

The title compound is prepared in a similar manner as described in Example 6E using 4-bromo-1-(2-methoxy-ethyl)-1H-indole-3-carboxylic acid and 2,2,2-trifluoro-N-(4-fluoro-3-piperidin-4-yl-benzyl)-acetamide hydrochloride as the starting materials.

¹H NMR (300 MHz, CD₃OD) δ 7.53-7.50 (m, 2H), 7.33-7.30 (m, 1H), 7.27-7.24 (m, 1H), 7.19-7.10 (m, 2H), 7.06-6.99 (m, 1H), 4.85 (s, 3H), 4.41-4.36 (m, 4H), 3.71 (t, 2H), 3.28 (s, 3H), 3.24-3.12 (m, 2H), 3.01-2.92 (m, 1H), 1.92 (br s, 2H), 1.68 (br s, 2H).

### E [4-(5-Aminomethyl-2-fluoro-phenyl)-piperidin-1-yl]-[4-bromo-1-(2-methoxy-ethyl)-1H-indol-3-yl]-methanone hydrochloride

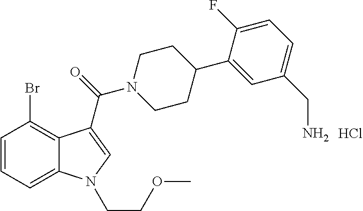

The title compound is prepared in a similar manner as described in Example 3B using N-(3-{1-[4-bromo-1-(2-methoxy-ethyl)-1H-indole-3-carbonyl]-piperidin-4-yl}-4-fluoro-benzyl)-2,2,2-trifluoro-acetamide as the starting material.

¹H NMR (300 MHz, CD₃OD) δ 7.55-7.52 (m, 2H), 7.44-7.42 (m, 1H), 7.36-7.31 (m, 2H), 7.17-7.11 (m, 2H), 4.93-4.88 (m, 2H), 4.40 (t, 2H), 4.10 (s, 2H), 3.72 (t, 2H), 3.28 (s, 3H), 3.26-3.18 (m, 2H), 3.07-2.98 (m, 1H), 1.96 (br s, 2H), 1.74 (br s, 2H). MS m/z: [M+H]⁺=488, 490.

### Example 49

### [4-(5-Aminomethyl-2-fluoro-phenyl)-piperidin-1-yl]-(2,3-dihydro-pyrrolo[1,2,3-de]-1,4-benzoxazin-6-yl)-methanone hydrochloride

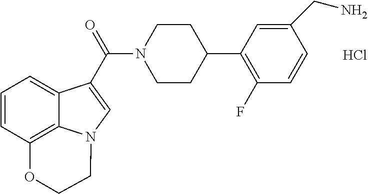

### A. 7-(2-Chloro-ethoxy)-1H-indole

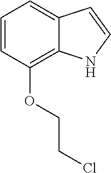

To a solution of 7-hydroxyindole (1.5 g, 11 mmol) in THF (60 mL) is added triphenylphosphine (5.8 g, 22 mmol), diisopropyl azodicarboxylate (4.4 g, 22 mmol) and 2-chloro-ethanol (1.47 mL, 22 mmol). The reaction mixture is stirred at room temperature overnight. The reaction mixture is concentrated in vacuo and purified by flash chromatography on SiO2 eluting with 10% ethyl acetate/heptane to give the titled compound (1.4 g, 65%).

¹H NMR (300 MHz, CDCl₃) δ 8.4 (bs, 1H), 7.3 (m, 1H), 7.2 (m, 1H), 7.0 (m, 1H), 6.6 (d, 1H), 6.5 (m, 1H), 4.4 (t, 2H), 3.9 (t, 2H).

MS m/z: [M+H]⁺=196.

### B. 2,3-Dihydro-pyrrolo[1,2,3-de]-1,4-benzoxazine

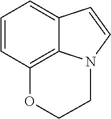

To a solution of 7-(2-chloro-ethoxy)-1H-indole (1.20 g, 6.12 mmol) in N,N-dimethylformamide (15 mL) under N2 at 0° C. is added NaH (0.49 g, 12.24 mmol). The reaction mixture is stirred at room temperature for 2 h then quenched with 1N HCl. The reaction mixture is poured into EtOAc and washed with H2O, brine, dried with MgSO4, filtered and concentrated in vacuo to yield the titled compound (0.89 g, 90%).

¹H NMR (300 MHz, CDCl₃) δ 7.3 (m, 1H), 7.1 (m, 1H), 7.0 (m, 1H), 6.7 (m, 1H), 6.5 (m, 1H), 4.6 (t, 2H), 4.3 (t, 2H).

MS m/z: [M+H]⁺=160.

### C. 1-(2,3-Dihydro-pyrrolo[1,2,3-de]-1,4-benzoxazin-6-yl)-2,2,2-trifluoro-ethanone

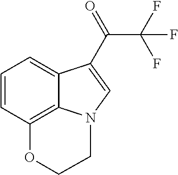

The title compound is prepared in a similar manner as described in Example 1F using 2,3-dihydro-pyrrolo[1,2,3-de]-1,4-benzoxazine as the starting material.

¹H NMR (300 MHz, DMSO-d6) δ 8.5 (s, 1H), 7.6 (d, 1H), 7.2 (m, 1H), 6.8 (d, 1H), 4.5 (m, 4H).

MS m/z: [M+H]⁺=256.

### D. 2,3-Dihydro-pyrrolo[1,2,3-de]-1,4-benzoxazine-6-carboxylic acid

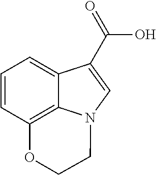

The title compound is prepared in a similar manner as described in Example 4C using 1-(2,3-dihydro-pyrrolo[1,2,3-de]-1,4-benzoxazin-6-yl)-2,2,2-trifluoro-ethanone as the starting material.

¹H NMR (300 MHz, DMSO-d6) δ 12.0 (s, 1H), 8.0 (s, 1H), 7.4 (d, 1H), 7.0 (m, 1H), 6.6 (d, 1H), 4.5 (t, 2H), 4.4 (t, 2H).

MS m/z: [M+H]⁺=204.

### E. N-{3-O-(2,3-Dihydro-pyrrolo[1,2,3-de]-1,4-benzoxazine-6-carbonyl)-piperidin-4-yl]-4-fluoro-benzyl}-2,2,2-trifluoro-acetamide

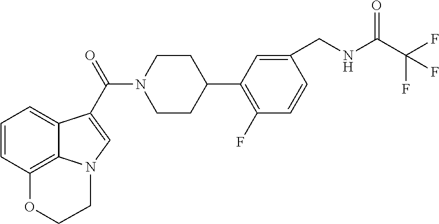

The title compound is prepared in a similar manner as described in Example 2I using 2,3-dihydro-pyrrolo[1,2,3-de]-1,4-benzoxazine-6-carboxylic acid and 2,2,2-trifluoro-N-(4-fluoro-3-piperidin-4-yl-benzyl)-acetamide hydrochloride as the starting materials.

¹H NMR (300 MHz, CDCl₃) δ 7.5 (s, 1H), 7.3-7.0 (m, 5H), 6.7 (d, 1H), 6.6 (bs, 1H), 4.6 (m, 2H), 4.5 (m, 4H), 4.3 (m, 2H), 3.1 (m, 3H), 1.9-1.7 (m, 4H).

MS m/z: [M+H]⁺=490.

### F. [4-(5-Aminomethyl-2-fluoro-phenyl)-piperidin-1-yl]-(2,3-dihydro-pyrrolo[1,2,3-de]-1,4-benzoxazin-6-yl)-methanone hydrochloride

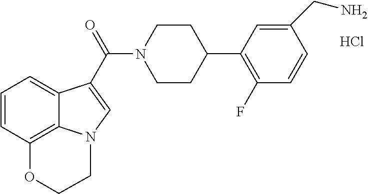

The title compound is prepared in a similar manner as described in Example 1K using N-{3-[1-(2,3-dihydro-pyrrolo[1,2,3-de]-1,4-benzoxazine-6-carbonyl)-piperidin-4-yl]-4-fluoro-benzyl}-2,2,2-trifluoro-acetamide as the starting material.

¹H NMR (300 MHz, DMSO-d6) δ 8.6 (bs, 2H), 7.8 (s, 1H), 7.6 (m, 1H), 7.4 (m, 1H), 7.2 (m, 2H), 7.0 (m, 1H), 6.6 (m, 1H), 4.4 (m, 4H), 4.0 (m, 2H), 3.7 (m, 2H), 3.1 (m, 3H), 1.8-1.6 (m, 4H).

MS m/z: [M+H]⁺=394.

### Example 50

### [4-(5-Aminomethyl-2-fluoro-phenyl)-piperidin-1-yl]-[1-(3-hydroxy-3-methyl-butyl)-1H-indol-3-yl]-methanone hydrochloride

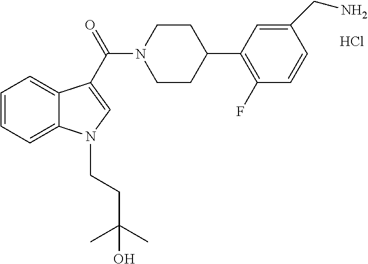

### A. 3-Indol-1-yl-propionic acid methyl ester

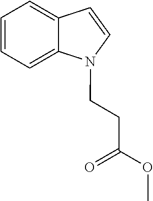

The title compound is prepared in a similar manner as described in Example 1E using 1H-indole and 3-bromo-propionic acid methyl ester as the starting materials. ¹H NMR (300 MHz, CDCl₃) δ 7.6 (d, 1H), 7.4 (m, 1H), 7.2 (m, 1H), 7.1 (m, 2H), 6.4 (m, 1H), 4.5 (t, 2H), 3.6 (s, 3H), 2.8 (t, 2H).

MS m/z: [M+H]⁺=204.

### B. 4-Indol-1-yl-2-methyl-butan-2-ol

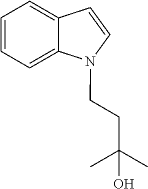

To a solution of 3-indol-1-yl-propionic acid methyl ester (3.0 g, 14.78 mmol) in THF (50 mL) at 0° C. is added 3.0 M methylmagnesium iodide solution in diethyl ether (9.81 mL, 29.56 mmol). The reaction mixture is allowed to slowly warm to room temperature and stir overnight. The reaction mixture is quenched with saturated NH₄Cl, poured into EtOAc and washed with H₂O, brine, dried with MgSO₄, filtered and concentrated in vacuo. The crude product is purified by flash chromatography on SiO₂ eluting with 20% ethyl acetate/heptane to give the titled compound (2.6 g, 87%).

¹H NMR (300 MHz, CDCl₃) δ 7.6 (d, 1H), 7.4 (d, 1H), 7.2 (m, 1H), 7.1 (m, 2H), 6.5 (m, 1H), 4.3 (m, 2H), 2.0 (m, 2H), 1.3 (s, 6H).

MS m/z: [M+H]⁺=204.

### C. Trifluoro-acetic acid 1,1-dimethyl-3-[3-(2,2,2-trifluoro-acetyl)-indol-1-yl]-propyl ester

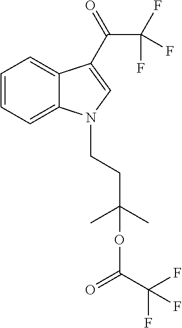

The title compound is prepared in a similar manner as described in Example 1F using 4-indol-1-yl-2-methyl-butan-2-ol as the starting material.

¹H NMR (300 MHz, CDCl₃) δ 8.4 (m, 1H), 7.9 (s, 1H), 7.4 (m, 3H), 4.4 (m, 2H), 2.4 (m, 2H), 1.7 (s, 6H).

MS m/z: [M+H]⁺=396.

### D. 1-(3-Hydroxy-3-methyl-butyl)-1H-indole-3-carboxylic acid

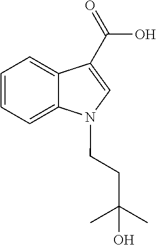

The title compound is prepared in a similar manner as described in Example 4C using trifluoro-acetic acid 1,1-dimethyl-3-[3-(2,2,2-trifluoro-acetyl)-indol-1-yl]-propyl ester as the starting material.

¹H NMR (300 MHz, DMSO-d6) δ 11.9 (s, 1H), 8.0 (m, 2H), 7.5 (d, 1H), 7.2 (m, 2H), 4.5 (s, H), 4.3 (m, 2H), 1.8 (m, 2H), 1.2 (s, 6H).

MS m/z: [M+H]⁺=248.

### E. 2,2,2-Trifluoro-N-(4-fluoro-3-{1-[1-(3-hydroxy-3-methyl-butyl)-1H-indole-3-carbonyl]-piperidin-4-yl}-benzyl)acetamide

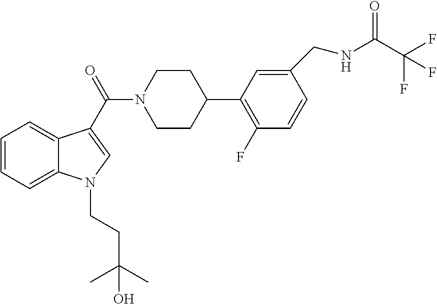

The title compound is prepared in a similar manner as described in Example 2I using 1-(3-hydroxy-3-methyl-butyl)-1H-indole-3-carboxylic acid and 2,2,2-trifluoro-N-(4-fluoro-3-piperidin-4-yl-phenyl)-acetamide hydrochloride as the starting materials.

¹H NMR (300 MHz, CDCl₃) δ 7.7 (d, 1H), 7.5 (s, 1H), 7.4 (m, 1H), 7.2 (m, 4H), 7.0 (m, 1H), 6.7 (bs, 1H), 4.6 (m, 2H), 4.5 (m, 2H), 4.3 (m, 2H), 3.1 (m, 3H), 2.0 (m, 3H), 1.9-1.6 (m, 4H), 1.3 (s, 6H).

MS m/z: [M+H]⁺=534.

### F. [4-(5-Aminomethyl-2-fluoro-phenyl)-piperidin-1-yl]-[1-(3-hydroxy-3-methyl-butyl)-1H-indol-3-yl]-methanone hydrochloride

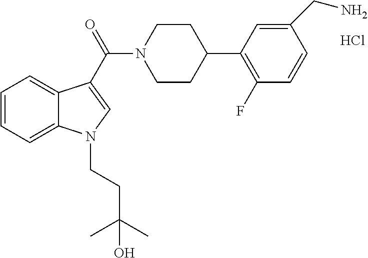

The title compound is prepared in a similar manner as described in Example 1K using 2,2,2-trifluoro-N-(4-fluoro-3-{1-[1-(3-hydroxy-3-methyl-butyl)-1H-indole-3-carbonyl]-piperidin-4-yl}-benzyl)-acetamide as the starting material.

¹H NMR (300 MHz, DMSO-d6) δ 8.3 (bs, 2H), 7.8 (s, 1H), 7.7 (d, 1H), 7.5 (m, 2H), 7.4 (m, 1H), 7.2 (m, 3H), 4.5 (m, 2H), 4.3 (m, 2H), 4.1 (m, 2H), 3.1 (m, 3H), 1.9-1.6 (m, 6H), 1.2 (s, 6H).

MS m/z: [M+H]⁺=438.

### Example 51

### [4-(5-Aminomethyl-2-fluoro-phenyl)-piperidin-1-yl]-[4-fluoro-1-(2-methoxy-ethyl)-1H-indol-3-yl]-methanone hydrochloride

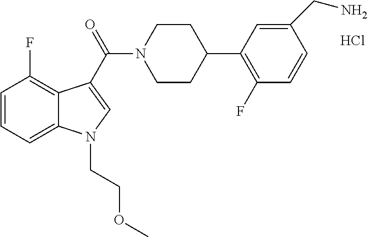

### A. 4-Fluoro-1-(2-methoxy-ethyl)-1H-indole

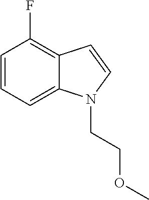

The title compound is prepared in a similar manner as described in Example 1E using 4-fluoroindole as the starting material.

¹H NMR (300 MHz, CDCl₃) δ 7.2 (m, 3H), 6.8 (m, 1H), 6.6 (m, 1H), 4.3 (t, 2H), 3.7 (t, 2H), 3.35 (s, 3H).

LCMS m/z: [M+H]⁺=194.

### B. 2,2,2-Trifluoro-1-[4-fluoro-1-(2-methoxy-ethyl)-1H-indol-3-yl]-ethanone

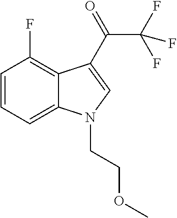

The title compound is prepared in a similar manner as described in Example 1F using 4-fluoro-1-(2-methoxy-ethyl)-1H-indole as the starting material.

¹H NMR (300 MHz, CDCl₃) δ 8.1 (s, 1H), 7.4 (m, 2H), 7.2 (m, 1H), 7.1 (m, 1H), 4.4 (t, 2H), 3.8 (t, 2H), 3.3 (s, 3H).

MS m/z: [M+H]⁺=290.

### C. 4-Fluoro-1-(2-methoxy-ethyl)-1H-indole-3-carboxylic acid

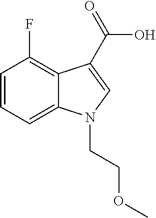

The title compound is prepared in a similar manner as described in Example 4C using 2,2,2-trifluoro-1-[4-fluoro-1-(2-methoxy-ethyl)-1H-indol-3-yl]-ethanone as the starting material.

¹H NMR (300 MHz, DMSO-d6) δ 11.7 (bs, 1H), 8.0 (s, 1H), 7.4 (d, 1H), 7.2 (m, 1H), 6.9 (m, 1H), 4.4 (t, 2H), 3.7 (t, 2H), 3.2 (s, 3H).

MS m/z: [M+H]⁺=238.

### D. 2,2,2-Trifluoro-N-(4-fluoro-3-{1-[4-fluoro-1-(2-methoxy-ethyl)-1H-indole-3-carbonyl]-piperidin-4-yl}-benzyl)acetamide

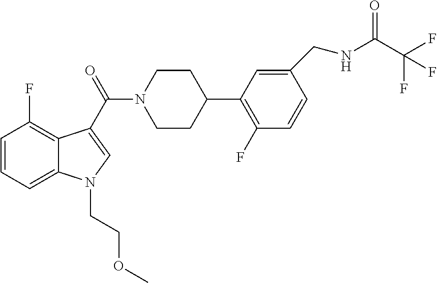

The title compound is prepared in a similar manner as described in Example 2I using 4-fluoro-1-(2-methoxy-ethyl)-1H-indole-3-carboxylic acid and 2,2,2-trifluoro-N-(4-fluoro-3-piperidin-4-yl-benzyl)-acetamide hydrochloride as the starting materials.

¹H NMR (300 MHz, CDCl₃) δ 7.5 (s, 1H), 7.2 (m, 4H), 7.1 (m, 1H), 6.9 (m, 1H), 6.6 (bs, 1H), 4.5 (m, 2H), 4.3 (m, 2H), 3.8 (m, 2H), 3.4 (s, 3H), 3.1 (m, 4H), 1.9-1.65 (m, 4H)

MS m/z: [M+H]⁺=524.

### E. [4-(5-Aminomethyl-2-fluoro-phenyl)-piperidin-1-yl]-[4-fluoro-1-(2-methoxy-ethyl)-1H-indol-3-yl]-methanone hydrochloride

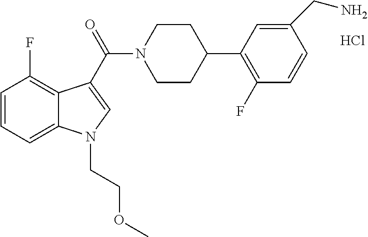

The title compound is prepared in a similar manner as described in Example 1K using 2,2,2-trifluoro-N-(4-fluoro-3-{1-[4-fluoro-1-(2-methoxy-ethyl)-1H-indole-3-carbonyl]-piperidin-4-yl}-benzyl)-acetamide as the starting material.

¹H NMR (300 MHz, DMSO-d6) δ 8.2 (bs, 2H), 7.6 (s, 1H), 7.5 (m, 1H), 7.4 (m, 1H), 7.3 (m, 1H), 7.2 (m, 2H), 6.9 (m, 1H), 4.4 (m, 2H), 4.0 (m, 2H), 3.7 (m, 2H), 3.2 (s, 3H), 3.1 (m, 4H), 1.8 (m, 2H), 1.6 (m, 2H).

MS m/z: [M+H]⁺=428.

### Example 52

### [4-(5-Aminomethyl-2-fluoro-phenyl)-piperidin-1-yl]-[4-cyclopropyl-1-(2-methoxy-ethyl)-1H-indol-3-yl]-methanone hydrochloride

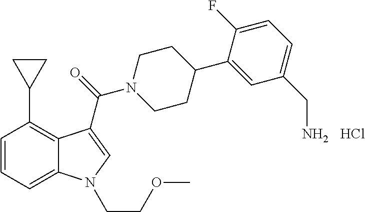

### A. 4-Cyclopropyl-1-(2-methoxy-ethyl)-1H-indole

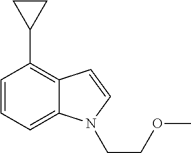

The title compound is prepared in a similar manner as described in Example 6B using 4-bromo-1-(2-methoxy-ethyl)-1H-indole and cyclopropylboronic acid as the starting materials.

¹H NMR (300 MHz, CDCl₃) δ 7.20-7.12 (m, 3H), 6.73-6.71 (d, 1H), 6.69-6.68 (m, 1H), 4.29 (t, 2H), 3.72 (t, 2H), 3.33 (s, 3H), 2.30-2.21 (m, 1H), 1.04-0.98 (m, 2H), 0.88-0.82 (m, 2H).

### B. 1-[4-Cyclopropyl-1-(2-methoxy-ethyl)-1H-indol-3-yl]-2,2,2-trifluoro-ethanone

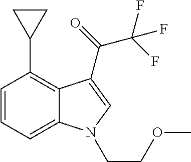

The title compound is prepared in a similar manner as described in Example 2G using 4-cyclopropyl-1-(2-methoxy-ethyl)-1H-indole as the starting material.

¹H NMR (300 MHz, CDCl₃) δ 8.10-8.08 (m, 1H), 7.30-7.19 (m, 2H), 6.94-6.92 (m, 1H), 4.34 (t, 2H), 3.75 (t, 2H), 3.33 (s, 3H), 2.24-2.15 (m, 1H), 1.05-0.98 (m, 2H), 0.74-0.69 (m, 2H).

### C. 4-Cyclopropyl-1-(2-methoxy-ethyl)-1H-indole-3-carboxylic acid

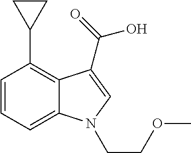

The title compound is prepared in a similar manner as described in Example 2H using 1-[4-cyclopropyl-1-(2-methoxy-ethyl)-1H-indol-3-yl]-2,2,2-trifluoro-ethanone as the starting material.

¹H NMR (300 MHz, DMSO-d6) δ 11.75 (s, 1H), 7.37-7.34 (m, 1H), 7.12 (t, 1H), 6.66 (d, 1H), 6.58-6.56 (m, 1H), 4.38 (t, 2H), 3.66 (t, 2H), 3.52-3.43 (m, 1H), 3.21 (s, 3H), 0.93-0.87 (m, 2H), 0.68-0.62 (m, 2H).

### D. N-(3-{1-[4-Cyclopropyl-1-(2-methoxy-ethyl)-1H-indole-3-carbonyl]-piperidin-4-yl}-4-fluoro-benzyl)-2,2,2-trifluoro-acetamide

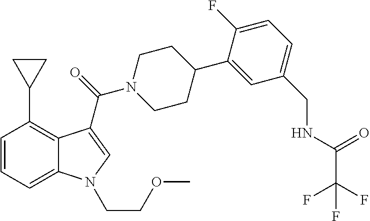

The title compound is prepared in a similar manner as described in Example 6E using 4-cyclopropyl-1-(2-methoxy-ethyl)-1H-indole-3-carboxylic acid and 2,2,2-trifluoro-N-(4-fluoro-3-piperidin-4-yl-benzyl)-acetamide hydrochloride as the starting materials.

¹H NMR (300 MHz, CDCl₃) δ 7.18 (m, 2H), 7.14-7.08 (m, 2H), 7.02-6.96 (m, 2H), 6.81-6.78 (m, 1H), 4.45 (d, 2H), 4.27 (t, 2H), 3.70 (t, 2H), 3.31 (s, 3H), 3.20-3.04 (m, 2H), 2.36 (br s, 1H), 1.94-1.73 (m, 4H), 0.92-0.90 (m, 2H), 0.74 (br s, 2H).

### E-[4-(5-Aminomethyl-2-fluoro-phenyl)-piperidin-1-yl]-[4-cyclopropyl-1-(2-methoxy-ethyl)-1H-indol-3-yl]-methanone hydrochloride

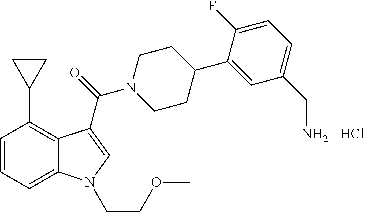

The title compound is prepared in a similar manner as described in Example 3B using N-(3-{1-[4-cyclopropyl-1-(2-methoxy-ethyl)-1H-indole-3-carbonyl]-piperidin-4-yl}-4-fluoro-benzyl)-2,2,2-trifluoro-acetamide as the starting material.

¹H NMR (300 MHz, DMSO-d6) δ 8.53 (br s, 3H), 7.57 (m, 1H), 7.51 (s, 1H), 7.40 (m, 1H), 7.36-7.33 (m, 1H), 7.26-7.17 (m, 1H), 7.11-7.06 (m, 1H), 6.68 (d, 1H), 4.35 (t, 2H), 3.99 (br d, 3H), 3.66 (t, 2H), 3.22 (s, 3H), 3.15-3.00 (m, 4H), 2.30 (m, 1H), 1.80-1.63 (m, 4H), 0.89-0.86 (m, 2H), 0.66 (br s, 2H).

MS m/z: [M+H]⁺=450.

### Example 53

### [4-(5-Aminomethyl-2-fluoro-phenyl)-piperidin-1-yl]-[1-(2-methoxy-ethyl)-4-trifluoromethyl-1H-indol-3-yl]-methanone hydrochloride

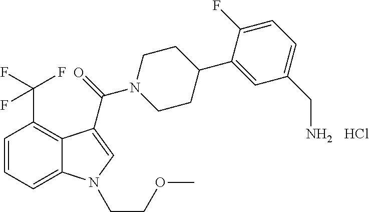

### A. 1-(2-Methoxy-ethyl)-4-trifluoromethyl-1H-indole

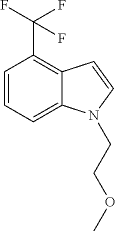

A mixture of 4-trifluoromethyl-1H-indole (105 mg, 0.57 mmol), powder KOH (159 mg, 2.83 mmol) in DMSO (6 mL) is stirred at r.t. for 5 min. 2-Methoxyethyl bromide (80 μL, 0.85 mmol) is added. After the reaction mixture is stirred at r.t. overnight, it is partitioned between H₂O and Et₂O. The two layers are separated, and the aqueous layer is extracted with Et2O (3×). The combined organic extracts are washed with H₂O and brine, dried over MgSO₄, filtered, and concentrated in vacuo. The crude material is purified on silica gel with heptane/EtOAc (100/0 to 70/30) as eluent to yield the product (100 mg, 72%) as a clear colorless liquid.

¹H NMR (300 MHz, CDCl₃) δ 7.53 (d, J=8.2 Hz, 1H), 7.40 (d, J=7.3 Hz, 1H), 7.35-7.20 (m, 2H), 6.69 (s, 1H), 4.32 (t, J=5.4 Hz, 2H), 3.71 (t, J=5.4 Hz, 2H), 3.31 (s, 3H),

¹⁹F NMR (300 MHz, CDCl₃) δ −60.99 (s, 3F);

LC Rt: 3.21 min; MS 244 (M+H, 100%).

### B. 2,2,2-Trifluoro-1-[1-(2-methoxy-ethyl)-4-trifluoromethyl-1H-indol-3-yl]-ethanone

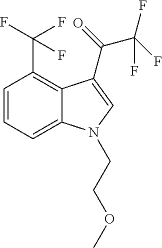

A mixture of 1-(2-methoxy-ethyl)-4-trifluoromethyl-1H-indole (95 mg, 0.39 mmol) and TFAA (0.16 mL, 1.17 mmol) in DMF (10 mL) is heated at 45° C. overnight. The mixture is then partitioned between H₂O and Et₂O. The two layers are separated, and the organic layer is washed with H2O and brine, dried over MgSO₄, filtered, and concentrated in vacuo. The crude material is purified on silica gel with hepatane/EtOAc (95/5 to 50/50) as eluent to yield the product (92 mg, 69%) as a yellow waxy solid.

¹H NMR (300 MHz, CDCl₃) δ 8.16 (d, J=1.8 Hz, 1H), 7.76 (d, J=7.7 Hz, 1H), 7.47 (d, J=8.1 Hz, 1H), 7.46 (t, J=8.1 Hz, 1H), 4.42 (t, J=5.1 Hz, 2H), 3.76 (t, J=5.1 Hz, 2H), 3.33 (s, 3H);

¹⁹F NMR (300 MHz, CDCl₃) δ −58.25 (s, 3F), −70.91 (s, 3F);

LC Rt: 3.28 min; MS 340 (M+H, 100%).

### C. 1-(2-Methoxy-ethyl)-4-trifluoromethyl-1H-indole-3-carboxylic acid

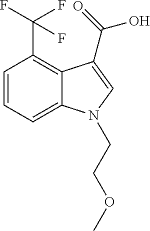

A mixture of 2,2,2-trifluoro-1-[1-(2-methoxy-ethyl)-4-trifluoromethyl-1H-indol-3-yl]-ethanone (90 mg, 0.27 mmol) in MeOH (10 mL) and NaOH (5 M, 5 mL) is heated at 80° C. overnight. This mixture is concentrated in vacuo to remove the methanol. The residue is diluted with H₂O, and then washed with Et₂O once. The aqueous layer is acidified to pH 2 with HCl (6 M). The acidified mixture is extracted with EtOAc (2×). The combined organic extracts are washed with H2O and brine, dried over MgSO₄, filtered, and concentrated in vacuo. The residue is coevaportated with CH₂Cl₂ and heptane to yield the product (56 mg, 73%) as a beige powder.

¹H NMR (300 MHz, CDCl₃) δ 8.14 (s, 1H), 7.68 (d, J=7.6 Hz, 1H), 7.61 (d, J=8.2 Hz, 1H), 7.36 (t, J=7.9 Hz, 1H), 4.36 (t, J=5.2 Hz, 2H), 3.74 (t, J=5.3 Hz, 2H), 3.32 (s, 3H);

19F NMR (300 MHz, CDCl3) −58.39 (s, 3F);

LC Rt: 2.52 min; MS 288 (M+H, 100%).

### D. 2,2,2-Trifluoro-N-(4-fluoro-3-{1-[1-(2-methoxy-ethyl)-4-trifluoromethyl-1H-indole-3-carbonyl]-piperidin-4-yl}-benzyl)-acetamide

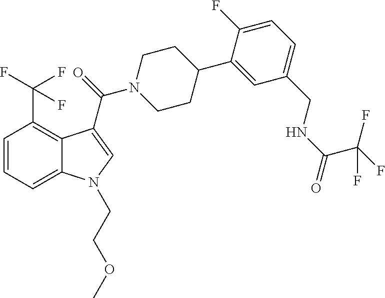

A mixture of 1-(2-methoxy-ethyl)-4-trifluoromethyl-1H-indole-3-carboxylic acid (50 mg, 0.17 mmol), Et₃N (73 μL, 0.55 mmol), 2,2,2-trifluoro-N-(4-fluoro-3-piperidin-4-yl-benzyl)-acetamide hydrochloride (77 mg, 0.23 mmol), and EDCI (50 mg, 0.26 mmol) in CH₂Cl₂ (10 mL) is stirred at r.t. overnight. The mixture is partitioned between H₂O and CH₂Cl₂. The two layers are separated, and the organic layer is washed with brine, dried over MgSO₄, filtered, and concentrated in vacuo. The crude material is purified on silica gel with heptane/EtOAc (50/50 to 0/100) as eluent to give the product (52 mg, 52%) as a white powder.

¹H NMR (300 MHz, CDCl₃) δ 7.65-7.45 (m, 2H), 7.35-7.25 (m, 2H), 7.20-7.10 (m, 1H), 7.10-6.95 (m, 1H), 6.69 (br s, 1H), 5.10-4.90 (br m, 1H), 4.55-4.40 (m, 2H), 4.40-4.25 (m, 2H), 4.40-3.75 (br m, 1H), 3.71 (t, J=5.1 Hz, 2H), 3.95-3.60 (m, 3H), 3.31 (s, 3H), 3.25-3.00 (m, 2H), 3.00-2.85 (m, 1H), 2.05-1.50 (m, 4H);

¹⁹F NMR (300 MHz, CDCl₃) δ −58.47 (br m, 3F), −75.36 (s, 3F), −118.84 (br m, 1F); LC Rt 3.32 min; MS 574 (M+H, 100%).

### E. [4-(5-Aminomethyl-2-fluoro-phenyl)-piperidin-1-yl]-[1-(2-methoxy-ethyl)-4-trifluoromethyl-1H-indol-3-yl]-methanone hydrochloride

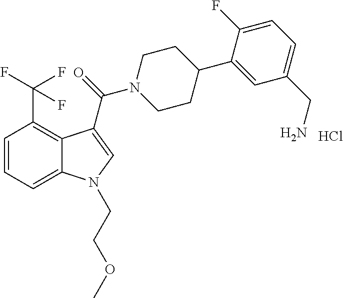

A mixture of 2,2,2-trifluoro-N-(4-fluoro-3-{1-[1-(2-methoxy-ethyl)-4-trifluoromethyl-1H-indole-3-carbonyl]-piperidin-4-yl}-benzyl)-acetamide (45 mg, 0.078 mmol) in MeOH (5 mL) is added aqueous K₂CO₃ (130 mg, 0.94 mmol, dissolved in 1.5 mL H₂O). This mixture is stirred at r.t. overnight. LC/MS indicates the reaction is completed. The reaction mixture is concentrated in vacuo to remove most of the methanol. The residue is partitioned between H₂O and EtOAc. The two layers are separated, and the organic layer is washed with H₂O and brine, dried over MgSO₄, filtered, and concentrated in vacuo. The residue is dissolved in Et₂O, and HCl in Et₂O (1.0 M, 3 mL) is added. The resulting suspension is concentrated in vacuo, and then dried in vacuo to yield the product (35 mg, 87%) as a beige solid.

¹H NMR (300 MHz, DMSO-d6) δ 8.35 (br, s 3H), 7.93 (d, J=8.2 Hz, 1H), 7.80-7.65 (m, 1H), 7.60-7.45 (m, 2H), 7.25-7.10 (m, 2H), 7.21 (t, J=10.2 Hz, 1H), 4.85-4.65 (br m, 1H), 4.55-4.45 (m, 2H), 4.10-3.90 (m, 2H), 3.90-3.60 (m, 3H), 3.22 (s, 3H), 3.20-3.00 (m, 1H), 3.00-2.85 (br m, 1H), 2.00-1.40 (m, 4H);

¹⁹F NMR (300 MHz, DMSO-d6) δ −57.72 (s, 3F), −119.29 (s, 1F);

LC 2.65 min; MS 478 (M+H, 100%).

### Example 54

### [4-(5-Aminomethyl-2-fluoro-phenyl)-piperidin-1-yl]-[1-(2-methoxy-ethyl)-4-trifluoromethoxy-1H-indol-3-yl]-methanone hydrochloride

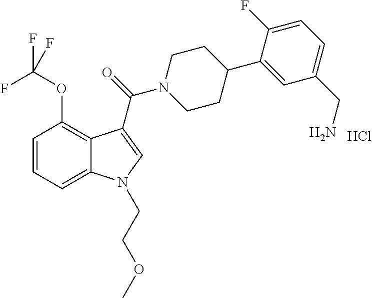

### A. 1-(2-Methoxy-ethyl)-4-trifluoromethoxy-1H-indole-3-carboxylic acid

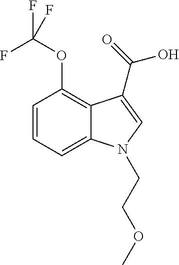

A mixture of 7-chloro-1-(2-methoxy-ethyl)-4-trifluoromethoxy-1H-indole-3-carboxylic acid (100 mg, 0.30 mmol) and Pd/C (10%, 75 mg) in MeOH is hydrogenated at 50 psi at r.t. for 4 h. The reaction is filtered through Celite, and the filtrate is concentrated in vacuo. The residue is redissolved in EtOAc. MgSO₄ and a small amount of activated charcoal are added. This mixture is then filtered, and the filtrate is concentrated in vacuo to yield the product (50 mg, 55%) as a white powder.

¹H NMR (300 MHz, CDCl₃) δ 8.08 (s, 1H), 7.45-7.15 (m, 3H), 4.33 (t, J=5.1 Hz, 2H), 3.75 (t, J=5.2 Hz, 2H), 3.34 (s, 3H);

¹⁹F NMR (300 MHz, CDCl₃) δ −57.28 (s, 3F);

LC Rt: 2.59 min; MS 304 (M+H, 100%).

### B. 2,2,2-Trifluoro-N-(4-fluoro-3-{1-[1-(2-methoxy-ethyl)-4-trifluoromethoxy-1H-indole-3-carbonyl]-piperidin-4-yl}-benzyl)-acetamide

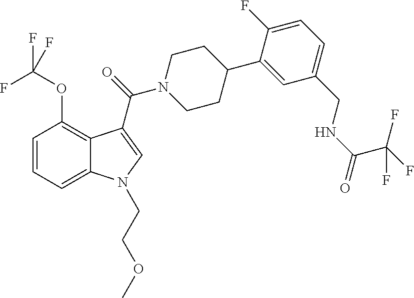

A mixture of 1-(2-methoxy-ethyl)-4-trifluoromethyl-1H-indole-3-carboxylic acid (50 mg, 0.17 mmol), Et3N (73 μL, 0.55 mmol), 2,2,2-trifluoro-N-(4-fluoro-3-piperidin-4-yl-benzyl)-acetamide hydrochloride (77 mg, 0.23 mmol), and EDCI (50 mg, 0.26 mmol) in CH₂Cl₂ (10 mL) is stirred at r.t. overnight. The mixture is partitioned between H₂O and CH₂Cl₂. The two layers are separated, and the organic layer is washed with brine, dried over MgSO₄, filtered, and concentrated in vacuo. The crude material is purified on silica gel with heptane/EtOAc (50/50 to 0/100) as eluent to give the product (52 mg, 52%) as a white powder.

¹H NMR (300 MHz, CDCl₃) δ 7.45 (s, 1H), 7.40-7.20 (m, 2H), 7.20-6.90 (m, 4H), 6.68 (br s, 1H), 5.10-4.90 (br m, 1H), 4.48 (d, J=5.7 Hz, 2H), 4.29 (t, J=5.3 Hz, 2H), 4.40-3.80 (br m, 1H), 3.74 (t, J=5.4 Hz, 2H), 3.34 (s, 3H), 3.25-2.70 (m, 3H), 2.05-1.50 (m, 4H);

¹⁹F NMR (300 MHz, CDCl₃) δ −57.12 (s, 3F), −75.38 (s, 3F), −119.13 (s, 1F);

LC Rt 3.36 min; MS 590 (M+H, 100%).

### C. [4-(5-Aminomethyl-2-fluoro-phenyl)-piperidin-1-yl]-[1-(2-methoxy-ethyl)-4-trifluoromethoxy-1H-indol-3-yl]-methanone hydrochloride

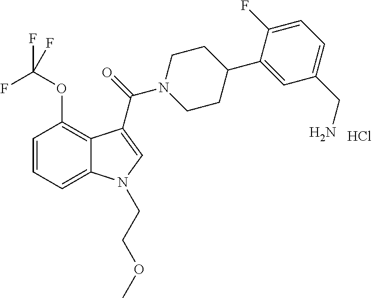

A mixture of 2,2,2-trifluoro-N-(4-fluoro-3-{1-[1-(2-methoxy-ethyl)-4-trifluoromethoxy-1H-indole-3-carbonyl]-piperidin-4-yl}-benzyl)-acetamide (45 mg, 0.076 mmol) in MeOH (3 mL) is added aqueous K₂CO₃ (84 mg, 0.61 mmol, dissolved in 1.5 mL H₂O). This mixture is stirred at r.t. overnight. LC/MS indicates the reaction is completed. The reaction mixture is concentrated in vacuo to remove most of the methanol. The residue is partitioned between H₂O and EtOAc. The two layers are separated, and the organic layer is washed with H₂O and brine, dried over MgSO₄, filtered, and concentrated in vacuo. The residue is dissolved in Et₂O and HCl in Et₂O (1.0 M, 3 mL) is added. The resulting suspension is concentrated in vacuo, and then dried in vacuo to yield the product (40 mg, 100%) as a beige solid.

¹H NMR (300 MHz, DMSO-d6) δ 8.30 (br, s 3H), 7.80-7.65 (m, 3H), 7.55-7.05 (m, 4H), 4.90-4.30 (m, 3H), 4.20-3.90 (m, 2H), 3.90-3.60 (m, 3H), 3.23 (s, 3H), 3.20-2.80 (m, 3H), 1.90-1.40 (m, 4H);

¹⁹F NMR (300 MHz, DMSO-d6) δ −56.20 (s, 3F), −119.18 (s, 1F);

LC 2.53 min; MS 494 (M+H, 100%).

### Example 55

### [4-(5-Aminomethyl-2-fluoro-phenyl)-piperidin-1-yl]-[4-(3-hydroxy-3-methyl-butyl)-1-(2-methoxy-ethyl)-1H-indol-3-yl]-methanone hydrochloride

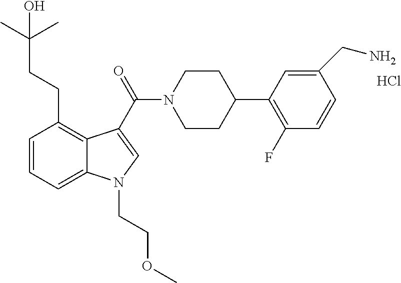

### A. (E)-3-(1H-Indol-4-yl)-acrylic acid methyl ester

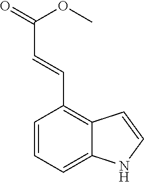

The title compound is prepared according to the procedure in Heterocycles, 1983, vol. 20(10), pp. 1983-5.

¹H NMR (300 MHz, CDCl₃) δ 8.4 (bs, 1H), 8.1 (d, H), 7.6-7.2 (m, 4H), 6.8 (m, 1H), 6.6 (d, 1H), 3.8 (s, 3H).

MS m/z: [M+H]⁺=202.

### B. 3-(1H-Indol-4-yl)-propionic acid methyl ester

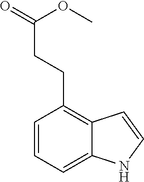

A solution of (E)-3-(1H-Indol-4-yl)-acrylic acid methyl ester (6.9 g, 34.33 mmol) dissolved in EtOAc (50 mL) is hydrogenated via a hydrogen paar shaker for 4 h at 50 psi with 10% Pd/C (1.0 g) as a catalyst. The reaction is filtered through celite and is concentrated in vacuo. The crude product is purified by flash chromatography on SiO₂ eluting with 20% ethyl acetate/heptane to give the titled compound (5.6 g, 80%).

¹H NMR (300 MHz, CDCl₃) δ 8.2 (bs, 1H), 7.3-7.1 (m, 3H), 6.9 (m, 1H), 6.6 (m, 1H), 3.7 (s, 3H), 3.3 (t, 2H), 2.8 (t, 2H).

MS m/z: [M+H]⁺=204.

### C. 3-[1-(2-Methoxy-ethyl)-1H-indol-4-yl]-propionic acid methyl ester

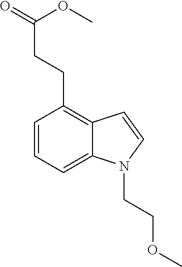

The title compound is prepared in a similar manner as described in Example 2F using 3-(1H-indol-4-yl)-propionic acid methyl ester and 2-methoxyethyl bromide in DMF as the starting materials.

¹H NMR (300 MHz, CDCl₃) δ 7.3-7.1 (m, 3H), 6.9 (d, 1H), 6.55 (m, 1H), 4.3 (t, 2H), 3.7 (t, 2H), 3.65 (s, 3H), 3.3 (s, 3H), 3.2 (m, 2H), 2.8 (m, 2H).

LCMS m/z: [M+H]⁺=262

### D. 4-[1-(2-Methoxy-ethyl)-1H-indol-4-yl]-2-methyl-butan-2-ol

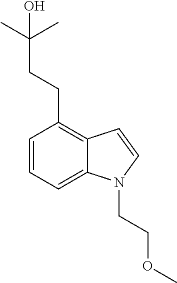

The title compound is prepared in a similar manner as described in Example 50B using 3-[1-(2-methoxy-ethyl)-1H-indol-4-yl]-propionic acid methyl ester as the starting material.

¹H NMR (300 MHz, CDCl₃) δ 7.3-7.1 (m, 3H), 6.9 (m, 1H), 6.5 (m, 1H), 4.3 (t, 2H), 3.7 (t, 2H), 3.3 (s, 3H), 3.0 (m, 2H), 1.9 (m, 2H), 1.6 (bs, 1H), 1.3 (s, 6H).

MS m/z: [M+H]⁺=262.

### E. Trifluoro-acetic acid 3-O-(2-methoxy-ethyl)-3-(2,2,2-trifluoro-acetyl)-1H-indol-4-yl]-1,1-dimethyl-propyl ester

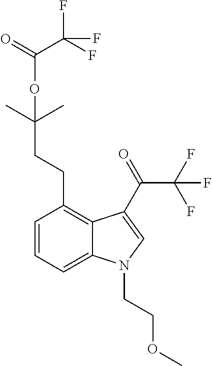

The title compound is prepared in a similar manner as described in Example 1F using 4-[1-(2-methoxy-ethyl)-1H-indol-4-yl]-2-methyl-butan-2-ol as the starting material.

¹H NMR (300 MHz, CDCl₃) δ 7.2 (m, 3H), 6.9 (m, 1H), 6.5 (m, 1H), 4.3 (t, 2H), 3.7 (t, 2H), 3.3 (s, 3H), 3.0 (m, 2H), 1.9 (m, 2H), 1.3 (s, 6H).

MS m/z: [M+H]⁺=262.

### F. 4-(3-Hydroxy-3-methyl-butyl)-1-(2-methoxy-ethyl)-1H-indole-3-carboxylic acid

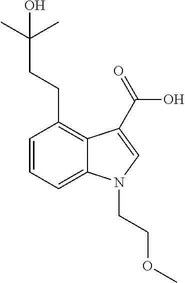

The title compound is prepared in a similar manner as described in Example 4C using trifluoro-acetic acid 3-O-(2-methoxy-ethyl)-3-(2,2,2-trifluoro-acetyl)-1H-indol-4-yl]-1,1-dimethyl-propyl ester as the starting material.

¹H NMR (300 MHz, DMSO-d6) δ 11.8 (s, 1H), 8.0 (s, 1H), 7.4 (d, 1H), 7.2 (m, 1H), 6.9 (d, 1H), 4.4 (t, 2H), 4.1 (s, 1H), 3.6 (t, 2H), 3.2 (m, 5H), 1.6 (m, 2H), 1.2 (s, 6H). MS m/z: [M+H]⁺=306.

### G. 2,2,2-Trifluoro-N-(4-fluoro-3-{1-[4-(3-hydroxy-3-methyl-butyl)-1-(2-methoxy-ethyl)-1H-indole-3-carbonyl]-piperidin-4-yl}-benzyl)-acetamide

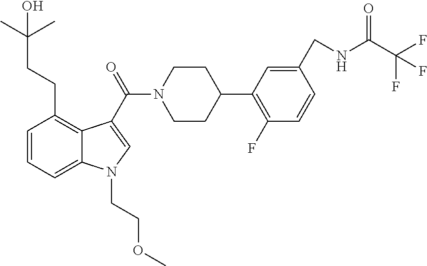

The title compound is prepared in a similar manner as described in Example 2I using 4-(3-hydroxy-3-methyl-butyl)-1-(2-methoxy-ethyl)-1H-indole-3-carboxylic acid and 2,2,2-trifluoro-N-(4-fluoro-3-piperidin-4-yl-phenyl)-acetamide hydrochloride as the starting materials and purified by RP-HPLC.

¹H NMR (300 MHz, CDCl₃) δ 7.3 (m, 4H), 7.1 (m, 1H), 7.0 (m, 2H), 6.9 (bs, 1H), 4.5 (m, 2H), 4.3 (t, 2H), 4.1 (m, 1H), 3.7 (t, 2H), 3.3 (s, 3H), 3.2-2.9 (m, 6H), 1.9-1.7 (m, 6H), 1.2 (m, 7H).

MS m/z: [M+H]⁺=592.

### H. [4-(5-Aminomethyl-2-fluoro-phenyl)-piperidin-1-yl]-[4-(3-hydroxy-3-methyl-butyl)-1-(2-methoxy-ethyl)-1H-indol-3-yl]-methanone hydrochloride

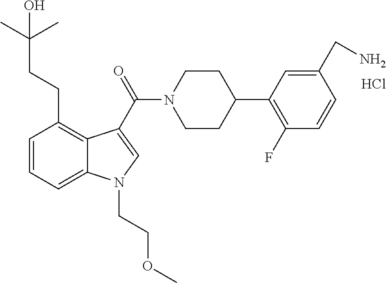

The title compound is prepared in a similar manner as described in Example 1K using 2,2,2-trifluoro-N-(4-fluoro-3-{1-[4-(3-hydroxy-3-methyl-butyl)-1-(2-methoxy-ethyl)-1H-indole-3-carbonyl]-piperidin-4-yl}-benzyl)-acetamide as the starting material.

¹H NMR (300 MHz, DMSO-d6) δ 8.3 (bs, 2H), 7.5 (m, 2H), 7.4 (m, 2H), 7.2 (m, 1H), 7.1 (m, 1H), 6.9 (m, 1H), 4.4 (m, 2H), 4.0 (m, 2H), 3.7 (m, 2H), 3.4 (m, 2H), 3.2 (s, 3H), 3.1-2.8 (m, 5H), 1.9-1.6 (m, 7H), 1.2 (s, 6H).

MS m/z: [M+H]⁺=496.

### Example 56

### [4-(5-Aminomethyl-2-fluoro-phenyl)-piperidin-1-yl]-[1-(2-methoxy-ethyl)-4-pyridin-4-yl-1H-indol-3-yl]-methanone

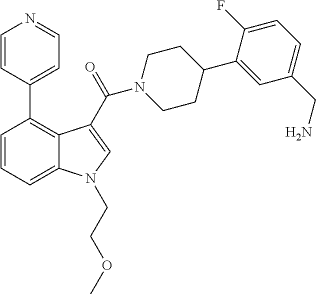

### A. 2,2,2-Trifluoro-N-(4-fluoro-3-{1-[1-(2-methoxy-ethyl)-4-pyridin-4-yl-1H-indole-3-carbonyl]-piperidin-4-yl}-benzyl)-acetamide

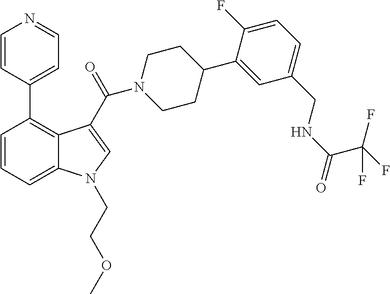

To a mixture of N-(3-{1-[4-bromo-1-(2-methoxy-ethyl)-1H-indole-3-carbonyl]-piperidin-4-yl}-4-fluoro-benzyl)-2,2,2-trifluoro-acetamide (0.30 g, 0.51 mmol) in dioxane (9 mL) and H₂O (1 mL) under nitrogen is added pyridine-4-boronic acid (0.12 g, 1.02 mmol) followed by of cesium fluoride (0.15 g, 1.02 mmol) and a catalytic amount of [1,1′-Bis(diphenylphosphino)-ferrocene]dichloropalladium(II). After stirring 6 h at reflux the reaction is quenched with H₂O solution and extracted with CH₂Cl₂. The combined organic layers are washed with aqueous saturated NaHCO₃ solution, dried over MgSO₄, filtered and concentrated in vacuo. The crude material is purified on silica gel with EtOAc as eluent to deliver the titled compound (0.21 g, 71%) as a beige solid. mp 152-155° C.

¹H NMR (300 MHz, CDCl₃) δ 8.61 (m, 1H), 7.61 (m, 2H), 7.50 (m, 2H), 7.39 (m, 2H), 7.18 (m, 2H), 6.94 (m, 2H), 4.65 (m, 2H), 4.48 (m, 2H), 4.35 (t, J=5.1 Hz, 2H), 3.78 (t, J=5.1 Hz, 2H), 3.35 (s, 3H), 3.30 (m, 2H), 2.96 (m, 1H), 2.83 (m, 1H), 1.66 (m, 2H), 1.35 (m, 1H);

MS 583 (M+1).

### B [4-(5-Aminomethyl-2-fluoro-phenyl)-piperidin-1-yl]-[1-(2-methoxy-ethyl)-4-pyridin-4-yl-1H-indol-3-yl]-methanone

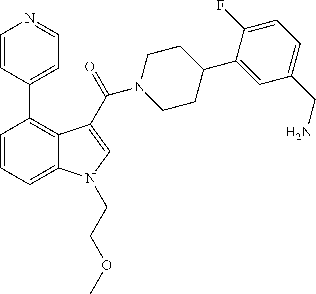

To a solution of 2,2,2-trifluoro-N-(4-fluoro-3-{1-[1-(2-methoxy-ethyl)-4-pyridin-4-yl-1H-indole-3-carbonyl]-piperidin-4-yl}-benzyl)-acetamide (0.17 g 0.29 mmol) in MeOH (10 mL) and H₂O (5 mL) is added aqueous 50% NaOH solution (1 mL). After stirring at ambient temperature for 1 h the mixture is concentrated in vacuo, diluted with H₂O and extracted with EtOAc as well as CH₂Cl₂. The combined organic layers are dried over MgSO₄, filtered and concentrated in vacuo diluted with MeOH and adsorbed onto silica gel. This material is purified on silica gel (5% 7N NH₃/MeOH:95% CH₂Cl₂ as eluent). Concentration of appropriate fractions delivers the titled compound (0.12 g, 85%) as a foam.

¹H NMR (300 MHz, CDCl₃) δ 8.69 (br s, 2H), 7.52 (m, 2H), 7.39 (m, 2H), 7.19 (m, 2H), 7.09 (m, 1H), 4.56 (m, 1H), 4.35 (t, J=4.5 Hz, 2H), 3.80 (t, J=4.5 Hz, 2H), 3.76 (m, 2H), 3.35 (s, 3H), 3.36 (m, 1H), 2.80 (m, 1H), 1.8-1.2 (m, 4H);

MS 487 (M+1).

### Example 57

### [4-(5-Aminomethyl-2-fluoro-phenyl)-piperidin-1-yl]-[4-ethoxy-1-(2-methoxy-ethyl)-1H-indol-3-yl]-methanone hydrochloride

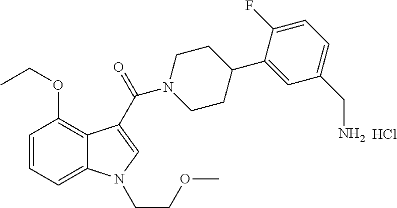

### A. 4-Ethoxy-1H-indole

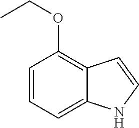

The title compound is prepared in a similar manner as described in Example 49A using 4-hydroxyindole and anhydrous ethanol as the starting materials.

¹H NMR (300 MHz, CDCl₃) δ 8.14 (br s, 1H), 7.14-7.09 (m, 2H), 7.02-7.00 (m, 1H), 6.70-6.68 (m, 1H), 6.55-6.53 (m, 1H), 4.21 (q, 2H), 1.51 (t, 3H).

### B. 4-Ethoxy-1-(2-methoxy-ethyl)-1H-indole

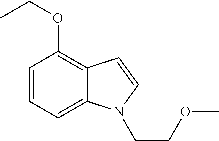

The title compound is prepared in a similar manner as described in Example 1E using 4-ethoxy-1H-indole as the starting material.

¹H NMR (300 MHz, CDCl₃) δ 7.14-7.09 (m, 1H), 6.98-6.95 (m, 1H), 6.63-6.61 (m, 1H), 6.52 (d, 1H), 4.27 (t, 2H), 4.20 (q, 2H), 3.70 (t, 2H), 3.31 (s, 3H), 1.50 (t, 3H).

### C. 1-[4-Ethoxy-1-(2-methoxy-ethyl)-1H-indol-3-yl]-2,2,2-trifluoro-ethanone

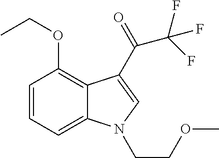

The title compound is prepared in a similar manner as described in Example 2G using 4-ethoxy-1-(2-methoxy-ethyl)-1H-indole as the starting material.

¹H NMR (300 MHz, CDCl₃) δ 7.89-7.88 (m, 1H), 7.24-7.18 (m, 1H), 6.93-6.90 (m, 1H), 6.69 (d, 1H), 4.25 (t, 2H), 4.22 (q, 2H), 3.67 (t, 2H), 3.24 (s, 3H), 1.50 (t, 3H).

### D, 4-Ethoxy-1-(2-methoxy-ethyl)-1H-indole-3-carboxylic acid

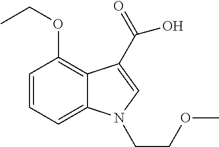

The title compound is prepared in a similar manner as describedin Example 2H using 1-[4-ethoxy-1-(2-methoxy-ethyl)-1H-indol-3-yl]-2,2,2-trifluoro-ethanone as the starting material.

¹H NMR (300 MHz, DMSO-d6) δ 11.69 (s, 1H), 8.00 (s, 1H), 7.27-7.16 (m, 2H), 6.79 (d, 1H), 4.38 (t, 2H), 4.26 (q, 2H), 3.66 (t, 2H), 3.21 (s, 3H), 1.42 (t, 3H).

### E. N-(3-{1-[4-Ethoxy-1-(2-methoxy-ethyl)-1H-indole-3-carbonyl]-piperidin-4-yl}-4-fluoro-benzyl)-2,2,2-trifluoro-acetamide

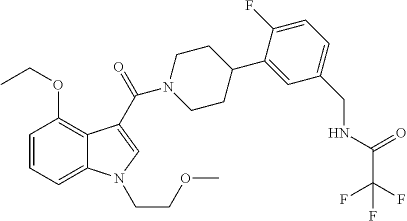

The title compound is prepared in a similar manner as described in Example 2I using 4-ethoxy-1-(2-methoxy-ethyl)-1H-indole-3-carboxylic acid and 2,2,2-trifluoro-N-(4-fluoro-3-piperidin-4-yl-benzyl)-acetamide hydrochloride as the starting materials.

¹H NMR (300 MHz, CDCl₃) δ 7.18-7.08 (m, 4H), 7.02-6.95 (m, 2H), 6.55 (d, 1H), 4.97 (br s, 1H), 4.44 (d, 2H), 4.24 (t, 2H), 4.16-4.08 (m, 3H), 3.90 (br s, 1H), 3.71 (t, 2H), 3.18-3.03 (m, 2H), 2.04 (s, 1H), 1.90-1.56 (m, 4H), 1.46 (t, 3H), 1.26 (t, 2H).

### F. [4-(5-Aminomethyl-2-fluoro-phenyl)-piperidin-1-yl]-[4-ethoxy-1-(2-methoxy-ethyl)-1H-indol-3-yl]-methanone hydrochloride

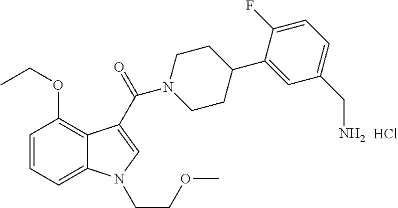

The title compound is prepared in a similar manner as described in Example 3B using N-(3-{1-[4-ethoxy-1-(2-methoxy-ethyl)-1H-indole-3-carbonyl]-piperidin-4-yl}-4-fluoro-benzyl)-2,2,2-trifluoro-acetamide as the starting material.

¹H NMR (300 MHz, DMSO-d6) δ 8.64 (br s, 3H), 7.54 (br s, 1H), 7.40 (m, 2H), 7.21-7.15 (m, 1H), 7.12-7.5 (m, 2H), 6.59-6.57 (m, 1H), 4.31 (t, 2H), 4.12-4.06 (m, 2H), 3.95 (br s, 2H), 3.67-3.64 (m, 3H), 3.22 (s, 3H), 3.16-2.99 (m, 3H), 2.82 (br s, 1H), 1.90-1.59 (m, 4H), 1.38 (t, 3H).

### Example 58

### [4-(5-Aminomethyl-2-fluoro-phenyl)-piperidin-1-yl]-[4-cyclopropoxy-1-(2-methoxy-ethyl)-1H-indol-3-yl]-methanone hydrochloride

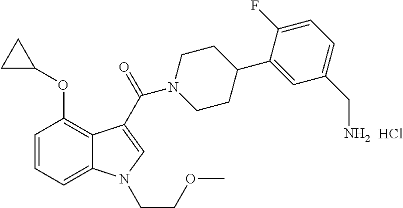

A Carbonic acid tert-butyl ester 1H-indol-4-yl ester

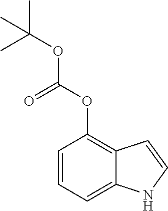

The title compound is prepared according to the procedure by Somei, M. et al., Chem. Pharm. Bull., 2002, vol. 50, pp. 92-99 using 4-hydroxy-1H-indole and di-tert-butyldicarbonate as the starting materials.

¹H NMR (300 MHz, CDCl₃) δ 8.26 (br s, 1H), 7.02 (m, 1H), 7.21-7.21 (m, 1H), 7.17-7.11 (m, 2H), 6.95-6.93 (m, 1H), 6.50-6.48 (m, 1H), 1.60 (s, 9H).

### B. Carbonic acid tert-butyl ester 1-(2-methoxy-ethyl)-1H-indol-4-yl ester

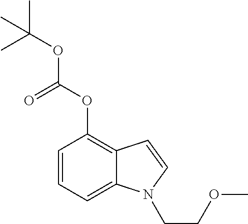

The title compound is prepared in a similar manner as described in Example 1E using carbonic acid tert-butyl ester 1H-indol-4-yl ester as the starting material.

¹H NMR (300 MHz, CDCl₃) δ 7.77 (d, 1H), 7.49 (d, 1H), 7.21 (t, 1H), 6.73 (m, 1H), 6.67 (d, 1H), 4.26 (t, 2H), 3.83 (t, 2H), 3.49 (s, 3H), 1.67 (s, 9H).

### C. 1-(2-Methoxy-ethyl)-1H-indol-4-ol

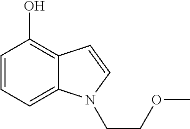

To a mixture of carbonic acid tert-butyl ester 1-(2-methoxy-ethyl)-1H-indol-4-yl ester (5.81 g, 19.94 mmol) in 1,4-dioxane (90 mL) is added a solution of 2 N HCl in water (50 mL). The resulting mixture is refluxed for ˜1 hour. The solvent is removed in vacuo and the residue is partitioned between water and EtOAc. The mixture is basified with 10 N NaOH and the organic layer is separated. The aqueous phase is extracted with EtOAc (×2). The organic phases are combined, washed with water and brine then separated and dried over MgSO₄. The organic phase is concentrated in vacuo and the crude residue is flash chromatographed over SiO₂ using Heptane:EtOAc (70:30) to afford the titled compound (900 mg, 24%) as an orange oil.

¹H NMR (300 MHz, CDCl₃) δ 8.15 (br s, 1H), 7.13-7.02 (m, 3H), 6.71-6.69 (m, 1H), 6.55-6.53 (m, 1H), 4.30 (t, 2H), 3.86 (t, 2H), 3.50 (s, 3H).

### D. 4-Cyclopropoxy-1-(2-methoxy-ethyl)-1H-indole

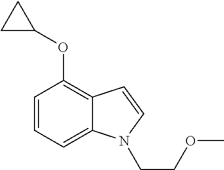

The title compound is prepared according to the procedure by Chiu, G. et al., Bioorg. Med. Chem. Lett., 2007, vol. 17, pp. 3930-3934 using 1-(2-methoxy-ethyl)-1H-indol-4-ol and cyclopropyl bromide in N,N-dimethyl-acetamide as the starting materials.

¹H NMR (300 MHz, CDCl₃) δ 7.21-7.09 (m, 2H), 7.02 (m, 1H), 6.56-6.53 (m, 2H), 4.28 (t, 2H), 3.83 (t, 2H), 3.48 (s, 3H), 3.36-3.29 (m, 1H), 1.05-1.00 (m, 4H).

### E. 1-[4-Cyclopropoxy-1-(2-methoxy-ethyl)-1H-indol-3-yl]-2,2,2-trifluoro-ethanone

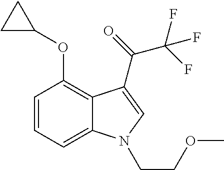

The title compound is prepared in a similar manner as described in Example 2G using 4-cyclopropoxy-1-(2-methoxy-ethyl)-1H-indole as the starting material.

¹H NMR (300 MHz, CDCl₃) δ 7.86 (m, 1H), 7.22-7.24 (m, 2H), 6.81 (m, 1H), 4.26 (t, 2H), 3.94 (t, 2H), 3.50 (s, 3H), 3.44 (sept, 1H), 1.26-1.05 (m, 4H).

### F. 4-Cyclopropoxy-1-(2-methoxy-ethyl)-1H-indole-3-carboxylic acid

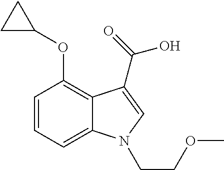

The title compound is prepared in a similar manner as described in Example 2H using 1-[4-cyclopropoxy-1-(2-methoxy-ethyl)-1H-indol-3-yl]-2,2,2-trifluoro-ethanone as the starting material.

¹H NMR (300 MHz, CD₃OD) δ 8.00 (s, 1H), 7.41-7.39 (m, 1H), 7.28 (t, 1H), 6.88 (d, 1H), 4.43-4.40 (m, 2H), 3.85-3.82 (m, 2H), 3.49 (sept, 1H), 3.44 (s, 3H), 1.21-1.14 (m, 2H), 1.06-1.01 (m, 2H).

### G. N-(3-{1-[4-Cyclopropoxy-1-(2-methoxy-ethyl)-1H-indole-3-carbonyl]-piperidin-4-yl}-4-fluoro-benzyl)-2,2,2-trifluoro-acetamide

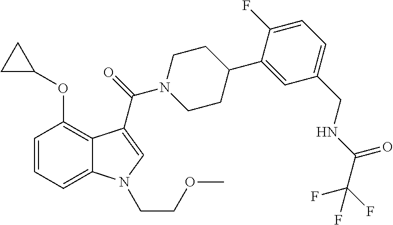

The title compound is prepared in a similar manner as described in Example 6E using 4-cyclopropoxy-1-(2-methoxy-ethyl)-1H-indole-3-carboxylic acid and 2,2,2-trifluoro-N-(4-fluoro-3-piperidin-4-yl-benzyl)-acetamide hydrochloride as the starting materials.

¹H NMR (300 MHz, CDCl₃) δ 7.24-7.10 (m, 5H), 7.03-6.97 (m, 2H), 6.59-6.57 (m, 1H), 4.46 (d, 2H), 4.22 (br s, 2H), 3.80 (br s, 3H), 3.46-3.30 (m, 5H), 3.15-3.03 (m, 1H), 2.96 (s, 3H), 1.97-1.71 (m, 3H), 1.08-1.02 (m, 4H).

### H. [4-(5-Aminomethyl-2-fluoro-phenyl)-piperidin-1-yl]-[4-cyclopropoxy-1-(2-methoxy-ethyl)-1H-indol-3-yl]-methanone hydrochloride

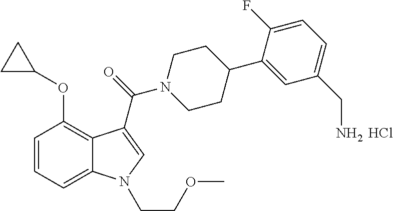

The title compound is prepared in a similar manner as described in Example 3B using N-(3-{1-[4-cyclopropoxy-1-(2-methoxy-ethyl)-1H-indole-3-carbonyl]-piperidin-4-yl}-4-fluoro-benzyl)-2,2,2-trifluoro-acetamide as the starting material.

¹H NMR (300 MHz, DMSO-d6) δ 8.40 (br s, 3H), 7.52-7.50 (m, 1H), 7.39-7.35 (m, 2H), 7.23-7.11 (m, 3H), 6.65-6.62 (m, 1H), 4.72 (br s, 1H), 4.43 (br s, 4H), 4.16 (br s, 2H), 3.98 (m, 2H), 3.72 (br s, 2H), 3.62 (br s, 1H), 3.48-3.42 (m, 1H), 3.36 (s, 3H), 3.10-3.02 (m, 1H), 2.84 (br s, 1H), 1.82-1.56 (m, 4H), 0.98 (m, 1H).

### Example 59

### [3-[4-(5-Aminomethyl-2-fluoro-phenyl)-piperidine-1-carbonyl]-1-(2-methoxy-ethyl)-1H-indol-4-yl]-morpholin-4-yl-methanone hydrochloride

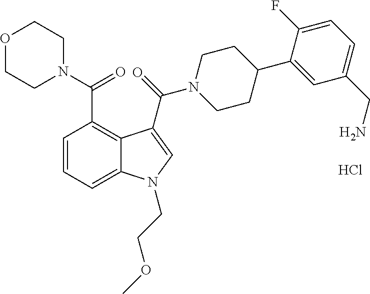

### A. (1H-Indol-4-yl)-morpholin-4-yl-methanone

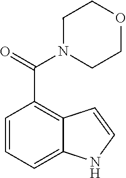

To a mixture of 1H-indole-4-carboxylic acid (1.0 g, 6.2 mmol) in THF (10 mL) under nitrogen is added carbonyl diimidazole (1.1 g, 6.8 mmol). After stirring 0.5 h at ambient temperature, morpholine (0.7 g, 8.1 mmol) is added. After stirring for 2 h, the reaction is quenched with 5% aqueous HCl and extracted with EtOAc. The combined organic layers are washed with aqueous saturated NaHCO₃ solution, dried over MgSO₄, filtered and concentrated in vacuo. The resulting solid is recrystallized from EtOAc/heptane to yield the titled compound (1.3 g, 92%) as a beige solid (mp 127-129° C.).

¹H NMR (300 MHz, CDCl₃) δ 8.36 (s, 1H), 7.45 (m, 1H), 7.24 (m, 4H), 3.43 (m, 8H); MS 231 (M+1).

### B. [1-(2-Methoxy-ethyl)-1H-indol-4-yl]-morpholin-4-yl-methanone

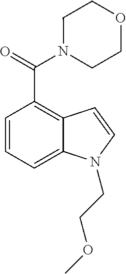

To a solution of (1H-indol-4-yl)-morpholin-4-yl-methanone (1.00 g, 4.3 mmol) in DMSO (10 mL) under nitrogen is added KOH (0.73 g, 13.0 mmol). After stirring for 10 minutes, 1-bromo-2-methoxy-ethane (0.72 g, 5.2 mmol) is added followed by a catalytic amount of KI. After stirring an additional 4 h the reaction is quenched with aqueous saturated NaCl solution and extracted with EtOAc. The combined organic layers are dried over MgSO₄, filtered and concentrated in vacuo to yield the titled compound (1.20 g, 96%) as a clear light yellow oil.

¹H NMR (300 MHz, CDCl₃) δ 7.42 (m, 1H), 7.25 (m, 2H), 7.12 (m, 1H), 6.49 (m, 1H), 4.28 (t, J=6.5 Hz, 2H), 3.65 (t, J=6.5 Hz, 2H), 3.59 (m, 8H), 3.31 (s, 3H);

MS 289 (M+1).

### C. 1-(2-Methoxy-ethyl)-4-(morpholine-4-carbonyl)-1H-indole-3-carbaldehyde

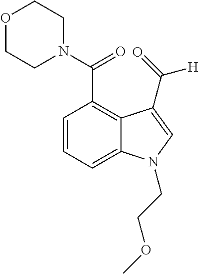

To DMF (5 mL) at 0° C. under nitrogen is added POCl3 (0.34 g, 0.21 mmol). After stirring 5 minutes, [1-(2-methoxy-ethyl)-1H-indol-4-yl]-morpholin-4-yl-methanone (0.50 g, 0.17 mmol) in DMF (3 mL) is added dropwise. After stirring at ambient temperature for 1 h the mixture is diluted with aqueous 1 N KOH (5 mL) solution and stirred an additional 1.5 h. The reaction is acidified with aqueous 10% HCl and extracted with EtOAc as well as CH₂Cl₂. The combined organic layers are dried over MgSO₄, filtered and concentrated in vacuo to yield the titled compound (0.50 g, 91%) as a yellow oil.

¹H NMR (300 MHz, CDCl₃) δ 9.95 (s, 1H), 7.92 (m, 1H), 7.39 (m, 2H), 7.22 (m, 1H), 4.38 (m, 2H), 3.98 (m, 3H), 3.75 (m, 3H), 3.54 (m, 2H), 3.34 (s, 3H), 3.21 (m, 2H);

MS 317 (M+1).

### D. 1-(2-Methoxy-ethyl)-4-(morpholine-4-carbonyl)-1H-indole-3-carboxylic acid

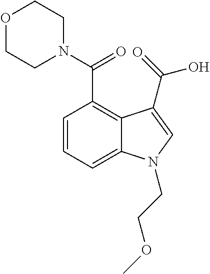

This compound is prepared via the Pinnick Oxidation method described in Tetrahedron 1981, 37, 2091. From 1-(2-methoxy-ethyl)-4-(morpholine-4-carbonyl)-1H-indole-3-carbaldehyde (0.20 g, 0.63 mmol) is obtained the titled compound (0.10 g, 48%) as solid mp 201-203° C. (from CH₂Cl₂/heptane).

¹H NMR (300 MHz, CDCl₃) δ 8.04 (s, 1H), 7.42 (m, 1H), 7.30 (m, 2H), 7.19 (m, 1H), 4.35 (m, 2H), 4.01 (m, 2H), 3.76 (m, 4H), 3.63 (m, 1H), 3.51 (m, 1H), 3.34 (s, 3H), 3.22 (m, 2H);

MS 333 (M+1).

### E. 2,2,2-Trifluoro-N-(4-fluoro-3-{1-O-(2-methoxy-ethyl)-4-(morpholine-4-carbonyl)-1H-indole-3-carbonyl]-piperidin-4-yl}-benzyl)-acetamide

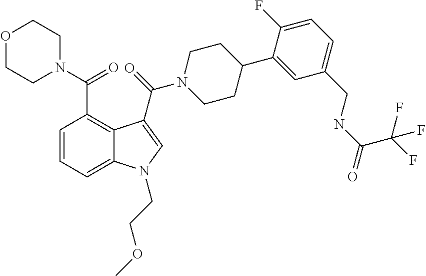

The title compound is prepared in a similar manner as described in Example 2I using 1-(2-methoxy-ethyl)-4-(morpholine-4-carbonyl)-1H-indole-3-carboxylic acid and 2,2,2-trifluoro-N-(4-fluoro-3-piperidin-4-yl-phenyl)-acetamide hydrochloride as the starting materials.

¹H NMR (300 MHz, CDCl₃) δ 7.42 (m, 2H), 7.08 (m, 3H), 6.99 (m, 2H), 4.47 (m, 2H), 4.29 (m, 2H), 3.79 (m, 2H), 3.70 (m, 4H), 3.32 (s, 3H), 3.10 (m, 1H), 2.00-1.60 (m, 8H),

MS 619 (M+1).

### F. [3-[4-(5-Aminomethyl-2-fluoro-phenyl)-piperidine-1-carbonyl]-1-(2-methoxy-ethyl)-1H-indol-4-yl]-morpholin-4-yl-methanone hydrochloride

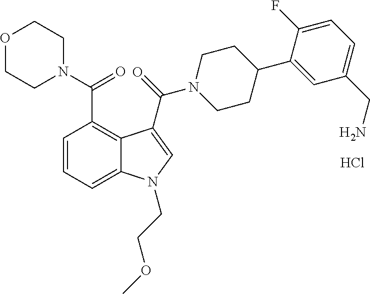

The title compound is prepared in a similar manner as described in Example 1K using 2,2,2-trifluoro-N-(4-fluoro-3-{1-[1-(2-methoxy-ethyl)-4-(morpholine-4-carbonyl)-1H-indole-3-carbonyl]-piperidin-4-yl}-benzyl)-acetamide (mp 142-147° C.) as the starting material.

¹H NMR (300 MHz, CDCl₃) δ 8.12 (br s, 3H), 7.64 (m, 2H), 7.54 (m, 1H), 7.35 (m, 1H), 7.24 (m, 1H), 7.01 (m, 1H), 4.41 (m, 3H), 4.38 (m, 2H), 4.00 (m, 6H), 3.67 (m, 2H), 3.50-3.00 (m, 11H), 1.80-1.60 (m, 2H);

MS 523 (M+1).

### Example 60

### [4-(5-Aminomethyl-2-fluoro-phenyl)-piperidin-1-yl]-[1-(2-methoxy-ethyl)-4-phenyl-1H-indol-3-yl]-methanone hydrochloride

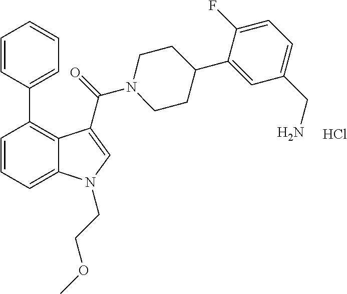

### A. 2,2,2-Trifluoro-N-(4-fluoro-3-{1-[1-(2-methoxy-ethyl)-4-phenyl-1H-indole-3-carbonyl]-piperidin-4-yl}-benzyl)-acetamide

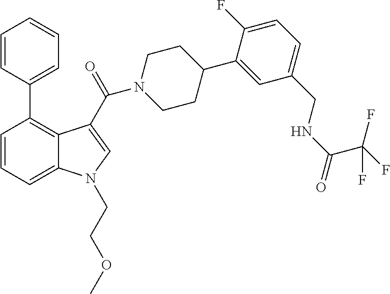

A mixture of N-(3-{1-[4-bromo-1-(2-methoxy-ethyl)-1H-indole-3-carbonyl]-piperidin-4-yl}-4-fluoro-benzyl)-2,2,2-trifluoro-acetamide (200 mg, 0.34 mmol), phenylboronic acid (50 mg, 0.41 mmol), cesium carbonate (223 mg, 0.68 mmol), and Pd(dppf)Cl₂.CH₂Cl₂ (28 mg, 10% mol) in dioxane/H₂O (10 mL/1 mL) is heated at 80° C. overnight. The reaction mixture is cooled to r.t., and then partitioned between EtOAc and 10% citric acid. The two layers are separated, and the aqueous layer is extracted with EtOAc once. The combined organic layers are washed with sat NaHCO₃, H₂O, and brine, dried over Na₂SO₄, filtered, and concentrated in vacuo. The crude material is purified on silica gel with CH₂Cl₂/MeOH (100/0 to 99/1) as eluent to give the product (175 mg, 88%) as a beige foam.

¹H NMR (300 MHz, CDCl₃) δ 7.75-7.15 (m, 8H), 7.15-6.90 (m, 4H), 6.50 (br s, 1H), 4.65-4.40 (m, 3H), 4.34 (t, J=5.3 Hz, 2H), 3.78 (t, J=5.5 Hz, 2H), 3.45 (m, 1H), 3.36 (s, 3H), 2.80-2.60 (m, 1H), 2.20-1.75 (m, 2H), 1.75-1.05 (m, 4H);

LC Rt 1.06 min; MS 582 (M+H, 100%).

### B. [4-(5-Aminomethyl-2-fluoro-phenyl)-piperidin-1-yl]-[1-(2-methoxy-ethyl)-4-phenyl-1H-indol-3-yl]-methanone hydrochloride

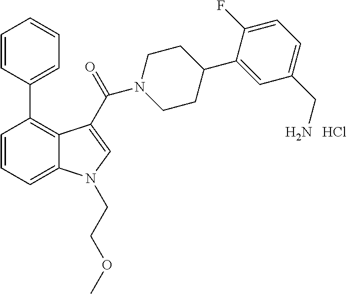

To a mixture of 2,2,2-trifluoro-N-(4-fluoro-3-{1-[1-(2-methoxy-ethyl)-4-phenyl-1H-indole-3-carbonyl]-piperidin-4-yl}-benzyl)-acetamide (120 mg, 0.29 mmol) in MeOH (5 mL) is added aqueous K₂CO₃ (323 mg, 2.33 mmol, dissolved in 1.0 mL H₂O). This mixture is heated at 45° C. for 2 h. LC/MS indicates the reaction is completed. The reaction mixture is concentrated in vacuo to remove most of the methanol. The residue is partitioned between H₂O and EtOAc. The two layers are separated, and the organic layer is washed with H₂O and brine, dried over Na₂SO₄, filtered, and concentrated in vacuo. 4.0 M HCl in dioxane is added. The resulting suspension is concentrated in vacuo, and then dried in vacuo to yield the product (156 mg, 100%) as a slightly pink solid.

¹H NMR (300 MHz, DMSO-d6) δ 8.16 (br, s 3H), 7.70-7.50 (m, 2H), 7.55-7.40 (m, 5H), 7.40-7.20 (m, 3H), 7.20-7.00 (m, 2H), 4.55-4.20 (m, 3H), 4.10-3.90 (m, 2H), 3.80-3.60 (m, 3H), 3.30 (m, 1H), 3.25 (s, 3H), 2.80-2.60 (m, 1H), 2.40-1.80 (m, 2H), 1.70-1.00 (m, 4H);

LC 0.77 min; MS 486 (M+H, 100%).

### Example 61

### [4-(5-Aminomethyl-2-fluoro-phenyl)-piperidin-1-yl]-[1-(2-methoxy-ethyl)-4-pyrimidin-5-yl-1H-indol-3-yl]-methanone dihydrochloride

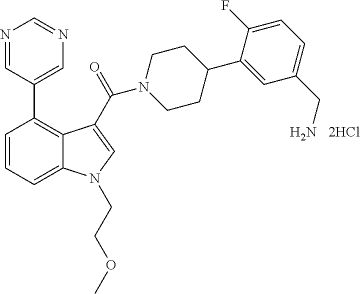

### A. 2,2,2-Trifluoro-N-(4-fluoro-3-{1-[1-(2-methoxy-ethyl)-4-pyrimidin-5-yl-1H-indole-3-carbonyl]-piperidin-4-yl}-benzyl)-acetamide

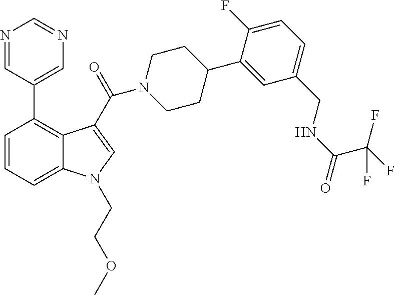

A mixture of N-(3-{1-[4-bromo-1-(2-methoxy-ethyl)-1H-indole-3-carbonyl]-piperidin-4-yl}-4-fluoro-benzyl)-2,2,2-trifluoro-acetamide (200 mg, 0.34 mmol), 5-pyrimidineboronic acid (51 mg, 0.21 mmol), cesium carbonate (223 mg, 0.68 mmol), and Pd(dppf)Cl₂.CH₂Cl₂ (28 mg, 10% mol) in dioxane/H₂O (10 mL/1 mL) is heated at 80° C. overnight. The reaction mixture is evaporated to dryness. The residue is dissolved in EtOAc and the resulting solution is washed with H₂O and brine, dried over Na₂SO₄, filtered, and concentrated in vacuo. The crude material is used in the next step without further purification.

### B. [4-(5-Aminomethyl-2-fluoro-phenyl)-piperidin-1-yl]-[1-(2-methoxy-ethyl)-4-pyrimidin-5-yl-1H-indol-3-yl]-methanone dihydrochloride

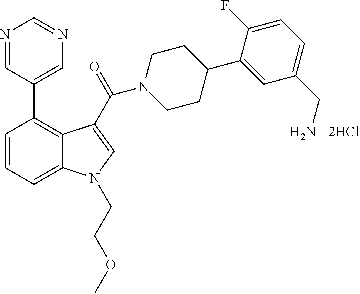

A mixture of 2,2,2-trifluoro-N-(4-fluoro-3-{1-[1-(2-methoxy-ethyl)-4-pyrimidin-5-yl-1H-indole-3-carbonyl]-piperidin-4-yl}-benzyl)-acetamide (from entry 61A) in MeOH (5 mL) is added aqueous K₂CO₃ (323 mg, 2.33 mmol, dissolved in 1.0 mL H₂O). This mixture is heated at 45° C. for 2 h. LC/MS indicates the reaction is completed. The reaction mixture is concentrated in vacuo to remove most of the methanol. The residue is partitioned between H₂O and EtOAc. The two layers are separated, and the organic layer is washed with H₂O and brine, dried over Na₂SO₄, filtered, and concentrated in vacuo. 4.0 M HCl in dioxane is added. The crude material is purified by RP-HPLC to yield 45 mg of the product as a beige solid.

¹H NMR (300 MHz, DMSO-d6) δ 9.22 (s, 1H), 8.84 (s, 1H), 8.08 (br s 4H), 7.95-7.70 (m, 2H), 7.0-7.30 (m, 3H), 7.30-7.10 (m, 3H), 4.50-4.40 (m, 2H), 4.15-3.95 (m, 3H), 3.80-3.65 (m, 3H), 3.30 (m, 1H), 3.24 (s, 3H), 3.05-2.85 (m, 1H), 2.60-2.40 (m, 2H), 1.75-1.05 (m, 4H);

LC 0.63 min; MS 488 (M+H, 100%).

### Example 62

### [4-(5-Aminomethyl-2-fluoro-phenyl)-piperidin-1-yl]-[1-(2-imidazol-1-yl-ethyl)-7-methyl-1H-indol-3-yl]-methanone dihydrochloride

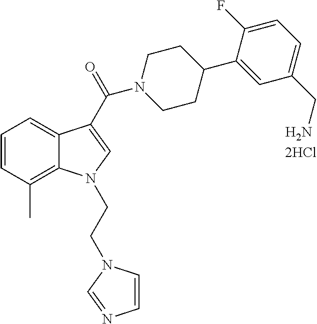

### A. 2-(7-Methyl-indol-1-yl)ethanol

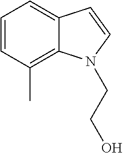

A mixture of 7-methyl-1H-indole (500 mg, 3.81 mmol) and powdered KOH (853 mg, 15.24 mmol) in DMSO (5 mL) is stirred at r.t. for 30 min then (2-bromo-ethoxy)-tert-butyl-dimethyl-silane (1.27 g, 5.72 mmol) is added. After the reaction mixture is stirred at r.t. for 2 h, it is partitioned between H₂O and Et₂O. The two layers are separated, and the aqueous layer is extracted with Et₂O (3×). The combined organic extracts are washed with H₂O and brine, dried over Na₂SO₄, filtered, and concentrated in vacuo. The crude material is purified on silica gel with heptane/EtOAc (95/5 to 60/40) as eluent to yield the titled product (232 mg, 34%) as a yellow liquid.

¹H NMR (300 MHz, CDCl₃) δ 7.48 (d, J=7.7 Hz, 1H), 7.11 (d, J=3.1 Hz, 1H), 7.10-6.90 (m, 2H), 6.51 (d, J=3.3 Hz, 1H), 4.51 (t, J=6.2 Hz, 2H), 3.93 (q, J=5.3 Hz, 2H), 2.71 (s, 3H), 1.42 (t, J=5.9 Hz, 1H);

LC Rt: 0.84 min; MS 176 (M+H, 100%).

### B. Methanesulfonic acid 2-(7-methyl-indol-1-yl)ethyl ester

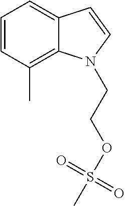

To a mixture of 2-(7-methyl-indol-1-yl)-ethanol (1.00 g, 5.71 mmol) and DIEA (1.49 mL, 8.56 mmol) in CH₂Cl₂ (10 mL) at 0° C. is added methansulfonyl chloride (0.53 mL, 6.85 mmol) dropwise. After the addition is completed, the cooling bath is removed. The reaction mixture is stirred at r.t. for 2 h. The mixture is then partitioned between sat 10% citric acid and CH₂Cl₂. The two layers are separated and the organic acid is washed with sat. NaHCO₃, and brine, dried over Na₂SO₄, filtered, and concentrated in vacuo to yield the crude titled product (1.55 g, >100%) as a yellow solid. This solid is used in the next step without further purification.

¹H NMR (300 MHz, CDCl₃) δ 7.47 (d, J=7.1 Hz, 1H), 7.15-6.85 (m, 3H), 6.52 (d, J=3.3 Hz, 1H), 4.69 (t, J=5.5 Hz, 2H), 4.46 (t, J=5.5 Hz, 2H), 2.70 (s, 3H), 2.58 (s, 3H),

LC Rt: 1.01 min; MS 254 (M+H, 100%).

### C. 1-(2-Imidazol-1-yl-ethyl)-7-methyl-1H-indole

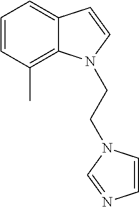

To a mixture of methanesulfonic acid 2-(7-methyl-indol-1-yl)-ethyl ester (1.55 g, 6.12 mmol) in DMF (12 mL) is added sodium imidazole (0.86 g, 9.18 mmol). After the reaction mixture is stirred at r.t. for 3 h, the reaction mixture is concentrated in vacuo, and the crude material is purified on silica gel with CH₂Cl₂/MeOH (100/0 to 86/14) as eluent to yield the titled product (1.38 g, 100%) as a light yellow oil.

¹H NMR (300 MHz, CDCl₃) δ 7.48 (d, J=7.7 Hz, 1H), 7.13 (s, 1H), 7.10-6.85 (m, 3H), 6.65-6.50 (m, 2H), 6.44 (d, J=3.3 Hz, 1H), 4.64 (t, J=5.7 Hz, 2H), 4.27 (t, J=6.0 Hz, 2H), 2.66 (s, 3H);

LC Rt: 0.67 min; MS 226 (M+H, 100%).

### D. 2,2,2-Trifluoro-1-[1-(2-imidazol-1-yl-ethyl)-7-methyl-1H-indol-3-yl]-ethanone

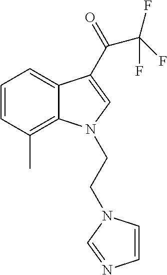

A mixture of 1-(2-imidazol-1-yl-ethyl)-7-methyl-1H-indole (460 mg, 2.04 mmol) and TFAA (0.77 mL, 5.55 mmol) in DMF (5 mL) is heated at 40° C. for 3 h. The reaction is cooled to r.t. and then partitioned between H₂O and Et₂O. The two layers are separated and the aqueous layer is extracted with Et₂O (2×). The combined organic layers are washed with sat. NaHCO₃ and brine, dried over Na₂SO₄, and concentrated in vacuo. The crude material is purified on silica gel with CH₂Cl₂/MeOH (100/0 to 98/2) as eluent to yield the titled product (512 mg, 78%) as a light brown solid.

¹H NMR (300 MHz, CDCl₃) δ 8.55-8.30 (m, 1H), 7.50-6.90 (m, 5H), 6.60 (bs, 1H)), 4.90-4.60 (m, 2H), 4.50-4.30 (m, 2H), 2.75 (s, 3H);

LC Rt: 0.66 min; MS 322 (M+H, 100%).

### E. 1-(2-Imidazol-1-yl-ethyl)-7-methyl-1H-indole-3-carboxylic acid

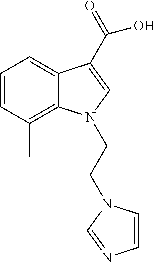

A mixture of 2,2,2-trifluoro-1-[1-(2-imidazol-1-yl-ethyl)-7-methyl-1H-indol-3-yl]-ethanone (510 mg, 1.58 mmol) in MeOH (3 mL) and 5.0 M NaOH (3 mL) is heated at 70° C. overnight. This mixture is concentrated in vacuo to remove the methanol. The residue is evaporated to dryness with a high vacuum pump. 3.0 M HCl is added to the residue until pH is ˜6. The resulting solution is concentrated to dryness in vacuo. The crude material is used in the next step without further purification.

LC Rt: 0.50 min; MS 270 (M+H, 100%).

### F. 2,2,2-Trifluoro-N-(4-fluoro-3-{1-[1-(2-imidazol-1-yl-ethyl)-7-methyl-1H-indole-3-carbonyl]-piperidin-4-yl}-benzyl)-acetamide

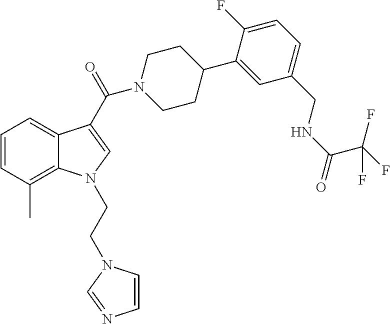

A mixture of crude 1-(2-imidazol-1-yl-ethyl)-7-methyl-1H-indole-3-carboxylic acid (1.58 mmol), DIEA (0.69 mL, 3.97 mmol), 2,2,2-trifluoro-N-(4-fluoro-3-piperidin-4-yl-benzyl)-acetamide hydrochloride (0.54 g, 1.58 mmol), and EDCI (0.46 g, 2.38 mmol) in CH₂Cl₂ (10 mL) is stirred at r.t. for 2 h. The mixture is partitioned between H₂O and CH₂Cl₂. The two layers are separated, and the organic layer is washed with brine, dried over Na₂SO₄, filtered, and concentrated in vacuo. The crude material is purified on silica gel with CH₂Cl₂/MeOH (100/0 to 95/5) as eluent to give the titled product (450 mg, 51%) as a white powder.

¹H NMR (300 MHz, CDCl₃) δ 8.65 (bs, 1H), 7.68 (d, J=7.7 Hz, 1H), 7.40-6.90 (m, 7H), 6.69 (s, 1H), 6.62 (s, 1H), 4.72 9t, J=5.0 Hz, 2H), 4.60-4.25 (m, 6H), 3.35-2.85 (m, 3H), 2.73 (s, 3H), 1.90-1.40 (m, 4H);

LC Rt 0.75 min; MS 556 (M+H, 100%).

### G. [4-(5-Aminomethyl-2-fluoro-phenyl)-piperidin-1-yl]-[1-(2-imidazol-1-yl-ethyl)-7-methyl-1H-indol-3-yl]-methanone dihydrochloride

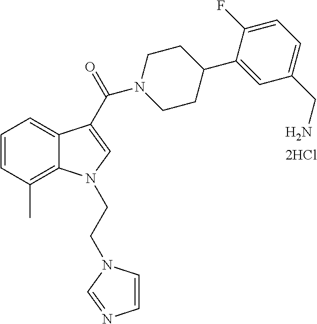

To a mixture of 2,2,2-trifluoro-N-(4-fluoro-3-{1-[1-(2-imidazol-1-yl-ethyl)-7-methyl-1H-indole-3-carbonyl]-piperidin-4-yl}-benzyl)-acetamide (445 mg, 0.80 mmol) in MeOH (10 mL) is added aqueous K₂CO₃ (885 mg, 6.40 mmol dissolved in 2.0 mL H₂O). This mixture is heated at 45° C. for 4 h. LC/MS indicates the reaction is completed. The reaction mixture is concentrated in vacuo to remove most of the methanol. The residue is partitioned between H₂O and CH₂Cl₂. The two layers are separated and the aqueous layer is extracted with CH₂Cl₂. The combined organic layers are washed with H₂O and brine, dried over Na₂SO₄, filtered, and concentrated in vacuo. 1.0 M HCl in Et₂O is added to the residue, and the resulting suspension is concentrated to dryness in vacuo. After the resulting solid is washed with Et₂O and heptane, it is dissolved in H₂O. The solution is lyophilized to dryness to give the titled product (274 mg, 64%) as a white powder.

¹H NMR (300 MHz, DMSO-d6) δ 8.55 (bs, 4H), 7.80-6.90 (m, 10H), 5.00-4.80 (m, 2H), 4.70-4.50 (m, 2H), 4.40-4.10 (m, 2H), 4.10-3.90 (m, 2H), 3.30-2.90 (m, 3H), 2.70 (s, 3H), 1.95-1.50 (m, 4H);

LC 0.49 min; MS 460 (M+H, 100%).

### Example 63

### [4-(5-Aminomethyl-2-fluoro-phenyl)-piperidin-1-yl]-[4-(3-fluoro-phenyl)-1-(2-methoxyethyl)-1H-indol-3-yl]-methanone hydrochloride

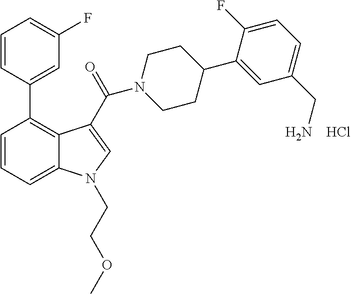

### A. 2,2,2-Trifluoro-N-(4-fluoro-3-{1-[4-(3-fluoro-phenyl)-1-(2-methoxy-ethyl)-1H-indole-3-carbonyl]-piperidin-4-yl}-benzyl)-acetamide

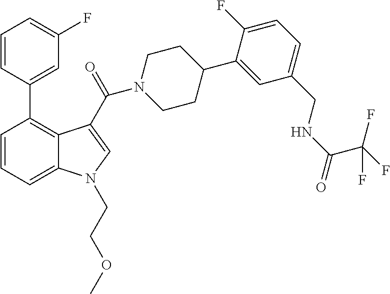

A mixture of N-(3-{1-[4-bromo-1-(2-methoxy-ethyl)-1H-indole-3-carbonyl]-piperidin-4-yl}-4-fluoro-benzyl)-2,2,2-trifluoro-acetamide (200 mg, 0.34 mmol), 3-fluorophenylboronic acid (57 mg, 0.41 mmol), cesium carbonate (223 mg, 0.68 mmol), and Pd(dppf)Cl₂.CH₂Cl₂ (28 mg, 10% mol) in dioxane/H₂O (10 mL/1 mL) is heated at 80° C. overnight. The reaction mixture is cooled to r.t., and then filtered through Celite. The filtrate is partitioned between EtOAc and 10% citric acid. The two layers are separated, and the aqueous layer is extracted with EtOAc once. The combined organic layers are washed with H₂O, and brine, dried over Na₂SO₄, filtered, and concentrated in vacuo. The crude material is purified on silica gel with CH₂Cl₂/MeOH (100/0 to 98/2) as eluent to give the product (140 mg, 68%) as a beige foam.

¹H NMR (300 MHz, CDCl₃) δ 7.60-6.95 (m, 11H), 6.55 (br s, 1H), 4.70-4.40 (m, 3H), 4.34 (t, J=5.4 Hz, 2H), 3.77 (t, J=5.3 Hz, 2H), 3.70-3.40 (m, 1H), 3.35 (s, 3H), 2.90-2.70 (m, 1H), 2.60-1.80 (m, 2H), 1.80-1.00 (m, 4H);

LC Rt 1.07 min; MS 600 (M+H, 100%).

### B. [4-(5-Aminomethyl-2-fluoro-phenyl)-piperidin-1-yl]-[4-(3-fluoro-phenyl)-1-(2-methoxyethyl)-1H-indol-3-yl]-methanone hydrochloride

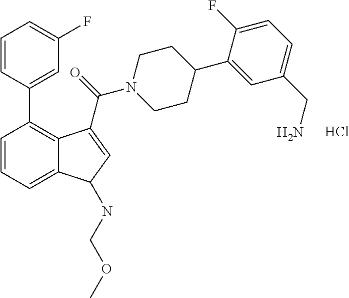

To a mixture of 2,2,2-trifluoro-N-(4-fluoro-3-{1-[4-(3-fluoro-phenyl)-1-(2-methoxy-ethyl)-1H-indole-3-carbonyl]-piperidin-4-yl}-benzyl)-acetamide (140 mg, 0.23 mmol) in MeOH (5 mL) is added aqueous K₂CO₃ (258 mg, 1.87 mmol, dissolved in 1.0 mL H₂O). This mixture is heated at 45° C. for 2 h. LC/MS indicates the reaction is completed. The reaction mixture is concentrated in vacuo to remove most of the methanol. The residue is partitioned between H₂O and EtOAc. The two layers are separated, and the organic layer is washed with H₂O and brine, dried over Na₂SO₄, filtered, and concentrated in vacuo. 4.0 M HCl in dioxane is added. The resulting suspension is concentrated in vacuo, and then dried in vacuo. The residue is redissolved in H₂O, and the resulting solution is lyophilized to yield the product (114 mg, 91%) as a slightly purple fluffy solid.

¹H NMR (300 MHz, DMSO-d6) δ 8.37 (br s, 3H), 7.80-7.05 (m, 11H), 4.55-4.30 (m, 3H), 4.10-3.90 (m, 2H), 3.80-3.60 (m, 2H), 3.30 (m, 1H), 3.25 (s, 3H), 2.90-2.65 (m, 1H), 2.50-1.95 (m, 2H), 1.80-1.05 (m, 4H);

LC 0.77 min; MS 486 (M+H, 100%).

### Example 64

### [4-(5-Aminomethyl-2-fluoro-phenyl)-piperidin-1-yl]-[1-(2-methoxy-ethyl)-4-thiophen-2-yl-1H-indol-3-yl]-methanone hydrochloride

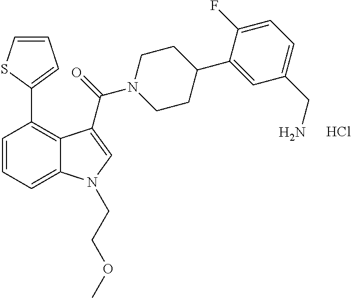

### A. 2,2,2-Trifluoro-N-(4-fluoro-3-{1-[1-(2-methoxy-ethyl)-4-thiophen-2-yl-1H-indole-3-carbonyl]-piperidin-4-yl}-benzyl)-acetamide

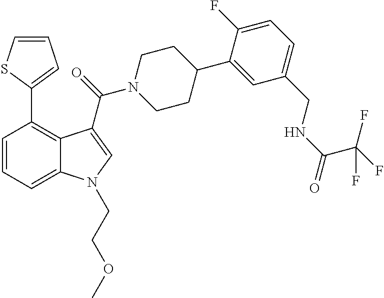

A mixture of N-(3-{1-[4-bromo-1-(2-methoxy-ethyl)-1H-indole-3-carbonyl]-piperidin-4-yl}-4-fluoro-benzyl)-2,2,2-trifluoro-acetamide (200 mg, 0.34 mmol), 2-thiophenelboronic acid (53 mg, 0.41 mmol), cesium carbonate (223 mg, 0.68 mmol), and Pd(dppf)Cl₂.CH₂Cl₂ (28 mg, 10% mol) in dioxane/H₂O (10 mL/1 mL) is heated at 80° C. overnight. The reaction mixture is cooled to r.t., and then filtered through Celite. The filtrate is partitioned between EtOAc and 10% citric acid. The two layers are separated, and the aqueous layer is extracted with EtOAc once. The combined organic layers are washed with H₂O, and brine, dried over Na₂SO₄, filtered, and concentrated in vacuo. The crude material is purified on silica gel with CH₂Cl₂/MeOH (100/0 to 98/2) as eluent to give the product (140 mg, 70%) as a beige foam.

¹H NMR (300 MHz, CDCl₃) δ 7.65-6.90 (m, 10H), 6.54 (br s, 1H), 4.80-4.65 (m, 1H), 4.55-4.45 (m, 2H), 4.40-4.20 (m, 2H), 3.85-3.70 (m, 2H), 3.60-3.40 (m, 1H), 3.35 (s, 3H), 2.95-2.75 (m, 1H), 2.40-2.00 (m, 2H), 2.00-1.00 (m, 4H);

LC Rt 1.06 min; MS 588 (M+H, 100%).

### B. [4-(5-Aminomethyl-2-fluoro-phenyl)-piperidin-1-yl]-[1-(2-methoxy-ethyl)-4-thiophen-2-yl-1H-indol-3-yl]-methanone hydrochloride

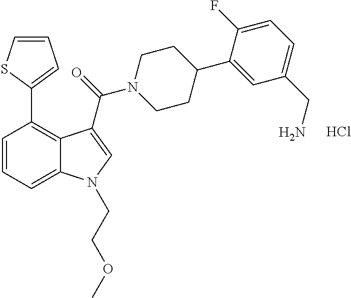

To a mixture of 2,2,2-trifluoro-N-(4-fluoro-3-{1-[1-(2-methoxy-ethyl)-4-thiophen-2-yl-1H-indole-3-carbonyl]-piperidin-4-yl}-benzyl)-acetamide (138 mg, 0.23 mmol) in MeOH (5 mL) is added aqueous K₂CO₃ (260 mg, 1.88 mmol, dissolved in 1.0 mL H₂O). This mixture is heated at 45° C. for 2 h. LC/MS indicates the reaction is completed. The reaction mixture is concentrated in vacuo to remove most of the methanol. The residue is partitioned between H₂O and EtOAc. The two layers are separated, and the organic layer is washed with H₂O and brine, dried over Na₂SO₄, filtered, and concentrated in vacuo. 4.0 M HCl in dioxane is added. The resulting suspension is concentrated in vacuo, and then dried in vacuo. The crude material is purified by RP-HPLC to yield the product (78 mg, 64%) as a white fluffy solid. LC 0.75 min; MS 492 (M+H, 100%).

### Example 65

### [4-(5-Aminomethyl-2-fluoro-phenyl)-piperidin-1-yl]-[4-(5-chloro-pyridin-3-yl)-1-(2-methoxy-ethyl)-1H-indol-3-yl]-methanone dihydrochloride

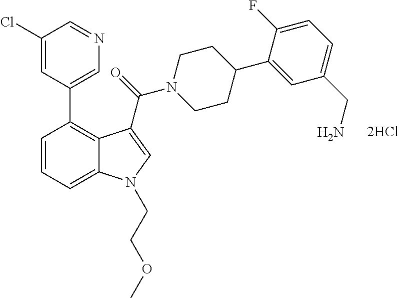

### A. N-(3-{1-[4-(5-Chloro-pyridin-3-yl)-1-(2-methoxy-ethyl)-1H-indole-3-carbonyl]-piperidin-4-yl}-4-fluoro-benzyl)-2,2,2-trifluoro-acetamide

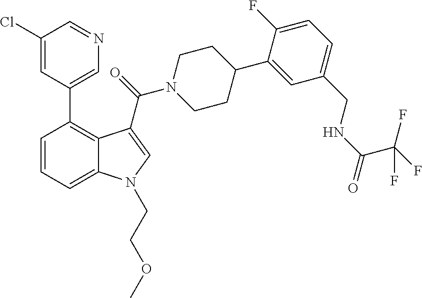

A mixture of N-(3-{1-[4-bromo-1-(2-methoxy-ethyl)-1H-indole-3-carbonyl]-piperidin-4-yl}-4-fluoro-benzyl)-2,2,2-trifluoro-acetamide (200 mg, 0.34 mmol), 3-chloropyridinelboronic acid (64 mg, 0.41 mmol), cesium carbonate (223 mg, 0.68 mmol), and Pd(dppf)Cl₂.CH₂Cl₂ (28 mg, 10% mol) in dioxane/H₂O (10 mL/1 mL) is heated at 80° C. overnight. The reaction mixture is cooled to r.t., and then filtered through Celite. The filtrate is partitioned between EtOAc and 10% citric acid. The two layers are separated, and the aqueous layer is extracted with EtOAc once. The combined organic layers are washed with H₂O, and brine, dried over Na₂SO₄, filtered, and concentrated in vacuo. The crude material is purified on silica gel with CH₂Cl₂/MeOH (100/0 to 98/2) as eluent to give the product (176 mg, 83%) as a yellow foam.

¹H NMR (300 MHz, CDCl₃) δ 8.75 (s, 1H), 8.55 (s, 1H), 8.05 (br s, 1H), 7.60-7.30 (m, 3H), 7.20-6.85 (m, 5H), 4.85-4.35 (m, 3H), 4.40-4.25 (m, 2H), 3.80-3.70 (m, 2H), 3.60-3.40 (m, 1H), 3.35 (s, 3H), 3.00-2.80 (m, 1H), 2.80-2.20 (m, 2H), 1.85-0.60 (m, 4H),

LC Rt 1.02 min; MS 617 (M+H, 100%).

### B. [4-(5-Aminomethyl-2-fluoro-phenyl)-piperidin-1-yl]-[4-(5-chloro-pyridin-3-yl)-1-(2-methoxy-ethyl)-1H-indol-3-yl]-methanone dihydrochloride

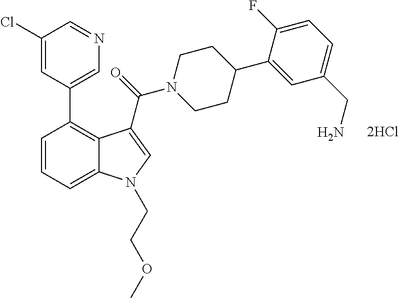

To a mixture of N-(3-{1-[4-(5-chloro-pyridin-3-yl)-1-(2-methoxy-ethyl)-1H-indole-3-carbonyl]-piperidin-4-yl}-4-fluoro-benzyl)-2,2,2-trifluoro-acetamide (176 mg, 0.29 mmol) in MeOH (5 mL) is added aqueous K₂CO₃ (315 mg, 2.29 mmol, dissolved in 1.0 mL H₂O). This mixture is heated at 45° C. for 2 h. LC/MS indicates the reaction is completed. The reaction mixture is concentrated in vacuo to remove most of the methanol. The residue is partitioned between H₂O and EtOAc. The two layers are separated, and the organic layer is washed with H₂O and brine, dried over Na₂SO₄, filtered, and concentrated in vacuo. 4.0 M HCl in dioxane is added. The resulting suspension is concentrated in vacuo, and then dried in vacuo. The crude material is purified by RP-HPLC to yield the product (88 mg, 51%) as a pale green solid.

¹H NMR (300 MHz, DMSO-d6) δ 8.75-8.55 (m, 2H), 8.22 (br s, 3H), 7.89 (s, 1H), 7.75-7.65 (m, 2H), 7.45-7.25 (m, 3H), 7.25-7.05 (m, 2H), 4.80-4.10 (m, 4H), 4.10-3.85 (m, 2H), 3.80-3.30 (m, 3H), 3.24 (s, 3H), 3.00-2.80 (m, 1H), 2.80-2.00 (m, 2H), 1.85-1.00 (m, 4H);

LC 0.71 min; MS 521 (M+H, 100%).

### Example 66

### 4-(5-Aminomethyl-2-fluoro-phenyl)-piperidin-1-yl]-[4-(3,5-dichloro-phenyl)-1-(2-methoxy-ethyl)-1H-indol-3-yl]-methanone hydrochloride

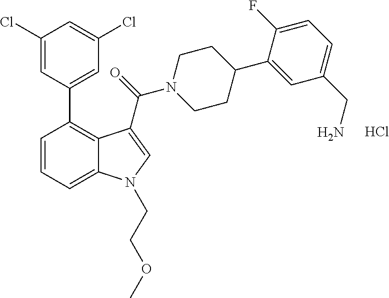

### A. N-(3-{1-[4-(3,5-Dichloro-phenyl)-1-(2-methoxy-ethyl)-1H-indole-3-carbonyl]-piperidin-4-yl}-4-fluoro-benzyl)-2,2,2-trifluoro-acetamide

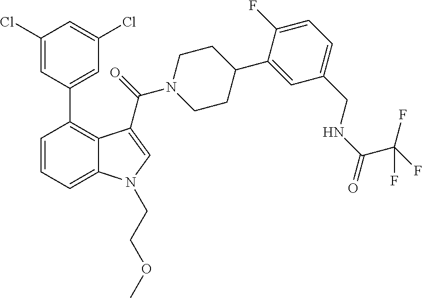

A mixture of N-(3-{1-[4-bromo-1-(2-methoxy-ethyl)-1H-indole-3-carbonyl]-piperidin-4-yl}-4-fluoro-benzyl)-2,2,2-trifluoro-acetamide (200 mg, 0.34 mmol), 3,5-dichlorophenyllboronic acid (78 mg, 0.41 mmol), cesium carbonate (223 mg, 0.68 mmol), and Pd(dppf)Cl₂.CH₂Cl₂ (28 mg, 10% mol) in dioxane/H₂O (10 mL/1 mL) is heated at 80° C. overnight. The reaction mixture is cooled to r.t., and then filtered through Celite. The filtrate is partitioned between EtOAc and 10% citric acid. The two layers are separated, and the aqueous layer is extracted with EtOAc once. The combined organic layers are washed with H₂O, and brine, dried over Na₂SO₄, filtered, and concentrated in vacuo. The crude material is purified on silica gel with CH₂Cl₂/MeOH (100/0 to 98/2) as eluent to give the product (190 mg, 85%) as a beige foam.

¹H NMR (300 MHz, CDCl₃) δ 7.60-6.90 (m, 10H), 6.53 (br s, 1H), 4.80-4.60 (m, 1H), 4.55-4.45 (m, 2H), 4.40-4.30 (m, 2H), 3.80-3.70 (m, 2H), 3.70-3.35 (m, 1H), 3.34 (s, 3H), 3.00-2.75 (m, 1H), 2.75-2.05 (m, 2H), 1.95-1.00 (m, 4H);

LC Rt 1.17 min; MS 650 (M+H, 100%).

### B. 4-(5-Aminomethyl-2-fluoro-phenyl)-piperidin-1-yl]-[4-(3,5-dichloro-phenyl)-1-(2-methoxy-ethyl)-1H-indol-3-yl]-methanone hydrochloride

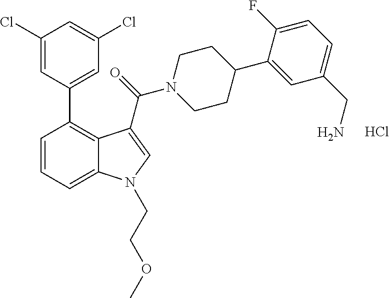

To a mixture of N-(3-{1-[4-(3,5-dichloro-phenyl)-1-(2-methoxy-ethyl)-1H-indole-3-carbonyl]-piperidin-4-yl}-4-fluoro-benzyl)-2,2,2-trifluoro-acetamide (185 mg, 0.28 mmol) in MeOH (5 mL) is added aqueous K₂CO₃ (314 mg, 2.27 mmol, dissolved in 1.0 mL H₂O). This mixture is heated at 45° C. for 2 h. LC/MS indicates the reaction is completed. The reaction mixture is concentrated in vacuo to remove most of the methanol. The residue is partitioned between H₂O and EtOAc. The two layers are separated, and the organic layer is washed with H₂O and brine, dried over Na₂SO₄, filtered, and concentrated in vacuo. 4.0 M HCl in dioxane is added. The resulting suspension is concentrated in vacuo, and then dried in vacuo. The crude material is purified by RP-HPLC to yield the product (82 mg, 52%) as a white fluffy solid.

¹H NMR (300 MHz, DMSO-d6) δ 8.22 (br, s 3H), 7.75-7.60 (m, 2H), 7.55-7.10 (m, 8H), 4.60-4.20 (m, 3H), 4.10-3.90 (m, 2H), 3.75-3.65 (m, 2H), 3.40-3.30 (m, 1H), 3.24 (s, 3H), 2.90-2.80 (m, 1H), 2.80-2.10 (m, 2H), 1.85-1.05 (m, 4H);

LC 0.86 min; MS 554 (M+H, 100%).

### Example 67

### [4-(5-Aminomethyl-2-fluoro-phenyl)-piperidin-1-yl]-[1-(2-methoxy-ethyl)-4-(1-propyl-1H-pyrazol-4-yl)-1H-indol-3-yl]-methanone hydrochloride

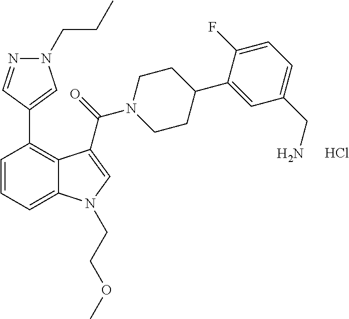

### A. 2,2,2-Trifluoro-N-(4-fluoro-3-{1-[1-(2-methoxy-ethyl)-4-(1-propyl-1H-pyrazol-4-yl)-1H-indole-3-carbonyl]-piperidin-4-yl}-benzyl)-acetamide

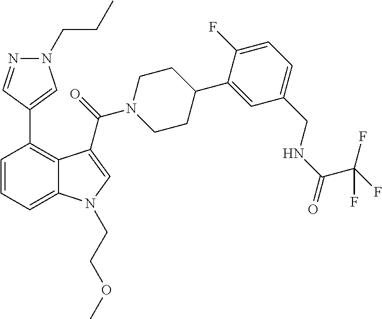

A mixture of N-(3-{1-[4-bromo-1-(2-methoxy-ethyl)-1H-indole-3-carbonyl]-piperidin-4-yl}-4-fluoro-benzyl)-2,2,2-trifluoro-acetamide (200 mg, 0.34 mmol), 1-propylpyrazoyl-3-boronic acid (68 mg, 0.44 mmol), cesium carbonate (223 mg, 0.68 mmol), and Pd(dppf)Cl₂.CH₂Cl₂ (28 mg, 10% mol) in dioxane/H₂O (10 mL/1 mL) is heated at 80° C. overnight. The reaction mixture is cooled to r.t., and then filtered through Celite. The filtrate is partitioned between EtOAc and 10% citric acid. The two layers are separated, and the aqueous layer is extracted with EtOAc once. The combined organic layers are washed with H₂O, and brine, dried over Na₂SO₄, filtered, and concentrated in vacuo. The crude material is purified on silica gel with CH₂Cl₂/MeOH (100/0 to 98/2) as eluent to give the product (187 mg, 89%) as a yellow foam.

¹H NMR (300 MHz, CDCl₃) δ 9.20 (br s, 1H), 8.00-7.90 (m, 1H), 8.80-8.60 (m, 2H), 7.80-6.85 (m, 6H), 4.90-4.70 (m, 1H), 4.65-4.40 (m, 2H), 4.35-4.20 (m, 2H), 4.20-4.00 (m, 2H), 3.80-3.60 (m, 2H), 3.55-3.40 (m, 1H), 3.35 (s, 3H), 3.05-2.55 (m, 3H), 2.10-0.75 (m, 9H);

LC Rt 1.00 min; MS 614 (M+H, 100%).

### B. [4-(5-Aminomethyl-2-fluoro-phenyl)-piperidin-1-yl]-[1-(2-methoxy-ethyl)-4-(1-propyl-1H-pyrazol-4-yl)-1H-indol-3-yl]-methanone hydrochloride

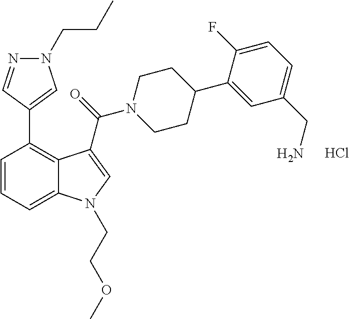

To a mixture of 2,2,2-trifluoro-N-(4-fluoro-3-{1-[1-(2-methoxy-ethyl)-4-(1-propyl-1H-pyrazol-4-yl)-1H-indole-3-carbonyl]-piperidin-4-yl}-benzyl)-acetamide (185 mg, 0.30 mmol) in MeOH (5 mL) is added aqueous K₂CO₃ (333 mg, 2.32 mmol, dissolved in 1.0 mL H₂O). This mixture is heated at 45° C. for 2 h. LC/MS indicates the reaction is completed. The reaction mixture is concentrated in vacuo to remove most of the methanol. The residue is partitioned between H₂O and EtOAc. The two layers are separated, and the organic layer is washed with H₂O and brine, dried over Na₂SO₄, filtered, and concentrated in vacuo. 4.0 M HCl in dioxane is added. The resulting suspension is concentrated in vacuo, and then dried in vacuo. The crude material is purified by RP-HPLC to yield the product (132 mg, 78%) as a pale green solid.

¹H NMR (300 MHz, DMSO-d6) δ 8.10 (br s, 3H), 7.80 (s 1H), 7.70-7.40 (m, 3H), 7.40-7.00 (m, 5H), 4.70-4.50 (m, 1H), 4.45-4.30 (m, 2H), 4.20-3.90 (m, 4H), 3.80-3.60 (m, 2H), 3.40 (m, 1H), 3.20 (s, 3H), 2.90-2.70 (m, 1H), 2.60-2.00 (m, 2H), 2.00-0.80 (m, 9H);

LC 0.69 min; MS 518 (M+H, 100%).

### Example 68

### [4-(5-Aminomethyl-2-fluoro-phenyl)-piperidin-1-yl]-[4-isopropoxy-1-(2-methoxy-ethyl)-1H-indol-3-yl]-methanone hydrochloride

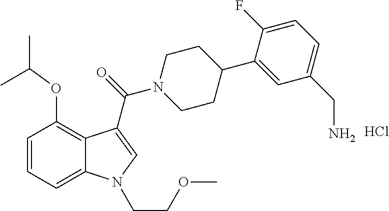

### A. 4-Isopropoxy-1H-indole

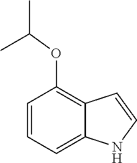

The title compound is prepared in a similar manner as described in Example 49A using 4-hydroxyindole and 2-propanol as the starting materials.

¹H NMR (300 MHz, CDCl₃) δ 8.11 (br s, 1H), 7.12-7.07 (m, 2H), 7.02-6.99 (m, 1H), 6.67-6.65 (m, 1H), 6.56 (d, 1H), 4.72 (sept, 1H), 1.43 (s, 3H), 1.41 (s, 3H).

### B. 4-Isopropoxy-1-(2-methoxy-ethyl)-1H-indole

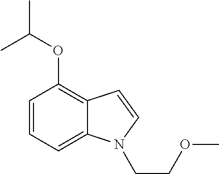

The title compound is prepared in a similar manner as described in Example 1E using 4-isopropoxy-1H-indole as the starting material.

¹H NMR (300 MHz, CDCl₃) δ 7.13-7.05 (m, 2H), 6.96-6.93 (m, 1H), 6.59 (d, 1H), 6.54 (d, 1H), 4.70 (sept, 1H), 4.26 (t, 2H), 3.70 (t, 2H), 3.31 (s, 3H), 1.42 (s, 3H), 1.40 (s, 3H).

### C. 2,2,2-Trifluoro-1-[4-isopropoxy-1-(2-methoxy-ethyl)-1H-indol-3-yl]-ethanone

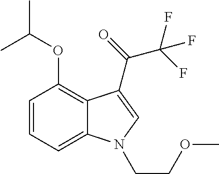

The title compound is prepared in a similar manner as described in Example 2G using 4-isopropoxy-1-(2-methoxy-ethyl)-1H-indole as the starting material.

¹H NMR (300 MHz, CDCl₃) δ 7.94 (q, 1H), 7.27 (t, 1H), 6.98-6.95 (m, 1H), 6.79 (d, 1H), 4.69 (sept, 1H), 4.31 (t, 2H), 3.74 (t, 2H), 3.32 (s, 3H), 1.46 (s, 3H), 1.44 (s, 3H).

### D. 4-Isopropoxy-1-(2-methoxy-ethyl)-1H-indole-3-carboxylic acid

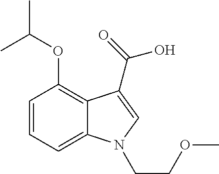

The title compound is prepared in a similar manner as described in Example 2H using 2,2,2-trifluoro-1-[4-isopropoxy-1-(2-methoxy-ethyl)-1H-indol-3-yl]-ethanone as the starting material.

¹H NMR (300 MHz, DMSO-d6) δ 11.84 (br s, 1H), 8.02 (s, 1H), 7.28-7.18 (m, 2H), 6.88-6.85 (m, 1H), 4.92 (sept, 1H), 4.39 (t, 2H), 3.66 (t, 2H), 3.22 (s, 3H), 1.38 (s, 3H), 1.36 (s, 3H).

### E. 2,2,2-Trifluoro-N-(4-fluoro-3-{1-[4-isopropoxy-1-(2-methoxy-ethyl)-1H-indole-3-carbonyl]-piperidin-4-yl}-benzyl)acetamide

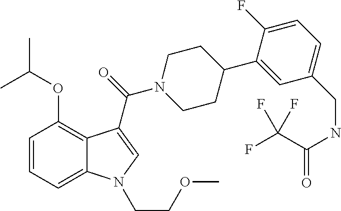

The title compound is prepared in a similar manner as described in Example 2I using 4-isopropoxy-1-(2-methoxy-ethyl)-1H-indole-3-carboxylic acid and 2,2,2-trifluoro-N-(4-fluoro-3-piperidin-4-yl-benzyl)-acetamide hydrochloride as the starting materials.

¹H NMR (300 MHz, CDCl₃) δ 7.2 (m, 3H), 6.9 (m, 2H), 6.6 (m, 2H), 5.0 (bs, 1H), 4.7 (m, 1H), 4.5 (m, 2H), 4.3 (m, 2H), 4.1 (m, 1H), 3.9 (bs, 1H), 3.7 (t, 2H), 3.35 (s, 3H), 3.2 (m, 2H), 2.9 (m, 1H), 1.9-1.7 (m, 4H), 1.5 (s, 6H).

MS m/z: [M+H]⁺=564.

### F. [4-(5-Aminomethyl-2-fluoro-phenyl)-piperidin-1-yl]-[4-isopropoxy-1-(2-methoxy-ethyl)-1H-indol-3-yl]-methanone hydrochloride

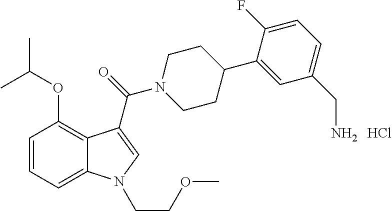

The title compound is prepared in a similar manner as described in Example 1K using 2,2,2-trifluoro-N-(4-fluoro-3-{1-[4-isopropoxy-1-(2-methoxy-ethyl)-1H-indole-3-carbonyl]-piperidin-4-yl}-benzyl)-acetamide as the starting material.

¹H NMR (300 MHz, DMSO-d6) δ 8.4 (bs, 2H), 7.5 (m, 1H), 7.35 (m, 2H), 7.2 (m, 1H), 7.1 (m, 2H), 6.8 (m, 1H), 4.8 (bs, 1H), 4.7 (m, 1H), 4.3 (m, 2H), 4.0 (m, 2H), 3.6 (m, 3H), 3.3 (s, 3H), 3.1 (m, 2H), 2.8 (m, 1H), 1.8 (m, 2H), 1.6 (m, 2H), 1.3 (s, 6H).

MS m/z: [M+H]⁺=468.

### Example 69

### [4-(5-Aminomethyl-2-fluoro-phenyl)-piperidin-1-yl]-[1-(2-methoxy-ethyl)-4-(4-trifluoromethoxy-phenyl)-1H-indol-3-yl]-methanone hydrochloride

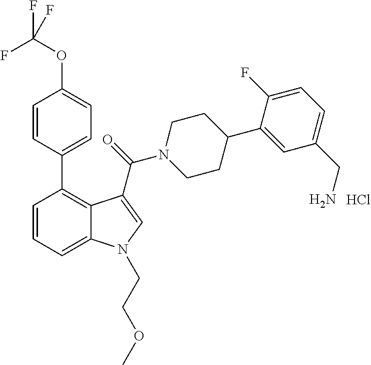

### A. 2,2,2-Trifluoro-N-(4-fluoro-3-{1-[1-(2-methoxy-ethyl)-4-(4-trifluoromethoxy-phenyl)-1H-indole-3-carbonyl]-piperidin-4-yl}-benzyl)-acetamide

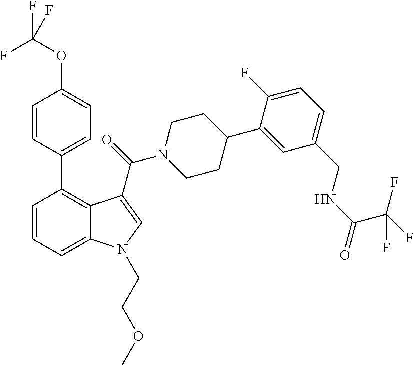

A mixture of N-(3-{1-[4-bromo-1-(2-methoxy-ethyl)-1H-indole-3-carbonyl]-piperidin-4-yl}-4-fluoro-benzyl)-2,2,2-trifluoro-acetamide (200 mg, 0.34 mmol), 4-trifluorormethoxyphenylboronic acid (85 mg, 0.41 mmol), cesium carbonate (223 mg, 0.68 mmol), and Pd(dppf)Cl₂.CH₂Cl₂ (28 mg, 10% mol) in dioxane/H₂O (10 mL/1 mL) is heated at 80° C. overnight. The reaction mixture is cooled to r.t., and then filtered through Celite. The filtrate is partitioned between EtOAc and 10% citric acid. The two layers are separated, and the aqueous layer is extracted with EtOAc once. The combined organic layers are washed with H₂O, and brine, dried over Na₂SO₄, filtered, and concentrated in vacuo. The crude material is purified on silica gel with CH₂Cl₂/MeOH (100/0 to 98/2) as eluent to give the product (186 mg, 82%) as a yellow foam.

¹H NMR (300 MHz, CDCl₃) δ 7.80-6.90 (m, 11H), 6.80 (br s, 1H), 4.60-4.25 (m, 5H), 4.65-4.40 (m, 2H), 3.80-3.70 (m, 2H), 3.55-3.40 (m, 1H), 3.35 (s, 3H), 2.80-2.60 (m, 1H), 2.40-1.80 (m, 2H), 1.70-1.00 (m, 4H);

LC Rt 1.14 min; MS 666 (M+H, 100%).

### B. [4-(5-Aminomethyl-2-fluoro-phenyl)-piperidin-1-yl]-[1-(2-methoxy-ethyl)-4-(4-trifluoromethoxy-phenyl)-1H-indol-3-yl]-methanone hydrochloride

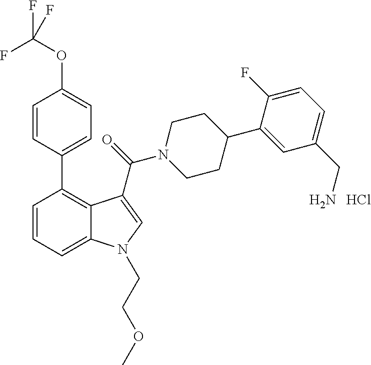

To a mixture of 2,2,2-trifluoro-N-(4-fluoro-3-{1-[1-(2-methoxy-ethyl)-4-(4-trifluoromethoxy-phenyl)-1H-indole-3-carbonyl]-piperidin-4-yl}-benzyl)-acetamide (184 mg, 0.27 mmol) in MeOH (5 mL) is added aqueous K₂CO₃ (305 mg, 2.21 mmol, dissolved in 1.0 mL H₂O). This mixture is heated at 45° C. for 2 h. LC/MS indicates the reaction is completed. The reaction mixture is concentrated in vacuo to remove most of the methanol. The residue is partitioned between H₂O and EtOAc. The two layers are separated, and the organic layer is washed with H₂O and brine, dried over Na₂SO₄, filtered, and concentrated in vacuo. 4.0 M HCl in dioxane is added. The resulting suspension is concentrated in vacuo, and then dried in vacuo. The crude material is purified by RP-HPLC to yield the product (130 mg, 79%) as a pale green solid.

¹H NMR (300 MHz, DMSO-d6) δ 8.19 (br s, 3H), 7.70-7.05 (m 11H), 4.50-4.10 (m, 3H), 4.05-3.85 (m, 2H), 3.80-3.60 (m, 2H), 3.80-3.60 (m, 2H), 3.40 (m, 1H), 3.25 (s, 3H), 2.80-2.60 (m, 1H), 2.60-1.80 (m, 2H), 1.75-1.10 (m, 4H);

LC 0.85 min; MS 570 (M+H, 100%).

### Example 70

### [4-(5-Aminomethyl-2-fluoro-phenyl)-piperidin-1-yl]-[1-(2-methoxy-ethyl)-4-(4-methoxy-phenyl)-1H-indol-3-yl]-methanone hydrochloride

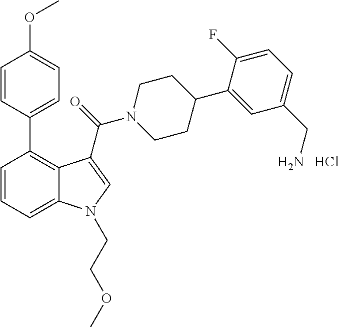

### A. 2,2,2-Trifluoro-N-(4-fluoro-3-{1-[1-(2-methoxy-ethyl)-4-(4-methoxy-phenyl)-1H-indole-3-carbonyl]-piperidin-4-yl}-benzyl)-acetamide

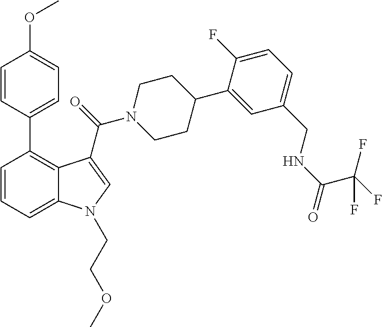

A mixture of N-(3-{1-[4-bromo-1-(2-methoxy-ethyl)-1H-indole-3-carbonyl]-piperidin-4-yl}-4-fluoro-benzyl)-2,2,2-trifluoro-acetamide (200 mg, 0.34 mmol), 4-methoxyphenylboronic acid (64 mg, 0.41 mmol), cesium carbonate (223 mg, 0.68 mmol), and Pd(dppf)Cl₂.CH₂Cl₂ (28 mg, 10% mol) in dioxane/H₂O (4.5 mL/0.5 mL) is heated at 80° C. overnight. The reaction mixture is concentrated in vacuo to remove the solvent. The crude material is purified on silica gel with CH₂Cl₂/MeOH (100/0 to 98/2) as eluent to give the product (180 mg, 86%).

¹H NMR (300 MHz, CDCl₃) δ 7.70-6.40 (m, 12H), 4.75-4.10 (m, 5H), 3.95-3.60 (m, 5H), 3.50-3.10 (m, 4H), 2.90-2.60 (m, 1H), 2.40-1.20 (m, 6H);

LC Rt 1.05 min; MS 612 (M+H, 100%).

### B. [4-(5-Aminomethyl-2-fluoro-phenyl)-piperidin-1-yl]-[1-(2-methoxy-ethyl)-4-(4-methoxy-phenyl)-1H-indol-3-yl]-methanone hydrochloride

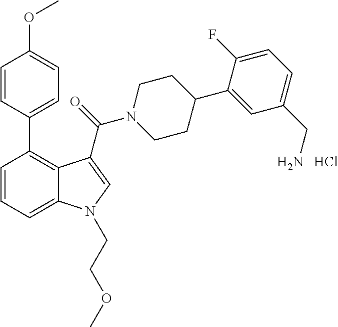

To a mixture of 2,2,2-trifluoro-N-(4-fluoro-3-{1-[1-(2-methoxy-ethyl)-4-(4-methoxy-phenyl)-1H-indole-3-carbonyl]-piperidin-4-yl}-benzyl)-acetamide (168 mg, 0.27 mmol) in MeOH (5 mL) is added aqueous K₂CO₃ (303 mg, 2.19 mmol, dissolved in 1.0 mL H₂O). This mixture is heated at 45° C. for 2 h. LC/MS indicates the reaction is completed. The reaction mixture is concentrated in vacuo to remove most of the methanol. The residue is partitioned between H₂O and EtOAc. The two layers are separated, and the organic layer is washed with H₂O and brine, dried over Na₂SO₄, filtered, and concentrated in vacuo. 4.0 M HCl in dioxane is added. The resulting suspension is concentrated in vacuo, and then dried in vacuo. The crude material is triturated with Et₂O, and the beige solid (126 mg, 85%) is collected by vacuum filtration.

¹H NMR (300 MHz, DMSO-d6) δ 8.25 (br s, 3H), 7.70-6.90 (m 11H), 4.50-4.30 (m, 3H), 4.10-3.85 (m, 2H), 3.81 (s, 3H), 3.75-3.60 (m, 2H), 3.50-3.10 (m, 4H), 2.80-2.60 (m, 1H), 2.40-1.10 (m, 6H);

LC 0.76 min; MS 516 (M+H, 100%).

### Example 71

### [4-(5-Aminomethyl-2-fluoro-phenyl)-piperidin-1-yl]-[4-(4-fluoro-phenyl)-1-(2-methoxy-ethyl)-1H-indol-3-yl]-methanone hydrochloride

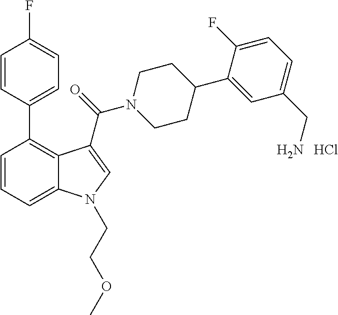

### A. 2,2,2-Trifluoro-N-(4-fluoro-3-{1-[4-(4-fluoro-phenyl)-1-(2-methoxy-ethyl)-1H-indole-3-carbonyl]-piperidin-4-yl}-benzyl)-acetamide

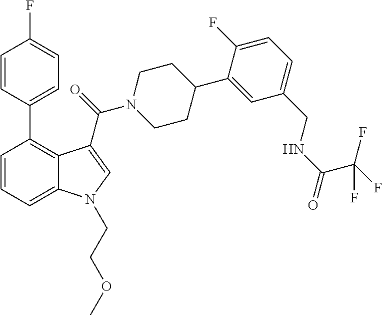

A mixture of N-(3-{1-[4-bromo-1-(2-methoxy-ethyl)-1H-indole-3-carbonyl]-piperidin-4-yl}-4-fluoro-benzyl)-2,2,2-trifluoro-acetamide (200 mg, 0.34 mmol), 4-fluorophenylboronic acid (57 mg, 0.41 mmol), cesium carbonate (223 mg, 0.68 mmol), and Pd(dppf)Cl₂.CH₂Cl₂ (28 mg, 10% mol) in dioxane/H₂O (4.5 mL/0.5 mL) is heated at 80° C. overnight. The reaction mixture is concentrated in vacuo to remove the solvent. The crude material is purified on silica gel with CH₂Cl₂/MeOH (100/0 to 98/2) as eluent to give the product (187 mg, 91%).

¹H NMR (300 MHz, CDCl₃) δ 7.80-6.90 (m, 11H), 6.70-6.50 (bs, 1H), 4.75-4.20 (m, 5H), 3.90-3.70 (m, 2H), 3.50-3.30 (m, 4H), 2.90-2.60 (m, 1H), 2.10-1.20 (m, 6H);

LC Rt 1.07 min; MS 600 (M+H, 100%).

### B. [4-(5-Aminomethyl-2-fluoro-phenyl)-piperidin-1-yl]-[4-(4-fluoro-phenyl)-1-(2-methoxy-ethyl)-1H-indol-3-yl]-methanone hydrochloride

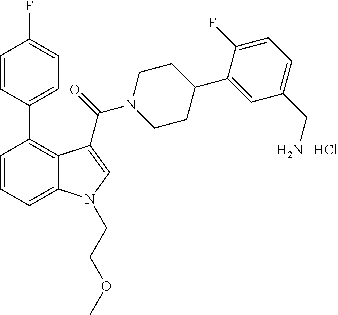

To a mixture of 2,2,2-trifluoro-N-(4-fluoro-3-{1-[4-(4-fluoro-phenyl)-1-(2-methoxy-ethyl)-1H-indole-3-carbonyl]-piperidin-4-yl}-benzyl)-acetamide (185 mg, 0.31 mmol) in MeOH (5 mL) is added aqueous K₂CO₃ (341 mg, 2.4 mmol, dissolved in 1.0 mL H₂O). This mixture is heated at 45° C. for 2 h. LC/MS indicates the reaction is completed. The reaction mixture is concentrated in vacuo to remove most of the methanol. The residue is partitioned between H₂O and EtOAc. The two layers are separated, and the organic layer is washed with H₂O and brine, dried over Na₂SO₄, filtered, and concentrated in vacuo. 4.0 M HCl in dioxane is added. The resulting suspension is concentrated in vacuo, and then dried in vacuo. The crude material is triturated with Et₂O and the beige solid (128 mg, 76%) is collected by vacuum filtration.

¹H NMR (300 MHz, DMSO-d6) δ 8.25 (br s, 3H), 7.80-7.00 (m 11H), 4.50-4.10 (m, 3H), 4.10-3.85 (m, 2H), 3.80-3.60 (m, 2H), 3.40-3.10 (m, 4H), 2.90-2.65 (m, 1H), 2.40-1.00 (m, 6H);

LC 0.75 min; MS 504 (M+H, 100%).

### Example 72

### [4-(5-Aminomethyl-2-fluoro-phenyl)-piperidin-1-yl]-[4-(3-hydroxy-phenyl)-1-(2-methoxy-ethyl)-1H-indol-3-yl]-methanone hydrochloride

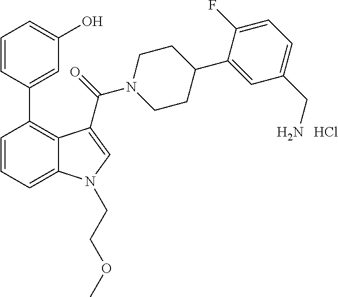

### A. 2,2,2-Trifluoro-N-(4-fluoro-3-{1-[4-(3-hydroxy-phenyl)-1-(2-methoxy-ethyl)-1H-indole-3-carbonyl]-piperidin-4-yl}-benzyl)-acetamide

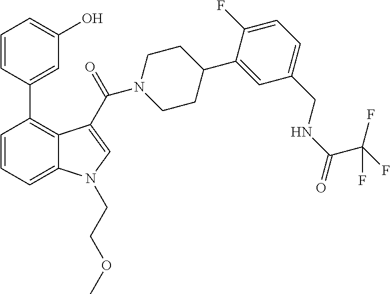

A mixture of N-(3-{1-[4-bromo-1-(2-methoxy-ethyl)-1H-indole-3-carbonyl]-piperidin-4-yl}-4-fluoro-benzyl)-2,2,2-trifluoro-acetamide (200 mg, 0.34 mmol), 3-hydroxyphenylboronic acid (57 mg, 0.41 mmol), cesium carbonate (223 mg, 0.68 mmol), and Pd(dppf)Cl₂.CH₂Cl₂ (28 mg, 10% mol) in dioxane/H₂O (4.5 mL/0.5 mL) is heated at 80° C. overnight. The reaction mixture is concentrated in vacuo to remove the solvent. The crude material is purified on silica gel with CH₂Cl₂/MeOH (100/0 to 98/2) as eluent to give the product (158 mg, 77%) as a beige foam. This material is used in the next step without further purification.

LC Rt 0.99 min; MS 598 (M+H, 100%).

### B. [4-(5-Aminomethyl-2-fluoro-phenyl)-piperidin-1-yl]-[4-(3-hydroxy-phenyl)-1-(2-methoxy-ethyl)-1H-indol-3-yl]-methanone hydrochloride

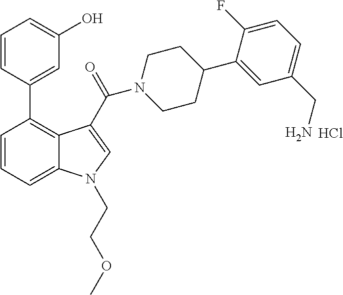

To a mixture of 2,2,2-trifluoro-N-(4-fluoro-3-{1-[4-(3-hydroxy-phenyl)-1-(2-methoxy-ethyl)-1H-indole-3-carbonyl]-piperidin-4-yl}-benzyl)-acetamide (156 mg, 0.26 mmol) in MeOH (5 mL) is added aqueous K₂CO₃ (288 mg, 2.1 mmol, dissolved in 1.0 mL H₂O). This mixture is heated at 45° C. for 2 h. LC/MS indicates the reaction is completed. The reaction mixture is concentrated in vacuo to remove most of the methanol. The residue is partitioned between H₂O and CH₂Cl₂. The two layers are separated, and the aqueous layer is extracted with CH₂Cl₂ (3×). The crude material is purified by RP-HPLC to give the product (74 mg, 52%) as a white fluffy solid.

¹H NMR (300 MHz, DMSO-d6) δ 9.43 (s, 1H), 8.07 (br s, 3H), 7.65-6.70 (m 11H), 4.60-4.25 (m, 3H), 4.10-3.90 (m, 2H), 3.80-3.60 (m, 2H), 3.40-3.10 (m, 4H), 2.85-2.65 (m, 1H), 2.40-1.00 (m, 6H);

LC 0.70 min; MS 502 (M+H, 100%).

### Example 73

### [4-(5-Aminomethyl-2-fluoro-phenyl)-piperidin-1-yl]-[1-(2-methoxy-ethyl)-4-pyridin-3-yl-1H-indol-3-yl]-methanone dihydrochloride

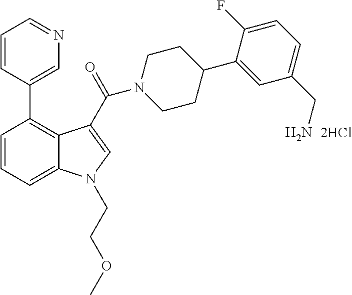

### A. 2,2,2-Trifluoro-1-[1-(2-methoxy-ethyl)-4-(4,4,5,5-tetramethyl-[1,3,2]dioxaborolan-2-yl)-1H-indol-3-yl]-ethanone

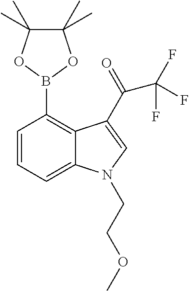

A mixture of 1-[4-bromo-1-(2-methoxy-ethyl)-1H-indol-3-yl]-2,2,2-trifluoro-ethanone (entry 48B) (700 mg, 2 mmol), bis-(pinacolate) diboron (776 mg, 3 mol), potassium acetate (784 mg, 8 mmol), Pd(dppf)Cl₂.CH₂Cl₂ (114 mg, 0.14 mmol) in anhydrous DMSO (15 mL) is heated at 70° C. for 5 h. The mixture is cooled to r.t., and is partitioned between water and EtOAc. The two layers are separated, and the organic layer is washed with water and brine, and concentrated in vacuo. The crude material is purified on silica gel with heptane/EtOAc (75/25 to 55/45) as eluent to give the product as a white solid (507 mg, 63%).

¹H NMR (300 MHz, CDCl₃) δ 8.01 (d, J=1.5 Hz, 1H), 7.60-7.25 (m, 3H), 4.35 (t, J=5.1 Hz, 2H), 3.70 (t, J=5.4 Hz, 2H), 3.28 (s, 3H), 1.48 (s, 12H);

LC Rt 1.09 min; MS 398 (M+H, 100%).

### B. 2,2,2-Trifluoro-1-[1-(2-methoxy-ethyl)-4-pyridin-3-yl-1H-indol-3-yl]-ethanone

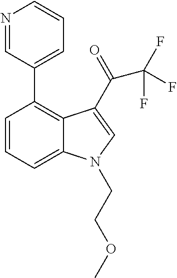

A mixture of 2,2,2-trifluoro-1-[1-(2-methoxy-ethyl)-4-(4,4,5,5-tetramethyl-[1,3,2]dioxaborolan-2-yl)-1H-indol-3-yl]-ethanone (370 mg, 0.93 mmol), 3-bromopyridine (0.1 mL, 1.11 mmol), Cs₂CO₃ (606 mg, 1.86 mmol), and Pd(dppf)Cl₂.CH₂Cl₂ (76 mg, 10% mmol) in dioxane (4.5 mL)/water (0.5 mL) is heated at 75° C. overnight. The solvent is removed in vacuo, and the crude material is purified on silica gel with heptane/EtOAC (70/30 to 50/50) as eluent to give the product (275 mg, 84%) as a light brown oil.

¹H NMR (300 MHz, CDCl₃) δ 8.70-8.50 (m, 2H), 8.20-8.10 (m, 1H), 7.70-7.55 (m, 1H), 7.50-7.40 (m, 2H), 7.40-7.20 (m, 2H), 4.44 (t, J=4.9 Hz, 2H), 3.81 (t, J=5.3

Hz, 2H), 3.36 (s, 3H);

LC Rt 0.67 min; MS 349 (M+H, 100%).

### C. 1-(2-Methoxy-ethyl)-4-pyridin-3-yl-1H-indole-3-carboxylic acid hydrochloride

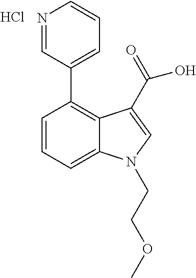

A mixture of 2,2,2-trifluoro-1-[1-(2-methoxy-ethyl)-4-pyridin-3-yl-1H-indol-3-yl]-ethanone (270 mg, 0.77 mmol) in MeOH (1.6 mL) and 5 M NaOH (1.6 mL) is heated at 70° C. overnight. The solvent is removed in vacuo, and the residue is dissolved in water. The pH of the solution is adjusted to ˜3 with 3 M HCl. The solution is concentrated to dryness in vacuo. The residue is used in the next step without further purification.

LC Rt 0.53 min; MS 297 (M+H, 100%).

### D. 2,2,2-Trifluoro-N-(4-fluoro-3-{1-[1-(2-methoxy-ethyl)-4-pyridin-3-yl-1H-indole-3-carbonyl]-piperidin-4-yl}-benzyl)-acetamide

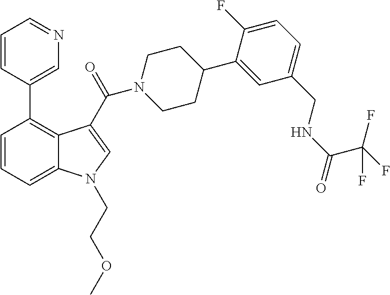

A mixture of 1-(2-methoxy-ethyl)-4-pyridin-3-yl-1H-indole-3-carboxylic acid hydrochloride (0.77 mmol), DIEA (0.47 mL, 2.7 mmol), 2,2,2-trifluoro-N-(4-fluoro-3-piperidin-4-yl-benzyl)-acetamide hydrochloride (317 mg, 0.93 mmol), and EDCI (193 mg, 1.0 mmol) in CH₂Cl₂ (20 mL) is stirred at r.t. for 5 h. The mixture is partitioned between H₂O and CH₂Cl₂. The two layers are separated, and the organic layer is washed with brine, dried over Na₂SO₄, filtered, and concentrated in vacuo. The crude material is purified on silica gel with CH₂Cl₂/MeOH (100/0 to 98/2) as eluent to give the product (166 mg, 36% from entry 73B) as a white foam.

¹H NMR (300 MHz, CDCl₃) δ 8.85-8.75 (m, 1H), 8.60-8.50 (m, 1H), 8.09 (bs, 1H), 7.60-6.80 (m, 9H), 4.80-4.30 (m, 5H), 3.80-3.70 (m, 2H), 3.50-3.25 (m, 4H), 3.00-2.20 (m, 3H), 1.70-0.80 (m, 4H);

LC Rt 0.85 min; MS 583 (M+H, 100%).

### E. [4-(5-Aminomethyl-2-fluoro-phenyl)-piperidin-1-yl]-[1-(2-methoxy-ethyl)-4-pyridin-3-yl-1H-indol-3-yl]-methanone dihydrochloride

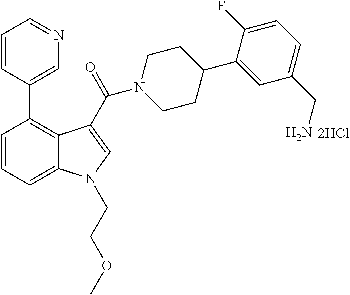

To a mixture of 2,2,2-trifluoro-N-(4-fluoro-3-{1-[1-(2-methoxy-ethyl)-4-pyridin-3-yl-1H-indole-3-carbonyl]-piperidin-4-yl}-benzyl)-acetamide (160 mg, 0.27 mmol) in MeOH (5 mL) is added aqueous K₂CO₃ (303 mg, 2.2 mmol, dissolved in 1.0 mL H₂O). This mixture is heated at 45° C. for 4 h. LC/MS indicates the reaction is completed. The reaction mixture is concentrated in vacuo to remove most of the methanol. The residue is partitioned between H₂O and CH₂Cl₂. The two layers are separated, and the organic layer is washed with water and brine, dried over Na₂SO₄, filtered, and concentrated in vacuo. The residue is suspended in 1M HCl in Et₂O. The solid is triturated with Et₂O. The slightly yellow solid (170 mg, quantitative) is collected by suction filteration.

¹H NMR (300 MHz, DMSO-d6) δ 8.80-8.20 (m, 4H), 8.10-7.00 (m, 11H), 4.60-3.60 (m, 7H), 3.30-3.20 (m, 4H), 2.90-2.60 (m, 1H), 2.40-1.00 (m, 6H);

LC 0.58 min; MS 487 (M+H, 100%).

### Example 74

### [4-(5-Aminomethyl-2-fluoro-phenyl)-piperidin-1-yl]-[1-(2-methoxy-ethyl)-4-pyridin-2-yl-1H-indol-3-yl]-methanone dihydrochloride

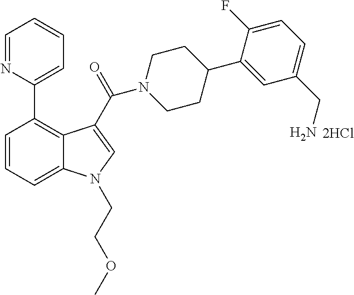

### A. 2,2,2-Trifluoro-1-[1-(2-methoxy-ethyl)-4-pyridin-2-yl-1H-indol-3-yl]-ethanone

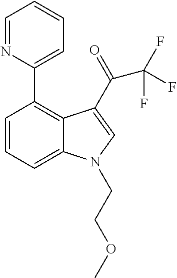

A mixture of 2,2,2-trifluoro-1-[1-(2-methoxy-ethyl)-4-(4,4,5,5-tetramethyl-[1,3,2]dioxaborolan-2-yl)-1H-indol-3-yl]-ethanone (entry 73A) (220 mg, 0.55 mmol), 2-bromopyridine (59 μL, 0.61 mmol), Cs₂CO₃ (360 mg, 1.1 mmol), and Pd(dppf)Cl₂.CH₂Cl₂ (45 mg, 10% mmol) in dioxane (4.5 mL)/water (0.5 mL) is heated at 80° C. for 4 h. The solvent is removed in vacuo, and the crude material is purified on silica gel with heptane/EtOAC (70/30 to 50/50) as eluent to give the product (87 mg, 46%) as a light yellow oil.

¹H NMR (300 MHz, CDCl₃) δ 8.61 (d, J=4.7 Hz, 1H), 8.80 (d, J=1.6 Hz, 1H), 7.90-7.80 (m, 1H), 7.60-7.40 (m, 3H), 7.40-7.10 (m, 2H), 4.42 (t, J=5.2 Hz, 2H), 3.77 (t, J=5.1 Hz, 2H), 3.34 (s, 3H);

LC Rt 0.67 min; MS 349 (M+H, 100%). LC Rt 0.66 min; MS 349 (M+H, 100%).

### B. 1-(2-Methoxy-ethyl)-4-pyridin-2-yl-1H-indole-3-carboxylic acid hydrochloride

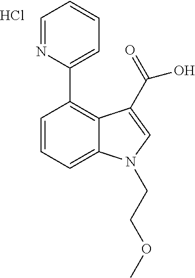

A mixture of 2,2,2-trifluoro-1-[1-(2-methoxy-ethyl)-4-pyridin-2-yl-1H-indol-3-yl]-ethanone (85 mg, 0.24 mmol) in MeOH (0.49 mL) and 5 M NaOH (0.49 mL) is heated at 70° C. overnight. The solvent is removed in vacuo, and the residue is dissolved in water. The pH of the solution is adjusted to ˜3 with 3 M HCl. The solution is concentrated to dryness in vacuo. The residue is used in the next step without further purification.

LC Rt 0.50 min; MS 297 (M+H, 100%).

### C. 2,2,2-Trifluoro-N-(4-fluoro-3-{1-[1-(2-methoxy-ethyl)-4-pyridin-2-yl-1H-indole-3-carbonyl]-piperidin-4-yl}-benzyl)-acetamide

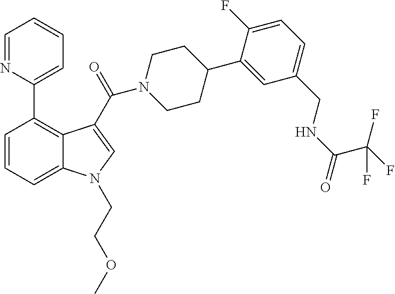

A mixture of 1-(2-methoxy-ethyl)-4-pyridin-2-yl-1H-indole-3-carboxylic acid hydrochloride (0.24 mmol), DIEA (0.15 mL, 0.85 mmol), 2,2,2-trifluoro-N-(4-fluoro-3-piperidin-4-yl-benzyl)-acetamide hydrochloride (100 mg, 0.29 mmol), and EDCI (60 mg, 0.32 mmol) in CH₂Cl₂ (10 mL) is stirred at r.t. overnight. The mixture is partitioned between H₂O and CH₂Cl₂. The two layers are separated, and the organic layer is washed with brine, dried over Na₂SO₄, filtered, and concentrated in vacuo. The crude material is purified on silica gel with CH₂Cl₂/MeOH (100/0 to 98/2) as eluent to give the product (110 mg, 77% from entry 74A) as a white foam.

¹H NMR (300 MHz, CDCl₃) δ 8.80-8.65 (m, 1H), 7.80-6.85 (m, 10H), 6.64 (bs, 1H), 4.80-4.20 (m, 5H), 3.80-3.70 (m, 2H), 3.70-3.45 (m, 1H), 3.34 (s, 3H), 2.90-2.60 (m, 1H), 2.50-1.10 (m, 6H);

LC Rt 0.84 min; MS 583 (M+H, 100%).

### D. [4-(5-Aminomethyl-2-fluoro-phenyl)-piperidin-1-yl]-[1-(2-methoxy-ethyl)-4-pyridin-2-yl-1H-indol-3-yl]-methanone dihydrochloride

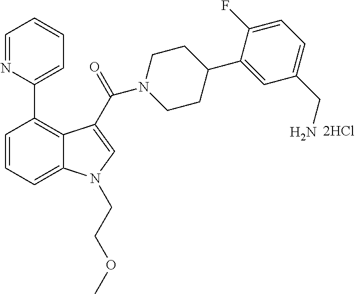

To a mixture of 2,2,2-trifluoro-N-(4-fluoro-3-{1-[1-(2-methoxy-ethyl)-4-pyridin-2-yl-1H-indole-3-carbonyl]-piperidin-4-yl}-benzyl)-acetamide (105 mg, 0.18 mmol) in MeOH (5 mL) is added aqueous K₂CO₃ (199 mg, 1.4 mmol, dissolved in 1.0 mL H₂O). This mixture is heated at 45° C. for 2 h. LC/MS indicates the reaction is completed. The reaction mixture is concentrated in vacuo to remove most of the methanol. The residue is partitioned between H₂O and CH₂Cl₂. The two layers are separated, and the organic layer is washed with water and brine, dried over Na₂SO₄, filtered, and concentrated in vacuo. The residue is suspended in 1M HCl in Et₂O. The solid is triturated with Et₂O. The yellowish green solid (74 mg, 73%) is collected by suction filteration.

¹H NMR (300 MHz, DMSO-d6) δ 8.90-8.10 (m, 4H), 7.90-7.10 (m, 11H), 4.60-3.60 (m, 7H), 3.30-3.10 (m, 4H), 3.00-2.80 (m, 1H), 2.40-1.00 (m, 6H);

LC 0.58 min; MS 487 (M+H, 100%).

### Example 75

### [4-(5-Aminomethyl-2-fluoro-phenyl)-piperidin-1-yl]-[4-amino-1-(2-methoxy-ethyl)-1H-indol-3-yl]-methanone hydrochloride

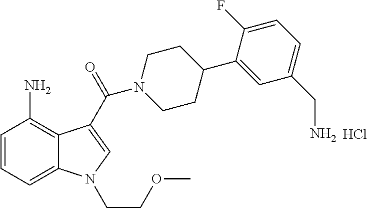

### A. 1-(2-methoxy-ethyl)-4-nitro-1H-indole

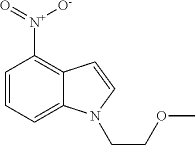

The title compound is prepared in a similar manner as described in Example 1E using 4-nitro-1H-indole as the starting material.

¹H NMR (300 MHz, CDCl₃) δ 8.2 (d, 1H), 7.47 (d, 2H), 7.5 (m, 1H), 7.3 (m, 2H), 4.4 (t, 2H), 3.8 (t, 2H), 3.3 (s, 3H).

MS m/z: [M+H]⁺=221.

### B. 2,2,2-Trifluoro-1-[1-(2-methoxy-ethyl)-4-nitro-1H-indol-3-yl]ethanone

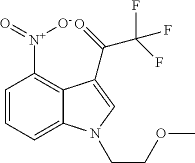

The title compound is prepared in a similar manner as described in Example 2G using 1-(2-methoxy-ethyl)-4-nitro-1H-indole as the starting material.

¹H NMR (300 MHz, CDCl₃) δ 8.2 (d, 1H), 7.8 (m, 2H), 7.5 (m, 1H), 4.5 (t, 2H), 3.8 (t, 2H), 3.4 (s, 3H).

MS m/z: [M+H]⁺=317.

### C. 1-(2-Methoxy-ethyl)-4-nitro-1H-indole-3-carboxylic acid

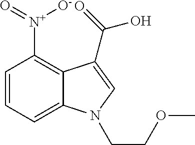

The title compound is prepared in a similar manner as described in Example 2H using 2,2,2-trifluoro-1-[1-(2-methoxy-ethyl)-4-nitro-1H-indol-3-yl]ethanone as the starting material.

¹H NMR (300 MHz, DMSO-d6) δ 12.2 (bs, 1H), 8.2 (s, 1H), 8.0 (d, 1H), 7.6 (d, 1H), 7.4 (m, 1H), 4.5 (t, 2H), 3.7 (t, 2H), 3.2 (s, 3H).

MS m/z: [M+H]⁺=265.

### D. 2,2,2-Trifluoro-N-(4-fluoro-3-{1-[1-(2-methoxy-ethyl)-4-nitro-1H-indole-3-carbonyl]-piperidin-4-yl}-benzyl)-acetamide

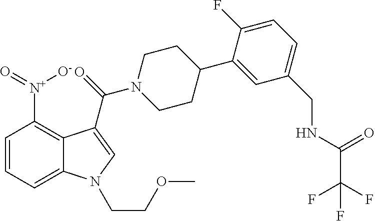

The title compound is prepared in a similar manner as described in Example 6E using 1-(2-methoxy-ethyl)-4-nitro-1H-indole-3-carboxylic acid and 2,2,2-trifluoro-N-(4-fluoro-3-piperidin-4-yl-benzyl)-acetamide hydrochloride as the starting materials.

¹H NMR (300 MHz, CDCl₃) δ 8.1 (d, 1H), 7.8 (d, 1H), 7.6 (s, 1H), 7.4 (m, 1H), 7.2 (m, 1H), 7.15 (m, 1H), 7.0 (m, 1H), 6.7 (bs, 1H), 5.0 (m, 1H), 4.5 (m, 2H), 4.35 (m, 2H), 3.8 (m, 1H), 3.7 (m, 2H), 3.3 (s, 3H), 3.20 (m, 2H), 3.0 (m, 1H), 1.9 (m, 2H), 1.6 (m, 2H)

MS m/z: [M+H]⁺=551.

### E. N-(3-{1-[4-Amino-1-(2-methoxy-ethyl)-1H-indole-3-carbonyl]-piperidin-4-yl}-4-fluoro-benzyl)-2,2,2-trifluoro-acetamide

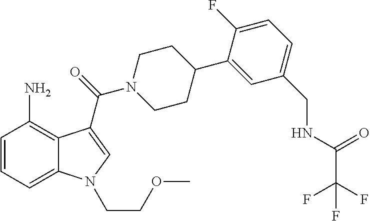

A solution of 2,2-trifluoro-N-(4-fluoro-3-{1-[1-(2-methoxy-ethyl)-4-nitro-1H-indole-3-carbonyl]-piperidin-4-yl}-benzyl)-acetamide (2.8 g, 5.1 mmol) and 10% Pd/C (1.0 g) in methanol (35 mL) is hydrogentated at 50 psi for 3 hours. The mixture is then filtered over a cake of celite and the cake is washed with excess methanol. The filtrate is then concentrated in vacuo to give 2.0 g of the title compound.

¹H NMR (300 MHz, CDCl₃) δ 7.3 (s, 1H), 7.2-7.0 (m, 4H), 6.8 (d, 1H), 6.7 (bs, 1H), 6.4 (d, 1H), 4.7 (m, 2H), 4.5 (m, 2H), 4.2 (m, 2H), 3.7 (m, 2H), 3.3 (s, 3H), 3.15 (m, 3H), 1.9 (m, 2H), 1.8 (m, 2H), 1.6 (bs, 2H).

MS m/z: [M+H]⁺=521.

### F-[4-(5-Aminomethyl-2-fluoro-phenyl)-piperidin-1-yl]-[4-amino-1-(2-methoxy-ethyl)-1H-indol-3-yl]-methanone hydrochloride

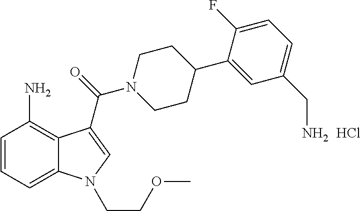

The title compound is prepared in a similar manner as described in Example 3B using N-(3-{1-[4-amino-1-(2-methoxy-ethyl)-1H-indole-3-carbonyl]-piperidin-4-yl}-4-fluoro-benzyl)-2,2,2-trifluoro-acetamide as the starting material.

¹H NMR (300 MHz, DMSO-d6) δ 8.3 (bs, 2H), 7.9 (m, 1H), 7.7 (m, 1H), 7.4 (m, 2H), 7.2 (m, 2H), 6.9 (bs, 1H), 4.6 (m, 2H), 4.4 (m, 2H), 4.0 (m, 2H), 3.6 (m, 2H), 3.3 (m, 3H), 3.2 (s, 3H), 2.6 (m, 2H), 1.9-1.7 (m, 4H).

MS m/z: [M+H]+=425.

### Example 76

### [4-(5-Aminomethyl-2-fluoro-phenyl)-piperidin-1-yl]-[4-methylamino-1-(2-methoxy-ethyl)-1H-indol-3-yl]-methanone hydrochloride

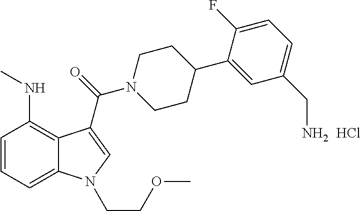

A-1. [N-(3-{1-[4-methylamino-1-(2-methoxy-ethyl)-1H-indole-3-carbonyl]-piperidin-4-yl}-4-fluoro-benzyl)-2,2,2-trifluoro-acetamide and A-2. N-(3-{1-[4-dimethylamino-1-(2-methoxy-ethyl)-1H-indole-3-carbonyl]-piperidin-4-yl}-4-fluoro-benzyl)-2,2,2-trifluoro-acetamide

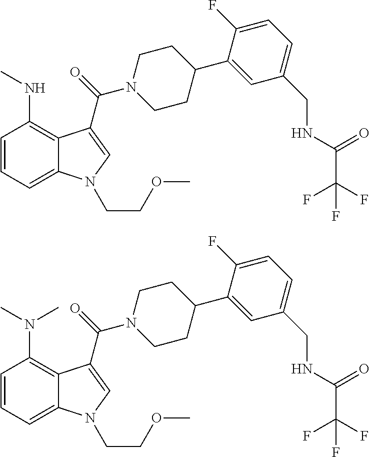

To a solution of N-(3-{1-[4-amino-1-(2-methoxy-ethyl)-1H-indole-3-carbonyl]-piperidin-4-yl}-4-fluoro-benzyl)-2,2,2-trifluoro-acetamide (1.5 g, 2.88 mmol) in 20 mL MeOH is added 1 mL of 37% formaldehyde. The reaction mixture is stirred for 5 min. A solution of zinc chloride (20 mg, 0.14 mmol) and sodium cyanoborohydride (0.18 g, 2.88 mmol) in MeOH (5 mL) is added. The mixture is stirred for 18 h at r.t. The mixture is diluted with 100 mL of water and 250 mL of ethyl acetate. The organic phase is separated and is washed with brine, dried with Na₂SO₄, filtered and is concentrated in vacuo. The crude residue is flash chromatographed over SiO₂ eluting with 100% EtOAc to afford 0.34 g of [N-(3-{1-[4-methylamino-1-(2-methoxy-ethyl)-1H-indole-3-carbonyl]-piperidin-4-yl}-4-fluoro-benzyl)-2,2,2-trifluoro-acetamide

¹H NMR (300 MHz, CDCl₃) δ 7.3 (m, 3H), 7.2 (m, 1H), 7.1 (m, 1H), 6.7 (d, 1H), 6.6 (bs, 1H), 6.4 (m, 1H), 6.3 (d, 1H), 4.7 (m, 2H), 4.5 (m, 2H), 4.2 (m, 2H), 3.7 (m, 2H), 3.3 (s, 3H), 3.2 (m, 3H), 2.9 (d, 3H), 1.9 (m, 2H), 1.8 (m, 2H). MS m/z: [M+H]+=535. and 0.51 g of [N-(3-{1-[4-dimethylamino-1-(2-methoxy-ethyl)-1H-indole-3-carbonyl]-piperidin-4-yl}-4-fluoro-benzyl)-2,2,2-trifluoro-acetamide. ¹H NMR (300 MHz, CDCl₃) δ 7.4 (m, 1H), 7.2 (m, 3H), 7.0 (m, 2H), 6.7 (m, 2H), 5.0 (bs, 1H), 4.5 (m, 2H), 4.3 (m, 3H), 3.8 (m, 3H), 3.3 (s, 3H), 3.2 (m, 2H), 2.8 (s, 6H), 1.9 (m, 2H), 1.8 (m, 2H). MS m/z: [M+H]+=549.

### B. [4-(5-Aminomethyl-2-fluoro-phenyl)-piperidin-1-yl]-[4-methylamino-1-(2-methoxy-ethyl)-1H-indol-3-yl]-methanone hydrochloride

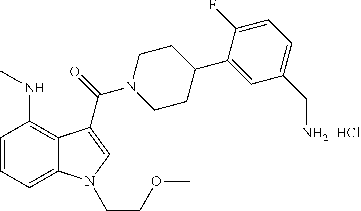

The title compound is prepared in a similar manner as described in Example 3B using N-(3-{1-[4-methylamino-1-(2-methoxy-ethyl)-1H-indole-3-carbonyl]-piperidin-4-yl}-4-fluoro-benzyl)-2,2,2-trifluoro-acetamide as the starting material.

¹H NMR (300 MHz, DMSO-d6) δ 8.4 (bs, 2H), 7.8 (m, 1H), 7.6 (m, 1H), 7.4 (m, 1H), 7.2 (m, 3H), 6.7 (bs, 1H), 4.6 (m, 2H), 4.4 (m, 2H), 4.0 (m, 4H), 3.7 (m, 2H), 3.3 (m, 3H), 3.2 (s, 3H), 2.8 (m, 3H), 1.9-1.7 (m, 4H).

MS m/z: [M+H]⁺=439.

### Example 77

### [4-(5-Aminomethyl-2-fluoro-phenyl)-piperidin-1-yl]-[4-dimethylamino-1-(2-methoxy-ethyl)-1H-indol-3-yl]-methanone hydrochloride

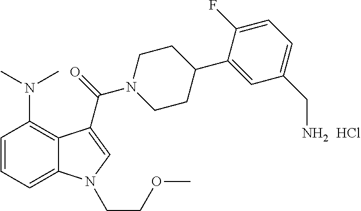

The title compound is prepared in a similar manner as described in Example 3B using N-(3-{1-[4-dimethylamino-1-(2-methoxy-ethyl)-1H-indole-3-carbonyl]-piperidin-4-yl}-4-fluoro-benzyl)-2,2,2-trifluoro-acetamide as the starting material.

¹H NMR (300 MHz, DMSO-d6) δ 8.4 (bs, 2H), 8.2 (m, 1H), 7.8 (m, 2H), 7.6 (m, 1H), 7.5 (m, 1H), 7.4 (m, 1H), 7.2 (m, 1H), 4.6 (bs, 1H), 4.5 (m, 2H), 4.0 (m, 2H), 3.7 (m, 2H), 3.5 (m, 4H), 3.3 (m, 3H), 3.2 (s, 6H), 1.9-1.7 (m, 4H).

MS m/z: [M+H]⁺=453.

### Example 78

### [4-(5-Aminomethyl-2-fluoro-phenyl)-piperidin-1-yl]-[4-pyridin-4-yl-1-(2-trifluoromethoxy-ethyl)-1H-indol-3-yl]-methanone dihydrochloride

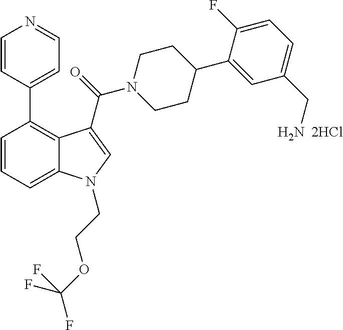

### A. 1-(4-Bromo-1H-indol-3-yl)-2,2,2-trifluoro-ethanone

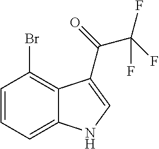

A mixture of 4-bromoindole (5.0 g, 25.5 mmol) and TFAA (10.6 mL, 76.5 mmol) in DMF (20 mL) is heated at 40° C. overnight. The reaction mixture is partitioned between water and Et₂O. The two layers are separated, and the organic layer is washed with saturated Na₂CO₃, water, and brine, dried over MgSO₄, filtered, and concentrated in vacuo to yield the product (5.64 g, 75%) as a brown powder.

¹H NMR (300 MHz, DMSO-d6) δ 12.91 (bs, 1H), 8.55 (s, 1H), 7.62 (d, J=8.0 Hz, 1H), 7.53 (d, J=7.5 Hz, 1H), 7.40-7.10 (m, 1H);

¹⁹F NMR (300 MHz, DMSO-d6) δ −70.12;

LC Rt 0.91 min; MS 293 (M+H, 100%).

### B. 1-[4-Bromo-1-(2-trifluoromethoxy-ethyl)-1H-indol-3-yl]-2,2,2-trifluoro-ethanone

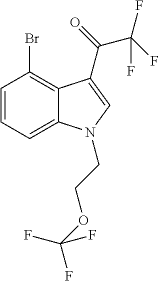

A mixture of 1-(4-bromo-1H-indol-3-yl)-2,2,2-trifluoro-ethanone (4.50 g, 22.0 mmol) and NaH (0.97 mg, 60% oil dispersion, 24.1 mmol) in THF (40 mL) is stirred at 0° C. for 5 min. Trifluoro-methanesulfonic acid 2-trifluoromethoxy-ethyl ester (J. Org. Chem 2001, 66, 1061-1062) (6.30 g, 24.1 mmol) is added. This mixture is stirred at 0° C. for 10 min and then at r.t. for 1 h. The mixture is partitioned between water and EtOAc. The two layers are separated, and the organic layer is washed with brine, dried over MgSO₄, filtered, and concentrated in vacuo. The crude material is purified on silica gel with heptane/EtOAc (100/0 to 50/50) as eluent to give the product (5.21 g, 80%).

¹H NMR (300 MHz, CDCl₃) δ 8.00 (d, J=1.7 Hz, 1H), 7.65-7.55 (m, 1H), 7.40-7.15 (m, 2H), 4.52 (t, J=5.1 Hz, 2H), 4.33 (t, J=5.3 Hz, 2H);

¹⁹F NMR (300 MHz, CDCl₃) δ −61.82 (s, 3F), −72.17 (s, 3F);

LC Rt: 1.18 min; MS 405 (M+1).

### C. 4-Bromo-1-(2-trifluoromethoxy-ethyl)-1H-indole-3-carboxylic acid

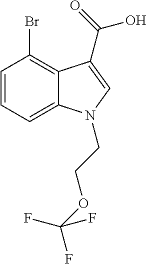

A mixture of 1-[4-bromo-1-(2-trifluoromethoxy-ethyl)-1H-indol-3-yl]-2,2,2-trifluoro-ethanone (5.0 g, 12.4 mmol) in MeOH (100 mL) and aqueous NaOH (5.0 M, 50 mL) is stirred at 80° C. for 1 h and then at 60° C. for 1.5 h. The mixture is concentrated in vacuo to remove the organic solvent. The residue is partitioned between water and Et₂O. The two layers are separated, and the aqueous layer is acidified to pH ˜2 with conc. HCl at 0° C. The acidified aqueous layer is extracted with EtOAc. The organic extract is washed with water and brine, dried over MgSO₄, filtered, and concentrated in vacuo to give the crude product (3.77 g, 86%) which is used in the next step without further purification.

¹H NMR (300 MHz, DMSO-d6) δ 12.10 (bs, 1H), 8.16 (s, 1H), 7.68 (d, J=7.7 Hz, 1H), 7.42 (d, J=7.1 Hz, 1H), 7.16 (t, J=8.1 Hz, 1H), 4.62 (t, J=4.7 Hz, 2H), 4.44 (t, J=5.2 Hz, 2H);

¹⁹F NMR (300 MHz, CDCl₃) δ −59.63;

LC Rt: 0.95 min; MS 353 (M+1).

### D. N-(3-{1-[4-Bromo-1-(2-trifluoromethoxy-ethyl)-1H-indole-3-carbonyl]-piperidin-4-yl}-4-fluoro-benzyl)-2,2,2-trifluoro-acetamide

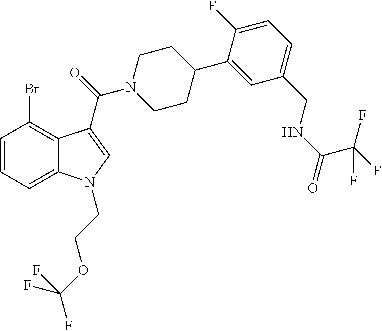

A mixture of 4-bromo-1-(2-trifluoromethoxy-ethyl)-1H-indole-3-carboxylic acid (1.0 g, 2.84 mmol), Et₃N (1.18 mL, 8.52 mmol), 2,2,2-trifluoro-N-(4-fluoro-3-piperidin-4-yl-benzyl)-acetamide hydrochloride (1.26 g, 3.69 mmol), and EDCI (817 mg, 4.26 mmol) in CH₂Cl₂ (20 mL) is stirred at r.t. for 3.5 h. The mixture is partitioned between H₂O and EtOAc. The two layers are separated, and the organic layer is washed with brine, dried over MgSO₄, filtered, and concentrated in vacuo. The crude material is purified on silica gel with heptane/EtOAc (30/70 to 0/100) as eluent to give the product (1.04 g, 57%).

¹H NMR (300 MHz, CDCl₃) δ 7.25-6.90 (m, 7H), 6.75 (br s, 1H), 5.15-4.90 (br m, 1H), 4.60-4.30 (m, 4H), 4.30-4.10 (m, 2H), 4.00-3.45 (m, 1H), 3.30-3.00 (m, 2H), 3.00-2.80 (m, 1H), 2.10-1.50 (m, 4H);

¹⁹F NMR (300 MHz, CDCl₃) δ −60.66 (s, 3F), −75.31 (s, 3F), −120.03 (s, 1F), −118.88 (m, 1F);

LC Rt 1.05 min; MS 638 (M+1, 100%).

### E. 2,2,2-Trifluoro-N-(4-fluoro-3-{1-[4-pyridin-4-yl-1-(2-trifluoromethoxy-ethyl)-1H-indole-3-carbonyl]-piperidin-4-yl}benzyl)-acetamide

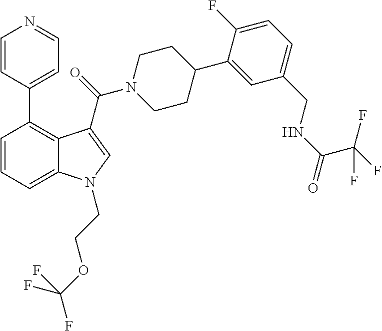

A mixture of N-(3-{1-[4-bromo-1-(2-trifluoromethoxy-ethyl)-1H-indole-3-carbonyl]-piperidin-4-yl}-4-fluoro-benzyl)-2,2,2-trifluoro-acetamide (200 mg, 0.34 mmol), 4-pyridineboronic acid (46 mg, 0.37 mmol), cesium carbonate (204 mg, 0.62 mmol), and Pd(dppf)Cl₂.CH₂Cl₂ (26 mg, 10% mol) in dioxane/H₂O (9 mL/1 mL) is heated at 80° C. for 5 h. The reaction mixture is concentrated in vacuo to remove the solvent. The crude material is purified on silica gel with CH₂Cl₂/MeOH (100/0 to 97/3) as eluent to give the product (187 mg, 94%) as a white solid.

¹H NMR (300 MHz, CDCl₃) δ 8.75-8.55 (m, 1H), 7.70-6.80 (m, 11H), 4.80-4.20 (m, 6H), 3.40-3.20 (m, 1H), 3.00-2.00 (m, 3H), 1.80-1.00 (m, 4H);

LC Rt 0.86 min; MS 637 (M+H, 100%).

### F. [4-(5-Aminomethyl-2-fluoro-phenyl)-piperidin-1-yl]-[4-pyridin-4-yl-1-(2-trifluoromethoxy-ethyl)-1H-indol-3-yl]-methanone dihydrochloride

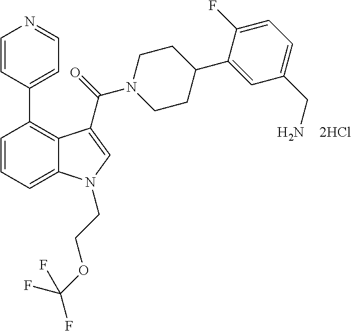

To a mixture of 2,2,2-trifluoro-N-(4-fluoro-3-{1-[4-pyridin-4-yl-1-(2-trifluoromethoxy-ethyl)-1H-indole-3-carbonyl]-piperidin-4-yl}benzyl)-acetamide (185 mg, 0.29 mmol) in MeOH (5 mL) is added aqueous K₂CO₃ (321 mg, 2.3 mmol, dissolved in 1.0 mL H₂O). This mixture is heated at 45° C. for 4 h. LC/MS indicates the reaction is completed. The reaction mixture is concentrated in vacuo to remove most of the methanol. The residue is partitioned between H₂O and CH₂Cl₂. The two layers are separated, and the organic layer is washed with H₂O and brine, dried over Na₂SO₄, filtered, and concentrated in vacuo. 4.0 M HCl in dioxane is added. The resulting suspension is concentrated in vacuo, and then dried in vacuo. The crude material is triturated with Et₂O, and the yellowish green solid (108 mg, 60%) is collected by vacuum filtration.

¹H NMR (300 MHz, DMSO-d6) δ 9.0-8.80 (m, 1H), 8.60-8.30 (bs, 4H), 8.00-7.80 (m, 2H), 7.70-7.10 (m, 8H), 4.90-4.60 (m, 2H), 4.60-3.80 (m, 6H), 3.00-2.85 (m, 1H), 2.40-2.00 (m, 2H), 1.80-1.30 (m, 4H);

LC 0.61 min; MS 541 (M+H, 100%).

### Example 79

### 4-(5-Aminomethyl-2-fluoro-phenyl)-piperidin-1-yl]-[4-bromo-1-(2-trifluoromethoxy-ethyl)-1H-indol-3-yl]-methanone hydrochloride

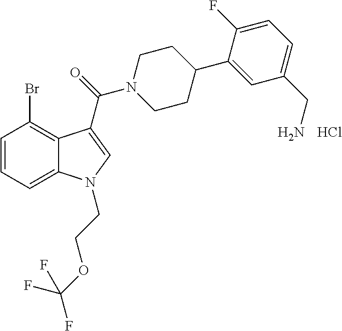

A mixture of N-(3-{1-[4-bromo-1-(2-trifluoromethoxy-ethyl)-1H-indole-3-carbonyl]-piperidin-4-yl}-4-fluoro-benzyl)-2,2,2-trifluoro-acetamide (entry 78D) (50 mg, 0.078 mmol) in MeOH (1.8 mL) is added aqueous K₂CO₃ (86 mg, 0.62 mmol, dissolved in 0.2 mL H₂O). This mixture is heated at 45° C. for 4 h. LC/MS indicates the reaction is completed. The reaction mixture is concentrated in vacuo to remove most of the methanol. The residue is partitioned between H₂O and CH₂Cl₂. The two layers are separated, and the organic layer is washed with H₂O and brine, dried over Na₂SO₄, filtered, and concentrated in vacuo. 4.0 M HCl in dioxane is added. The resulting suspension is concentrated in vacuo, and then dried in vacuo. The crude is triturated with Et₂O, and the beige solid is collected by vacuum filtration to yield the title product (25 mg, 55%).

¹H NMR (300 MHz, DMSO-d6) δ 7.80-7.00 (m, 7H), 5.10 (bs, 3H), 4.90-4.30 (m, 5H), 4.00-3.50 (m, 3H), 3.20-2.70 (m, 3H), 2.40-2.00 (m, 2H), 2.00-1.40 (m, 4H);

LC 0.75 min; MS 543 (M+H, 100%).

### Example 80

### [4-(5-Aminomethyl-2-fluoro-phenyl)-piperidin-1-yl]-[4-phenyl-1-(2-trifluoromethoxy-ethyl)-1H-indol-3-yl]-methanone hydrochloride

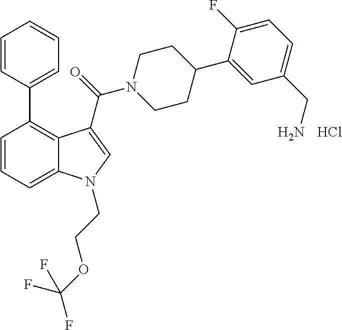

### A. 2,2,2-Trifluoro-N-(4-fluoro-3-{1-[4-phenyl-1-(2-trifluoromethoxy-ethyl)-1H-indole-3-carbonyl]-piperidin-4-yl}-benzyl)-acetamide

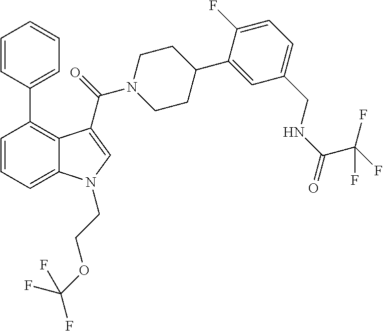

A mixture of N-(3-{1-[4-bromo-1-(2-trifluoromethoxy-ethyl)-1H-indole-3-carbonyl]-piperidin-4-yl}-4-fluoro-benzyl)-2,2,2-trifluoro-acetamide (entry 78D) (200 mg, 0.34 mmol), phenylboronic acid (46 mg, 0.37 mmol), cesium carbonate (204 mg, 0.62 mmol), and Pd(dppf)Cl₂.CH₂Cl₂ (26 mg, 10% mol) in dioxane/H₂O (9 mL/1 mL) is heated at 80° C. for 5 h. The reaction mixture is concentrated in vacuo to remove the solvent. The crude material is purified on silica gel with CH₂Cl₂/MeOH (100/0 to 97/3) as eluent to give the product (196 mg, 96%) as a white foam.

¹H NMR (300 MHz, CDCl₃) δ 7.80-6.80 (m, 12H), 6.50 (bs, 1H), 4.65-4.20 (m, 5H), 3.50-3.20 (m, 1H), 2.80-2.60 (m, 1H), 2.20-1.00 (m, 6H);

LC Rt 1.11 min; MS 636 (M+H, 100%).

### B. [4-(5-Aminomethyl-2-fluoro-phenyl)-piperidin-1-yl]-[4-phenyl-1-(2-trifluoromethoxy-ethyl)-1H-indol-3-yl]-methanone hydrochloride

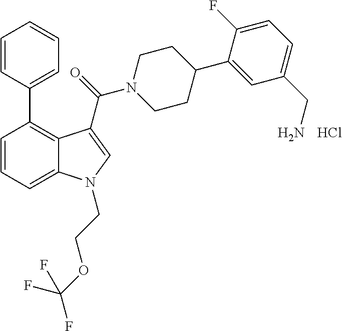

To a mixture of 2,2,2-trifluoro-N-(4-fluoro-3-{1-[4-phenyl-1-(2-trifluoromethoxy-ethyl)-1H-indole-3-carbonyl]-piperidin-4-yl}-benzyl)-acetamide (195 mg, 0.31 mmol) in MeOH (5 mL) is added aqueous K₂CO₃ (339 mg, 2.4 mmol, dissolved in 1.0 mL H₂O). This mixture is heated at 45° C. for 4 h. LC/MS indicates the reaction is completed. The reaction mixture is concentrated in vacuo to remove most of the methanol. The residue is partitioned between H₂O and CH₂Cl₂. The two layers are separated, and the organic layer is washed with H₂O and brine, dried over Na₂SO₄, filtered, and concentrated in vacuo. 4.0 M HCl in dioxane is added. The resulting suspension is concentrated in vacuo, and then dried in vacuo. The crude material is triturated with Et₂O, and the yellowish green solid (122 mg, 44%) is collected by vacuum filtration.

¹H NMR (300 MHz, DMSO-d6) δ 8.20 (bs, 3H), 7.80-7.00 (m, 12H), 4.80-4.10 (m, 5H), 4.05-3.85 (m, 2H), 3.20-3.00 (m, 1H), 2.80-2.60 (m, 1H), 2.40-0.08 (m, 6H);

LC 0.82 min; MS 540 (M+H, 100%).

### Example 81

### 4-([5-Aminomethyl-2-fluoro-phenyl)-piperidin-1-yl]-[1-(2-trifluoromethoxy-ethyl)-1H-indol-3-yl]-methanone hydrochloride

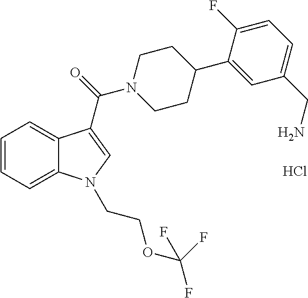

### A. 2,2,2-Trifluoro-1-(1H-indol-3-yl)-ethanone

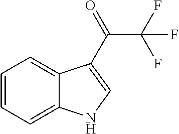

To a solution of 1H-indole (0.38 g, 3.2 mmol) in DMF (15 mL) at r.t. is added TFAA (0.44 mL, 16.2 mmol). After 2 h at 40° C. the reaction mixture is poured into 400 mL 10% sodium bicarbonate solution and the precipitate is filtered, and washed with water (100 mL). The solid is dissolved in EtOAc (200 mL), dried over Na₂SO₄, filtered, and concentrated in vacuo to yield the title product (0.40 g).

¹H NMR (300 MHz, CDCl₃) δ 8.35 (d, 1H), 8.0 (s, 1H), 7.4 (m, 1H), 7.2 (m, 2H); MS m/z: [M+H]⁺=214.

### B. 1H-Indole-3-carboxylic acid

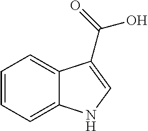

A solution of 2,2,2-trifluoro-(1H-indol-3-yl)-ethanone (0.32 g) in 5 N NaOH (20 mL) is heated at 140° C. for 1.5 hour. The solution is diluted with water (100 mL), extracted with ether (100 mL) and brought to a pH=1 with conc. HCl (10 mL). The solution is extracted with EtOAC (2×100 mL). The organic solution is washed with brine, dried over Na₂SO₄, filtered, and concentrated in vacuo to give the title product (0.27 g).

¹H NMR (300 MHz, CD₃OD) δ 8.0 (d, 1H), 7.9 (s, 1H), 7.2 (d, 2H), 7.1 (m, 1H); MS m/z: [M+H]⁺=161.

### C. 1H-Indole-3-carboxylic acid methyl ester

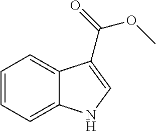

A solution of 1H-indole-3-carboxylic acid (0.49 g) in saturated HCl in MeOH (50 mL) is stirred at r.t. for one hour. The solution is evaporated in vacuo, treated with 10% sodium bicarbonate (100 mL) and extracted with EtOAc (200 mL). The organic solution is washed with brine, dried over Na₂SO₄, filtered, and concentrated in vacuo to give the title product (0.50 g).

¹H NMR (300 MHz, CD₃OD) δ 8.1 (d, 2H), 7.9 (s, 1H), 7.5 (m, 1H), 7.2 (m, 2H), 3.9 (s, 3H);

MS m/z: [M+H]⁺=176.

### D. 1-(2-Trifluoromethoxy-ethyl)-1H-indole-3-carboxylic acid methyl ester

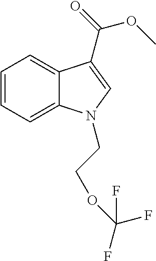

1H-Indole-3-carboxylic acid methyl ester (0.32 g, 1.8 mmol) is dissolved in THF (50 mL) and NaH (0.11 g, 2.7 mmol) is added under N2 at r.t. in one portion. The suspension is stirred at r. t. for 15 minutes. Trifluoromethanesulfonic acid 2-trifluoromethoxy-ethyl ester (0.51 g, 1.8 mmol) is added in one portion to the reaction mixture. The solution is stirred at r.t. for 15 minutes. The suspension is diluted with N HCl (100 mL) and extracted with EtOAc (100 mL). The organic solution is washed with brine, dried over Na2SO4, filtered, and concentrated in vacuo to give the title product (0.36 g).

¹H NMR (300 MHz, CD₃OD) δ 8.1 (d, 1H), 8.0 (s, 1H), 7.5 (m, 1H), 7.3-7.2 (m, 2H), 4.6 (t, 2H), 4.4 (t, 2H) 3.9 (s, 3H);

MS m/z: [M+H]⁺=288.

### E. 1-(2-Trifluoromethoxy-ethyl)-1H-indole-3-carboxylic acid

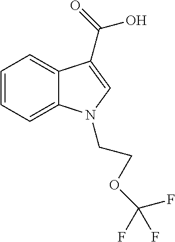

1-(2-Trifluoromethoxy-ethyl)-1H-indole-3-carboxylic acid methyl ester (0.30 g) in 2 N NaOH/MeOH/THF (25 mL/25 mL/25 mL) is stirred at r.t. After 16 hours, the solution is evaporated in vacuo, treated with water (100 mL) and extracted with ether (200 mL). The aqueous solution is brought to pH=1-2 and extracted with EtOAc (100 mL). The organic solution is washed with brine, dried over Na₂SO₄, filtered, and concentrated in vacuo to give the title product (0.27 g).

¹H NMR (300 MHz, CD₃OD) δ 8.1 (d, 1H), 8.0 (s, 1H), 7.5 (m, 1H), 72-7.3 (m, 2H), 4.6 (t, 2H), 4.4 (t, 2H);

MS m/z: [M+H]⁺=274.

### F. 2,2,2-Trifluoro-N-(4-fluoro-3-{1-[1-(2-trifluoromethoxy-ethyl)-1H-indole-3-carbonyl]-piperidin-4-yl}-benzyl)acetamide

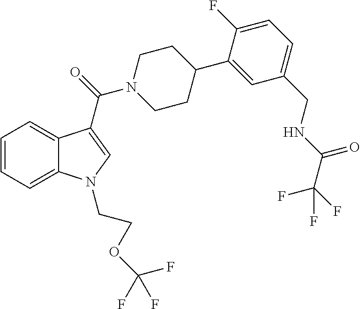

To a suspension of 1-(2-trifluoromethoxy-ethyl)-1H-indole-3-carboxylic acid

(0.30 g, 1.13 mmol), 2,2,2-trifluoro-N-(4-fluoro-3-piperidin-4-yl-benzyl)-acetamide hydrochloride (0.41 g, 1.17 mmol), and EDCI (0.33 g, 1.7 mmol) in CH₂Cl₂ (50 mL) is added Et3N (0.49 mL, 3.5 mmol). The reaction is stirred at room temperature overnight. The reaction mixture is poured into EtOAc and the organic layer washed with sat. NH₄Cl, water and brine. The organic layer is dried over MgSO₄, filtered and concentrated in vacuo to give the crude product. Purification by flash chromatography on SiO₂ eluting with 50% ethyl acetate/heptane gives 0.32 g, (49%) of the desired product (0.32 g, 49%).

¹H NMR (300 MHz, CD₃OD) δ 7.8 (d, 1H), 7.7 (s, 1H), 7.5 (d, 1H), 7.2-7.4 (m, 4H), 7.0 (m, 1H), 4.6 (t, 2H), 4.4 (t, 2H), 3.1-3.3 (m, 3H), 1.7-1.9 (m, 3H), 1.3 (m, 2H), 0.8 (m, 1H);

LCMS m/z: [M+H]⁺=560.

### G. [4-(5-Aminomethyl-2-fluoro-phenyl)-piperidin-1-yl]-[1-(2-trifluoromethoxy-ethyl)-1H-indol-3-yl]-methanone hydrochloride

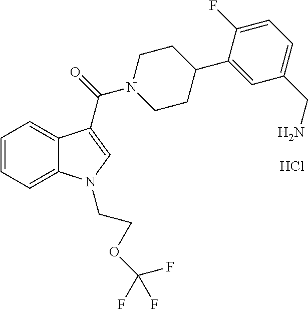

A solution of 2,2,2-trifluoro-N-(4-fluoro-3-{1-[1-(2-trifluoromethoxy-ethyl)-1H-indole-3-carbonyl]-piperidin-4-yl}-benzyl)-acetamide (0.20 g, 0.36 mmol) in MeOH (100 mL) is prepared. To this solution, aqueous K₂CO₃ (0.40 g, 2.8 mmol dissolved in 20 mL water) is added dropwise and the solution is stirred at r.t. overnight. The solution is diluted with water (400 mL) and extracted with EtOAc (2×100 mL). The organic layers are washed with brine, dried over Na₂SO₄, filtered, and concentrated in vacuo. The residue is dissolved in Et₂O (30 mL) and 1 N HCl solution (0.40 mL) is added. The precipitate is filtered, washed with ether and dried under vacuum to give the title product (0.22 g, 80%)

¹H NMR (300 MHz, CD₃OD) δ 7.7 (d, 1H), 7.6 (s, 2H), 7.5-7.4 (m, 2H), 7.0-7.3 (m, 3H), 4.6 (t, 2H), 4.45 (t, 2H), 3.2 (m, 3H), 1.9-1.7 (m, 4H) 1.3 (m, 1H), 0.8 (m, 1H);

MS m/z: [M+H]⁺=464.

### Example 82

### [4-(5-Aminomethyl-2-fluoro-phenyl)-piperidin-1-yl]-[1-(2-methoxy-ethyl)-4-(5-methyl-pyridin-3-yl)-1H-indol-3-yl]-methanone dihydrochloride

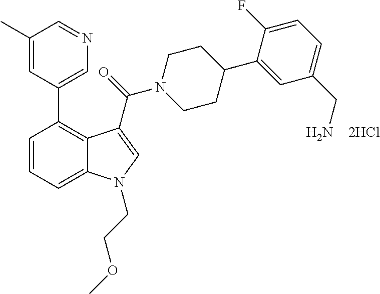

### A. 2,2,2-Trifluoro-N-(4-fluoro-3-{1-[1-(2-methoxy-ethyl)-4-(5-methyl-pyridin-3-yl)-1H-indole-3-carbonyl]-piperidin-4-yl}-benzyl)-acetamide

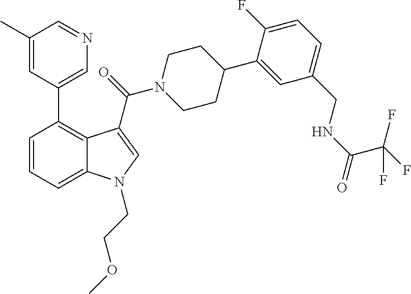

A mixture of N-(3-{1-[4-bromo-1-(2-methoxy-ethyl)-1H-indole-3-carbonyl]-piperidin-4-yl}-4-fluoro-benzyl)-2,2,2-trifluoro-acetamide (entry 48D) (200 mg, 0.34 mmol), 5-methylpyridine-3-boronic acid (56 mg, 0.41 mmol), cesium carbonate (223 mg, 0.68 mmol), and Pd(dppf)Cl₂.CH₂Cl₂ (28 mg, 10% mol) in dioxane/H2O (4.5 mL/0.5 mL) is heated at 80° C. overnight. The reaction mixture is concentrated in vacuo to remove the solvent. The crude material is purified on silica gel with CH₂Cl₂/MeOH (100/0 to 98/2) as eluent to give the product (171 mg, 84%).

¹H NMR (300 MHz, CDCl₃) δ 8.64 (s, 1H), 8.37 (s, 1H), 7.97 (bs, 1H), 7.60-6.80 (m, 8H), 4.80-4.30 (m, 5H), 3.85-3.70 (m, 2H), 3.55-3.30 (m, 4H), 3.00-2.45 (m, 3H), 2.41 (s, 3H), 1.80-0.80 (m, 4H);

LC Rt 0.87 min; MS 597 (M+H, 100%).

### B. [4-(5-Aminomethyl-2-fluoro-phenyl)-piperidin-1-yl]-[1-(2-methoxy-ethyl)-4-(5-methyl-pyridin-3-yl)-1H-indol-3-yl]-methanone dihydrochloride

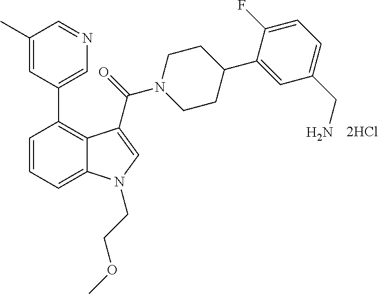

To a mixture of 2,2,2-trifluoro-N-(4-fluoro-3-{1-[1-(2-methoxy-ethyl)-4-(5-methyl-pyridin-3-yl)-1H-indole-3-carbonyl]-piperidin-4-yl}-benzyl)-acetamide (168 mg, 0.28 mmol) in MeOH (5 mL) is added aqueous K₂CO₃ (311 mg, 2.25 mmol, dissolved in 1.0 mL H₂O). This mixture is heated at 45° C. for 3 h. LC/MS indicates the reaction is completed. The reaction mixture is concentrated in vacuo to remove most of the methanol. The residue is partitioned between H₂O and CH₂Cl₂. The two layers are separated, and the organic layer is washed with H2O and brine, dried over Na₂SO₄, filtered, and concentrated in vacuo. 4.0 M HCl in dioxane is added. The resulting suspension is concentrated in vacuo, and then dried in vacuo. The crude material is triturated with Et₂O, and the beige solid (88 mg, 54%) is collected by vacuum filtration.

¹H NMR (300 MHz, DMSO-d6) δ 8.60-8.40 (m, 2H), 8.20-7.80 (br s, 4H), 7.70-6.90 (m, 8H), 4.50-4.20 (m, 3H), 4.05-3.85 (m, 2H), 3.80-3.60 (m, 2H), 3.50-3.10 (m, 4H), 2.80-2.60 (m, 1H), 2.40-2.00 (m, 5H), 1.80-1.10 (m, 4H);

LC 0.62 min; MS 501 (M+H, 100%).

### Example 83

### [4-(5-Aminomethyl-2-fluoro-phenyl)-piperidin-1-yl]-[1-(2-methoxy-ethyl)-4-(2-methyl-pyridin-4-yl)-1H-indol-3-yl]-methanone dihydrochloride

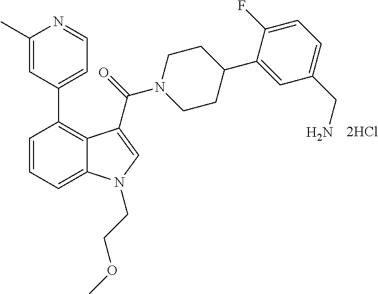

### A. 2,2,2-Trifluoro-N-(4-fluoro-3-{1-[1-(2-methoxy-ethyl)-4-(2-methyl-pyridin-4-yl)-1H-indole-3-carbonyl]-piperidin-4-yl}-benzyl)acetamide

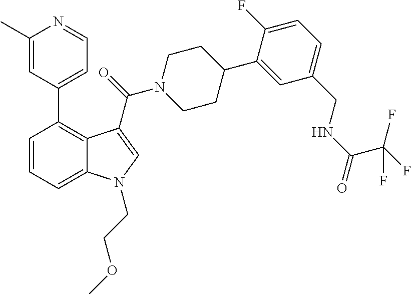

A mixture of N-(3-{1-[4-bromo-1-(2-methoxy-ethyl)-1H-indole-3-carbonyl]-piperidin-4-yl}-4-fluoro-benzyl)-2,2,2-trifluoro-acetamide (entry 48D) (200 mg, 0.34 mmol), 2-picoline-4-boronic acid (56 mg, 0.41 mmol), cesium carbonate (223 mg, 0.68 mmol), and Pd(dppf)Cl₂.CH₂Cl₂ (28 mg, 10% mol) in dioxane/H₂O (4.5 mL/0.5 mL) is heated at 80° C. overnight. The reaction mixture is concentrated in vacuo to remove the solvent. The crude material is purified on silica gel with CH₂Cl₂/MeOH (100/0 to 97/3) as eluent to give the product (180 mg, 88%).

¹H NMR (300 MHz, CDCl₃) δ 8.60-8.40 (m, 1H), 7.60-6.80 (m, 10H), 4.80-4.20 (m, 5H), 3.85-3.65 (m, 2H), 3.55-3.30 (m, 4H), 2.90-2.70 (m, 1H), 2.70-2.20 (m, 5H), 1.80-1.10 (m, 4H);

LC Rt 0.78 min; MS 597 (M+H, 100%).

### B. [4-(5-Aminomethyl-2-fluoro-phenyl)-piperidin-1-yl]-[1-(2-methoxy-ethyl)-4-(2-methyl-pyridin-4-yl)-1H-indol-3-yl]-methanone dihydrochloride

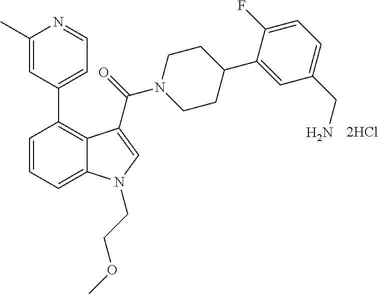

To a mixture of 2,2,2-trifluoro-N-(4-fluoro-3-{1-[1-(2-methoxy-ethyl)-4-(2-methyl-pyridin-4-yl)-1H-indole-3-carbonyl]-piperidin-4-yl}-benzyl)-acetamide (175 mg, 0.29 mmol) in MeOH (5 mL) is added aqueous K₂CO₃ (324 mg, 2.34 mmol, dissolved in 1.0 mL H₂O). This mixture is heated at 45° C. for 3 h. LC/MS indicates the reaction is completed. The reaction mixture is concentrated in vacuo to remove most of the methanol. The residue is partitioned between H₂O and CH₂Cl₂. The two layers are separated, and the organic layer is washed with H₂O and brine, dried over Na₂SO₄, filtered, and concentrated in vacuo. 4.0 M HCl in dioxane is added. The resulting suspension is concentrated in vacuo, and then dried in vacuo. The crude material is triturated with Et₂O, and the slightly yellow solid (105 mg, 63%) is collected by vacuum filtration.

¹H NMR (300 MHz, DMSO-d6) δ 8.80-8.70 (m, 1H), 8.41 (br s, 4H), 8.00-7.00 (m, 9H), 4.60-4.10 (m, 3H), 4.05-3.90 (m, 2H), 3.80-3.60 (m, 2H), 3.50-3.10 (m, 4H), 3.05-2.85 (m, 1H), 2.73 (s, 3H), 2.40-2.10 (m, 2H), 1.80-1.20 (m, 4H);

LC 0.56 min; MS 501 (M+H, 100%).

### Example 84

### [4-(5-Aminomethyl-2-fluoro-phenyl)-piperidin-1-yl]-[4-(5-fluoro-pyridin-3-yl)-1-(2-methoxy-ethyl)-1H-indol-3-yl]-methanone dihydrochloride

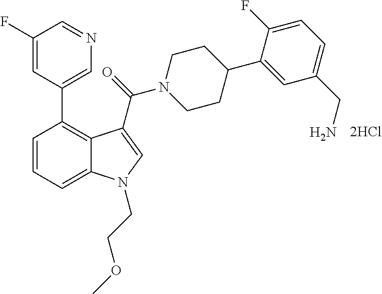

### A. 2,2,2-Trifluoro-N-(4-fluoro-3-{1-[4-(5-fluoro-pyridin-3-yl)-1-(2-methoxy-ethyl)-1H-indole-3-carbonyl]-piperidin-4-yl}-benzyl)-acetamide

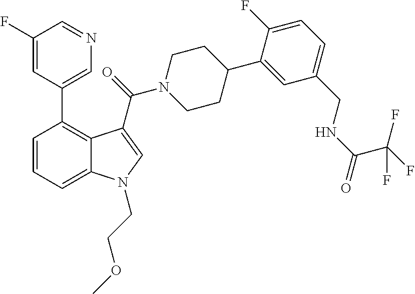

A mixture of N-(3-{1-[4-bromo-1-(2-methoxy-ethyl)-1H-indole-3-carbonyl]-piperidin-4-yl}-4-fluoro-benzyl)-2,2,2-trifluoro-acetamide (entry 48D) (200 mg, 0.34 mmol), 5-fluoropyridine-3-boronic acid (58 mg, 0.41 mmol), cesium carbonate (223 mg, 0.68 mmol), and Pd(dppf)Cl₂.CH₂Cl₂ (28 mg, 10% mol) in dioxane/H₂O (4.5 mL/0.5 mL) is heated at 80° C. overnight. The reaction mixture is concentrated in vacuo to remove the solvent. The crude material is purified on silica gel with CH₂Cl₂/MeOH (100/0 to 97/3) as eluent to give the product (142 mg, 69%).

¹H NMR (300 MHz, CDCl₃) δ 8.65-8.60 (m, 1H), 8.50-8.40 (m, 1H), 7.90-7.70 (m, 1H), 7.60-6.80 (m, 8H), 4.75-4.25 (m, 5H), 3.80-3.70 (m, 2H), 3.70-3.55 (m, 1H), 3.35 (s, 3H), 3.00-2.80 (m, 1H), 2.80-2.20 (m, 2H), 1.90-0.80 (m, 4H);

LC Rt 1.00 min; MS 601 (M+H, 100%).

### B. [4-(5-Aminomethyl-2-fluoro-phenyl)-piperidin-1-yl]-[4-(5-fluoro-pyridin-3-yl)-1-(2-methoxy-ethyl)-1H-indol-3-yl]-methanone dihydrochloride

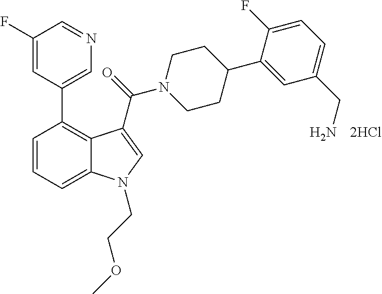

To a mixture of 2,2,2-trifluoro-N-(4-fluoro-3-{1-[4-(5-fluoro-pyridin-3-yl)-1-(2-methoxy-ethyl)-1H-indole-3-carbonyl]-piperidin-4-yl}-benzyl)-acetamide (140 mg, 0.23 mmol) in MeOH (5 mL) is added aqueous K₂CO₃ (324 mg, 2.34 mmol, dissolved in 1.0 mL H₂O). This mixture is heated at 45° C. for 2 h. LC/MS indicates the reaction is completed. The reaction mixture is concentrated in vacuo to remove most of the methanol. The residue is partitioned between H₂O and CH₂Cl₂. The two layers are separated, and the organic layer is washed with H₂O and brine, dried over Na₂SO₄, filtered, and concentrated in vacuo. 4.0 M HCl in dioxane is added. The resulting suspension is concentrated in vacuo, and then dried in vacuo. The crude material is triturated with Et₂O, and the yellow solid (135 mg, quantitative) is collected by vacuum filtration.

¹H NMR (300 MHz, DMSO-d6) δ 8.70-8.20 (m, 6H), 7.90-7.00 (m, 8H), 4.20-4.10 (m, 1H), 4.05-3.90 (m, 2H), 4.05-3.90 (m, 2H), 3.60-3.40 (m, 3H), 3.25 (s, 3H), 3.00-2.80 (m, 1H), 2.40-2.10 (m, 2H), 1.80-1.20 (m, 4H);

LC 0.71 min; MS 505 (M+H, 100%).

### Example 85

### [4-(5-Aminomethyl-2-fluoro-phenyl)-piperidin-1-yl]-[1-(2-methoxy-ethyl)-4-(2-methoxy-pyridin-4-yl)-1H-indol-3-yl]-methanone dihydrochloride

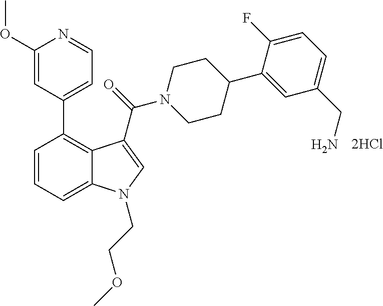

### A. 2,2,2-Trifluoro-N-(4-fluoro-3-{1-[1-(2-methoxy-ethyl)-4-(2-methoxy-pyridin-4-yl)-1H-indole-3-carbonyl]-piperidin-4-yl}-benzyl)acetamide

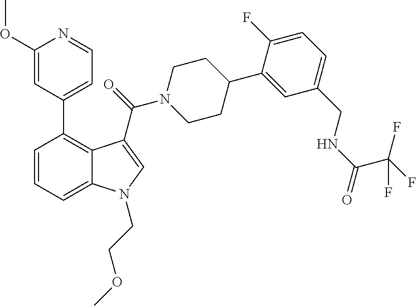

A mixture of N-(3-{1-[4-bromo-1-(2-methoxy-ethyl)-1H-indole-3-carbonyl]-piperidin-4-yl}-4-fluoro-benzyl)-2,2,2-trifluoro-acetamide (entry 48D) (200 mg, 0.34 mmol), 2-methoxypyridine-4-boronic acid (63 mg, 0.41 mmol), cesium carbonate (223 mg, 0.68 mmol), and Pd(dppf)Cl₂.CH₂Cl₂ (28 mg, 10% mol) in dioxane/H₂O (4.5 mL/0.5 mL) is heated at 80° C. for 5 h. The reaction mixture is concentrated in vacuo to remove the solvent. The crude material is purified on silica gel with CH₂Cl₂/MeOH (100/0 to 97/3) as eluent to give the product (124 mg, 59%) as a beige foam.

¹H NMR (300 MHz, CDCl₃) δ 8.20-8.10 (m, 1H), 7.60-6.80 (m, 10H), 4.80-4.40 (m, 3H), 4.35 (d, J=5.4 Hz, 2H), 3.94 (s, 3H), 3.77 (d, J=5.5 Hz, 2H), 3.60-3.40 (m, 1H), 3.35 (s, 3H), 3.00-2.80 (m, 1H), 2.80-2.20 (m, 2H), 1.80-1.20 (m, 4H);

LC Rt 1.04 min; MS 613 (M+H, 100%).

### B. [4-(5-Aminomethyl-2-fluoro-phenyl)-piperidin-1-yl]-[1-(2-methoxy-ethyl)-4-(2-methoxy-pyridin-4-yl)-1H-indol-3-yl]-methanone dihydrochloride

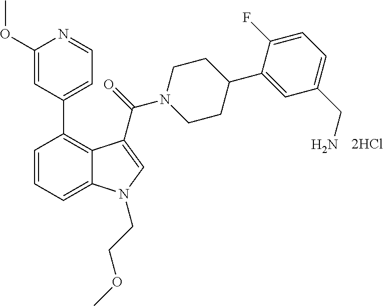

To a mixture of 2,2,2-trifluoro-N-(4-fluoro-3-{1-[1-(2-methoxy-ethyl)-4-(2-methoxy-pyridin-4-yl)-1H-indole-3-carbonyl]-piperidin-4-yl}-benzyl)-acetamide (121 mg, 0.19 mmol) in MeOH (5 mL) is added aqueous K₂CO₃ (218 mg, 1.58 mmol, dissolved in 1.0 mL H₂O). This mixture is heated at 45° C. for 3 h. LC/MS indicates the reaction is completed. The reaction mixture is concentrated in vacuo to remove most of the methanol. The residue is partitioned between H₂O and CH₂Cl₂. The two layers are separated, and the organic layer is washed with H₂O and brine, dried over Na₂SO₄, filtered, and concentrated in vacuo. 4.0 M HCl in dioxane is added. The resulting suspension is concentrated in vacuo, and then dried in vacuo. The crude material is triturated with Et₂O, and the yellow solid (136 mg) is collected by vacuum filtration.

¹H NMR (300 MHz, DMSO-d6) δ 8.50-8.10 (m, 5H), 7.80-6.80 (m, 9H), 4.60-4.20 (m, 3H), 4.00-3.75 (m, 5H), 3.70-3.60 (m, 2H), 3.60-3.40 (m, 1H), 3.24 (s, 3H), 3.00-2.80 (m, 1H), 2.80-2.10 (m, 2H), 1.80-1.20 (m, 4H);

LC 0.75 min; MS 517 (M+H, 100%).

### Example 86

### [4-(5-Aminomethyl-2-fluoro-phenyl)-piperidin-1-yl]-[7-fluoro-1-(2-trifluoromethoxy-ethyl)-1H-indol-3-yl]-methanone hydrochloride

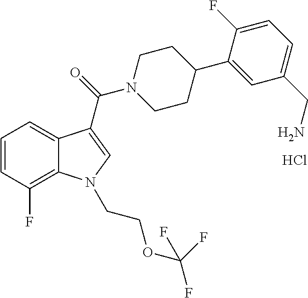

### A. 2,2,2-Trifluoro-1-(7-fluoro-1H-indol-3-yl)-ethanone

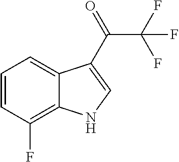

To a solution of 7-fluoro-1H-indole (0.66 g, 4.9 mmol) in DMF (20 mL) at r.t. is added TFAA (2.0 mL). After 2 h at 40° C. the reaction mixture is poured into 10% sodium bicarbonate solution (400 mL) and the precipitate is filtered and washed with water (100 mL). The solid is dissolved in EtOAc (200 mL), dried over Na₂SO₄, filtered and concentrated in vacuo to afford the product (0.66 g).

¹H NMR (300 MHz, DMSO-d6) δ 8.45 (m, 2H), 7.3 (m, 1H), 7.0-7.1 (m, 1H),

MS m/z: [M+H]⁺=232.

### B. 7-Fluoro-1H-indole-3-carboxylic acid

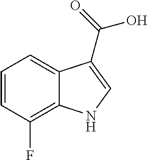

A solution of 2,2,2-trifluoro-1-(7-fluoro-1H-indol-3-yl)-ethanone (0.66 g) in 5 N NaOH (20 mL) is heated at 140° C. for one hour. The solution is diluted with water (100 mL), extracted with ether (100 mL) and brought to pH=1 with conc HCl (10 mL). The solution is extracted with EtOAc (2×100 mL). The organic layer is washed with brine, dried over Na₂SO₄, filtered and concentrated in vacuo to afford the product (0.66 g).

¹H NMR (300 MHz, CD₃OD) δ 8.0 (s, 1H), 7.8 (d, 1H), 7.1 (m, 1H), 6.9-7.0 (m, 1H);

MS m/z: [M+H]⁺=1809.

### C. 7-Fluoro-1H-indole-3-carboxylic acid methyl ester

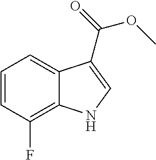

A solution 7-fluoro-1H-indole-3-carboxylic acid (0.66 g) in saturated HCl in MeOH (50 mL) is stirred at r.t. for one hour. The solution is evaporated in vacuo, treated with 10% sodium bicarbonate solution (100 mL) and extracted with EtOAc (200 mL). The organic layer is washed with brine, dried over Na₂SO₄, filtered and concentrated in vacuo to afford the title product (0.65 g).

¹H NMR (300 MHz, DMSO-d6) δ 8.0 (s, 1H), 7.8 (d, 1H), 7.1 (m, 1H), 6.9-7.0 (m, 1H), 3.8 (s, 3H);

MS m/z: [M+H]⁺=194.

### D. 7-Fluoro-1-(2-trifluoromethoxy-ethyl)-1H-indole-3-carboxylic acid methyl ester

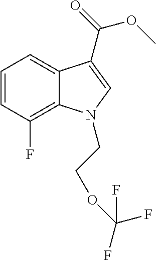

To a solution of 7-fluoro-1H-indole-3-carboxylic acid methyl ester (0.41 g, 2.1 mmol) in THF (50 mL) under N2 is added NaH (0.16 g, 4.2 mmol) at r.t. in one portion. The suspension is stirred at r.t. for 15 min then trifluoromethanesulfonic acid 2-trifluoromethoxy-ethyl ester (0.55 g, 2.1 mmol) is added in one portion. The reaction mixture is stirred at r.t. for 15 min then the suspension is diluted with 1 N HCl (100 mL) and extracted with EtOAc (100 mL). The organic layer is washed with brine, dried over Na₂SO₄, filtered and concentrated in vacuo to afford the product (0.42 g).

¹H NMR (300 MHz, CDCl₃) δ 8.0 (d, 1H), 7.8 (s, 1H), 7.0 (m, 1H), 6.9-7.0 (m, 1H), 4.6 (t, 2H), 4.3 (t, 2H), 3.9 (s, 3H),

LCMS m/z: [M+H]⁺=306.

### E. 7-Fluoro-1-(2-trifluoromethoxy-ethyl)-1H-indole-3-carboxylic acid

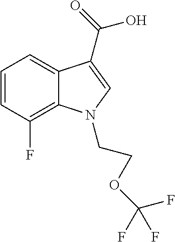

A solution of 7-fluoro-1-(2-trifluoromethoxy-ethyl)-1H-indole-3-carboxylic acid methyl ester (0.47 g) in 2 N NaOH/MeOH/THF (25 mL/25 mL/25 mL) is stirred at r.t. After 16 hours the reaction mixture is evaporated in vacuo, treated with water (100 mL) and extracted with ether (200 mL). The aqueous layer is brought to pH=1-2 and extracted with EtOAc (100 mL). The organic layer is washed with brine, dried over Na₂SO₄, filtered and concentrated in vacuo to afford the product (0.47 g).

¹H NMR (300 MHz, CD₃OD) δ 8.1 (d, 1H), 7.9 (s, 1H), 7.3 (m, 1H), 7.1 (m, 1H), 4.8 (t, 2H), 4.4 (t, 2H);

MS m/z: [M+H]⁺=292.

### F. 2,2,2-Trifluoro-N-(4-fluoro-3-{1-[7-fluoro-1-(2-trifluoromethoxy-ethyl)-1H-indole-3-carbonyl]-piperidin-4-yl}-benzyl)-acetamide

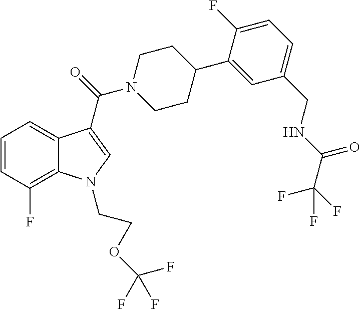

To a suspension of 7-fluoro-1-(2-trifluoromethoxy-ethyl)-1H-indole-3-carboxylic acid (0.47 g, 1.6 mmol), 2,2,2-trifluoro-N-(4-fluoro-3-piperidin-4-yl-benzyl)-acetamide hydrochloride (0.70 g, 2.1 mmol), and EDCI (0.41 g, 2.1 mmol) in CH₂Cl₂ (50 mL) is added Et₃N (0.68 mL, 4.9 mmol). The reaction is stirred at room temperature overnight. The reaction mixture is poured into EtOAc and the organic layer washed with sat. NH₄Cl, water and brine. The organic layer is dried over MgSO₄, filtered and concentrated in vacuo to give the crude product. Purification by flash chromatography on SiO₂ eluting with 50% ethyl acetate/heptane gives of the desired product (0.55 g, 59%).

¹H NMR (300 MHz, CD₃OD) δ 7.65 (s, 1H), 7.5 (d, 1H), 7.3 (d, 1H), 7.2-6.9 (m, 4H), 4.6 (t, 2H), 4.4 (m, 4H), 3.2 (m, 2H), 2.0 (m, 2H), 1.7-1.9 (m, 2H), 1.3 (m, 2H), 0.8 (m, 1H),

MS m/z: [M+H]⁺=587.

### G. [4-(5-Aminomethyl-2-fluoro-phenyl)-piperidin-1-yl]-[7-fluoro-1-(2-trifluoromethoxy-ethyl)-1H-indol-3-yl]-methanone hychloride

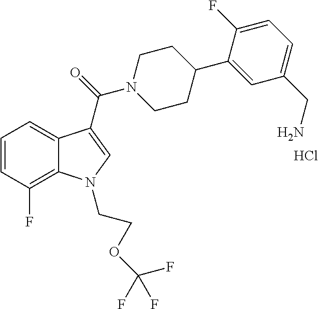

To a solution of 2,2,2-trifluoro-N-(4-fluoro-3-{1-[7-fluoro-1-(2-trifluoromethoxy-ethyl)-1H-indole-3-carbonyl]-piperidin-4-yl}-benzyl)-acetamide (0.40 g, 0.86 mmol) in MeOH (100 mL) is added aqueous K₂CO₃ (0.94 g dissolved in 20 mL water). The solution is stirred at r.t. overnight. The reaction mixture is diluted with water (400 mL) and extracted with EtOAc (2×100 mL). The organic layer is washed with brine, dried over Na₂SO₄, filtered and concentrated in vacuo. The residue is dissolved in Et₂O (50 mL) and 1N HCl (0.90 mL) is added. The resulting precipitate is filtered, washed with ether and dried under vacuum to afford the product (0.33 g, 80%).

¹H NMR (300 MHz, CD₃OD) δ 7.6 (s, 1H), 7.6-7.5 (m, 2H), 7.4-7.3 (m, 1H), 7.1-7.2 (m, 2H), 7.1-6.9 (m, 2H), 4.7 (t, 2H), 4.6-4.5 (m, 1H), 4.4 (t, 2H), 4.1 (m, 2H), 3.2 (m, 3H), 2.0-1.7 (m, 3H);

MS m/z: [M+H]⁺=483.

### Example 87

### 4-([5-Aminomethyl-2-fluoro-phenyl)-piperidin-1-yl]-[7-chloro-1-(2-trifluoromethoxy-ethyl)-1H-indol-3-yl]-methanone hydrochloride

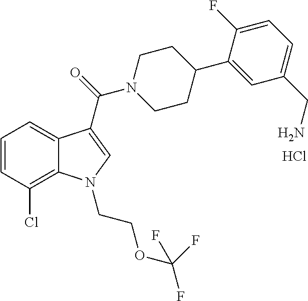

### A. 2,2,2-Trifluoro-1-(7-Chloro-1H-indol-3-yl)-ethanone

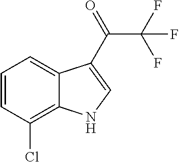

To a solution of 7-chloro-1H-indole (0.16 g, 0.71 mmol) in DMF (5 mL) at r.t. is added TFAA (0.30 mL, 2.14 mmol). After 2 h at 40° C. the reaction mixture is poured into 10% sodium bicarbonate solution (400 mL) and the precipitate is filtered and washed with water (100 mL). The solid is dissolved in EtOAc (200 mL), dried over Na₂SO₄, filtered and concentrated in vacuo to afford the product (0.16 g).

¹H NMR (300 MHz, DMSO-d6) δ 8.35 (m, 2H), 7.3 (m, 1H), 7.0-7.1 (m, 1H);

MS m/z: [M+H]⁺=248.

### B. 7-Chloro-1H-indole-3-carboxylic acid

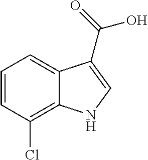

A solution of 2,2,2-trifluoro-1-(7-chloro-1H-indol-3-yl)-ethanone (0.32 g) in 5 N NaOH (20 mL) is heated at 140° C. for 1.5 hour. The reaction mixture is diluted with water (100 mL), extracted with ether (100 mL) and the aqueous layer is brought to pH=1 with conc HCl (10 mL). The aqueous layer is extracted with EtOAc (2×100 mL) and the organic layers are washed with brine, dried over Na₂SO₄, filtered and concentrated in vacuo to afford the product (0.27 g).

¹H NMR (300 MHz, CD₃OD) δ 8.0 (s, 1H), 7.9 (d, 1H), 7.2 (d, 1H), 7.1 (m, 1H);

MS m/z: [M+H]⁺=196.

### C. 7-Chloro-1H-indole-3-carboxylic acid methyl ester

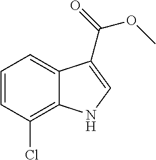

A solution of 7-chloro-1H-indole-3-carboxylic acid (0.49 g) in sat HCl in MeOH (50 mL) is stirred at r.t. for one hour. The reaction mixture is evaporated in vacuo, treated with 10% sodium bicarbonate solution (100 mL) and extracted with EtOAc (200 mL). The organic layer is washed with brine, dried over Na₂SO₄, filtered and concentrated in vacuo to afford the product (0.50 g).

¹H NMR (300 MHz, CD₃OD) δ 8.0 (m, 2H), 7.3 (m, 1H), 7.2 (m, 1H), 3.9 (s, 3H);

MS m/z: [M+H]⁺=210.

### D. 7-Chloro-1-(2-trifluoromethoxy-ethyl)-1H-indole-3-carboxylic acid methyl ester

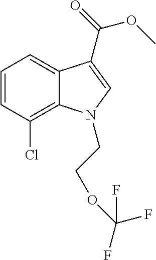

To a solution of 7-chloro-1H-indole-3-carboxylic acid methyl ester (0.30 g, 1.4 mmol) in THF (50 mL) under N₂ is added NaH (0.12 g, 2.9 mmol) at r.t. in one portion. The suspension is stirred at r.t. for 15 min then trifluoromethanesulfonic acid 2-trifluoromethoxy-ethyl ester (0.36 g, 1.4 mmol) is added in one portion. The reaction mixture is stirred at r.t. for another 15 min then the suspension is diluted with 1 N HCl (100 mL) and extracted with EtOAc (100 mL). The organic layer is washed with brine, dried over Na₂SO₄, filtered and concentrated in vacuo to afford the product (0.33 g).

¹H NMR (300 MHz, CD₃OD) δ 8.1 (d, 1H), 8.0 (s, 1H), 7.5 (m, 1H), 72-7.3 (m, 1H), 4.6 (t, 2H), 4.4 (t, 2H), 3.9 (s, 3H);

MS m/z: [M+H]⁺=322.

### E. 7-Chloro-1-(2-trifluoromethoxy-ethyl)-1H-indole-3-carboxylic acid

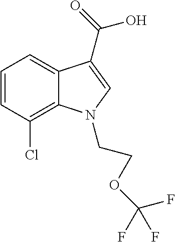

A solution of 7-chloro-1-(2-trifluoromethoxy-ethyl)-1H-indole-3-carboxylic acid methyl ester (0.29 g) in 2 N NaOH/MeOH/THF (25 mL/25 mL/25 mL) is stirred at r.t. After 16 hours the reaction mixture is evaporated in vacuo, treated with water (100 mL) and extracted with ether (100 mL). The aqueous layer is brought to pH=1-2 with conc. HCl and extracted with EtOAc (100 mL). The organic layer is washed with brine, dried over Na₂SO₄, filtered and concentrated in vacuo to afford the product (0.27 g).

¹H NMR (300 MHz, CD₃OD) δ 8.1 (d, 1H), 7.9 (s, 1H), 7.3-7.1 (m, 2H), 4.9 (t, 2H), 4.4 (t, 2H);

MS m/z: [M+H]⁺=308.

### F. 2,2,2-Trifluoro-N-(4-fluoro-3-{1-[7-Chloro-1-(2-trifluoromethoxy-ethyl)-1H-indole-3-carbonyl]-piperidin-4-yl}-benzyl)-acetamide

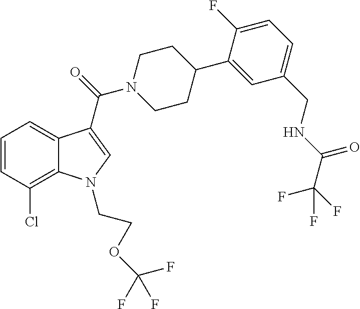

To a suspension of 7-chloro-1-(2-trifluoromethoxy-ethyl)-1H-indole-3-carboxylic acid (0.36 g, 1.16 mmol), 2,2,2-trifluoro-N-(4-fluoro-3-piperidin-4-yl-benzyl)-acetamide hydrochloride (0.39 g, 1.17 mmol), and EDCI (0.31 g, 1.6 mmol) in CH₂Cl₂ (50 mL) is added Et₃N (0.34 mL, 2.5 mmol). The reaction is stirred at r.t. overnight then the reaction mixture is poured into EtOAc and the organic layer washed with sat NH₄Cl, water and brine. The organic layer is dried over MgSO₄, filtered and concentrated in vacuo to give the crude product. Purification by flash chromatography on SiO₂ eluting with 50% ethyl acetate/heptane gives of the desired product (0.38 g, 49%).

¹H NMR (300 MHz, CD₃OD) δ 7.7 (d, 1H), 7.6 (s, 1H), 7.3-7.0 (d, 5H), 4.9 (t, 2H), 4.6-4.4 (m, 2H), 3.3-3.1 (m, 1H), 1.9-1.7 (m, 4H), 1.3 (m, 1H), 0.8 (m, 1H);

MS m/z: [M+H]⁺=594.

### G. [4-(5-Aminomethyl-2-fluoro-phenyl)-piperidin-1-yl]-[7-chloro-1-(2-trifluoromethoxy-ethyl)-1H-indol-3-yl]-methanone hydrochloride

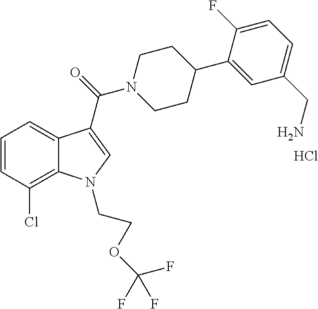

To a solution of 2,2,2-trifluoro-N-(4-fluoro-3-{1-[7-chloro-1-(2-trifluoromethoxy-ethyl)-1H-indole-3-carbonyl]-piperidin-4-yl}-benzyl)-acetamide (0.30 g, 0.50 mmol) in MeOH (100 mL), aqueous K₂CO₃ (0.55 g, 4.0 mmol in 20 mL water) is added dropwise and the reaction mixture is stirred at r.t. overnight. The solution is diluted with water (400 mL) and extracted with EtOAc (2×100 mL). The organic layers are washed with brine, dried over Na₂SO₄, filtered and concentrated in vacuo. The residue is dissolved in Et₂O (30 mL) and 1 N HCl (0.60 mL) is added. The precipitate is filtered, washed with ether and dried under vacuum to afford the titled product (0.19 g, 73%).

¹H NMR (300 MHz, CD₃OD) δ7.7 (d, 1H), 7.6 (d, 2H), 7.5-7.2 (m, 3H), 7.1-7.0 (m, 1H), 4.6 (t, 2H), 4.45 (t, 2H), 3.2 (m, 2H), 1.9-1.7 (m, 3H) 1.3 (m, 1H), 0.8 (m, 1H); MS m/z: [M+H]⁺=498.

### Example 88

### 4-([5-Aminomethyl-2-fluoro-phenyl)-piperidin-1-yl]-[7-methyl-1-(2-trifluoromethoxy-ethyl)-1H-indol-3-yl]-methanone hydrochloride

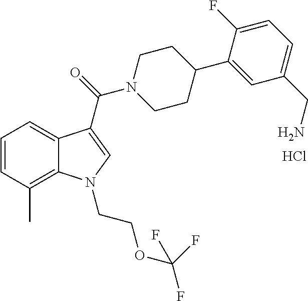

### A. 7-Methyl-1-(2-trifluoromethoxy-ethyl)-1H-indole-3-carboxylic acid methyl ester

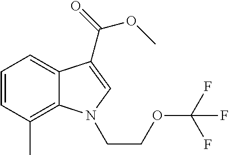

NaH (0.05 g, 2.0 mmol) is added in one portion to a solution of 7-methyl-1H-indole-3-carboxylic acid methyl ester (0.20 g, 0.98 mmol) in THF (20 mL) under N₂ at r.t. and stirred for 15 min. Trifluoromethanesulfonic acid 2-trifluoromethoxy-ethyl ester (0.26 g, 0.98 mmol) is added to the reaction mixture and the solution stirred at r.t. for an additional 15 min. The suspension is then diluted with 1 N HCl (100 mL) and extracted with EtOAc (100 mL). The organic layer is washed with brine, dried over Na₂SO₄, filtered and concentrated in vacuo to yield the titled product (0.26 g).

¹H NMR (300 MHz, CDCl₃) δ 8.1 (d, 1H), 7.8 (s, 1H), 7.2 (m, 1H), 7.0 (m, 1H), 4.6 (t, 2H), 4.4 (t, 2H) 3.9 (s, 3H), 2.6 (s, 3H);

MS m/z: [M+H]⁺=302.

### B. 7-methyl-1-(2-trifluoromethoxy-ethyl)-1H-indole-3-carboxylic acid

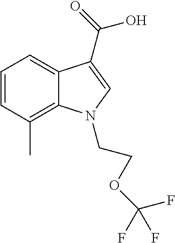

A solution of 7-methyl-1-(2-trifluoromethoxy-ethyl)-1H-indole-3-carboxylic acid methyl ester (0.32 g) in 2 N NaOH/MeOH/THF (25 mL/25 mL/25 mL) is stirred at r.t. for 16 hours. The reaction mixture is then evaporated in vacuo, treated with water (100 mL) and extracted with ether (200 mL). The aqueous layer is brought to pH=1-2 with conc.HCl and extracted with EtOAc (100 mL). The organic layer is washed with brine, dried over Na₂SO₄, filtered and concentrated in vacuo to yield the titled product (0.24 g).

¹H NMR (300 MHz, CD₃OD) δ 8.0 (d, 1H), 7.9 (s, 1H), 7.1 (m, 1H), 6.9 (m, 1H), 4.8 (t, 2H), 4.4 (t, 2H), 2.9 (s, 3H);

MS m/z: [M+H]⁺=288.

### C. 2,2,2-Trifluoro-N-(4-fluoro-3-{1-[7-methyl-1-(2-trifluoromethoxy-ethyl)-1H-indole-3-carbonyl]-piperidin-4-yl}-benzyl)-acetamide

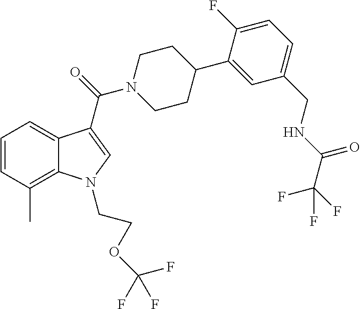

To a suspension of 7-methyl-1-(2-trifluoromethoxy-ethyl)-1H-indole-3-carboxylic acid (0.20 g, 0.69 mmol), 2,2,2-trifluoro-N-(4-fluoro-3-piperidin-4-yl-benzyl)-acetamide hydrochloride (0.35 g, 0.69 mmol) and EDCI (0.33 g, 1.7 mmol) in CH₂Cl₂ (50 mL) is added Et3N (0.20 mL, 2.1 mmol). The reaction mixture is stirred at r.t. overnight then the reaction mixture is poured into EtOAc and the organic layer washed with sat NH₄Cl, water and brine. The organic layer is dried over MgSO₄, filtered and concentrated in vacuo to give the crude product. Purification by flash chromatography on SiO₂ eluting with 50% ethyl acetate/heptanes gives the desired product (0.17 g, 43%).

¹H NMR (300 MHz, CD₃OD) δ 7.6 (m, 2H), 7.4 (d, 1H), 7.2 (m, 1H), 7.1-6.9 (m, 2H), 4.8 (t, 2H), 4.6-4.3 (m, 3H), 3.3-3.1 (m, 3H), 2.6 (s, 3H), 1.9-1.3 (m, 3H), 1.3 (m, 1H), 0.8 (m, 1H);

MS m/z: [M+H]⁺=574.

### D. [4-(5-Aminomethyl-2-fluoro-phenyl)-piperidin-1-yl]-[7-methyl-1-(2-trifluoromethoxy-ethyl)-1H-indol-3-yl]-methanone hydrochloride

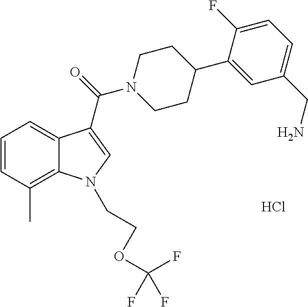

Aqueous K₂CO₃ (0.38 g, 2.7 mmol dissolved in 20 mL water) is added dropwise to a solution of 2,2,2-trifluoro-N-(4-fluoro-3-{1-[7-methyl-1-(2-trifluoro-methoxy-ethyl)-1H-indole-3-carbonyl]-piperidin-4-yl}-benzyl)-acetamide (0.22 g, 0.35 mmol) in MeOH (100 mL) and stirred at r.t. overnight. The reaction mixture is diluted with water (400 mL) and extracted with EtOAc (2×100 mL). The organic layers are washed with brine, dried over Na₂SO₄, filtered and concentrated in vacuo. The residue is dissolved in Et₂O (20 mL) and 1 N HCl solution (0.40 mL) is added. The precipitate is filtered, washed with ether and dried under vacuum to provide the titled product (0.16 g, 80%)

¹H NMR (300 MHz, CD₃OD) δ 7.5 (m, 2H), 7.3 (m, 1H), 7.2 (m, 1H), 7.1-6.9 (m, 3H), 4.8 (t, 2H), 4.5-4.4 (m, 3H), 3.2-3.1 (m, 3H), 2.6 (s, 3H), 1.9-1.7 (m, 4H), 1.3 (m, 1H), 0.8 (m, 1H);

MS m/z: [M+H]⁺=477.

### Example 89

### [4-(5-Aminomethyl-2-fluoro-phenyl)-piperidin-1-yl]-[1-(2-methoxy-ethyl)-4-(2-methyl-2H-pyrazol-3-yl)-1H-indol-3-yl]-methanone dihydrochloride

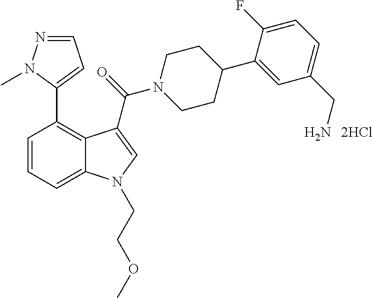

### A. 2,2,2-Trifluoro-N-(4-fluoro-3-{1-[1-(2-methoxy-ethyl)-4-(2-methyl-2H-pyrazol-3-yl)-1H-indole-3-carbonyl]-piperidin-4-yl}-benzyl)-acetamide

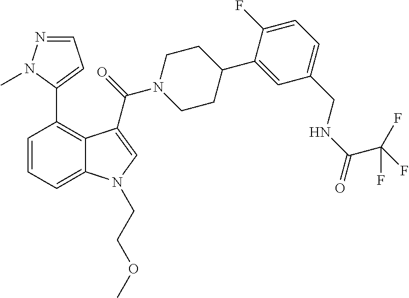

A mixture of N-(3-{1-[4-bromo-1-(2-methoxy-ethyl)-1H-indole-3-carbonyl]-piperidin-4-yl}-4-fluoro-benzyl)-2,2,2-trifluoro-acetamide (entry 48D) (200 mg, 0.34 mmol), 1-methyl-1H-pyrazole-5-boronic acid (85 mg, 0.41 mmol), cesium carbonate (223 mg, 0.68 mmol), and Pd(dppf)Cl₂.CH₂Cl₂ (28 mg, 10% mol) in dioxane/H₂O (4.5 mL/0.5 mL) is heated at 80° C. overnight. The reaction mixture is concentrated in vacuo to remove the solvent. The crude material is purified on silica gel with CH₂Cl₂/MeOH (100/0 to 97/3) as eluent to give the product as a beige foam (107 mg, 52%).

¹H NMR (300 MHz, CDCl₃) δ 7.70-6.85 (m, 9H), 6.56 (bs, 1H), 4.85-4.45 (m, 3H), 4.34 (d, J=5.3 Hz, 2H), 3.85-3.60 (m, 5H), 3.60-3.40 (m, 1H), 3.36 (s, 3H), 3.00-2.40 (m, 3H), 1.90-1.20 (m, 4H);

LC Rt 0.98 min; MS 586 (M+H, 100%).

### B. [4-(5-Aminomethyl-2-fluoro-phenyl)-piperidin-1-yl]-[1-(2-methoxy-ethyl)-4-(2-methyl-2H-pyrazol-3-yl)-1H-indol-3-yl]-methanone dihydrochloride

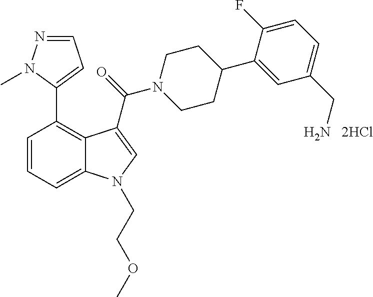

A mixture of 2,2,2-trifluoro-N-(4-fluoro-3-{1-[1-(2-methoxy-ethyl)-4-(2-methyl-2H-pyrazol-3-yl)-1H-indole-3-carbonyl]-piperidin-4-yl}-benzyl)-acetamide (105 mg, 0.17 mmol) in MeOH (5 mL) is added aqueous K₂CO₃ (198 mg, 1.43 mmol, dissolved in 1.0 mL H₂O). This mixture is heated at 45° C. for 3 h. LC/MS indicates the reaction is completed. The reaction mixture is concentrated in vacuo to remove most of the methanol. The residue is partitioned between H₂O and CH₂Cl₂. The two layers are separated, and the organic layer is washed with H₂O and brine, dried over Na₂SO₄, filtered, and concentrated in vacuo. 4.0 M HCl in dioxane is added. The resulting suspension is concentrated in vacuo, and then dried in vacuo. The crude material is triturated with Et₂O, and the beige solid (66 mg, 69%) is collected by vacuum filtration.

¹H NMR (300 MHz, DMSO-d6) δ 8.41 (bs, 4H), 7.80-7.00 (m, 8H), 4.60-4.20 (m, 5H), 3.80-3.60 (m, 5H), 3.65-3.30 (m, 1H), 3.25 (s, 3H), 3.00-2.75 (m, 1H), 2.80-2.10 (m, 2H), 1.80-1.10 (m, 4H);

LC 0.68 min; MS 490 (M+H, 100%).

### Example 90

### [4-(5-Aminomethyl-2-fluoro-phenyl)-piperidin-1-yl]-[4-fluoro-(2-trifluoromethoxy-ethyl)-1H-indol-3-yl]-methanone hydrochloride

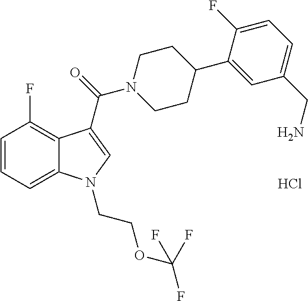

### A. 2,2,2-Trifluoro-1-(4-fluoro-1H-indol-3-yl)-ethanone

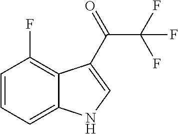

To a solution of 4-fluoro-1H-indole (0.66 g, 4.9 mmol) in DMF (20 mL) is added TFAA (2.0 mL). After 2 h the reaction mixture is poured into 10% sodium bicarbonate solution (400 mL) and the precipitate is filtered, and washed with water (100 mL). The solid is dissolved in EtOAc (200 mL) and dried over Na₂SO₄, filtered and concentrated in vacuo to afford the titled product (0.66 g).

¹H NMR (300 MHz, DMSO-d6) δ 8.5 (s, 1H), 7.4 (d, 1H), 7.3 (m, 1H), 7.0-7.1 (m, H);

MS m/z: [M+H]⁺=233.

### B. 4-Fluoro-1H-indole-3-carboxylic acid

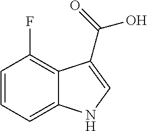

A solution of 2,2,2-trifluoro-1-(4-fluoro-1H-indol-3-yl)-ethanone (0.66 g) in 5 N NaOH (20 mL) is heated at 14° C. for 1 hour. The reaction mixture is allowed to cool, diluted with water (100 mL) and extracted with ether (100 mL). The aqueous layer is brought to pH=1 using conc HCl (10 mL) and extracted with EtOAc (2×100 mL). The organic layers are washed with brine, dried over Na₂SO₄, filtered and contentrated in vacuo to provide the titled product (0.63 g).

¹H NMR (300 MHz, DMSO-d6) δ 8.0 (s, 1H), 7.2 (d, 1H), 7.1 (m, 1H), 7.0-7.1 (m, 1H);

MS m/z: [M+H]⁺=180.

### C. 4-Fluoro-1H-indole-3-carboxylic acid methyl ester

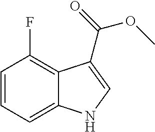

A solution of 4-fluoro-1H-indole-3-carboxylic acid (0.60 g) in sat HCl in MeOH (50 mL) is stirred at r.t. for 1 hour. The reaction mixture is evaporated in vacuo, treated with 10% sodium bicarbonate solution (100 mL) and extracted with EtOAc (200 mL). The organic layer is washed with brine, dried over Na₂SO₄, filtered and concentrated in vacuo to give the titled product (0.60 g).

¹H NMR (300 MHz, DMSO-d6) δ 8.0 (s, 1H), 7.2 (d, 1H), 7.1 (m, 1H), 7.0-7.1 (m, 1H), 3.8 (s, 3H);

LCMS m/z: [M+H]⁺=194.

### D. 4-Fluoro-1-(2-trifluoromethoxy-ethyl)-1H-indole-3-carboxylic acid methyl ester

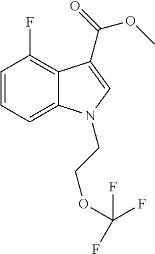

To a solution of 4-fluoro-1H-indole-3-carboxylic acid methyl ester (0.30 g, 1.3 mmol) in THF (20 mL) under N₂ is added NaH (0.10 g, 2.6 mmol) at r.t. in one portion. The suspension is stirred at r.t. for 15 min then trifluoromethanesulfonic acid 2-trifluoromethoxy-ethyl ester (0.34 g, 1.3 mmoles) is added in one portion. The reaction mixture is stirred at r.t. for an additional 15 min then the suspension is diluted with 1 N HCl (100 mL) and extracted with EtOAc (100 mL). The organic layer is washed with brine, dried over Na₂SO₄, filtered and concentrated in vacuo to provide the titled product (0.34 g).

¹H NMR (300 MHz, CD₃OD) δ 7.8 (s, 1H), 7.1 (d, 1H), 7.0 (m, 1H), 7.0-6.9 (m, 1H), 4.5 (t, 2H), 4.25 (t, 2H) 3.8 (s, 3H);

MS m/z: [M+H]⁺=306.

### E. 4-Fluoro-1-(2-trifluoromethoxy-ethyl)-1H-indole-3-carboxylic acid

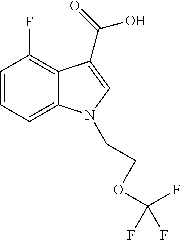

A solution of 4-fluoro-1-(2-trifluoromethoxy-ethyl)-1H-indole-3-carboxylic acid methyl ester (0.16 g) in 2 N NaOH/MeOH/THF (25 mL/25 mL/25 mL) is stirred at r.t. for 16 hours. The reaction mixture is then evaporated in vacuo and the residue is treated with water (100 mL) and extracted with ether (100 mL). The aqueous solution is acidified to pH=1-2 with conc HCl and extracted with EtOAc (100 mL). The organic layer is washed with brine, dried over Na₂SO₄, filtered and concentrated in vacuo to yield the titled product (0.12 g).

¹H NMR (300 MHz, CD₃OD) δ 8.0 (s, 1H), 7.4 (d, 1H), 7.2 (m, 1H), 6.8 (m, 1H), 4.6 (t, 2H), 4.4 (t, 2H);

MS m/z: [M+H]⁺=292.

### F. 2,2,2-Trifluoro-N-(4-fluoro-3-{1-[4-fluoro-1-(2-trifluoromethoxy-ethyl)-1H-indole-3-carbonyl]-piperidin-4-yl}-benzyl)-acetamide

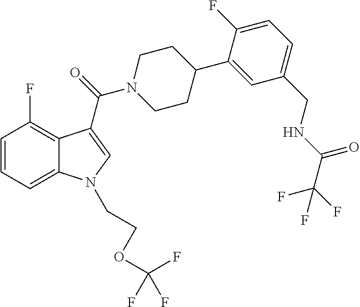

To a suspension of 4-fluoro-1-(2-trifluoromethoxy-ethyl)-1H-indole-3-carboxylic acid 0.1 g, 0.34 mmol), 2,2,2-trifluoro-N-(4-fluoro-3-piperidin-4-yl-benzyl)-acetamide hydrochloride (0.15 g, 0.44 mmol), and EDCI (0.097 g, 0.50 mmol) in CH₂Cl₂ (50 mL) is added Et₃N (0.14 mL, 1.0 mmol). The reaction mixture is stirred at r.t. overnight. The reaction mixture is then poured into EtOAc and the organic layer is washed with sat NH₄Cl, water and brine. The organic layer is dried over MgSO₄, filtered and concentrated in vacuo to give the crude product. Purification by flash chromatography on SiO₂ eluting with 50% ethyl acetate/heptane affords the desired product (0.097 g, 49%).

¹H NMR (300 MHz, CD₃OD) δ 8.0 (s, 1H), 7.5 (s, 1H), 7.3 (d, 1H), 7.2-7.1 (m, 3H), 6.95 (t, 1H), 6.8 (t, 1H), 4.6 (t, 2H), 4.4 (m, 4H), 3.2 (m, 2H), 2.0 (m, 2H), 1.8-1.6 (m, 2H), 1.3 (m, 2H), 0.8 (m, 1H);

MS m/z: [M+H]⁺=578.

### G. [4-(5-Aminomethyl-2-fluoro-phenyl)-piperidin-1-yl]-[4-fluoro-1-(2-trifluoromethoxy-ethyl)-1H-indol-3-yl]-methanone hydrochloride

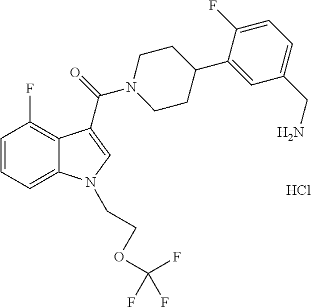

Aqueous K₂CO₃ (0.74 g dissolved in 5 mL water) is added dropwise to a solution of 2,2,2-trifluoro-N-(4-fluoro-3-{1-[4-fluoro-1-(2-trifluoromethoxy-ethyl)-1H-indole-3-carbonyl]-piperidin-4-yl}-benzyl)-acetamide (0.31 g, 0.54 mmol) in MeOH (30 mL). The reaction mixture is stirred at r.t. overnight. The solution is then diluted with water (200 mL) and extracted with EtOAc (2×100 mL). The organic layers are washed with brine, dried over Na₂SO₄, filtered and concentrated in vacuo. The residue is dissolved in Et₂O (10 mL) and 1N HCl (0.60 mL) is added. The precipitate is filtered, washed with ether and dried under vacuum to yield the titled product (0.21 g, 80%).

¹H NMR (300 MHz, CD₃OD) δ 7.7 (s, 1H), 7.5 (d, 1H), 7.4-7.1 (m, 4H), 7.0-6.9 (m, 1H), 4.7 (t, 2H), 4.6-4.5 (m, 1H), 4.3 (t, 2H), 4.1 (m, 2H), 3.8 (m, 3H), 3.2 (m, 2H), 2.1-1.8 (m, 3H);

MS m/z: [M+H]⁺=483.

### Example 91

### 3-[4-(5-Aminomethyl-2-fluoro-phenyl)-piperidine-1-carbonyl]-1-(2-methoxy-ethyl)-1H-indol-4-carboxylic acid dimethylamide hydrochloride

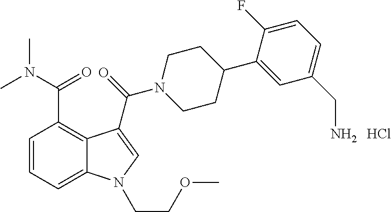

### A. 1-(2-methoxy-ethyl)-1H-indole-4-carboxylic acid methyl ester

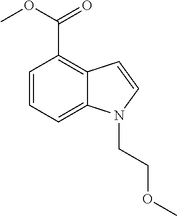

The title compound is prepared in a similar manner as described in Example 1E using 1H-indole-4-carboxylic acid methyl ester as the starting material.

¹H NMR (300 MHz, CDCl₃) δ 7.9 (d, 1H), 7.6 (d, 1H), 7.2 (m, 2H), 7.1 (m, 1H), 4.4 (t, 2H), 4.0 (s, 3H), 3.7 (t, 2H), 3.3 (s, 3H);

MS m/z: [M+H]⁺=234.

### B. 1-(2-Methoxy-ethyl)-3-(2,2,2-trifluoro-acetyl)-1H-indole-4-carboxylic acid methyl ester

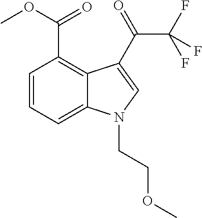

The title compound is prepared in a similar manner as described in Example 1F using 1-(2-methoxy-ethyl)-1H-indole-4-carboxylic acid methyl ester as the starting material.

¹H NMR (300 MHz, CDCl₃) δ 8.1 (s, 1H), 7.6 (m, 2H), 7.3 (m, 1H), 4.4 (t, 2H), 4.0 (s, 3H), 3.8 (t, 2H), 3.3 (s, 3H);

MS m/z: [M+H]⁺=330.

### C. 1-(2-Methoxy-ethyl)-3-(2,2,2-trifluoro-acetyl)-1H-indole-4-carboxylic acid

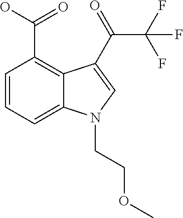

The title compound is prepared in a similar manner as described in Example 5D using 1-(2-methoxy-ethyl)-3-(2,2,2-trifluoro-acetyl)-1H-indole-4-carboxylic acid methyl ester as the starting material.

¹H NMR (300 MHz, DMSO-d6) δ 12.8 (s, 1H), 8.5 (s, 1H), 7.9 (d, 1H), 7.5 (m, 2H), 4.6 (t, 2H), 3.7 (t, 2H), 3.2 (s, 3H);

MS m/z: [M+H]⁺=316.

### D. 1-(2-Methoxy-ethyl)-3-(2,2,2-trifluoro-acetyl)-1H-indole-4-carboxylic acid dimethylamide

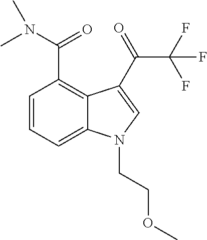

The title compound is prepared in a similar manner as described in Example 59A using 1-(2-methoxy-ethyl)-3-(2,2,2-trifluoro-acetyl)-1H-indole-4-carboxylic acid and dimethylamine as the starting materials.

¹H NMR (300 MHz, CDCl₃) δ 8.1 (s, 1H), 7.4 (m, 2H), 7.2 (m, 1H), 4.4 (t, 2H), 3.8 (t, 2H), 3.4 (s, 3H), 3.3 (s, 3H), 2.8 (s, 3H).

MS m/z: [M+H]⁺=343.

### E. 4-Dimethylcarbamoyl-1-(2-methoxy-ethyl)-1H-indole-3-carboxylic acid

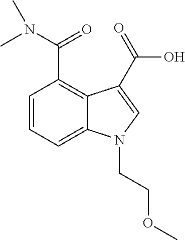

The title compound is prepared in a similar manner as described in Example 4C using 1-(2-methoxy-ethyl)-3-(2,2,2-trifluoro-acetyl)-1H-indole-4-carboxylic acid dimethylamide as the starting material.

¹H NMR (300 MHz, DMSO-d6) δ 11.9 (bs, 1H), 8.0 (s, 1H), 7.6 (d, 1H), 7.3 (m, 1H), 7.0 (d, 1H), 4.4 (t, 2H), 3.8 (t, 2H), 3.3 (s, 3H), 3.2 (s, 3H), 3.0 (s, 3H).

LCMS m/z: [M+H]⁺=291.

### F. 3-(4-{2-Fluoro-5[(2,2,2-trifluoro-acetylamino)-methyl]-phenyl}-piperidine-1-carbonyl)-1-(2-methoxy-ethyl)-1H-indole-4-carboxylic acid dimethylamide

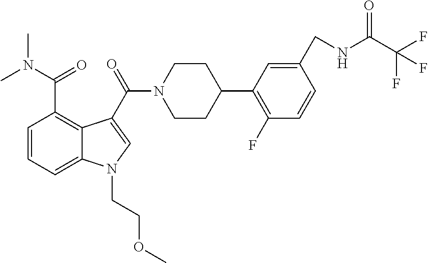

The title compound is prepared in a similar manner as described in Example 2I using 4-dimethylcarbamoyl-1-(2-methoxy-ethyl)-1H-indole-3-carboxylic acid and 2,2,2-trifluoro-N-(4-fluoro-3-piperidin-4-yl-benzyl)acetamide hydrochloride as the starting materials.

¹H NMR (300 MHz, CDCl₃) δ 7.6 (m, 1H), 7.4 (m, H), 7.2 (m, 2H), 7.0 (m, 2H), 4.5 (m, 2H), 4.3 (t, 2H), 3.7 (t, 2H), 3.3 (s, 3H), 3.2 (m, 3H), 3.1 (s, 3H), 3.0 (m, 2H), 2.9 (s, 3H), 1.9-1.7 (m, 4H).

LCMS m/z: [M+H]⁺=577.

### G. 3-[4-(5-Aminomethyl-2-fluoro-phenyl)-piperidine-1-carbonyl]-1-(2-methoxy-ethyl)-1H-indole-4-carboxylic acid dimethylamide hydrochloride

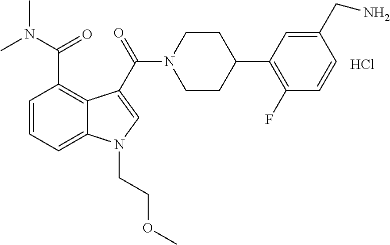

The title compound is prepared in a similar manner as described in Example 1K using 3-(4-{2-fluoro-5[(2,2,2-trifluoro-acetylamino)-methyl]-phenyl}-piperidine-1-carbonyl)-1-(2-methoxy-ethyl)-1H-indole-4-carboxylic acid dimethylamide as the starting material.

¹H NMR (300 MHz, DMSO-d6) δ 8.3 (bs, 2H), 7.6 (m, 3H), 7.4 (m, 1H), 7.2 (m, 2H), 7.0 (m, 1H), 4.4 (m, 2H), 4.2 (bs, 1H), 4.0 (m, 2H), 3.7 (m, 2H), 3.4 (m, 2H), 3.3 (2, 3H), 3.1 (m, 2H), 3.0 (s, 3H), 2.9 (s, 3H), 1.8-1.6 (m, 4H).

MS m/z: [M+H]⁺=481.

### Example 92

### [4-(5-Aminomethyl-2-fluoro-phenyl)-piperidin-1-yl]-[4-pyrimidin-5-yl-1-(2-trifluoromethoxy-ethyl)-1H-indol-3-yl]-methanone dihydrochloride

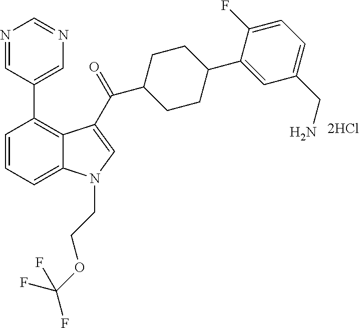

### A. 2,2,2-Trifluoro-N-(4-fluoro-3-{1-[4-pyrimidin-5-yl-1-(2-trifluoromethoxy-ethyl)-1H-indole-3-carbonyl]-piperidin-4-yl}-benzyl)-acetamide

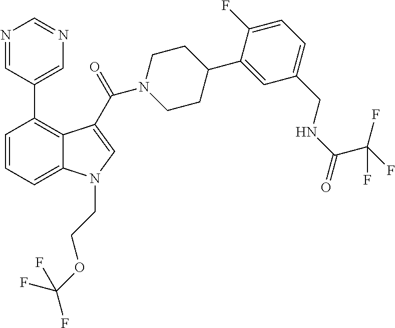

A mixture of N-(3-{1-[4-bromo-1-(2-trifluoromethoxy-ethyl)-1H-indole-3-carbonyl]-piperidin-4-yl}-4-fluoro-benzyl)-2,2,2-trifluoro-acetamide (entry 78D) (200 mg, 0.34 mmol), 5-pyrimidylboronic acid (46 mg, 0.37 mmol), cesium carbonate (204 mg, 0.62 mmol), and Pd(dppf)Cl₂.CH₂Cl₂ (26 mg, 10% mol) in dioxane/H₂O (9 mL/1 mL) is heated at 80° C. for 18 h. The reaction mixture is concentrated in vacuo to remove the solvent. The crude material is purified on silica gel with CH₂Cl₂/MeOH (100/0 to 98/2) as eluent to give the product (188 mg, 92%) as a white foam.

¹H NMR (300 MHz, CDCl₃) δ 9.23 (s, 1H), 8.95 (s, 2H), 7.60-6.80 (m, 8H), 4.80-4.20 (m, 7H), 3.70-3.40 (m, 1H), 3.10-2.40 (m, 3H), 1.80-0.80 (m, 4H);

LC Rt 0.97 min; MS 638 (M+H, 100%).

### B. [4-(5-Aminomethyl-2-fluoro-phenyl)-piperidin-1-yl]-[4-pyrimidin-5-yl-1-(2-trifluoromethoxy-ethyl)-1H-indol-3-yl]-methanone dihydrochloride

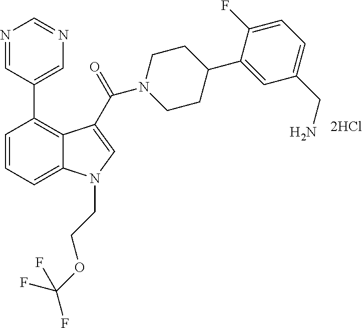

To a mixture of 2,2,2-trifluoro-N-(4-fluoro-3-{1-[4-pyrimidin-5-yl-1-(2-trifluoromethoxy-ethyl)-1H-indole-3-carbonyl]-piperidin-4-yl}-benzyl)-acetamide (185 mg, 0.29 mmol) in MeOH (5 mL) is added aqueous K₂CO₃ (320 mg, 2.3 mmol, dissolved in 1.0 mL H₂O). This mixture is heated at 45° C. for 4 h. LC/MS indicates the reaction is completed. The reaction mixture is concentrated in vacuo to remove most of the methanol. The residue is partitioned between H₂O and CH₂Cl₂. The two layers are separated, and the organic layer is washed with H2O and brine, dried over Na₂SO₄, filtered, and concentrated in vacuo. 4.0 M HCl in dioxane is added. The crude material is purified by RP-HPLC to give a whilte fluffy solid as the titled product (126 mg, 70%).

¹H NMR (300 MHz, DMSO-d6) δ 9.22 (s, 1H), 8.85 (s, 2H), 8.11 (bs, 4H), 7.80-7.60 (m, 2H), 7.40-7.05 (m, 4H), 4.80-3.70 (m, 8H), 3.00-2.00 (m, 3H), 1.80-1.00 (m, 4H);

LC 0.68 min; MS 542 (M+H, 100%).

### Example 93

### [4-(5-Aminomethyl-2-fluoro-phenyl)-piperidin-1-yl]-[4-(1-propyl-1H-pyrazol-4-yl)-1-(2-trifluoromethoxy-ethyl)-1H-indol-3-yl]-methanone dihydrochloride

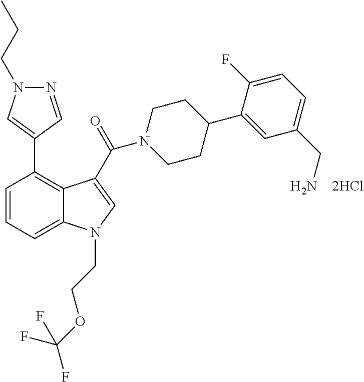

### A. 2,2,2-Trifluoro-N-(4-fluoro-3-{1-[4-(1-propyl-1H-pyrazol-4-yl)-1-(2-trifluoromethoxy-ethyl)-1H-indole-3-carbonyl]-piperidin-4-yl}-benzyl)-acetamide

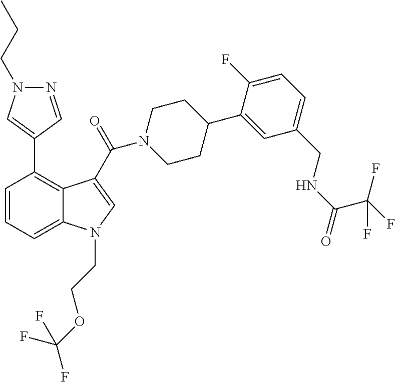

A mixture of N-(3-{1-[4-bromo-1-(2-trifluoromethoxy-ethyl)-1H-indole-3-carbonyl]-piperidin-4-yl}-4-fluoro-benzyl)-2,2,2-trifluoro-acetamide (entry 78D) (200 mg, 0.34 mmol), 1-propylpyrazol-4-boronic acid (58 mg, 0.37 mmol), cesium carbonate (204 mg, 0.62 mmol), and Pd(dppf)Cl₂.CH₂Cl₂ (26 mg, 10% mol) in dioxane/H₂O (9 mL/1 mL) is heated at 80° C. for 5 h. The reaction mixture is concentrated in vacuo to remove the solvent. The crude material is purified on silica gel with CH₂Cl₂/MeOH (100/0 to 98/2) as eluent to give the product (137 mg, 92%) as a beige foam.

¹H NMR (300 MHz, CDCl₃) δ 9.20-9.05 (m, 1H), 8.00-6.80 (m, 9H), 4.90-4.00 (m, 9H), 3.50-2.20 (m, 4H), 2.00-0.80 (m, 9H);

LC Rt 1.05 min; MS 667 (M+H, 100%).

### B. [4-(5-Aminomethyl-2-fluoro-phenyl)-piperidin-1-yl]-[4-(1-propyl-1H-pyrazol-4-yl)-1-(2-trifluoromethoxy-ethyl)-1H-indol-3-yl]-methanone dihydrochloride

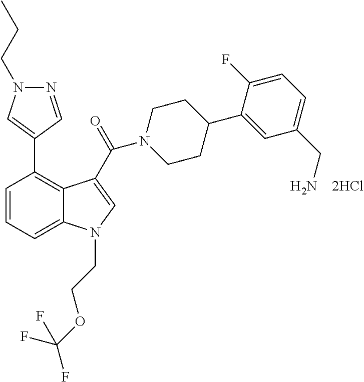

To a mixture of 2,2,2-trifluoro-N-(4-fluoro-3-{1-[4-(1-propyl-1H-pyrazol-4-yl)-1-(2-trifluoromethoxy-ethyl)-1H-indole-3-carbonyl]-piperidin-4-yl}-benzyl)-acetamide (135 mg, 0.20 mmol) in MeOH (5 mL) is added aqueous K₂CO₃ (233 mg, 1.6 mmol, dissolved in 1.0 mL H₂O). This mixture is heated at 45° C. for 4 h. LC/MS indicates the reaction is completed. The reaction mixture is concentrated in vacuo to remove most of the methanol. The residue is partitioned between H₂O and CH₂Cl₂. The two layers are separated, and the organic layer is washed with H2O and brine, dried over Na₂SO₄, filtered, and concentrated in vacuo. 4.0 M HCl in dioxane is added. The crude material is purified by RP-HPLC to give a whilte fluffy solid as the titled product (586 mg, 44%).

¹H NMR (300 MHz, DMSO-d6) δ 8.09 (bs, 4H), 7.90-7.00 (m, 9H), 5.00-4.50 (m, 5H), 4.30-3.75 (m, 4H), 3.20-3.00 (m, 1H), 2.90-2.00 (m, 3H), 2.00-0.80 (m, 9H);

LC 0.75 min; MS 572 (M+H, 100%).

### Example 94

### [4-(5-Aminomethyl-2-fluoro-phenyl)-piperidine-1-yl]-[1-(2-methoxy-ethyl)-4-(piperidine-1-carbonyl)-1H-indol-3-yl]-methanone hydrochloride

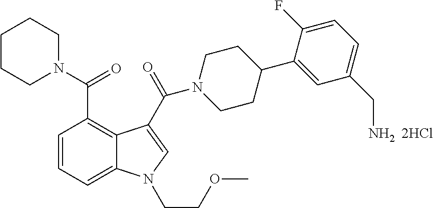

### A. 2,2,2-Trifluoro-1-[1-(2-methoxy-ethyl)-4-(piperidine-1-carbonyl)-1H-indol-3-yl]-ethanone

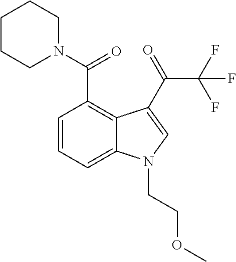

The title compound is prepared in a similar manner as described in Example 59A using 1-(2-methoxy-ethyl)-3-(2,2,2-trifluoro-acetyl)-1H-indole-4-carboxylic acid (example 91C) and piperidine as the starting materials.

¹H NMR (300 MHz, CDCl₃) δ 8.1 (s, 1H), 7.4 (m, 2H), 7.2 (m, 1H), 4.4 (t, 2H), 4.1 (m, 1H), 3.8 (t, 2H), 3.7 (m, 1H), 3.4 (s, 3H), 3.3 (m, 1H), 3.1 (m, 1H), 1.9 (m, 1H), 1.8-1.6 (m, 3H), 1.4 (m, 1H).

MS m/z: [M+H]⁺=383.

### B. 1-(2-Methoxy-ethyl)-4-(piperidine-1-carbonyl)-1H-indol-3-carboxylic acid

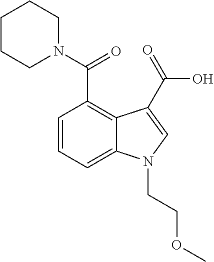

The title compound is prepared in a similar manner as described in Example 4C using 2,2,2-trifluoro-1-[1-(2-methoxy-ethyl)-4-(piperidine-1-carbonyl)-1H-indol-3-yl]-ethanone as the starting material.

¹H NMR (300 MHz, DMSO-d6) δ 12.0 (bs, 1H), 8.0 (s, 1H), 7.6 (d, 1H), 7.3 (m, 1H), 7.0 (d, 1H), 4.4 (t, 2H), 3.8 (m, 1H), 3.7 (t, 2H), 3.4 (m, 1H), 3.2 (s, 3H), 3.1 (m, 1H), 2.9 (m, 1H), 1.8 (m, 1H), 1.6 (m, 1H), 1.5 (m, 3H), 1.3 (m, 1H).

MS m/z: [M+H]⁺=331.

### C. (2,2,2-Trifluoro-N-(4-fluoro-3-{1-[1-(2-methoxy-ethyl)-4(piperidine-1-carbonyl)-1H-indole-3-carbonyl]-piperidine-4-yl}-benzyl)-acetamide

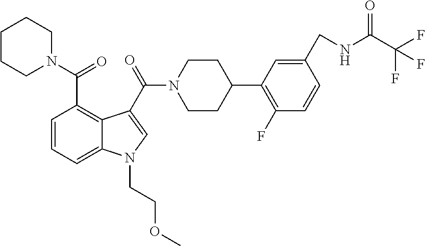

The title compound is prepared in a similar manner as described in Example 2I using 1-(2-methoxy-ethyl)-4-(piperidine-1-carbonyl)-1H-indol-3-carboxylic acid and 2,2,2-trifluoro-N-(4-fluoro-3-piperidin-4-yl-benzyl)acetamide hydrochloride as the starting materials.

¹H NMR (300 MHz, CDCl₃) δ 7.4 (m, 1H), 7.3 (m, 1H), 7.2-7.0 (m, 5H), 6.7 (bs, 1H), 4.8 (m, 1H), 4.5 (m, 3H), 4.3 (t, 2H), 4.0 (m, 1H), 3.7 (t, 2H), 3.4 (s, 3H), 3.3-3.0 (m, 5H), 2.6 (m, 1H), 2.0-1.8 (m, 2H), 1.8-1.5 (m, 8H).

MS m/z: [M+H]⁺=617.

### D. [4-(5-Aminomethyl-2-fluoro-phenyl)-piperidine-1-yl]-[1-(2-methoxy-ethyl)-4-(piperidine-1-carbonyl)-1H-indol-3-yl]-methanone hydrochloride

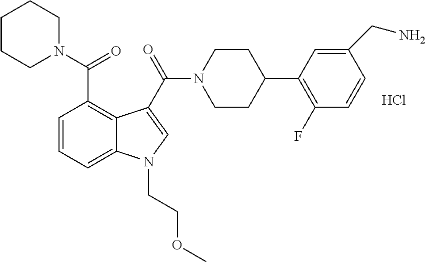

The title compound is prepared in a similar manner as described in Example 1K using (2,2,2-trifluoro-N-(4-fluoro-3-{1-[1-(2-methoxy-ethyl)-4(piperdine-1-carbonyl)-1H-indole-3-carbonyl]-piperidine-4-yl}-benzyl)-acetamide as the starting material.

¹H NMR (300 MHz, DMSO-d6) δ 8.4 (bs, 2H), 7.6 (m, 3H), 7.4 (m, 1H), 7.2 (m, 2H), 7.0 (m, 1H), 4.4 (m, 2H), 4.2-4.0 (m, 5H), 3.7 (t, 2H), 3.6 (m, 2H), 3.3 (s, 3H), 3.2-2.9 (m, 6H), 1.8-1.4 (m, 10H).

MS m/z: [M+H]⁺=521.

### Example 95

Tetrahydro-pyran-4-carboxylic acid [3-[4-(5-aminomethyl-2-fluoro-phenyl)-piperidine-1-carbonyl]-1-(2-methoxy-ethyl)-1H-indol-4-yl]-amide hydrochloride

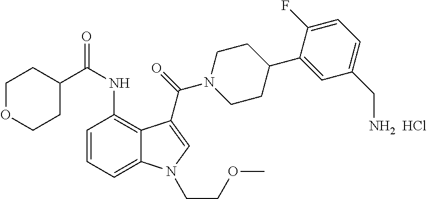

### A. (Tetrahydro-pyran-4-carboxylic acid [3-(4-{2-fluoro-5-[(2,2,2-trifluoroacetylamino)methyl]-phenyl}-piperidine-1-carbonyl)-1-(2-methoxy-ethyl)-1H-indol-4-yl]amide

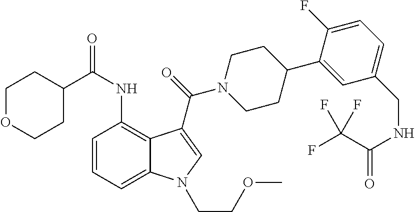

The title compound is prepared in a similar manner as described in Example 6E using tetrahydropyran-4-carboxylic acid and N-(3-{1-[4-amino-1-(2-methoxy-ethyl)-1H-indole-3-carbonyl]-piperidin-4-yl}-4-fluoro-benzyl)-2,2,2-trifluoro-acetamide (example 75E) as the starting materials.

¹H NMR (300 MHz, DMSO-d6) δ 10.80 (s, 1H), 9.95 (t, 1H), 7.92 (d, 1H), 7.80 (s, 1H), 7.33-7.15 (m, 5H), 4.60 (d, 2H), 4.40 (t, 2H), 4.35 (d, 2H), 3.92-3.85 (m, 2H), 3.66 (t, 2H), 3.41-3.33 (m, 3H), 3.25-3.13 (m, 6H), 1.89-1.63 (m, 8H).

### B. Tetrahydro-pyran-4-carboxylic acid [3-[4-(5-aminomethyl-2-fluoro-phenyl)-piperidine-1-carbonyl]-1-(2-methoxy-ethyl)-1H-indol-4-yl]-amide hydrochloride

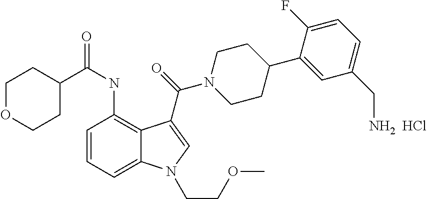

The title compound is prepared in a similar manner as described in Example 3B with (tetrahydro-pyran-4-carboxylic acid [3-(4-{2-fluoro-5-[(2,2,2-trifluoroacetylamino)methyl]-phenyl}-piperidine-1-carbonyl)-1-(2-methoxy-ethyl)-1H-indol-4-yl]amide as the starting material.

¹H NMR (300 MHz, DMSO-d6) δ 10.82 (s, 1H), 8.35 (br s, 3H), 7.94 (d, 1H), 7.81 (s, 1H), 7.56-7.51 (m, 1H), 7.42-7.36 (m, 1H), 7.34-7.31 (m, 1H), 7.27-7.17 (m, 2H), 4.60 (d, 2H), 4.41 (t, 2H), 4.02-3.95 (m, 2H), 3.92-3.85 (m, 2H), 3.66 (t, 2H), 3.42-3.34 (m, 3H), 3.21 (br s, 6H), 1.89-1.65 (m, 8H).

MS m/z: [M+H]⁺=537.

### Example 96

### N-[3-[4-(5-Aminomethyl-2-fluoro-phenyl)-piperidine-1-carbonyl]-1-(2-methoxy-ethyl)-1H-indol-4-yl]-acetamide hydrochloride

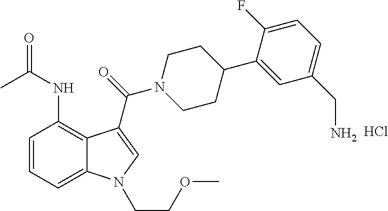

### A. N-(3-{1-[4-Acetylamino-1-(2-methoxy-ethyl)-1H-indole-3-carbonyl]-piperidin-4-yl}-4-fluoro-benzyl)-2,2,2-trifluoro-acetamide

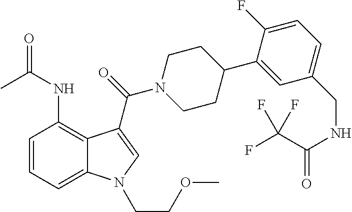

To a solution of acetic acid (29 L, 0.51 mmol), N-(3-{1-[4-amino-1-(2-methoxy-ethyl)-1H-indole-3-carbonyl]-piperidin-4-yl}-4-fluoro-benzyl)-2,2,2-trifluoro-acetamide (264 mg, 0.51 mmol) and EDCI (117 mg, 0.61 mmol) in CH₂Cl₂ (5 mL) is added triethylamine (170 μL, 1.2 mmol). The resulting mixtue is stirred under N₂ at r.t. overnight. The reaction mixture was then diluted with CH₂Cl₂ and washed with brine. The organic layer is dried over MgSO₄, filtered, and concentrated in vacuo. The crude material is purified on silica gel with 3% MeOH/CH₂Cl₂ as eluent to give the title product as a foamy white solid (240 mg, 84%).

¹H NMR (300 MHz, CDCl₃) δ 10.50 (s, 1H), 8.02 (d, 1H), 7.37-7.00 (m, 6H), 4.75-4.62 (br d, 2H), 4.46 (d, J=5.4 Hz, 2H), 4.30 (t, J=5.1 Hz, 2H), 3.69 (t, J=5.1 Hz, 2H), 3.30 (s, 3H), 3.20-3.00 (m, 4H), 2.17 (s, 3H), 1.95-1.80 (m, 2H), 1.80-1.63 (m, 2H).

¹⁹F NMR (300 MHz, CDCl₃) δ −75.42.

MS m/z: [M+H]⁺=563.

### B. N-[3-[4-(5-Aminomethyl-2-fluoro-phenyl)-piperidine-1-carbonyl]-1-(2-methoxy-ethyl)-1H-indol-4-yl]-acetamide hydrochloride

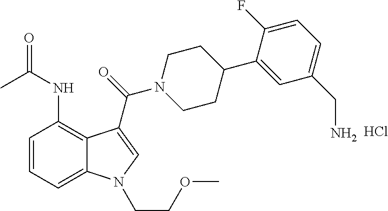

The title compound is prepared in a similar manner as described in Example 1K using N-(3-{1-[4-acetylamino-1-(2-methoxy-ethyl)-1H-indole-3-carbonyl]-piperidin-4-yl}-4-fluoro-benzyl)-2,2,2-trifluoro-acetamide as the starting material.

¹H NMR (300 MHz, DMSO-d6) δ 11.04 (s, 1H), 8.51 (br s, 2H), 7.94 (d, 1H), 7.84 (s, 1H), 7.63 (d, 1H), 7.40 (m, 1H), 7.30 (d, 1H), 7.21 (m, 2H), 4.60 (br d, 2H), 4.42 (t, 2H), 3.99 (m, 2H), 3.67 (t, 2H), 3.40 (s, 3H), 3.22 (br m, 3H), 2.07 (s, 3H), 1.89-1.65 (m, 4H).

¹⁹F NMR (300 MHz, DMSO-d6) δ −119.86.

MS m/z: [M+H]⁺=467.

### Example 97

### 1-Methyl-piperidine-4-carboxylic acid [3-[4-(5-aminomethyl-2-fluoro-phenyl)-piperidine-1-carbonyl]-1-(2-methoxy-ethyl)-1H-indol-4-yl]-amide hydrochloride

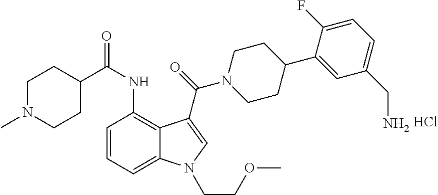

### A. 1-Methyl-piperidine-4-carboxylic acid [3-(4-{2-fluoro-5-[(2,2,2-trifluoro-acetylamino)-methyl]-phenyl}-piperidine-1-carbonyl)-1-(2-methoxy-ethyl)-1H-indol-4-yl]-amide

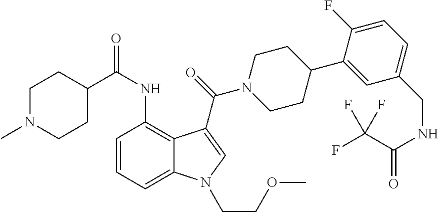

The title compound is prepared in a similar manner as described in Example 6E using N-methylisonipecotic acid and N-(3-{1-[4-amino-1-(2-methoxy-ethyl)-1H-indole-3-carbonyl]-piperidin-4-yl}-4-fluoro-benzyl)-2,2,2-trifluoro-acetamide as the starting materials.

¹H NMR (300 MHz, DMSO-d6) δ 10.81 (s, 1H), 9.96 (t, 1H), 7.92 (d, 1H), 7.79 (s, 1H), 7.33-7.30 (m, 1H), 7.27 (d, 1H), 7.22-7.15 (m, 3H), 4.60 (d, 2H), 4.40 (t, 2H), 4.35 (d, 2H), 3.66 (t, 2H), 3.32 (br s, 2H), 3.18-3.16 (m, 4H), 2.89 (br s, 2H), 2.24 (br s, 4H), 2.09 (br s, 2H), 1.90-1.65 (m, 8H).

### B. 1-Methyl-piperidine-4-carboxylic acid [3-[4-(5-aminomethyl-2-fluoro-phenyl)-piperidine-1-carbonyl]-1-(2-methoxy-ethyl)-1H-indol-4-yl]-amide hydrochloride

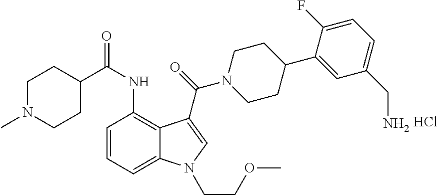

The title compound is prepared in a similar manner as described in Example 3B with 1-methyl-piperidine-4-carboxylic acid [3-(4-{2-fluoro-5-[(2,2,2-trifluoro-acetylamino)-methyl]-phenyl}-piperidine-1-carbonyl)-1-(2-methoxy-ethyl)-1H-indol-4-yl]-amide as the starting material.

¹H NMR (300 MHz, DMSO-d6) δ 10.97 (s, 1H), 8.84 (br s, 3H), 7.93 (d, 1H), 7.84 (s, 1H), 7.64-7.62 (m, 1H), 7.41-7.33 (m, 2H), 7.26-7.18 (m, 2H), 4.61 (d, 2H), 4.42 (t, 2H), 4.01 (s, 2H), 3.66 (t, 2H), 3.35-3.25 (br s, 4H), 3.21 (s, 4H), 2.95 (br t, 2H), 2.66 (s, 3H), 2.55 (br s, 1H), 2.12-1.96 (m, 4H), 1.89-1.69 (m, 4H).

MS m/z: [M+H]⁺=550.

### Example 98

Bicyclo[2.2.1]heptane-2-carboxylic acid [3-[4-(5-aminomethyl-2-fluoro-phenyl)-piperidine-1-carbonyl]-1-(2-methoxy-ethyl)-1H-indol-4-yl]-amide hydrochloride

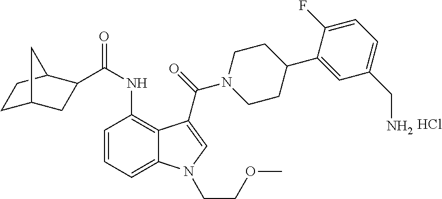

### A. Bicyclo[2.2.1]heptane-2-carboxylic acid [3-(4-{2-fluoro-5-[(2,2,2-trifluoro-acetylamino)-methyl]-phenyl}-piperidine-1-carbonyl)-1-(2-methoxy-ethyl)-1H-indol-4-yl]-amide

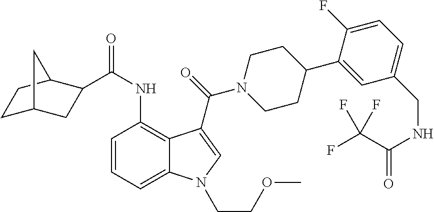

The title compound is prepared in a similar manner as described in Example 6E using norbornane-2-carboxylic acid (predominantly endo isomer) and N-(3-{1-[4-amino-1-(2-methoxy-ethyl)-1H-indole-3-carbonyl]-piperidin-4-yl}-4-fluoro-benzyl)-2,2,2-trifluoro-acetamide as the starting materials.

¹H NMR (300 MHz, DMSO-d6) δ 10.67 (s, 1H), 9.96 (br s, 1H), 7.96 (d, 1H), 7.78 (s, 1H), 7.31-7.25 (m, 2H), 7.21-7.15 (m, 3H), 4.62 (br s, 2H), 4.40 (t, 2H), 4.35 (d, 2H), 3.66 (t, 2H), 3.22 (s, 3H), 3.21-3.14 (br s, 3H), 2.87-2.81 (m, 1H), 2.70 (br s, 1H), 2.36-2.31 (m, 1H), 2.25-2.20 (m, 1H), 1.85 (br s, 2H), 1.75-1.55 (m, 4H), 1.48-1.39 (m, 3H), 1.35-1.24 (m, 2H).

### B. Bicyclo[2.2.1]heptane-2-carboxylic acid [3-[4-(5-aminomethyl-2-fluoro-phenyl)-piperidine-1-carbonyl]-1-(2-methoxy-ethyl)-1H-indol-4-yl]-amide hydrochloride

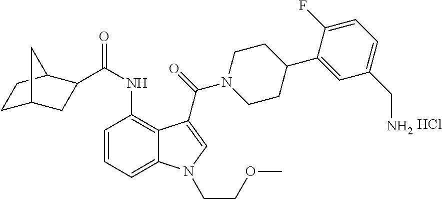

The title compound is prepared in a similar manner as described in Example 3B with bicyclo[2.2.1]heptane-2-carboxylic acid [3-(4-{2-fluoro-5-[(2,2,2-trifluoro-acetylamino)-methyl]-phenyl}-piperidine-1-carbonyl)-1-(2-methoxy-ethyl)-1H-indol-4-yl]-amide as the starting material.

¹H NMR (300 MHz, DMSO-d6) δ 10.71 (s, 1H), 8.42 (br s, 3H), 7.96 (d, 1H), 7.80 (s, 1H), 7.57-7.56 (m, 1H), 7.43-7.39 (m, 2H), 7.28 (t, 1H), 7.23-7.16 (m, 1H), 4.62 (d, 2H), 4.41 (t, 2H), 3.98 (br s, 2H), 3.76 (br s, 1H), 3.66 (t, 2H), 3.21 (br s, 6H), 2.86-2.80 (m, 1H), 2.70 (br s, 1H), 2.24 (br s, 1H), 1.90-1.71 (m, 5H), 1.48-1.15 (m, 6H).

MS m/z: [M+H]⁺=547.

### Example 99

### Morpholine-4-carboxylic acid [3-[4-(5-aminomethyl-2-fluoro-phenyl)-piperidine-1-carbonyl]-1-(2-methoxy-ethyl)-1H-indol-4-yl]-amide hydrochloride

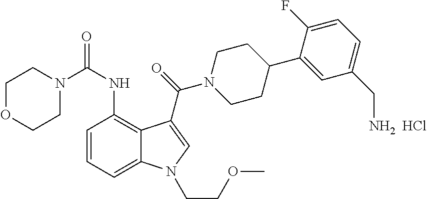

### A. Morpholine-4-carboxylic acid [3-(4-{2-fluoro-5-[(2,2,2-trifluoro-acetylamino)-methyl]-phenyl}-piperidine-1-carbonyl)-1-(2-methoxy-ethyl)-1H-indol-4-yl]-amide

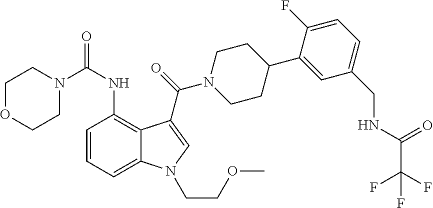

To a solution of N-(3-{1-[4-amino-1-(2-methoxy-ethyl)-1H-indole-3-carbonyl]-piperidin-4-yl}-4-fluoro-benzyl)-2,2,2-trifluoro-acetamide (1.00 g, 1.92 mmol) and Et₃N (0.64 mL, 4.6 mmol) in CH₂Cl₂ (50 mL) is added morpholine-4-carbonyl chloride (0.66 mL, 5.7 mmol). The reaction mixture is stirred at 40° C. for 12 h. The mixture is diluted with CH₂Cl₂ (100 mL) and is washed with water, brine, dried with MgSO₄, filtered and is concentrated in vacuo. The crude residue is flash chromatographed over SiO₂ eluted with 70% EtOAc/heptane to afford the titled compound (0.82 g).

¹H NMR (300 MHz, CDCl₃) δ 9.6 (s, 1H), 7.8 (d, 1H), 7.3 (m, 1H), 7.2-7.0 (m, 5H), 4.7 (m, 2H), 4.4 (m, 2H), 4.3 (m, 2H), 3.8-3.6 (m, 10H), 3.3 (s, 3H), 3.2 (m, 3H), 1.9 (m, 2H), 1.7 (m, 2H), 1.5 (m, 2H).

MS m/z: [M+H]⁺=634.

### B—Morpholine-4-carboxylic acid [3-[4-(5-aminomethyl-2-fluoro-phenyl)-piperidine-1-carbonyl]-1-(2-methoxy-ethyl)-1H-indol-4-yl]-amide hydrochloride

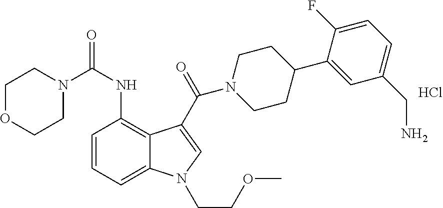

The title compound is prepared in a similar manner as described in Example 3B using N-(3-{1-[4-methylamino-1-(2-methoxy-ethyl)-1H-indole-3-carbonyl]-piperidin-4-yl}-4-fluoro-benzyl)-2,2,2-trifluoro-acetamide as the starting material.

¹H NMR (300 MHz, DMSO-d6) δ 10.0 (s, 1H), 8.2 (bs, 2H), 7.8 (m, 2H), 7.6 (m, 1H), 7.4 (m, 1H), 7.2 (m, 3H), 4.6 (m, 2H), 4.4 (m, 2H), 4.0 (m, 2H), 3.7 (m, 10H), 3.5 (m, 3H), 3.2 (s, 3H), 1.9 (m, 2H), 1.8 (m, 2H).

MS m/z: [M+H]⁺=538

### Example 100

### 3-[3-[4-(5-Aminomethyl-2-fluoro-phenyl)-piperidine-1-carbonyl]-1-(2-methoxy-ethyl)-1H-indol-4-yl]-1,1-dimethyl-urea hydrorchloride

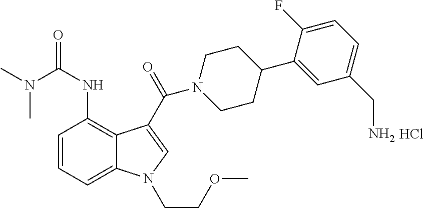

### A. N-(3-{1-[4-(3,3-Dimethyl-ureido)-1-(2-methoxy-ethyl)-1H-indole-3-carbonyl]-piperidin-4-yl}-4-fluoro-benzyl)-2,2,2-trifluoro-acetamide

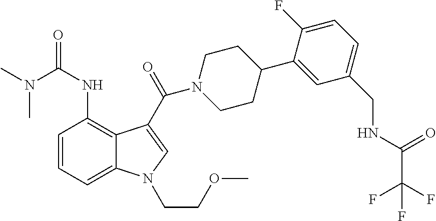

The title compound is prepared in a similar manner as described in Example 99A using N-(3-{1-[4-amino-1-(2-methoxy-ethyl)-1H-indole-3-carbonyl]-piperidin-4-yl}-4-fluoro-benzyl)-2,2,2-trifluoro-acetamide and dimethylcarbamyl chloride as the starting materials.

¹H NMR (300 MHz, CDCl₃) δ 9.4 (s, 1H), 7.8 (d, 1H), 7.3 (m, 1H), 7.2-7.0 (m, 5H), 4.7 (m, 2H), 4.5 (m, 2H), 4.3 (m, 2H), 3.6 (m, 2H), 3.3 (s, 3H), 3.2 (m, 3H), 3.0 (s, 6H), 1.9 (m, 2H), 1.7 (m, 2H).

MS m/z: [M+H]⁺=592.

### B—3-[3-[4-(5-Aminomethyl-2-fluoro-phenyl)-piperidine-1-carbonyl]-1-(2-methoxy-ethyl)-1H-indol-4-yl]-1,1-dimethyl-urea hydrorchloride

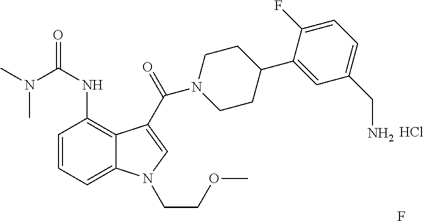

The title compound is prepared in a similar manner as described in Example 3B using N-(3-{1-[4-(3,3-dimethyl-ureido)-1-(2-methoxy-ethyl)-1H-indole-3-carbonyl]-piperidin-4-yl}-4-fluoro-benzyl)-2,2,2-trifluoro-acetamide as the starting material.

¹H NMR (300 MHz, DMSO-d6) δ 9.7 (s, 1H), 8.3 (bs, 2H), 7.8 (d, 1H), 7.8 (m, 1H), 7.6 (m, 1H), 7.4 (m, 1H), 7.3-7.0 (m, 3H), 4.6 (m, 2H), 4.4 (m, 2H), 4.0 (m, 6H), 3.6 (m, 2H), 3.2 (s, 3H), 3.0 (s, 6H), 1.9 (m, 2H), 1.7 (m, 2H).

MS m/z: [M+H]⁺=496.

### Example 101

### 1-N-[3-[4-(5-Aminomethyl-2-fluoro-phenyl)-piperidine-1-carbonyl]-1-(2-methoxy-ethyl)-1H-indol-4-yl]-isonicotinamide hydrochloride

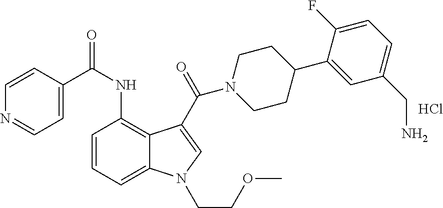

### A. N-[3-(4-{2-Fluoro-5-[(2,2,2-trifluoro-acetylamino)-methyl]-phenyl}-piperidine-1-carbonyl)-1-(2-methoxy-ethyl)-1H-indol-4-yl]-isonicotinamide

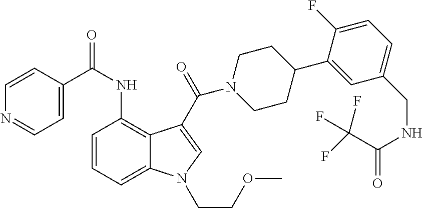

The title compound is prepared in a similar manner as described in Example 6E using isonicotinic acid and N-(3-{1-[4-amino-1-(2-methoxy-ethyl)-1H-indole-3-carbonyl]-piperidin-4-yl}-4-fluoro-benzyl)-2,2,2-trifluoro-acetamide as the starting materials.

¹H NMR (300 MHz, DMSO-d6) δ 11.88 (s, 1H), 9.93 (t, 1H), 8.82-8.80 (m, 2H), 8.19 (d, 1H), 8.00-7.98 (m, 2H), 7.88 (s, 1H), 7.46-7.43 (m, 1H), 7.39-7.29 (m, 1H), 7.27-7.24 (m, 1H), 7.15 (d, 2H), 4.64 (br d, 2H), 4.45 (t, 2H), 4.33 (d, 2H), 3.69 (t, 2H), 3.23 (s, 3H), 3.17 (br s, 3H), 1.88-1.84 (m, 2H), 1.76-1.69 (m, 2H).

### B. N-[3-[4-(5-Aminomethyl-2-fluoro-phenyl)-piperidine-1-carbonyl]-1-(2-methoxy-ethyl)-1H-indol-4-yl]-isonicotinamide hydrochloride

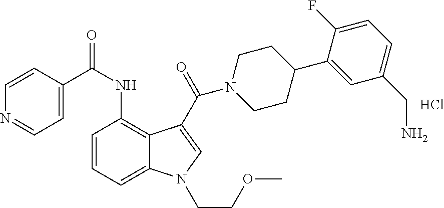

The title compound is prepared in a similar manner as described in Example 3B with N-[3-(4-{2-fluoro-5-[(2,2,2-trifluoro-acetylamino)-methyl]-phenyl}-piperidine-1-carbonyl)-1-(2-methoxy-ethyl)-1H-indol-4-yl]-isonicotinamide as the starting material.

¹H NMR (300 MHz, DMSO-d6) δ12.10 (s, 1H), 8.95 (d, 2H), 8.42 (br s, 3H), 8.22-8.18 (m, 3H), 7.93 (s, 1H), 7.58-7.56 (m, 1H), 7.48-7.45 (m, 1H), 7.41-7.37 (m, 1H), 7.31 (t, 1H), 7.21 (dd, 1H), 4.65 (d, 2H), 4.47 (t, 2H), 3.97-3.95 (m, 2H), 3.70 (t, 2H), 3.23 (br s, 6H), 1.90-1.70 (m, 4H).

MS m/z: [M+H]⁺=496.

### Example 102

### 1-Pyrimidine-5-carboxylic acid [3-[4-(5-aminomethyl-2-fluoro-phenyl)-piperidine-1-carbonyl]-1-(2-methoxy-ethyl)-1H-indol-4-yl]-amide hydrochloride

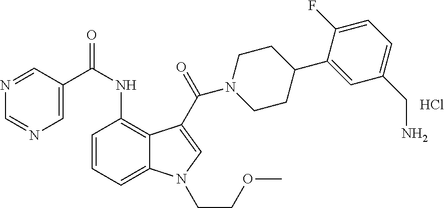

### A. Pyrimidine-5-carboxylic acid [3-(4-{2-fluoro-5-[(2,2,2-trifluoro-acetylamino)-methyl]-phenyl}-piperidine-1-carbonyl)-1-(2-methoxy-ethyl)-1H-indol-4-yl]-amide

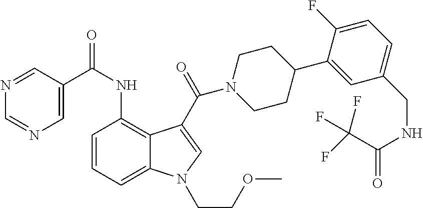

The title compound is prepared in a similar manner as described in Example 6E using pyrimidine-5-carboxylic acid and N-(3-{1-[4-amino-1-(2-methoxy-ethyl)-1H-indole-3-carbonyl]-piperidin-4-yl}-4-fluoro-benzyl)-2,2,2-trifluoro-acetamide as the starting materials.

¹H NMR (300 MHz, DMSO-d6) δ 10.02 (s, 1H), 9.93 (t, 1H), 9.39 (s, 2H), 9.38 (s, 1H), 8.18 (d, 1H), 7.89 (s, 1H), 7.46-7.44 (m, 1H), 7.28 (q, 2H), 7.14 (d, 2H), 4.61 (br d, 2 H), 4.45 (t, 2H), 4.34 (d, 2H), 3.69 (t, 2H), 3.23 (s, 3H), 3.19 (br s, 3H), 1.87-1.62 (m, 4H).

### B. Pyrimidine-5-carboxylic acid [3-[4-(5-aminomethyl-2-fluoro-phenyl)-piperidine-1-carbonyl]-1-(2-methoxy-ethyl)-1H-indol-4-yl]-amide hydrochloride

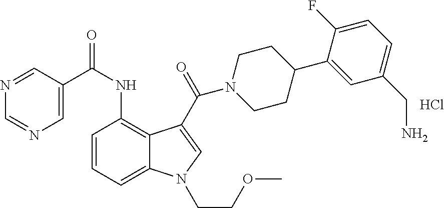

The title compound is prepared in a similar manner as described in Example 3B with pyrimidine-5-carboxylic acid [3-(4-{2-fluoro-5-[(2,2,2-trifluoro-acetylamino)-methyl]-phenyl}-piperidine-1-carbonyl)-1-(2-methoxy-ethyl)-1H-indol-4-yl]-amide as the starting material.

¹H NMR (300 MHz, DMSO-d6) δ 12.05 (s, 1H), 9.39 (s, 3H), 8.39 (br s, 3H), 8.18 (d, 1H), 7.92 (s, 1H), 7.54 (d, 1H), 7.47-7.44 (m, 1H), 7.41-7.38 (m, 1H), 7.30 (t, 1H), 7.25-7.18 (m, 1H), 4.61 (br d, 2H), 4.46 (t, 2H), 3.98-3.96 (m, 2H), 3.70 (t, 2H), 3.23 (s, 6H), 1.88-1.70 (m, 4H).

MS m/z: [M+H]⁺=530.

### Example 103

Isoxazole-5-carboxylic acid [3-[4-(5-aminomethyl-2-fluoro-phenyl)-piperidine-1-carbonyl]-1-(2-methoxy-ethyl)-1H-indol-4-yl]-amide hydrochloride

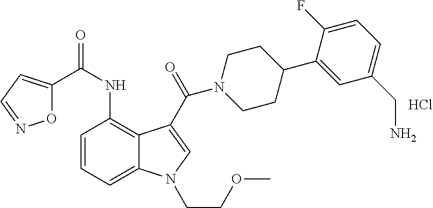

### A. Isoxazole-5-carboxylic acid [3-(4-{2-fluoro-5-[(2,2,2-trifluoro-acetylamino)-methyl]-phenyl}-piperidine-1-carbonyl)-1-(2-methoxy-ethyl)-1H-indol-4-yl]-amide

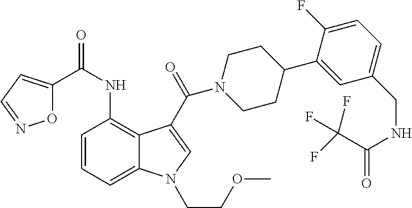

The title compound is prepared in a similar manner as described in Example 96A using isoxazole-5-carboxylic acid and N-(3-{1-[4-amino-1-(2-methoxy-ethyl)-1H-indole-3-carbonyl]-piperidin-4-yl}-4-fluoro-benzyl)-2,2,2-trifluoro-acetamide as the starting materials.

¹H NMR (300 MHz, CDCl₃) δ 11.62 (s, 1H), 8.38 (s, 1H), 8.25 (d, 1H), 7.44 (s, 1H), 7.38 (t, 1H), 7.18 (m, 3H), 7.03 (d, 2H), 4.72 (br d, 2H), 4.44 (m, 2H), 4.32 (t, 2H), 3.71 (t, 2H), 3.31 (s, 3H), 3.20 (m, 1H), 1.90 (m, 2H), 1.70 (m, 1H), 1.53 (m, 4H). MS m/z: [M+H]⁺=616.

### B. Isoxazole-5-carboxylic acid [3-[4-(5-aminomethyl-2-fluoro-phenyl)-piperidine-1-carbonyl]-1-(2-methoxy-ethyl)-1H-indol-4-yl]-amide hydrochloride

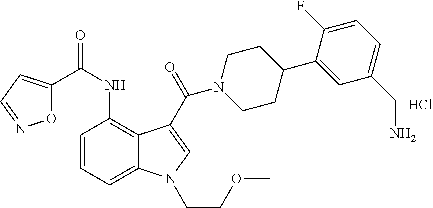

The title compound is prepared in a similar manner as described in Example 1K using isoxazole-5-carboxylic acid [3-(4-{2-fluoro-5-[(2,2,2-trifluoro-acetylamino)-methyl]-phenyl}-piperidine-1-carbonyl)-1-(2-methoxy-ethyl)-1H-indol-4-yl]-amide as the starting material.

¹H NMR (300 MHz, DMSO-d6) δ 10.82 (s, 1H), 8.39 (br s, 2H), 7.80 (m, 1H), 7.60 (m, 1H), 7.40-7.21 (m, 4H), 7.20-7.10 (m, 3H), 4.70 (br s, 2H), 4.45 (br s, 2H), 4.00 (br m, 2H), 3.70 (br s, 2H), 3.50-3.30 (m, 3H), 3.23 (s, 3H), 1.80 (br m, 2H), 1.50 (br m, 2H).

MS m/z: [M+H]⁺=520.

### Example 104

### Dimethylamino-1-sulfonic acid [3-[4-(5-aminomethyl-2-fluoro-phenyl)-piperidine-1-carbonyl]-1-(2-methoxy-ethyl)-1H-indol-4-yl]-amide

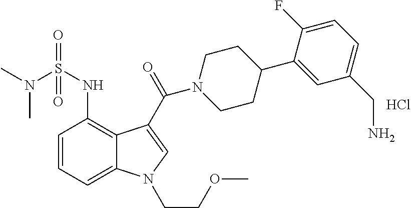

### A. N-(3-{1-4-(Dimethylamino-1-sulfonylamino)-1-(2-methoxy-ethyl)-1H-indole-3-carbonyl}-piperidine-4-yl)-4-fluoro-benzyl)-2,2,2-trifluoro-acetamide

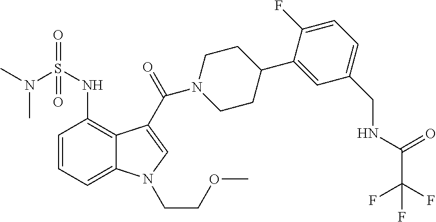

The title compound is prepared in a similar manner as described in Example 99A using N-(3-{1-[4-amino-1-(2-methoxy-ethyl)-1H-indole-3-carbonyl]-piperidin-4-yl}-4-fluoro-benzyl)-2,2,2-trifluoro-acetamide and dimethylsulfamoyl chloride as the starting materials.

¹H NMR (300 MHz, CDCl₃) δ 9.7 (s, 1H), 7.4 (m, 2H), 7.3 (m, 1H), 7.2 (m, 3H), 7.0 (m, 1H), 4.7 (m, 2H), 4.4 (m, 2H), 4.3 (m, 2H), 3.7 (m, 2H), 3.3 (s, 3H), 3.2 (m, 3H), 3.0 (s, 6H), 1.8 (m, 4H).

MS m/z: [M+H]⁺=628.

### B—Dimethylamino-1-sulfonic acid [3-[4-(5-aminomethyl-2-fluoro-phenyl)-piperidine-1-carbonyl]-1-(2-methoxy-ethyl)-1H-indol-4-yl]-amide

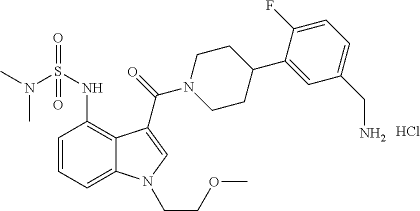

The title compound is prepared in a similar manner as described in Example 3B using N-(3-{1-4-(dimethylamino-1-sulfonylamino)-1-(2-methoxy-ethyl)-1H-indole-3-carbonyl}-piperdine-4-yl)-4-fluoro-benzyl)-2,2,2-trifluoro-acetamide as the starting material.

¹H NMR (300 MHz, DMSO-d6) δ 10.5 (s, 1H), 8.4 (bs, 2H), 7.9 (d, 1H), 7.6 (m, 1H), 7.4 (m, 2H), 7.2 (m, 3H), 4.6 (m, 2H), 4.4 (m, 2H), 4.0 (m, 3H), 3.7 (m, 2H), 3.5 (m, 2H), 3.2 (s, 3H), 2.6 (s, 6H), 1.9 (m, 2H), 1.7 (m, 2H).

MS m/z: [M+H]⁺=532.

### Example 105

### 1-Methyl-1H-imidazole-4-sulfonic acid [3-[4-(5-aminomethyl-2-fluoro-phenyl)-piperidine-1-carbonyl]-1-(2-methoxy-ethyl)-1H-indol-4-yl]-amide hydrochloride

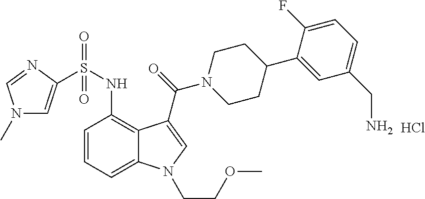

### A. 2,2,2-Trifluoro-N-(4-fluoro-3-{1-[1-(2-methoxy-ethyl)-4-(1-methyl-1H-imidazole-4-sulfonylamino)-1H-indole-3-carbonyl]-piperidin-4-yl}-benzyl)-acetamide

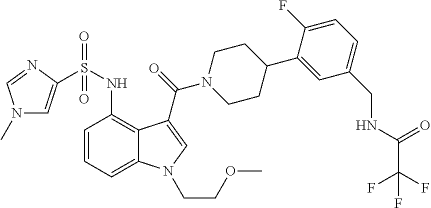

The title compound is prepared in a similar manner as described in Example 99A using N-(3-{1-[4-amino-1-(2-methoxy-ethyl)-1H-indole-3-carbonyl]-piperidin-4-yl}-4-fluoro-benzyl)-2,2,2-trifluoro-acetamide and 1-methyl-1H-imidazole-4-sulfonyl chloride as the starting materials.

¹H NMR (300 MHz, CDCl₃) δ 10.1 (s, 1H), 7.5-7.3 (m, 5H), 7.2 (m, 3H), 7.0 (m, 2H), 4.6 (m, 2H), 4.4 (m, 2H), 4.2 (m, 2H), 3.6 (m, 5H), 3.3 (s, 3H), 3.2 (m, 3H), 1.9 (m, 2H), 1.8 (m, 2H).

MS m/z: [M+H]⁺=665.

### B—1-Methyl-1H-imidazole-4-sulfonic acid [3-[4-(5-aminomethyl-2-fluoro-phenyl)-piperidine-1-carbonyl]-1-(2-methoxy-ethyl)-1H-indol-4-yl]-amide hydrochloride

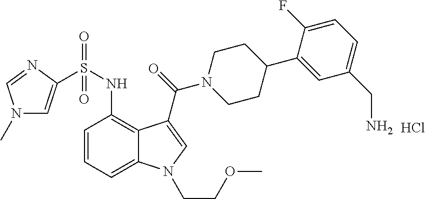

The title compound is prepared in a similar manner as described in Example 3B using 2,2,2-trifluoro-N-(4-fluoro-3-{1-[1-(2-methoxy-ethyl)-4-(1-methyl-1H-imidazole-4-sulfonylamino)-1H-indole-3-carbonyl]-piperidin-4-yl}-benzyl)-acetamide as the starting material.

¹H NMR (300 MHz, DMSO-d6) δ10.9 (s, 1H), 8.4 (bs, 2H), 7.8 (m, 2H), 7.6 (m, 2H), 7.4 (m, 1H), 7.3-7.0 (m, 3H), 4.6 (m, 4H), 4.4 (m, 2H), 4.0 (m, 2H), 3.5 (m, 4H), 3.2 (m, 6H), 2.0-1.7 (m, 4H).

MS m/z: [M+H]⁺=567.

### Example 106

### Pyridine-3-sulfonic acid [3-[4-(5-aminomethyl-2-fluoro-phenyl)-piperidine-1-carbonyl]-1-(2-methoxy-ethyl)-1H-indol-4-yl]-amide hydrochloride

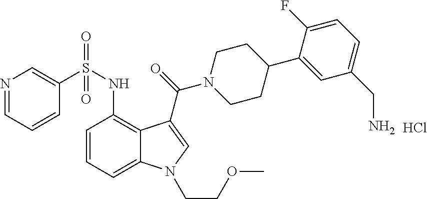

### A. 2,2,2-Trifluoro-N-(4-fluoro-3-{1-[1-(2-methoxy-ethyl)-4-(pyridine-3-sulfonylamino)-1H-indole-3-carbonyl]-piperidin-4-yl}-benzyl)-acetamide

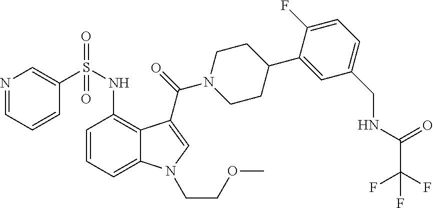

The title compound is prepared in a similar manner as described in Example 99A using N-(3-{1-[4-amino-1-(2-methoxy-ethyl)-1H-indole-3-carbonyl]-piperidin-4-yl}-4-fluoro-benzyl)-2,2,2-trifluoro-acetamide and pyridine-3-sulfonyl chloride as the starting materials.

¹H NMR (300 MHz, CDCl₃) δ 10.8 (s, 1H), 9.1 (s, 1H), 8.6 (m, 1H), 8.0 (m, 1H), 7.4-7.0 (m, 7H), 4.6 (m, 2H), 4.5 (m, 2H), 4.3 (m, 2H), 4.2 (m, 2H), 3.6 (m, 2H), 3.3 (s, 3H), 3.2 (m, 3H), 1.9 (m, 2H), 1.7 (m, 2H).

MS m/z: [M+H]⁺=662.

### B. Pyridine-3-sulfonic acid [3-[4-(5-aminomethyl-2-fluoro-phenyl)-piperidine-1-carbonyl]-1-(2-methoxy-ethyl)-1H-indol-4-yl]-amide hydrochloride

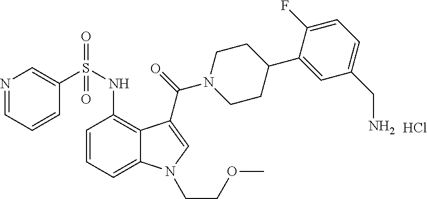

The title compound is prepared in a similar manner as described in Example 3B using 2,2,2-trifluoro-N-(4-fluoro-3-{1-[1-(2-methoxy-ethyl)-4-(pyridine-3-sulfonylamino)-1H-indole-3-carbonyl]-piperidin-4-yl}-benzyl)-acetamide as the starting material.

¹H NMR (300 MHz, DMSO-d6) δ 11.2 (s, 1H), 8.75 (bs, 2H), 8.4 (m, 1H), 7.9 (m, 1H), 7.8 (m, 1H), 7.7 (m, 1H), 7.5 (m, 1H), 7.4 (m, 2H), 7.0 (m, 2H), 4.4 (m, 5H), 4.0 (m, 2H), 3.6 (m, 2H), 3.3 (m, 3H), 3.2 (s, 3H), 1.8 (m, 2H), 1.7 (m, 2H).

MS m/z: [M+H]⁺=566.

### Example 107

### Butane-1-sulfonic acid [3-[4-(5-aminomethyl-2-fluoro-phenyl)-piperidine-1-carbonyl]-1-(2-methoxy-ethyl)-1H-indol-4-yl]-amide hydrochloride

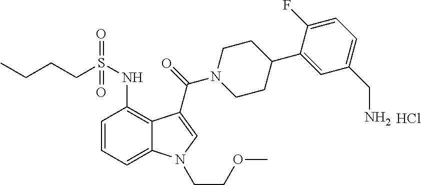

### A. N-(3-{1-[4-(Butane-1-sulfonylamino)-1-(2-methoxy-ethyl)-1H-indole-3-carbonyl]-piperidin-4-yl}-4-fluoro-benzyl)-2,2,2-trifluoro-acetamide

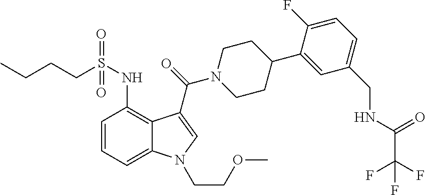

The title compound is prepared in a similar manner as described in Example 99A using N-(3-{1-[4-amino-1-(2-methoxy-ethyl)-1H-indole-3-carbonyl]-piperidin-4-yl}-4-fluoro-benzyl)-2,2,2-trifluoro-acetamide and 1-butanesulfonyl chloride as the starting materials.

¹H NMR (300 MHz, CDCl₃) δ 9.8 (s, 1H), 7.4 (m, 1H), 7.3 (m, 2H), 7.2-7.0 (m, 4H), 4.7 (m, 2H), 4.5 (m, 2H), 4.3 (m, 2H), 4.1 (m, 2H), 3.7 (m, 2H), 3.3 (s, 3H), 3.2 (m, 2H), 3.1 (m, 3H), 2.0-1.8 (m, 4H), 1.6 (m, 2H), 1.4 (m, 2H), 0.9 (m, 3H).

MS m/z: [M+H]⁺=545.

### B. Butane-1-sulfonic acid [3-[4-(5-aminomethyl-2-fluoro-phenyl)-piperidine-1-carbonyl]-1-(2-methoxy-ethyl)-1H-indol-4-yl]-amide hydrochloride

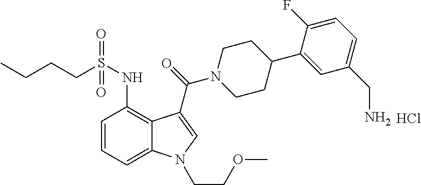

The title compound is prepared in a similar manner as described in Example 3B using N-(3-{1-[4-(butane-1-sulfonylamino)-1-(2-methoxy-ethyl)-1H-indole-3-carbonyl]-piperidin-4-yl}-4-fluoro-benzyl)-2,2,2-trifluoro-acetamide as the starting material.

¹H NMR (300 MHz, DMSO-d6) δ 10.6 (s, 1H), 8.3 (bs, 2H), 7.8 (d, 1H), 7.6 (m, 1H), 7.4 (m, 2H), 7.2 (m, 3H), 4.6 (m, 2H), 4.4 (m, 2H), 4.0 (m, 2H), 3.7 (m, 2H), 3.5 (m, 2H), 3.2 (s, 3H), 3.1 (m, 2H), 1.9 (m, 2H), 1.8 (m, 2H), 1.6 (m, 2H), 1.3 (m, 2H), 0.8 (m, 3H).

MS m/z: [M+H]⁺=641.

### Example 108

### 1-[4-(5-Aminomethyl-2-fluoro-phenyl)-piperidin-1-yl]-[1-(2-methoxy-ethyl)-4-(pyrrolidine-1-carbonyl)-1H-indol-3-yl]-methanone hydrochloride

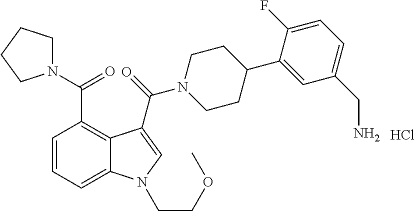

### A. 1-(2-Methoxy-ethyl)-1H-indole-4-carboxylic acid

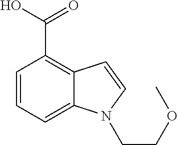

The title compound is prepared in a similar manner as described in Example 1E using indole-4-carboxylic acid and 2-bromoethylmethylether as the starting material.

¹H NMR (300 MHz, CDCl₃) δ 8.03 (d, 1H), 7.63 (d, 1H), 7.35-7.34 (m, 1H), 7.32-7.29 (m, 1H), 7.24-7.23 (m, 1H), 4.35 (t, 2H), 3.73 (t, 2H), 3.32 (s, 3H).

### B. [1-(2-Methoxy-ethyl)-1H-indol-4-yl]-pyrrolidin-1-yl-methanone

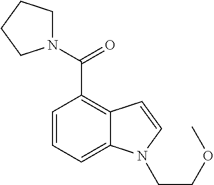

The title compound is prepared in a similar manner as described in Example 6E with pyrrolidine and 1-(2-methoxy-ethyl)-1H-indole-4-carboxylic acid as the starting materials.

¹H NMR (300 MHz, CDCl₃) δ 7.40-7.36 (m, 1H), 7.25-7.15 (m, 3H), 6.55-6.53 (m, 1H), 4.30 (t, 2H), 3.76-3.68 (m, 4H), 3.35 (t, 2H), 3.30 (s, 3H), 1.98 (quin, 2H), 1.83 (quin, 2H).

### C. 3-Formyl-1-(2-methoxy-ethyl)-1H-indole-4-carboxylic acid ethyl-propyl-amide

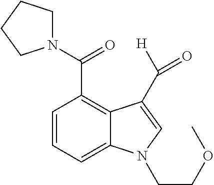

The title compound is prepared in a similar manner as described in Example 59C with [1-(2-methoxy-ethyl)-1H-indol-4-yl]-pyrrolidin-1-yl-methanone and phosphorus oxychloride (POCl₃) as the starting materials. The material was used in the next step without any further purification.

MS 301 (M+1).

### D. 1-(2-Methoxy-ethyl)-4-(pyrrolidine-1-carbonyl)-1H-indole-3-carboxylic acid

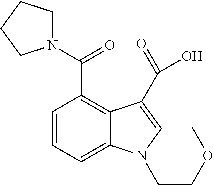

The title compound is prepared in a similar manner as described in Example 59D with 3-formyl-1-(2-methoxy-ethyl)-1H-indole-4-carboxylic acid ethyl-propyl-amide and sodium chlorite (NaClO₂) as the starting materials. The material was used in the next step without any further purification.

MS 317 (M+1).

### E. 2,2,2-Trifluoro-N-(4-fluoro-3-{1-[1-(2-methoxy-ethyl)-4-(pyrrolidine-1-carbonyl)-1H-indole-3-carbonyl]-piperidin-4-yl}-benzyl)-acetamide

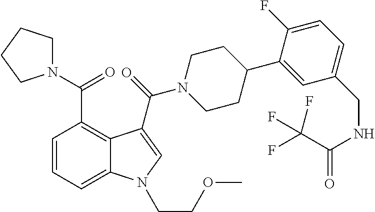

The title compound is prepared in a similar manner as described in Example 6E using 1-(2-methoxy-ethyl)-4-(pyrrolidine-1-carbonyl)-1H-indole-3-carboxylic acid and 2,2,2-trifluoro-N-(4-fluoro-3-piperidin-4-yl-benzyl)-acetamide hydrochloride as the starting materials.

¹H NMR (300 MHz, DMSO-d6) δ 9.95 (t, 1H), 7.61-7.58 (m, 2H), 7.39 (d, 1H), 7.25-7.20 (m, 1H), 7.15-7.12 (m, 2H), 7.03-7.01 (m, 1H), 4.40-4.35 (m, 5H), 3.69 (t, 2H), 3.45 (t, 2H), 3.23 (s, 3H), 3.17-3.12 (m, 3H), 3.09-2.97 (m, 3H), 1.90-1.83 (m, 2H), 1.81-1.74 (m, 3H), 1.71-1.65 (m, 3H).

### F. [4-(5-Aminomethyl-2-fluoro-phenyl)-piperidin-1-yl]-[1-(2-methoxy-ethyl)-4-(pyrrolidine-1-carbonyl)-1H-indol-3-yl]-methanone hydrochloride

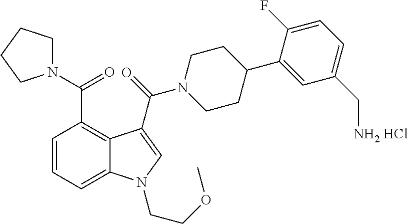

The title compound is prepared in a similar manner as described in Example 3B using 2,2,2-trifluoro-N-(4-fluoro-3-{1-[1-(2-methoxy-ethyl)-4-(pyrrolidine-1-carbonyl)-1H-indole-3-carbonyl]-piperidin-4-yl}-benzyl)-acetamide as the starting material.

¹H NMR (300 MHz, DMSO-d6) δ 8.37 (br s, 3H), 7.58 (s, 2H), 7.55 (s, 1H), 7.38-7.33 (m, 1H), 7.21-7.13 (m, 2H), 7.00-6.98 (m, 1H), 4.36 (t, 2H), 4.06 (br s, 2H), 3.95-3.93 (m, 2H), 3.65 (t, 2H), 3.40 (t, 2H), 3.19 (s, 3H), 3.13-3.05 (m, 3H), 2.96 (br s, 2H), 1.85-1.64 (m, 8H).

### Example 109

### 3-[4-(5-Aminomethyl-2-fluoro-phenyl)-piperidine-1-carbonyl]-1-(2-methoxy-ethyl)-1H-indole-4-carboxylic acid trifluoroacetate

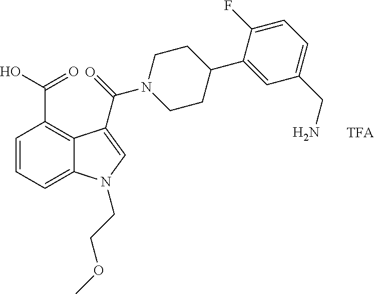

### A. 1-(2-Methoxy-ethyl)-1H-indole-4-carbaldehyde

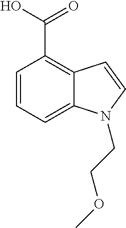

A mixture of 4-formyl-1H-indole (1.0 g, 6.89 mmol) and powdered KOH (1.16 g, 20.7 mmol) in DMSO (10 mL) is stirred at r.t. for 5 min then 2-methoxyethyl bromide (972 μL, 10.3 mmol) is added. After the reaction mixture is stirred at r.t. 15 min, it is partitioned between H₂O and Et₂O. The two layers are separated, and the aqueous layer is extracted with Et₂O (3×). The combined organic extracts are washed with H₂O and brine, dried over MgSO₄, filtered, and concentrated in vacuo. The crude material is purified on silica gel with heptane/EtOAc (75/25 to 50/50) as eluent to yield the titled product (1.25 g, 89%) as a yellow liquid.

¹H NMR (300 MHz, CDCl₃) δ 10.25 (s, 1H), 7.70-7.50 (m, 2H), 7.45-7.20 (m, 3H), 4.35 (t, J=5.4 Hz, 2H), 3.71 (t, J=5.4 Hz, 2H), 3.30 (s, 3H);

LC Rt: 0.82 min.

### B. 1-(2-Methoxy-ethyl)-3-(2,2,2-trifluoro-acetyl)-1H-indole-4-carbaldehyde

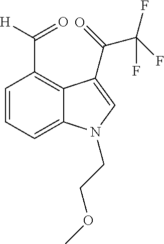

A mixture of 1-(2-methoxy-ethyl)-1H-indole-4-carbaldehyde (1.25 g, 6.15 mmol) and TFAA (2.57 mL, 18.5 mmol) in DMF (15 mL) is heated at r.t. for 3 days. The mixture is then partitioned between sat. Na₂CO₃ and Et₂O. The two layers are separated and the organic layer is washed with H₂O and brine, dried over MgSO₄, filtered, and concentrated in vacuo. The crude material is purified on silica gel with heptane/EtOAc (75/25 to 40/60) as eluent. The residue is recrystallized from CH₂Cl₂/heptane to provide the product (1.42 g, 77%) as a yellow waxy solid.

¹H NMR (300 MHz, CDCl₃) δ 11.24 (s, 1H), 8.25 (d, J=1.6 Hz, 1H), 8.03 (d, J=7.5 Hz, 1H), 7.68 (d, J=8.1 Hz, 1H), 7.51 (t, J=7.8 Hz, 1H), 4.46 (t, J=5.0 Hz, 2H), 3.78 (t, J=5.1 Hz, 2H), 3.34 (s, 3H);

¹⁹F NMR (300 MHz, CDCl₃) δ −69.88 (s, 3F);

LC Rt: 0.90 min; MS 300 (M+H, 100%).

### C. 4-Formyl-1-(2-methoxy-ethyl)-1H-indole-3-carboxylic acid

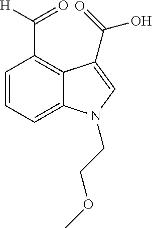

A mixture of 1-(2-methoxy-ethyl)-3-(2,2,2-trifluoro-acetyl)-1H-indole-4-carbaldehyde (1.40 g, 4.68 mmol) in MeOH (10 mL) and NaOH (5 M, 10 mL) is heated at 80° C. overnight. This mixture is concentrated in vacuo to remove the methanol. The residue is diluted with H₂O, and then washed with EtOAc once. The aqueous layer at 0° C. is acidified to pH 1 with conc. HCl. The acidified mixture is extracted with EtOAc (2×). The combined organic extracts are washed with H₂O and brine, dried over MgSO₄, filtered, and concentrated in vacuo to yield the product (1.56 g, 100%) as a beige powder. This material is used in the next step without further purification.

LC Rt: 0.69 min.

### D. 2,2,2-Trifluoro-N-(4-fluoro-3-{1-[4-formyl-1-(2-methoxy-ethyl)-1H-indole-3-carbonyl]-piperidin-4-yl}-benzyl)-acetamide

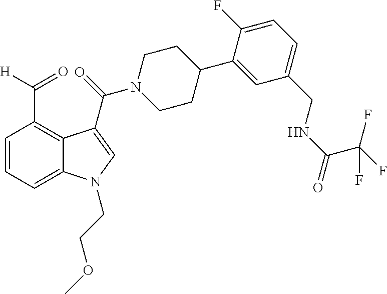

A mixture of 4-formyl-1-(2-methoxy-ethyl)-1H-indole-3-carboxylic acid (736 mg, 2.98 mmol), Et₃N (1.04 mL, 7.45 mmol), 2,2,2-trifluoro-N-(4-fluoro-3-piperidin-4-yl-benzyl)-acetamide hydrochloride (1.22 g, 3.57 mmol), and EDCI (0.86 g, 4.47 mmol) in CH₂Cl₂ (20 mL) is stirred at r.t. overnight. The mixture is partitioned between H₂O and CH₂Cl₂. The two layers are separated, and the organic layer is washed with brine, dried over MgSO₄, filtered, and concentrated in vacuo. The crude material is purified on silica gel with EtOAc/MeOH (100/0 to 80/20) as eluent to give the product (918 mg, 57%) as a white powder.

¹H NMR (300 MHz, CDCl₃) δ 10.34 (s, 1H), 7.75 (d, J=7.2 Hz, 1H), 7.68 (d, J=8.2 Hz, 1H), 7.48 (s, 1H), 7.40 (t, J=7.7 Hz, 1H), 7.25.7.10 (m, 2H), 7.10-6.90 (m, 2H), 5.30-4.60 (br m, 1H), 4.60-4.20 (m, 5H), 3.73 (t, J=5.3 Hz, 2H), 3.32 (s, 3H), 3.25-2.80 (m, 3H), 2.10-1.50 (m, 4H);

¹⁹F NMR (300 MHz, CDCl₃) δ −75.30 (s, 3F), −119.49 (br m, 1F);

LC Rt 0.95 min; MS 534 (M+H, 100%).

### E. 3-(4-{2-Fluoro-5-[(2,2,2-trifluoro-acetylamino)-methyl]-phenyl}-piperidine-1-carbonyl)-1-(2-methoxy-ethyl)-1H-indole-4-carboxylic acid

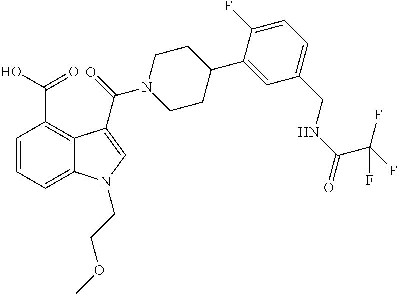

To a solution of 2,2,2-trifluoro-N-(4-fluoro-3-{1-[4-formyl-1-(2-methoxy-ethyl)-1H-indole-3-carbonyl]-piperidin-4-yl}-benzyl)-acetamide (900 mg, 1.69 mmol) and 2-methyl-2-butene (0.8 mL) in THF (10 mL)/t-BuOH (5 mL) is added a solution of sodium chlorite (764 mg, 8.45 mmol) and sodium dihydrogen phosphate (1.26 g, 10.1 mol) in water (4 mL). This mixture is stirred at r.t. for 2 h. The mixture is concentrated in vacuo to remove the organic solvents. The residue is partitioned between water and EtOAc. The two layers are separated, and the organic layer is washed with brine, dried over MgSO₄, filtered, and concentrated in vacuo. The crude material is purified on silica gel with EtOAc/MeOH (100/0 to 80/20) as eluent to give the product (780 mg, 84%) as a beige powder.

¹H NMR (300 MHz, CDCl₃) δ 8.61 (bs, 1H), 7.90-7.70 (m, 1H), 7.70-7.50 (m, 1H), 7.50-7.30 (m, 2H), 7.30-7.10 (m, 1H), 7.10-6.95 (m, 1H), 6.88 (d, J=9.9 Hz, 1H), 5.10-4.95 (br m, 1H), 4.45-4.30 (m, 2H), 4.10-3.80 (m, 2H), 3.70 (t, J=4.9 Hz, 2H), 3.31 (s, 3H), 3.25-3.00 (m, 2H), 3.00-2.75 (m, 1H), 2.30-1.50 (m, 4H);

¹⁹F NMR (300 MHz, CDCl₃) δ −76.01 (s, 3F), −122.34 (br s, 1F);

LC Rt 0.90 min; MS 550 (M+H, 100%).

### F. 3-[4-(5-Aminomethyl-2-fluoro-phenyl)-piperidine-1-carbonyl]-1-(2-methoxy-ethyl)-1H-indole-4-carboxylic acid trifluoroacetate

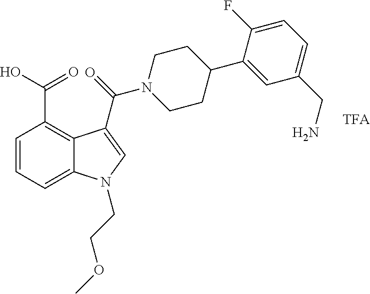

To a mixture of 3-(4-{2-fluoro-5-[(2,2,2-trifluoro-acetylamino)-methyl]-phenyl}-piperidine-1-carbonyl)-1-(2-methoxy-ethyl)-1H-indole-4-carboxylic acid (30 mg, 0.055 mmol) in MeOH (5 mL) is added aqueous K₂CO₃ (200 mg dissolved in 2.0 mL H₂O). This mixture is stirred at r.t. overnight. LC/MS indicates the reaction is completed. The reaction mixture is concentrated in vacuo to remove most of the methanol. The residue is partitioned between H₂O and EtOAc. The residue is diluted with water, and 3 M HCl is added until pH˜1. The suspension is concentrated to dryness in vacuo. The residue is purified by RP-HPLC to give the product (12 mg, 38%) as a white powder.

¹H NMR (300 MHz, DMSO-d6) δ 8.17 (br, s 3H), 7.81 (d, J=8.1 Hz, 1H), 7.70-7.55 (m, 2H), 7.50-7.40 (m, 1H), 7.40-7.15 (m, 3H), 4.90-4.50 (br m, 1H), 4.50-4.30 (m, 2H), 4.20-3.80 (m, 3H), 3.75-3.65 (m, 2H), 3.22 (s, 3H), 3.15-2.65 (m, 3H), 1.95-1.45 (m, 4H);

¹⁹F NMR (300 MHz, DMSO-d6) δ −73.37 (s, 3F), −119.49 (s, 1F);

LC 0.63 min; MS 454 (M+H, 100%).

### Example 110

### [4-(5-Aminomethyl-2-trifluoromethyl-phenyl)-piperidin-1-yl]-[1-(2-methoxy-ethyl)-7-methyl-1H-indol-3-yl]-methanone hydrochloride

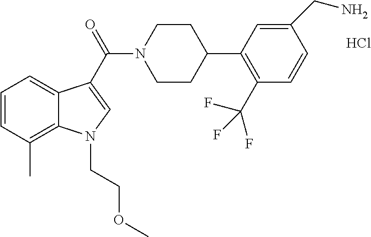

### A. 3-Bromo-4-trifluoromethyl-benzoic acid

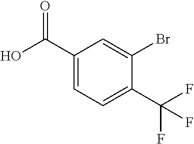

To a 0° C. solution of copper (II) bromide (7.0 g, 48.6 mmol) in acetonitrile (150 mL) is added t-butylnitrite (5.6 mL, 46.4 mmol) followed by addition of 3-amino-4-trifluoromethyl-benzoic acid (5.0 g, 24.4 mmol) over a 5 min period. The reaction mixture is stirred at 0° C. for 2 h and then at r.t. overnight. The reaction mixture is poured into EtOAc, washed with 1N HCl (2×), brine, dried over MgSO₄, filtered, and concentrated in vacuo to give the titled compound (6.22 g, 95%).

¹H NMR (300 MHz, DMSO-d6) δ 13.8 (bs, 1H), 8.3 (s, 1H), 8.1 (m, 2H).

### B. (3-Bromo-4-trifluoromethyl-phenyl)-methanol

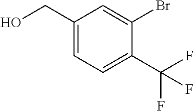

To a 0° C. solution of 3-bromo-4-trifluoromethyl-benzoic acid (6.2 g, 23 mmol) in THF (50 mL) is added a 1.0M borane/THF solution (39 mL, 39 mmol). The resulting mixture is allowed to warm to r.t. and stir overnight. To the reaction mixture is added MeOH (7 mL) and 1 N HCl (7 mL). The resulting mixture is heated to reflux for 1 h and then cooled to r.t. The reaction mixture is concentrated in vacuo and the residue is taken up in ethyl acetate, washed with H₂O, sat. NaHCO₃, brine, dried over Na₂SO₄, filtered and concentrated in vacuo. Purification by flash chromatography on SiO₂ eluting with 30% ethyl acetate/heptane yields the titled compound (4.75 g, 81%).

¹H NMR (300 MHz, DMSO-d6) δ 7.8 (m, 2H), 7.5 (m, 1H), 5.5 (m, 2H).

### C. (3-Bromo-4-trifluoromethyl-benzyloxy)-tert-butyl-dimethyl-silane

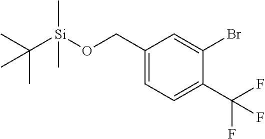

To a solution of (3-bromo-4-trifluoromethyl-phenyl)-methanol (4.7 g, 18.43 mmol) in CH₂Cl₂ (250 mL) is added tert-butyldimethylsilyl chloride (5.6 g, 36.86 mmol) and dropwise addition of 1,8-diazabicyclo[5.4.0]undec-7-ene (3.3 mL, 22.12 mmol). The reaction mixture is stirred at r.t. overnight. The reaction mixture is poured into Et₂O, washed with sat NaHCO₃, brine, dried over Na₂SO₄, filtered, and concentrated in vacuo to give the crude product. Purification by flash chromatography on SiO₂ eluting with 1% ethyl acetate/heptane yields the titled compound (6.35 g, 93% yield). ¹H NMR (300 MHz, CDCl₃) δ 7.8 (m, 2H), 7.5 (s, 1H), 4.8 (s, 2H), 0.9 (s, 9H), 0.5 (s, 6H).

### D. 4-[5-(tert-Butyl-dimethyl-silanyloxymethyl)-2-trifluoromethyl-phenyl]-3,6-dihydro-2H-pyridine-1-carboxylic acid benzyl ester

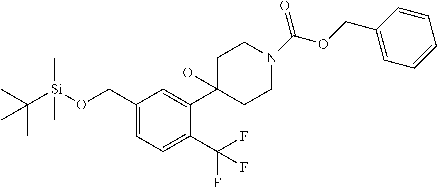

To a −78° C. solution of (3-bromo-4-trifluoromethyl-benzyloxy)-tert-butyl-dimethyl-silane (5.0 g, 14.3 mmol) in THF (100 mL) is added a 1.7 M tert-butyllithium/pentane solution (8.6 mL, 14.6 mmol). The resulting mixture is stirred at −78° C. for 15 min then N-CBZ-piperidin-4-one (3.3 g, 14.3 mmol) as a solution in THF (25 mL) is added. The reaction is allowed to warm to r.t. and stirred overnight. The reaction mixture is heated at 40° C. for 2 h and is then cooled to r.t. The reaction is poured into ethyl acetate, washed with sat NH₄Cl, H₂O, brine, dried over MgSO₄, filtered and concentrated in vacuo. Purification by flash chromatography on SiO₂ eluting with 20% ethyl acetate/heptane yields the titled compound (2.52 g, 36%).

¹H NMR (300 MHz, DMSO-d6) δ 7.8 (d, 1H), 7.6 (s, 1H), 7.4 (m, 6H), 5.1 (s, 2H), 4.8 (s, 2H), 3.9 (m, 2H), 3.3 (m, 2H), 3.2 (bs, 1H), 1.9 (m, 2H), 1.8 (m, 2H), 0.9 (s, 9H), 0.5 (s, 6H).

MS m/z: [M+H]⁺=524.

### E. 4-(5-Hydroxymethyl-2-trifluoromethyl-phenyl)-3,6-dihydro-2H-pyridine-1-carboxylic acid benzyl ester

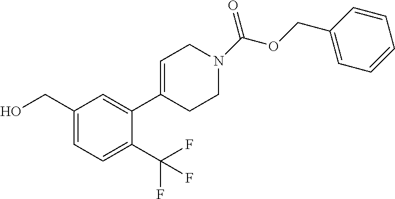

To a solution of 4-[5-(tert-butyl-dimethyl-silanyloxymethyl)-2-trifluoromethyl-phenyl]-3,6-dihydro-2H-pyridine-1-carboxylic acid benzyl ester (2.00 g, 3.82 mmol) in CH₂Cl₂ (100 mL) is added a solution of boron trifluoride diethyl etherate (4.84 mL, 38.2 mmol). The resulting mixture is stirred at r.t. overnight. The reaction mixture is quenched with sat NaHCO₃ (150 mL) and stirred at r.t. for 3 h. The reaction mixture is extracted with ethyl acetate (3×) and the combined organic extracts are washed with brine, dried over Na₂SO₄, filtered and concentrated in vacuo. Purification by flash chromatography on SiO₂ eluting with 40% ethyl acetate/heptane yields the titled compound (0.39 g, 26%).

¹H NMR (300 MHz, CDCl₃) δ 7.6 (d, 1H), 7.4 (m, 6H), 7.2 (s, 1H), 5.6 (m, 1H), 5.2 (s, 2H), 4.8 (m, 2H), 4.1 (m, 2H), 3.7 (m, 2H), 2.4 (m, 2H), 1.8 (m, 1H).

MS m/z: [M+H]⁺=392.

### F. 4-(5-Azidomethyl-2-trifluoromethyl-phenyl)-3,6-dihydro-2H-pyridine-1-carboxylic acid benzyl ester

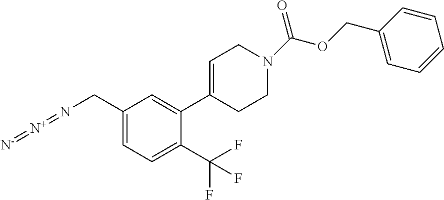

A solution of 4-(5-hydroxymethyl-2-trifluoromethyl-phenyl)-3,6-dihydro-2H-pyridine-1-carboxylic acid benzyl ester (0.5 g, 1.28 mmol), triethylamine (0.34 mL, 2.43 mmol) and diphenylphosphoryl azide (0.55 mL, 2.56 mmol) in THF (5 mL) is subjected to a microwave apparatus at 80° C. for 1 h. The reaction mixture is concentrated in vacuo and the residue is diluted with a solution of EtOH (20 mL) and 6 N NaOH (20 mL) and stirred for 30 min. The reaction mixture is poured into EtOAc, washed with H₂O, brine, dried over Na₂SO₄, filtered and concentrated in vacuo. Purification by flash chromatography on SiO₂ eluting with 20% ethyl acetate/heptane yields the titled compound (0.18 g, 34%).

¹H NMR (300 MHz, CDCl₃) δ 7.7 (d, 1H), 7.4 (m, 6H), 7.2 (s, 1H), 5.6 (m, 1H), 5.2 (s, 2H), 4.4 (m, 2H), 4.1 (m, 2H), 3.7 (m, 2H), 2.4 (m, 2H).

MS m/z: [M+H]⁺=417.

### G. 4-(5-Aminomethyl-2-trifluoromethyl-phenyl)-3,6-dihydro-2H-pyridine-1-carboxylic acid benzyl ester

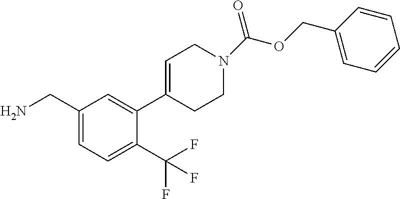

The titled compound is prepared according to the procedure by Lin, Wenqing et al. Synthetic Communications, 2002, 32(21), pp. 3279-3284 using 4-(5-azidomethyl-2-trifluoromethyl-phenyl)-3,6-dihydro-2H-pyridine-1-carboxylic acid benzyl ester as the starting material.

¹H NMR (300 MHz, CDCl₃) δ 7.6 (d, 1H), 7.4 (m, 6H), 7.2 (s, 1H), 5.6 (m, 1H), 5.2 (s, 2H), 4.2 (m, 2H), 3.9 (m, 2H), 3.7 (m, 2H), 2.4 (m, 2H).

MS m/z: [M+H]⁺=391.

### H. 4-[5-(tert-Butoxycarbonylamino-methyl)-2-trifluoromethyl-phenyl]-3,6-dihydro-2H-pyridine-1-carboxylic acid benzyl ester

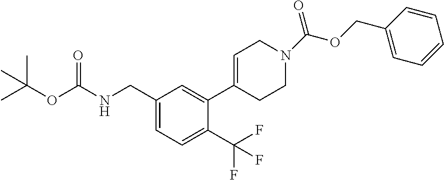

To a solution of 4-(5-aminomethyl-2-trifluoromethyl-phenyl)-3,6-dihydro-2H-pyridine-1-carboxylic acid benzyl ester (1.10 g, 2.82 mmol) in THF (50 mL) is added Boc-anhydride (1.23 g, 5.64 mmol) and triethylamine (0.55 mL, 3.95 mmol). The resulting mixture is stirred at r.t. overnight. The reaction is poured into ethyl acetate, washed with 0.5 N NaOH, brine, dried over MgSO₄, filtered and concentrated in vacuo. Purification by flash chromatography on SiO₂ eluting with 20% ethyl acetate/heptane yields the titled compound (1.0 g, 72%).

¹H NMR (300 MHz, CDCl₃) δ 7.6 (d, 1H), 7.4 (m, 6H), 7.1 (s, 1H), 5.6 (m, 1H), 5.2 (s, 2H), 4.9 (m, 1H), 4.4 (m, 2H), 4.1 (m, 2H), 3.7 (m, 2H), 2.4 (m, 2H), 1.5 (s, 9H). MS m/z: [M+H]⁺=491.

### I. [3-(1,2,3,6-Tetrahydro-pyridin-4-yl)-4-trifluoromethyl-benzyl]-carbamic acid tert-butyl ester

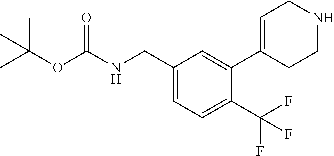

A solution of 4-[5-(tert-butoxycarbonylamino-methyl)-2-trifluoromethyl-phenyl]-3,6-dihydro-2H-pyridine-1-carboxylic acid benzyl ester (0.88 g, 1.78 mmol) and 10% Pd/C (0.25 g) in THF (5 mL) is subjected to H₂ at 50 psi for 6 h. The reaction mixture is filtered through Celite and concentrated in vacuo to yield the titled compound (0.63 g, 99%).

¹H NMR (300 MHz, CDCl₃) δ 7.6 (d, 1H), 7.4 (d, 1H), 7.1 (s, 1H), 5.6 (m, 1H), 4.9 (m, 1H), 4.4 (m, 2H), 3.5 (m, 1H), 3.1 (m, 1H), 2.3 (m, 1H), 1.9-1.7 (m, 4H), 1.5 (s, 9H).

MS m/z: [M+H]+=357.

### J. (3-{1-[1-(2-Methoxy-ethyl)-7-methyl-1H-indole-3-carbonyl]-1,2,3,6-tetrahydro-pyridin-4-yl}-4-trifluoromethyl-benzyl)-carbamic acid tert-butyl ester

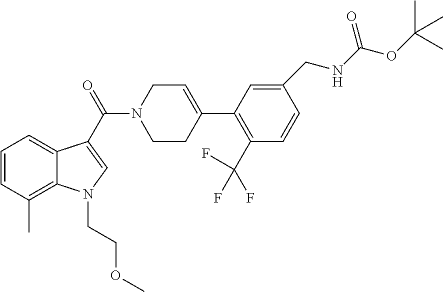

The title compound is prepared in a similar manner as described in Example 2I using [3-(1,2,3,6-tetrahydro-pyridin-4-yl)-4-trifluoromethyl-benzyl]-carbamic acid tert-butyl ester and 1-(2-methoxy-ethyl)-7-methyl-1H-indole-3-carboxylic acid as the starting materials.

¹H NMR (300 MHz, CDCl₃) δ 7.6 (m, 2H), 7.4 (s, 1H), 7.1 (m, 3H), 7.0 (m, 1H), 5.6 (m, 1H), 4.9 (m, 1H), 4.5 (t, 2H), 4.3 (m, 2H), 3.9 (m, 2H), 3.7 (t, 2H), 3.3 (s, 3H), 2.7 (s, 3H), 2.5 (m, 2H), 1.6 (m, 2H), 1.5 (m, 9H).

MS m/z: [M+H]⁺=572.

### K. 4-(5-Aminomethyl-2-trifluoromethyl-phenyl)-3,6-dihydro-2H-pyridin-1-yl]-[1-(2-methoxy-ethyl)-7-methyl-1H-indol-3-yl]-methanone hydrochloride

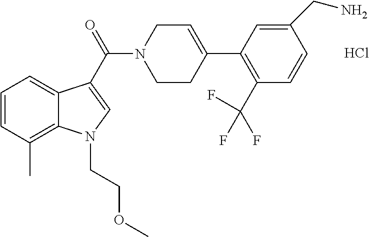

To a 2M HCl/Et₂O solution (25 mL) is added (3-{1-[1-(2-methoxy-ethyl)-7-methyl-1H-indole-3-carbonyl]-1,2,3,6-tetrahydro-pyridin-4-yl}-4-trifluoromethyl-benzyl)-carbamic acid tert-butyl ester (0.65 g, 1.1 mmol). The resulting mixture is stirred at r.t. overnight. The precipitate is collected to give the titled compound (0.5 g, 93%).

### L. 4-(5-Aminomethyl-2-trifluoromethyl-phenyl)-piperidin-1-yl]-[1-(2-methoxy-ethyl)-7-methyl-1H-indol-3-yl]-methanone hydrochloride

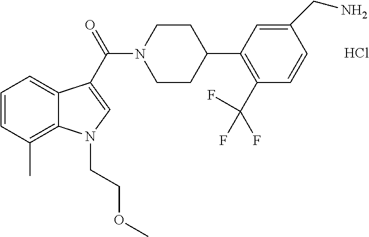

To a solution of 4-(5-aminomethyl-2-trifluoromethyl-phenyl)-3,6-dihydro-2H-pyridin-1-yl]-[1-(2-methoxy-ethyl)-7-methyl-1H-indol-3-yl]-methanone hydrochloride (0.50 g, 1.1 mmol) in MeOH (20 mL) is added ammonium formate (0.63 g, 10 mmol) and 10% Pd/C (0.4 g). The reaction mixture is heated to reflux for 8 h and is concentrated in vacuo. The residue is treated with 2 M HCl/Et₂O (10 mL) and the resulting mixture is stirred at r.t. overnight. The precipitate is collected to yield the titled compound (0.43 g, 85%).

¹H NMR (300 MHz, DMSO-d6) δ 8.4 (bs, 2H), 7.9 (s, 1H), 7.8 (m, 1H), 7.7 (m, 1H), 7.5 (m, 1H), 7.0 (m, 2H), 4.6 (t, 2H), 4.5 (m, 1H), 4.1 (m, 2H), 3.7 (t, 2H), 3.2 (s, 3H), 3.1 (m, 4H), 2.7 (s, 3H), 1.9-1.7 (m, 4H).

MS m/z: [M+H]⁺=474.

### Example 111

### [4-(5-Aminomethyl-2,4-difluoro-phenyl)-piperidin-1-yl]-[1-(2-methoxy-ethyl)-7-methyl-1H-indol-3-yl]-methanone hydrochloride

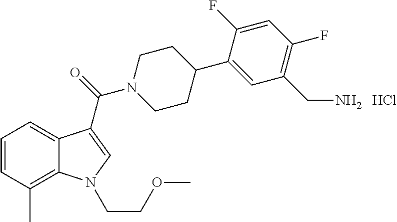

### A. 5-Bromo-2,4-difluorobenzoic acid

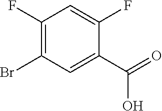

The title compound is prepared according to the procedure by Tochon-Danguy, H. J. et al., Nuclear Medicine and Biology, 2004, vol. 31, p. 839 with 2,4-difluorobenzoic acid. The titled compound is obtained as a white solid.

¹H NMR (300 MHz, CDCl₃) δ 8.29 (t, 1H), 7.01 (dd, 1H).

¹⁹F NMR (300 MHz, CDCl₃) δ −93.4 (m), −103.9 (m).

LCMS m/z: [M+H]⁺=234, 236.

### B. 5-Bromo-2,4-difluorobenzoic acid methyl ester

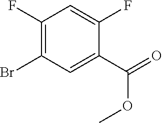

The title compound is prepared according to the procedure by Shioiri, T et al., Chem. Pharm. Bull., 1981, vol. 29, pp. 1475-1478 with 5-bromo-2,4-difluoro-benzoic acid. The titled compound was obtained as an amber solid.

¹H NMR (300 MHz, CDCl₃) δ 8.20 (t, 1H), 6.97 (dd, 1H), 3.94 (s, 3H).

¹⁹F NMR (300 MHz, CDCl₃) δ −95.6 (m), −105.5 (m).

LCMS m/z: [M+H]⁺=249, 251.

### C. (5-Bromo-2,4-difluorophenyl)methanol

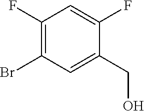

To a solution of diisobutylaluminum hydride (105 mL, 157.5 mmol)) in toluene (50 mL) cooled at 0° C. is added a solution of 2,4-difluoro-5-bromomethyl benzoate (19.20 g, 76.48 mmol) in DCM dropwise over ˜15 min. The resulting mixture is stirred at 0° C. for ˜2½ hours under nitrogen atmosphere. The mixture is poured into an Erlenmeyer flask containing a cold saturated solution of Rochelle's salt. The resulting mixture is stirred until the mixture became clear. The aqueous phase is extracted ethyl acetate (×3). The combined organic layers are washed with brine then separated and dried (MgSO₄). The organic phase is concentrated in vacuo without any further purification to afford the titled compound (17.05 g, 99%) as an orange oil.

¹H NMR (300 MHz, CDCl₃) δ 7.64 (t, 1H), 6.92-6.86 (m, 1H), 4.71 (s, 2H).

¹⁹F NMR (300 MHz, CDCl₃) δ −104.6 (m), −116.9 (m).

### D. 1-Bromo-5-bromomethyl-2,4-difluorobenzene

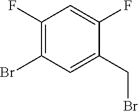

To a solution of phosphorus (V) oxybromide (26.50 g, 92.43 mmol) in dichloromethane (300 mL) at 0° C. is added N,N-dimethylformamide (150 mL) dropwise over ˜20 min. To the resulting white suspension at 0° C. is added a solution of (5-bromo-2,4-difluorophenyl)methanol (17.05 g, 76.45 mmol) in dichloromethane dropwise over ˜15 min. The resulting mixture is stirred at 0° C. for ˜¾ hour under nitrogen atmosphere. The aqueous phase is extracted ethyl acetate (×3). The combined organic phases are washed with brine then separated and dried (MgSO₄). The organic phase is concentrated in vacuo and the crude residue is flash chromatographed over SiO₂ using (heptane:EtOAc (90:10) to afford the titled compound (17.43 g, 80%) as a colorless oil.

¹H NMR (300 MHz, CDCl₃) δ 7.77-7.71 (m, 1H), 7.15-7.08 (m, 1H), 4.52 (s, 2H).

¹⁹F NMR (300 MHz, CDCl₃) δ −104.2 (m), −114.9 (m).

MS m/z: [M+H]⁺=283, 285, 287.

### E. 1-Azidomethyl-5-bromo-2,4-difluorobenzene

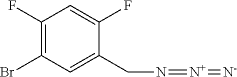

To a solution of 1-bromo-5-bromomethyl-2,4-difluorobenzene (17.12 g, 59.88 mmol) in N,N-dimethylformamide (75 mL) at r.t. is added sodium azide (7.87 g, 121.1 mmol) and the mixture is stirred at r.t. over night. The mixture is poured into water and the aqueous phase is extracted ethyl acetate (×3). The combined organic phases are washed with brine then separated and dried (MgSO₄). The organic phase is concentrated in vacuo without any further purification to afford the titled compound (14.55 g, 98%) as a yellow oil.

¹H NMR (300 MHz, CDCl₃) δ 7.56 (t, 1H), 6.99-6.93 (m, 1H), 4.38 (s, 2H).

¹⁹F NMR (300 MHz, CDCl₃) δ −102.6 (m), −114.8 (m).

### F. 5-Bromo-2,4-difluorobenzylamine

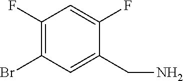

To a solution of 1-azidomethyl-5-bromo-2,4-difluoro-benzene (14.55 g, 58.66 mmol) in THF and water (10:1) at r.t. is added triphenylphosphine (30.81 g, 117.4 mmol) and the mixture is stirred at r.t. for one hour. THF is removed in vacuo and the syrup is acidified with 10% HCl. The aqueous phase is extracted ethyl acetate. The aqueous phase is basified with 50% NaOH and the aqueous phase is extracted with ethyl acetate (3×) and the combined organic phases are washed with brine then separated and dried (MgSO₄). The organic phase is concentrated in vacuo and the crude residue is flash chromatographed over SiO₂ using CH₂Cl₂:MeOH (90:10) to afford the titled compound (4.53 g, 35%) as a pale yellow oil.

¹H NMR (300 MHz, CDCl₃) δ 7.57 (t, 1H), 6.90-6.84 (m, 1H), 3.86 (s, 2H), 1.54 (br s, 2H).

¹⁹F NMR (300 MHz, CDCl₃) δ −106.1 (m), −117.1 (m).

MS m/z: [M+H]⁺=221, 223.

### G. 2,4-Difluoro-5-pyridin-4-yl-benzylamine

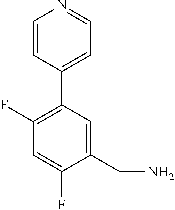

A solution of 5-bromo-2,4-difluoro-benzylamine (2.39 g, 10.76 mmol), 4-pyridineboronic acid (1.65 g, 13.42 mmol) and sodium bicarbonate (2.84 g, 33.76 mmol) in iso-propanol and water (1:1) is degasses with nitrogen. Dichloro[1,1′-bis(diphenylphosphino)ferrocene]palladium(II) dichloromethane complex is added (450 mg. 0.55 mmol) and the mixture is purged again with nitrogen. The mixture is heated at 90° C. under nitrogen stream over night. The solvent is removed in vacuo and the residue is acidified with 10% HCl. The aqueous phase is extracted with DCM. The aqueous phase is basified with 50% NaOH and the aqueous phase is extracted with ethyl acetate (×3) and the combined organic phases are washed with brine then separated and dried (MgSO₄). The organic phase is concentrated in vacuo without any further purification to afford the titled compound (2.30 g, 97%) as a brown solid.

¹H NMR (300 MHz, CDCl₃) δ 8.68 (d, 2H), 7.56-7.45 (m, 3H), 6.94 (t, 1H), 3.96 (s, 2H), 1.69 (br s, 2H).

¹⁹F NMR (300 MHz, CDCl₃) δ −115.1 (m), −115.8 (m).

MS m/z: [M+H]⁺=221.

### H. N-(2,4-Difluoro-5-pyridin-4-yl-benzyl)-2,2,2-trifluoro-acetamide

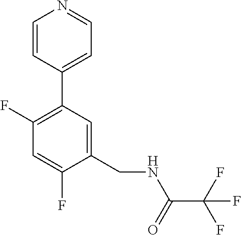

The title compound is prepared in a similar manner as described in Example 1F using 2,4-difluoro-5-pyridin-4-yl-benzylamine as a beige solid.

¹H NMR (300 MHz, CD₃OD) δ 8.84 (br s, 2H), 8.11 (br s, 2H), 7.81 (t, 1H), 7.29 (t, 1H), 4.57 (s, 2H).

¹⁹F NMR (300 MHz, CDCl₃) δ −77.0 (s), −111.9 (m), −114.5 (m).

MS m/z: [M+H]⁺=317.

### I. N-(2,4-Difluoro-5-piperidin-4-yl-benzyl)-2,2,2-trifluoro-acetamide hydrochloride

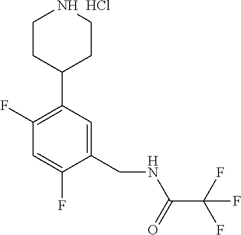

N-(2,4-Difluoro-5-pyridin-4-yl-benzyl)-2,2,2-trifluoro-acetamide is treated with 2.0 M HCl in ether (10 mL, 20.0 mmol) and stirred for 15 minutes. The mixture is vacuum dry and the residue is suspended ether overnight. The suspension is filtered and the cake is rinsed with ether twice. The solid is dried under vacuum.

To a solution of N-(2,4-difluoro-5-pyridin-4-yl-benzyl)-2,2,2-trifluoro-acetamide hydrochloride (1.71 g, 4.85 mmol) in methanol (50 mL) is added 5% Pt/C (1.06 g, 0.27 mmol) and concentrated HCl (5 drops). The resulting mixture is hydrogenated under 60 psi of hydrogen over night. The mixture is filtered on a bed of Celite and rinsed with methanol. The solvent is removed in vacuo to afford the titled compound (1.50 g, 86%) as a gum.

¹H NMR (300 MHz, DMSO-d6) δ10.04 (t, 1H), 8.74 (br s, 2H), 7.30-7.23 (m, 2H), 4.41 (d, 2H), 3.42-3.27 (m, 2H), 3.14-2.80 (m, 3H), 188-1.76 (m, 4H).

¹⁹F NMR (300 MHz, CDCl₃) δ −74.7 (s), −116.2 (m), −117.2 (m).

MS m/z: [M+H]⁺=323.

### J. N-(2,4-Difluoro-5-{1-[1-(2-methoxy-ethyl)-7-methyl-1H-indole-3-carbonyl]-piperidin-4-yl}-benzyl)-2,2,2-trifluoro-acetamide

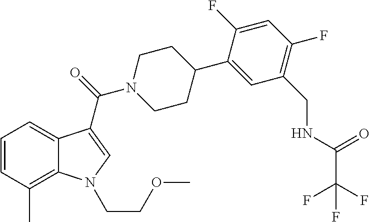

By proceeding in a similar manner to the method described in Example 2I with 7-methyl-1-(2-methoxy-ethyl)-1H-indole-3-carboxylic acid and N-(2,4-difluoro-5-piperidin-4-yl-benzyl)-2,2,2-trifluoro-acetamide hydrochloride as the starting material, the titled compound is prepared as a gum.

¹H NMR (300 MHz, CDCl₃) δ 7.57 (br d, 1H), 7.43 (s, 1H), 7.21 (t, 1H), 7.09 (t, 1H), 6.98 (m, 1H), 6.86-6.79 (m, 2H), 4.60-4.51 (m, 6H), 3.71 (t, 2H), 3.31 (s, 3H), 3.11-3.01 (m, 3H), 2.72 (s, 3H), 1.88-1.65 (m, 4H).

¹⁹F NMR (300 MHz, CDCl₃) δ −76.1 (s), −115.6 (m), −118.0 (m).

MS m/z: [M+H]⁺=538.

### K. [4-(5-Aminomethyl-2,4-difluoro-phenyl)-piperidin-1-yl]-[1-(2-methoxy-ethyl)-7-methyl-1H-indol-3-yl]-methanone hydrochloride

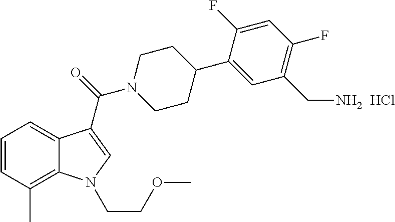

By proceeding in a similar manner to the method described in Example 3B with N-(2,4-difluoro-5-{1-[1-(2-methoxy-ethyl)-7-methyl-1H-indole-3-carbonyl]-piperidin-4-yl}-benzyl)-2,2,2-trifluoro-acetamide as the starting material, the titled compound is prepared as an off-white solid.

¹H NMR (300 MHz, DMSO-d6) δ 8.39 (br s, 3H), 7.67 (t, 1H), 7.61 (s, 1H), 7.51 (d, 1H), 7.29 (t, 1H), 7.01-6.96 (m, 1H), 6.92-6.90 (m, 1H), 4.55 (t, 2H), 4.39 (br d, 2H), 4.01 (br s, 2H), 3.64 (t, 2H), 3.20 (s, 3H), 3.14-2.96 (m, 3H), 2.65 (s, 3H), 1.80-1.57 (m, 4H).

¹⁹F NMR (300 MHz, DMSO-d6) δ −115.3 (m), −116.5 (m).

MS m/z: [M+H]⁺=442.

### Example 112

### [4-(3-Aminomethyl-4-fluoro-phenyl)-piperidin-1-yl]-[1-(2-methoxy-ethyl)-7-methyl-1H-indol-3-yl]-methanone hydrochloride

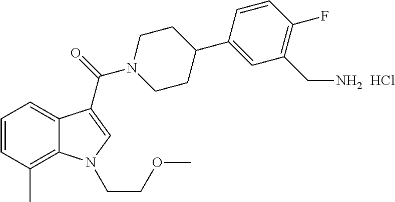

### A. 2-Fluoro-5-pyridin-4-yl-benzylamine

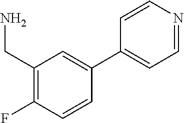

By proceeding in a similar manner to the method described in Example 112G with 5-bromo-2-fluorobenzylamine hydrochloride and 4-pyridineboronic acid as the starting material, the titled compound is prepared as a brown oil.

¹H NMR (300 MHz, CDCl₃) δ 8.63 (d, 2H), 7.66-7.62 (m, 1H), 7.54-7.46 (m, 3H), 7.14 (t, 1H), 3.98 (s, 2H), 1.94 (br s, 2H).

### B. 2,2,2-Trifluoro-N-(2-fluoro-5-pyridin-4-yl-benzyl)-acetamide

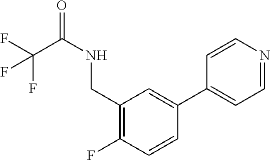

By proceeding in a similar manner to the method described in Example 1F with 2-fluoro-5-pyridin-4-yl-benzylamine as the starting material, the titled compound is prepared as a beige solid.

¹H NMR (300 MHz, CD₃OD) δ8.82 (d, 2H), 8.25-2.83 (m, 2H), 8.00-7.94 (m, 2H), 7.42-7.36 (m, 1H), 4.62 (s, 2H).

### C. 2,2,2-Trifluoro-N-(2-fluoro-5-piperidin-4-yl-benzyl)-acetamide hydrochloride

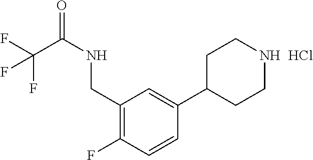

By proceeding in a similar manner to the method described in Example 112I with 2,2,2-trifluoro-N-(2-fluoro-5-pyridin-4-yl-benzyl)-acetamide as the starting material, the titled compound is prepared as an amber solid.

¹H NMR (300 MHz, DMSO-d6) δ 10.08-10.04 (m, 1H), 9.05 (br s, 1H), 7.21-7.15 (m, 3H), 4.42 (d, 2H), 3.42-3.32 (m, 2H), 3.03-2.79 (m, 3H), 1.90-1.73 (m, 4H).

### D. 2,2,2-Trifluoro-N-(2-fluoro-5-{1-[1-(2-methoxy-ethyl)-7-methyl-1H-indole-3-carbonyl]-piperidin-4-yl}-benzyl)-acetamide

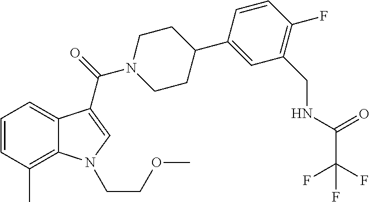

By proceeding in a similar manner to the method described in Example 2I with 7-methyl-1-(2-methoxy-ethyl)-1H-indole-3-carboxylic acid and 2,2,2-trifluoro-N-(2-fluoro-5-piperidin-4-yl-benzyl)-acetamide hydrochloride as the starting material, the titled compound is prepared as a white solid.

¹H NMR (300 MHz, CDCl₃) δ 7.58-7.55 (m, 1H), 7.41 (s, 1H), 7.19-7.14 (m, 2H), 7.10-6.96 (m, 4H), 4.55-4.51 (m, 6H), 3.70 (t, 2H), 3.30 (s, 3H), 3.05-2.97 (m, 2H), 2.81-2.74 (m, 1H), 2.71 (s, 3H), 1.87-1.83 (m, 2H), 1.73-1.59 (m, 2H).

### E. [4-(3-Aminomethyl-4-fluoro-phenyl)-piperidin-1-yl]-[1-(2-methoxy-ethyl)-7-methyl-1H-indol-3-yl]-methanone hydrochloride

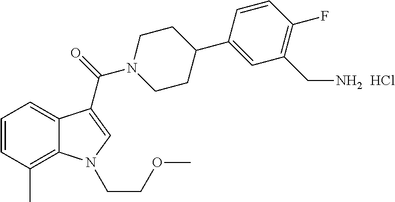

By proceeding in a similar manner to the method described in Example 3B with 2,2,2-trifluoro-N-(2-fluoro-5-{1-[1-(2-methoxy-ethyl)-7-methyl-1H-indole-3-carbonyl]-piperidin-4-yl}-benzyl)-acetamide as the starting material, the titled compound is prepared as an off-white solid.

¹H NMR (300 MHz, DMSO-d6) δ 8.42 (br s, 3H), 7.63 (s, 1H), 7.55-7.52 (m, 2H), 7.38-7.33 (m, 1H), 7.24-7.18 (m, 1H), 7.03-6.98 (m, 1H), 6.94-6.92 (m, 1H), 4.57 (t, 2H), 4.40 (br d, 2H), 4.09-4.02 (m, 2H), 3.67 (t, 2H), 3.22 (s, 3H), 3.08-3.00 (m, 2H), 2.88-2.81 (m, 1H), 2.67 (s, 3H), 1.83-1.79 (m, 2H), 1.67-1.58 (m, 2H).

MS m/z: [M+H]⁺=424.

### Example 113

### (6-Aminomethyl-3′,4′,5′,6′-tetrahydro-2′H-[2,4]bipyridinyl-1′-yl)-[1-(2-methoxy-ethyl)-7-methyl-1H-indol-3-yl]-methanone dihydrochloride

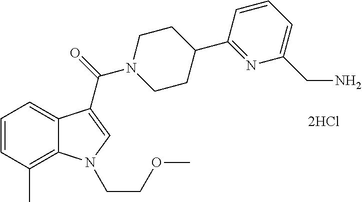

### A. 6-Aminomethyl-3′,6′-dihydro-2′H-[2,4]bipyridinyl-1′-carboxylic acid tert-butyl ester

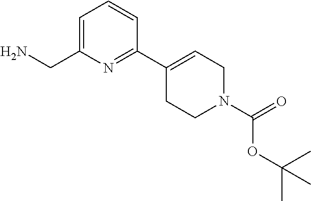

The title compound is prepared according to the procedure by Eastwood, P. R., Tet. Lett., 2000, vol. 41, pp. 3705-3708 & WO 2007/092435 (page 140-145) with (6-bromo-pyridin-2-yl)-methylamine and 4-(4,4,5,5-tetramethyl[1,3,2]dioxaborolan-2-yl)-3,6-dihydro-2H-pyridine-1-carboxylic acid tert-butyl ester as the starting materials to yield a brown oil.

¹H NMR (300 MHz, CDCl₃) δ 7.59 (t, 1H), 7.21 (d, 1H), 7.08 (d, 1H), 6.64 (br s, 1H), 3.96 (s, 2H), 3.62 (t, 2H), 2.74 (s, 4H), 2.63 (br s, 2H), 1.47 (s, 9H).

### B. 6-[(2,2,2-Trifluoro-acetylamino)-methyl]-3′,6′-dihydro-2′H-[2,4]bipyridinyl-1′-carboxylic acid tert-butyl ester

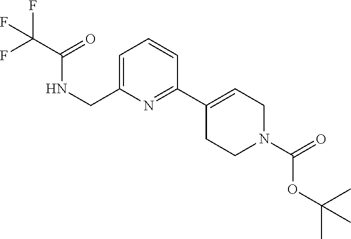

By proceeding in a similar manner to the method described in Example 1F with 6-aminomethyl-3′,6′-dihydro-2′H-[2,4′]bipyridinyl-1′-carboxylic acid tert-butyl ester as the starting material, the titled compound is prepared as a yellow solid.

¹H NMR (300 MHz, CDCl₃) δ 9.55 (br s, 1H), 8.09-8.05 (t, 1H), 7.59-7.55 (m, 2H), 6.76 (br s, 1H), 4.77 (d, 2H), 4.20 (m, 2H), 3.68 (t, 2H), 2.64 (m, 2H), 1.50 (s, 9H).

### C. 6-[(2,2,2-Trifluoro-acetylamino)-methyl]-3′,4′,5′,6′-tetrahydro-2′H-[2,4′]bipyridinyl-1′-carboxylic acid tert-butyl ester

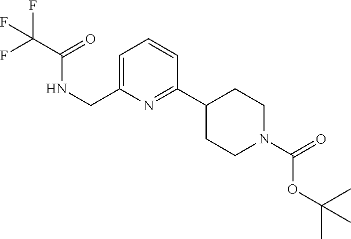

6-[(2,2,2-Trifluoro-acetylamino)-methyl]-3′,6′-dihydro-2′H-[2,4′]bipyridinyl-1′-carboxylic acid tert-butyl ester (1.68 g, 4.36 mmol) in methanol and 10% Pd/C (48 mg, 0.04 mmol) is hydrogenated under 1 atm of hydrogen for 30 min. The mixture is filtered on a Celite bed and rinsed with methanol. The solvent is removed in vacuo to give the titled compound as a yellow semi-solid.

¹H NMR (300 MHz, CDCl₃) δ 9.61 (br s, 1H), 8.09-8.04 (m, 1H), 7.60 (d, 1H), 7.43 (d, 1H), 4.73 (d, 2H), 4.29 (br s, 2H), 3.26-3.17 (tt, 1H), 2.92-2.84 (m, 2H), 2.00-1.95 (m, 2H), 1.74-1.63 (m, 2H), 1.48 (s, 9H).

### D. 2,2,2-Trifluoro-N-(1′,2′,3′,4′,5′,6′-hexahydro-[2,4′]bipyridinyl-6-ylmethyl)-acetamide dihydrochloride

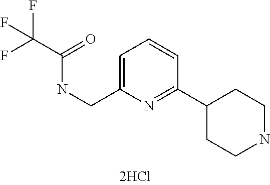

By proceeding in a similar manner to the method described in Example 110K with 6-[(2,2,2-trifluoro-acetylamino)-methyl]-3′,4′,5′,6′-tetrahydro-2′H-[2,4]bipyridinyl-1′-carboxylic acid tert-butyl ester the titled product is prepared as a beige solid.

¹H NMR (300 MHz, DMSO-d6) δ 10.20 (t, 1H), 9.25 (br s, 1H), 7.98 (t, 1H), 7.34 (dd, 2H), 4.58 (d, 2H), 3.35 (m, 2H), 3.18-3.10 (m, 1H), 3.05-2.94 (m, 2H), 2.08-1.88 (m, 4H).

### E. 2,2,2-Trifluoro-N-{1′-[1-(2-methoxy-ethyl)-7-methyl-1H-indole-3-carbonyl]-1′,2′,3′,4′,5′,6′-hexahydro-[2,4′]bipyridinyl-6-ylmethyl}-acetamide

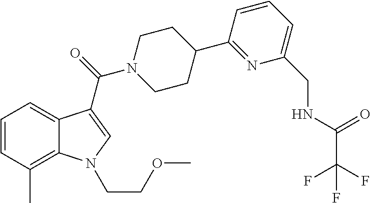

By proceeding in a similar manner to the method described in Example 2I with 7-methyl-1-(2-methoxy-ethyl)-1H-indole-3-carboxylic acid and 2,2,2-trifluoro-N-(1′,2′,3′,4′,5′,6′-hexahydro-[2,4′]bipyridinyl-6-ylmethyl)-acetamide dihydrochloride as the starting material, the titled compound is prepared as a gum.

¹H NMR (300 MHz, CDCl₃) δ 8.10 (br s, 1H), 7.66 (t, 1H), 7.59 (d, 1H), 7.43 (s, 1H), 7.16-7.06 (m, 3H), 6.98 (m, 1H), 4.62-4.52 (m, 6H), 3.71 (t, 2H), 3.30 (s, 3H), 3.14-2.98 (m, 3H), 2.72 (s, 3H), 2.01-1.98 (m, 2H), 1.91-1.77 (m, 2H).

### F. (6-Aminomethyl-3′,4′,5′,6′-tetrahydro-2′H-[2,4]bipyridinyl-1′-yl)-[1-(2-methoxy-ethyl)-7-methyl-1H-indol-3-yl]-methanone dihydrochloride

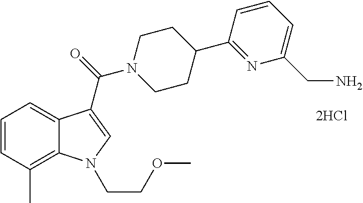

By proceeding in a similar manner to the method described in Example 3B with 2,2,2-trifluoro-N-{1′-[1-(2-methoxy-ethyl)-7-methyl-1H-indole-3-carbonyl]-1′,2′,3′,4′,5′,6′-hexahydro-[2,4]bipyridinyl-6-ylmethyl}-acetamide the titled compound is prepared as a white solid.

¹H NMR (300 MHz, DMSO-d6) δ 8.38 (br s, 3H), 7.85 (t, 1H), 7.62 (s, 1H), 7.53 (d, 1H), 7.37 (d, 2H), 7.02-6.97 (m, 1H), 6.94-6.92 (m, 1H), 4.57 (t, 2H), 4.42 (br d, 2H), 4.21-4.15 (m, 2H), 3.66 (t, 2H), 3.57 (s, 3H), 3.16-2.99 (m, 3H), 2.67 (s, 3H), 1.93-1.73 (m, 4H).

MS m/z: [M+H]⁺=407.

### Example 114

### [4-(5-Aminomethyl-2-trifluoromethyl-phenyl)-piperidin-1-yl]-[1-(2-methoxy-ethyl)-7-methyl-1H-indol-3-yl]-methanone hydrochloride

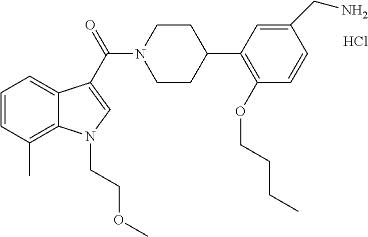

### A. 3-Bromo-4-butoxy-benzonitrile

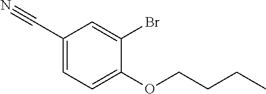

To a solution of 3-bromo-4-hydroxy-benzonitrile (5.0 g, 25.25 mmol) and potassium carbonate (7.0 g, 50.5 mmol) in DMF (100 mL) is added 1-bromo-butane. The reaction mixture is stirred at room temperature overnight. The reaction mixture is poured into EtOAc, washed with H₂O (2×), brine, dried over MgSO₄, filtered, and concentrated in vacuo to give the crude product. Purification by flash chromatography on SiO₂ eluting with 10% ethyl acetate/heptane gives the titled compound (5.9 g, 92%).

¹H NMR (300 MHz, CDCl₃) δ 7.8 (s, 1H), 7.6 (d, 1H), 6.9 (d, 1H), 4.1 (m, 2H), 1.85 (m, 2H), 1.5 (m, 2H), 1.0 (m, 3H).

MS m/z: [M+H]⁺=254

### B. 3-Bromo-4-butoxy-benzylamine

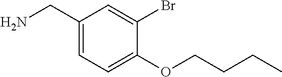

To a solution of 3-bromo-4-butoxy-benzonitrile (5.85 g, 23 mmol) in THF (100 mL) is added a 1.0 M borane.THF solution (28 mL, 28 mmol). The resulting mixture is heated to reflux for 3 h and is quenched with 6 N HCl (16 mL, 96 mmol). The resulting mixture is heated to reflux for 1 h and is stirred at room temperature overnight. The reaction mixture is diluted with H₂O (20 mL) and the solvent is removed in vacuo. The aqueous layer is diluted with H₂O (20 mL) and is washed with ethyl acetate, basified with 6 N NaOH to pH 13 and is extracted with ethyl acetate (2×). The extracts are combined and is washed with brine, dried over MgSO₄, filtered and concentrated in vacuo to yield the titled compound (3.75 g, 63%).

¹H NMR (300 MHz, CDCl₃) δ 7.5 (s, 1H), 7.2 (d, 1H), 6.8 (d, 1H), 4.0 (m, 2H), 3.8 (bs, 2H), 1.8 (m, 2H), 1.5 (m, 4H), 1.0 (m, 3H).

MS m/z: [M+H]⁺=258

### C. 4-Butoxy-3-pyridin-4-yl-benzylamine

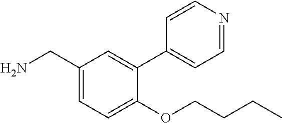

A flask is charged with NaHCO3 (2.86 g, 34.1 mmol), 3-bromo-4-butoxy-benzylamine (4 g, 15.5 mmol) and pyridine-4-boronic acid (2.1 g, 17.05 mmol) and isopropyl alcohol (40 mL) and water (20 mL) at r.t. The suspension is degassed with N₂ for 1.0 h at 10° C. Into the mixture is added 1,1′-bis(diphenylphosphino)ferrocene-palladium(II)dichloride dichloromethane complex (PdCl₂dppf-CH₂Cl₂ (0.5 g, 0.62 mmol). The reaction mixture is heated to 85° C. and stirred for 10 h. After the reaction is completed (HPLC analysis), the mixture is cooled to room temperature, and aqueous 1 N HCl (50 mL) is added, and stirred for 0.5 h. The solution is washed with EtOAc (2×) and the aqueous phase is basified to pH=13 with 6 N NaOH, and extracted with ethyl acetate (2×). The extracts are combined and is washed with brine, dried over MgSO₄, filtered and concentrated down in vacuo to yield the titled compound (4.0 g, 100%).

¹H NMR (300 MHz, CDCl₃) δ 8.6 (m, 2H), 7.5 (m, 2H), 7.3 (m, 2H), 7.0 (d, 1H), 4.0 (m, 2H), 3.8 (bs, 2H), 1.8 (m, 4H), 1.4 (m, 2H), 0.9 (m, 3H).

### D. N-(4-Butoxy-3-pyridin-4-yl-benzyl)-2,2,2-trifluoro-acetamide

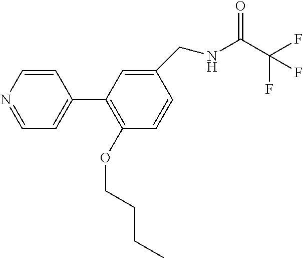

The title compound is prepared in a similar manner as described in Example 1F using 4-butoxy-3-pyridin-4-yl-benzylamine as the starting material.

¹H NMR (300 MHz, CDCl₃) δ 8.8 (m, 2H), 8.0 (m, 2H), 7.4 (m, 2H), 7.0 (d, 1H), 6.9 (bs, 1H), 4.5 (m, 2H), 4.0 (m, 2H), 1.9 (m, 2H), 1.4 (m, 2H), 0.9 (m, 3H).

### E. N-(4-Butoxy-3-piperidin-4-yl-benzyl)-2,2,2-trifluoro-acetamide hydrochloride

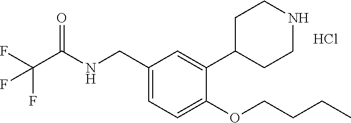

The title compound is prepared in a similar manner as described in Example 1I using N-(4-butoxy-3-pyridin-4-yl-benzyl)-2,2,2-trifluoro-acetamide as the starting material.

¹H NMR (300 MHz, DMSO-d6) δ 8.7 (bs, 1H), 8.4 (bs, 1H), 7.2 (m, 2H), 7.0 (m, 1H), 4.3 (m, 2H), 4.0 (m, 2H), 3.4 (m, 4H), 3.1 (m, 3H), 1.9 (m, 2H), 1.8 (m, 2H), 1.4 (m, 2H), 0.9 (m, 3H).

### F. N-(4-Butoxy-3-{1-[1-(2-methoxy-ethyl)-7-methyl-1H-indole-3-carbonyl]-piperidin-4-yl}-benzyl)-2,2,2-trifluoro-acetamide

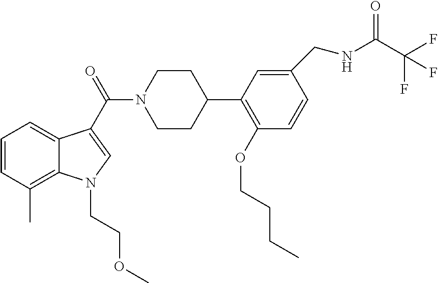

The title compound is prepared in a similar manner as described in Example 2I using N-(4-butoxy-3-piperidin-4-yl-benzyl)-2,2,2-trifluoro-acetamide hydrochloride and 1-(2-methoxy-ethyl)-7-methyl-1H-indole-3-carboxylic acid as the starting materials.

¹H NMR (300 MHz, CDCl₃) δ 7.6 (d, 1H), 7.4 (s, 1H), 7.1 (m, 3H), 7.0 (m, 1H), 6.8 (d, 1H), 6.5 (bs, 1H), 4.5 (m, 4H), 4.4 (m, 2H), 4.0 (t, 2H), 3.7 (t, 2H), 3.3 (s, 3H), 3.2 (m, 1H), 3.1 (m, 2H), 2.7 (s, 3H), 1.9-1.7 (m, 6H), 1.5 (m, 2H), 1.0 (m, 3H).

MS m/z: [M+H]⁺=574

### G. [4-(5-Aminomethyl-2-butoxy-phenyl)-piperidin-1-yl]-[1-(2-methoxy-ethyl)-7-methyl-1H-indol-3-yl]-methanone hydrochloride

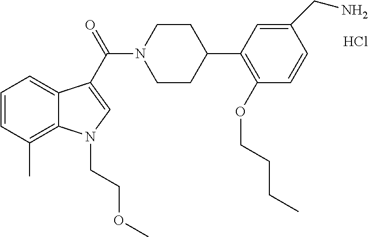

The title compound is prepared in a similar manner as described in Example 3B using N-(4-butoxy-3-{1-[1-(2-methoxy-ethyl)-7-methyl-1H-indole-3-carbonyl]-piperidin-4-yl}-benzyl)-2,2,2-trifluoro-acetamide as the starting material.

¹H NMR (300 MHz, DMSO-d6) δ 8.1 (bs, 2H), 7.6 (s, 1H), 7.5 (m, 1H), 7.4 (m, 3H), 7.2 (m, 1H), 7.0 (m, 3H), 4.6 (m, 2H), 4.4 (m, 2H), 4.0 (m, 2H), 3.9 (m, 2H), 3.6 (m, 2H), 3.2 (s, 3H), 3.1 (m, 1H), 3.0 (m, 2H), 2.6 (s, 3H), 1.9-1.7 (m, 6H), 1.4 (m, 2H), 1.0 (m, 3H).

MS m/z: [M+H]⁺=478

### Example 115

### [4-(5-Aminomethyl-2-phenethyloxy-phenyl)-piperidin-1-yl]-[1-(2-methoxy-ethyl)-7-methyl-1H-indol-3-yl]-methanone hydrochloride

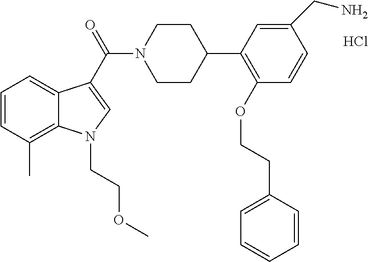

### A. 3-Bromo-4-phenethyloxy-benzonitrile

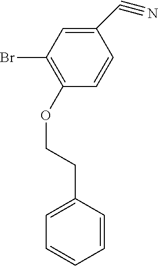

The title compound is prepared in a similar manner as described in Example 115A using (2-bromo-ethyl)-benzene as the starting material.

¹H NMR (300 MHz, CDCl₃) δ 7.8 (s, 1H), 7.6 (d, 1H), 7.4 (m, 5H), 6.8 (d, 1H), 4.3 (t, 2H), 3.2 (t, 2H). MS m/z: [M+H]⁺=303

### B. 4-Phenethyloxy-3-pyridin-4-yl-benzonitrile

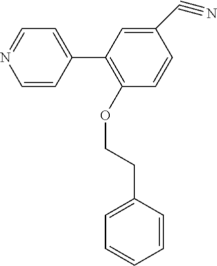

The title compound is prepared in a similar manner as described in Example 115C using 3-bromo-4-phenethyloxy-benzonitrile as the starting material.

¹H NMR (300 MHz, CDCl₃) δ 8.6 (d, 2H), 7.7 (d, 2H), 7.6 (s, 1H), 7.3 (m, 5H), 7.2 (m, 2H), 7.0 (d, 1H), 4.3 (t, 2H), 3.1 (t, 2H).

MS m/z: [M+H]⁺=301.

### C. 4-Phenethyloxy-3-pyridin-4-yl-benzylamine

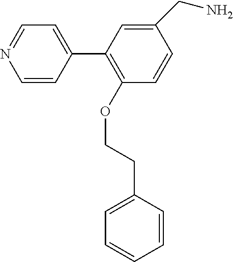

The title compound is prepared in a similar manner as described in Example 115B using 4-phenethyloxy-3-pyridin-4-yl-benzonitrile as the starting material.

¹H NMR (300 MHz, CDCl₃) δ 8.6 (d, 2H), 7.7 (d, 2H), 7.6 (s, 1H), 7.3 (m, 5H), 7.2 (m, 2H), 7.0 (d, 1H), 4.3 (t, 2H), 4.1 (m, 2H), 3.1 (t, 2H).

### D. 2,2,2-Trifluoro-N-(4-phenethyloxy-3-pyridin-4-yl-benzyl)-acetamide

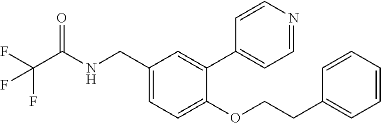

The title compound is prepared in a similar manner as described in Example 1F using 4-phenethyloxy-3-pyridin-4-yl-benzylamine as the starting material.

¹H NMR (300 MHz, CDCl₃) δ 8.6 (d, 2H), 7.8 (d, 2H), 7.6 (m, 1H), 7.3 (m, 5H), 7.2 (m, 2H), 7.0 (d, 1H), 4.5 (m, 2H), 4.3 (t, 2H), 3.1 (t, 2H).

MS m/z: [M+H]⁺=401

### E. 2,2,2-Trifluoro-N-(4-phenethyloxy-3-piperidin-4-yl-benzyl)-acetamide hydrochloride

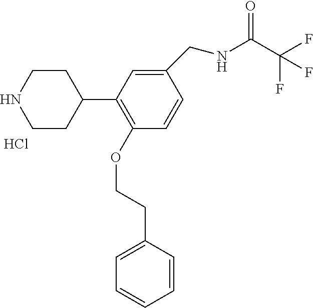

The title compound is prepared in a similar manner as described in Example 1I using 2,2,2-trifluoro-N-(4-phenethyloxy-3-pyridin-4-yl-benzyl)-acetamide as the starting material. Crude material is used in next step without purification.

MS m/z: [M+H]⁺=407

### F. 2,2,2-Trifluoro-N-(3-{1-[1-(2-methoxy-ethyl)-7-methyl-1H-indole-3-carbonyl]-piperidin-4-yl}-4-phenethyloxy-benzyl)-acetamide

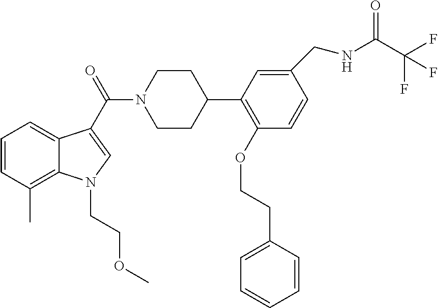

The title compound is prepared in a similar manner as described in Example 2I using 2,2,2-trifluoro-N-(4-phenethyloxy-3-piperidin-4-yl-benzyl)-acetamide hydrochloride and 1-(2-methoxy-ethyl)-7-methyl-1H-indole-3-carboxylic acid as the starting materials.

¹H NMR (300 MHz, CDCl₃) δ 7.6 (d, 1H), 7.4 (s, 1H), 7.3 (m, 5H), 7.1 (m, 3H), 7.0 (m, 1H), 6.8 (d, 1H), 6.4 (bs, 1H), 4.5 (m, 4H), 4.4 (m, 2H), 4.2 (t, 2H), 3.7 (t, 2H), 3.3 (s, 3H), 3.1 (m, 3H), 3.0 (m, 2H), 2.7 (s, 3H), 1.8-1.6 (m, 4H).

MS m/z: [M+H]⁺=622

### G. [4-(5-Aminomethyl-2-phenethyloxy-phenyl)-piperidin-1-yl]-[1-(2-methoxy-ethyl)-7-methyl-1H-indol-3-yl]-methanone hydrochloride

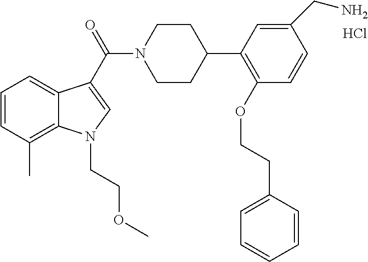

The title compound is prepared in a similar manner as described in Example 3B using 2,2,2-trifluoro-N-(3-{1-[1-(2-methoxy-ethyl)-7-methyl-1H-indole-3-carbonyl]-piperidin-4-yl}-4-phenethyloxy-benzylyacetamide as the starting material.

¹H NMR (300 MHz, DMSO-d6) δ 8.4 (bs, 2H), 7.7 (s, 1H), 7.55 (m, 1H), 7.3 (m, 6H), 7.2 (m, 1H), 7.0 (m, 3H), 4.6 (m, 2H), 4.4 (m, 2H), 4.2 (m, 2H), 3.9 (m, 2H), 3.7 (m, 2H), 3.2 (s, 3H), 3.2 (m, 3H), 3.0 (m, 2H), 2.7 (s, 3H), 1.7-1.4 (m, 4H).

MS m/z: [M+H]⁺=526

### Example 116

### [4-(5-Aminomethyl-3-fluoro-phenyl)-piperidin-1-yl]-[1-(2-methoxy-ethyl)-7-methyl-1H-indol-3-yl]-methanone hydrochloride

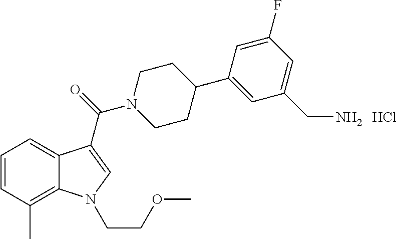

### A. 3-Bromo-5-fluoro-benzylamine

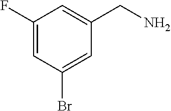

To a solution of 3-bromo-5-fluorobenzonitrile (5.05 g, 25.23 mmol) in tetrahydrofuran (120 mL) is added the borane•THF complex (31 mL, 31.00 mmol)) dropwise. The resulting mixture is refluxed for ˜2 hr, and then 2 N HCl solution is added. The resulting mixture is refluxed for an additional ˜1 hr. THF is removed in vacuo and the aqueous residue is extracted with ethyl acetate. The aqueous layer is basified with 50% NaOH and the aqueous phase is extracted with ethyl acetate (3×) and the combined organic phases are washed with brine then separated and dried

(MgSO₄). The organic phase is concentrated in vacuo to afford without purification the titled compound (2.66 g, 52%) as yellow oil.

¹H NMR (300 MHz, CDCl₃) δ 7.77 (m, 1H), 7.15-7.10 (m, 1H), 7.01-6.98 (m, 1H), 3.86 (s, 2H), 1.51 (br s, 2H).

### B. 3-Fluoro-5-pyridin-4-yl-benzylamine

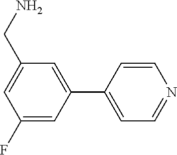

By proceeding in a similar manner to the method described in Example 112G using 3-fluoro-5-pyridin-4-yl-benzylamine and 4-pyridineboronic acid as the starting material, the titled compound is prepared as a brown viscous oil.

¹H NMR (300 MHz, CDCl₃) δ 8.67 (d, 2H), 7.49 (d, 2H), 7.40 (s, 1H), 7.20 (m, 1H), 7.13 (m, 1H), 3.98 (s, 2H), 1.96 (br s, 2H).

### C. N-(3-fluoro-5-pyridin-4-yl-benzyl)-2,2,2-trifluoro-acetamide

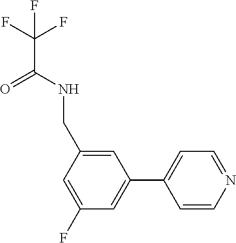

By proceeding in a similar manner to the method described in Example 1F using 3-fluoro-5-pyridin-4-yl-benzylamine as the starting material, the titled product is prepared as a yellow solid.

¹H NMR (300 MHz, CD₃CN) δ 8.77 (m, 2H), 7.99 (m, 3H), 7.59 (s, 1H), 7.52 (m, 1H), 7.26 (m, 1H), 4.53 (s, 2H).

### D. N-(3-fluoro-5-piperidin-4-yl-benzyl)-2,2,2-trifluoro-acetamide hydrochloride

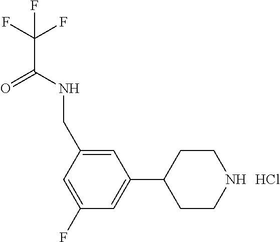

By proceeding in a similar manner to the method described in Example 112I using N-(3-fluoro-5-pyridin-4-yl-benzyl)-2,2,2-trifluoro-acetamide as the starting material, the titled product is prepared as a yellow semi-solid and used in the next step.

### E. 2,2,2-Trifluoro-N-(3-fluoro-5-{1-[1-(2-methoxy-ethyl)-7-methyl-1H-indole-3-carbonyl]-piperidin-4-yl}-benzyl)-acetamide

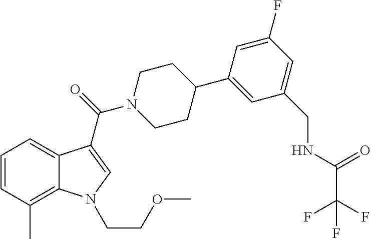

By proceeding in a similar manner to the method described in Example 2I using 7-methyl-1-(2-methoxy-ethyl)-1H-indole-3-carboxylic acid and N-(3-fluoro-5-piperidin-4-yl-benzyl)-2,2,2-trifluoro-acetamide hydrochloride as the starting material, the titled product is prepared as a white solid.

¹H NMR (300 MHz, CDCl₃) δ 7.57 (br d, 1H), 7.43 (s, 1H), 7.11-7.06 (m, 1H), 6.99-6.83 (m, 4H), 6.70 (br s, 1H), 4.60-4.94 (m, 6H), 3.71 (t, 2H), 3.31 (s, 3H), 3.07-2.99 (m, 2H), 2.83-2.75 (m, 1H), 2.71 (s, 3H), 1.90-1.66 (m, 2H), 1.75-1.61 (m, 2H).

### F. [4-(5-Aminomethyl-3-fluoro-phenyl)-piperidin-1-yl]-[1-(2-methoxy-ethyl)-7-methyl-1H-indol-3-yl]-methanone hydrochloride

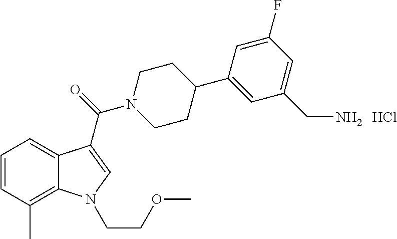

By proceeding in a similar manner to the method described in Example 3B with 2,2,2-trifluoro-N-(3-fluoro-5-{1-[1-(2-methoxy-ethyl)-7-methyl-1H-indole-3-carbonyl]-piperidin-4-yl}-benzyl)-acetamide as the starting material, the titled product is prepared as a white solid.

¹H NMR (300 MHz, DMSO-d6) δ 8.43 (br s, 3H), 7.62 (s, 1H), 7.53 (d, 1H), 7.30 (s, 1H), 7.23-7.16 (m, 2H), 7.03-6.98 (m, 1H), 6.94-6.92 (m, 1H), 4.57 (t, 2H), 4.43-3.38 (m, 2H), 4.02 (br s, 2H), 3.67 (t, 2H), 3.22 (s, 3H), 3.07-2.99 (m, 2H), 2.91-2.83 (m, 1H), 2.67 (s, 3H), 1.85-1.81 (m, 2H), 1.69-1.58 (m, 2H).

MS m/z: [M+H]⁺=424.

### Example 117

### [4-(3-Aminomethyl-2-fluoro-phenyl)-piperidin-1-yl]-[1-(2-methoxy-ethyl)-7-methyl-1H-indol-3-yl]-methanone hydrochloride

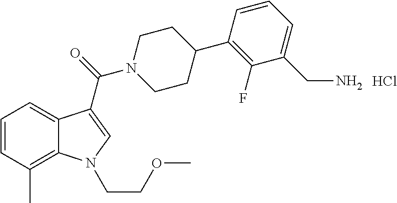

### A. N-(3-Bromo-2-fluoro-benzyl)-2,2,2-trifluoro-acetamide

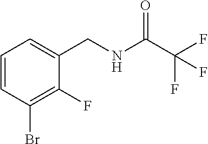

By proceeding in a similar manner to the method described in Example 115B using 3-bromo-2-fluorobenzonitrile as the starting material, 3-bromo-2-fluorobenzylamine is prepared.

The crude 3-bromo-2-fluorobenzylamine is treated with trifluoroacetic anhydride following the procedure of Example 1F, to yield the titled compound as a yellow oil.

¹H NMR (300 MHz, CDCl₃) δ 1.65 (br s, 1H), 7.56-7.51 (m, 3H), 7.33-7.28 (m, 1H), 7.07-7.01 (m, 1H), 4.59 (d, 2H).

### B. 2,2,2-Trifluoro-N-(2-fluoro-3-pyridin-4-yl-benzyl)-acetamide

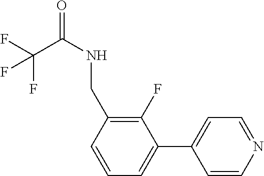

By proceeding in a similar manner to the method described in Example 112G using N-(3-bromo-2-fluoro-benzyl)-2,2,2-trifluoro-acetamide as the starting material, 2-fluoro-3-pyridin-4-yl-benzylamine is prepared. The crude 2-fluoro-3-pyridin-4-yl-benzylamine is treated with trifluoroacetic anhydride following the protocol of Example 1F to give the titled compound as an off-white solid.

¹H NMR (300 MHz, CD₃CN) δ 8.82 (br s, 1H), 8.00 (br s, 4H), 7.62-7.51 (m, 2H), 7.38-7.32 (m, 1H), 4.57 (s, 2H).

### C. 2,2,2-Trifluoro-N-(2-fluoro-3-piperidin-4-yl-benzyl)-acetamide hydrochloride

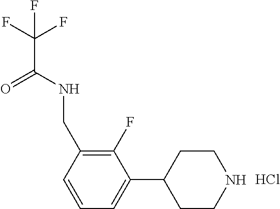

By proceeding in a similar manner to the method described in Example 112I using 2,2,2-trifluoro-N-(2-fluoro-3-pyridin-4-yl-benzyl)-acetamide as the starting material, the titled product is prepared as an off-white solid and used for the next step.

### D. 2,2,2-Trifluoro-N-(2-fluoro-3-{1-[1-(2-methoxy-ethyl)-7-methyl-1H-indole-3-carbonyl]-piperidin-4-yl}-benzyl)acetamide

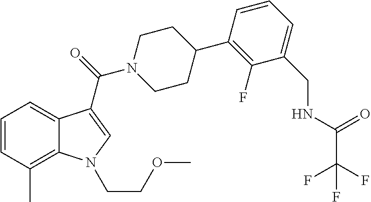

By proceeding in a similar manner to the method described in Example 2I with 7-methyl-1-(2-methoxy-ethyl)-1H-indole-3-carboxylic acid and 2,2,2-trifluoro-N-(2-s fluoro-3-piperidin-4-yl-benzyl)-acetamide hydrochloride as starting materials, the titled product is prepared as a brown gum.

¹H NMR (300 MHz, CDCl₃) δ 7.58 (br d, 1H), 7.43 (s, 1H), 7.23-7.20 (m, 2H), 7.14-7.06 (m, 2H), 6.98 (m, 1H), 6.78 (br s, 1H), 4.59-4.52 (m, 6H), 3.71 (t, 2H), 3.31 (s, 3H), 3.20-3.03 (m, 3H), 2.72 (s, 3H), 1.89-1.63 (m, 4H).

### E. [4-(3-Aminomethyl-2-fluoro-phenyl)-piperidin-1-yl]-[1-(2-methoxy-ethyl)-7-methyl-1H-indol-3-yl]-methanone hydrochloride

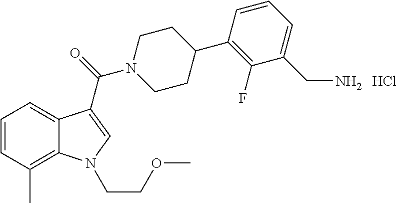

By proceeding in a similar manner to the method described in Example 3B using 2,2,2-trifluoro-N-(2-fluoro-3-{1-[1-(2-methoxy-ethyl)-7-methyl-1H-indole-3-carbonyl]-piperidin-4-yl}-benzyl)-acetamide as the starting material, the titled product is prepared as a white solid.

¹H NMR (300 MHz, DMSO-d6) δ 8.36 (br s, 3H), 7.63 (s, 1H), 7.53 (d, 1H), 7.46-7.40 (m, 2H), 7.26-7.21 (m, 1H), 7.02-6.98 (m, 1H), 6.94-6.92 (m, 1H), 4.57 (t, 2H), 4.42 (br d, 2H), 4.06 (m, 2H), 3.67 (t, 2H), 3.22 (s, 3H), 3.19-2.99 (m, 3H), 2.67 (s, 3H), 1.80-1.63 (m, 4H).

MS m/z: [M+H]⁺=424.

### Example 118

### [4-(3-Aminomethyl-4-chloro-phenyl)-piperidin-1-yl]-[1-(2-methoxy-ethyl)-7-methyl-1H-indol-3-yl]-methanone hydrochloride

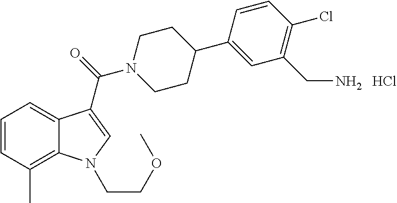

### A. N-(5-Bromo-2-chloro-benzyl)-2,2,2-trifluoro-acetamide

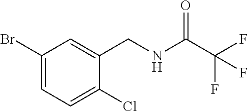

The title compound is prepared according to the procedure by Kuramochi, T. et al., Bioorg. & Med. Chem., 2005, vol. 13, pp. 4022-4036, using 5-bromo-2-chlorobenzyl alcohol as the starting material, 5-bromo-2-chlorobenzylamine is obtained as a white semi-solid and used in the next step without further purification. By proceeding in a similar manner to the method described in Example 1F using 5-bromo-2-chlorobenzylamine as the starting material, the titled product is prepared as a white solid.

¹H NMR (300 MHz, CDCl₃) δ 7.52 (d, 1H), 7.43 (dd, 1H), 7.28 (d, 1H), 6.71 (br s, 1H), 4.59 (d, 2H).

¹⁹F NMR (300 MHz, CDCl₃) δ −76.2 (s).

MS m/z: [M+H]⁺=313, 315, 317.

### B. N-(2-Chloro-5-pyridin-4-yl-benzyl)-2,2,2-trifluoro-acetamide

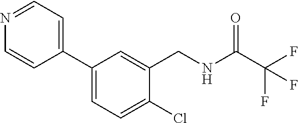

By proceeding in a similar manner to the method described in Example 112G using N-(5-bromo-2-chloro-benzyl)-2,2,2-trifluoro-acetamide as the starting material, 2-chloro-5-pyridin-4-yl-benzylamine is obtained. The crude 2-chloro-5-pyridin-4-yl-benzylamine is treated with trifluoroacetic anhydride following the protocol of Example 1F to afford the titled compound as a brownish solid.

¹H NMR (300 MHz, CD₃CN) δ 8.05 (br s, 3H), 7.82 (m, 1H), 7.79-7.75 (m, 1H), 7.63 (d, 1H), 4.63 (d, 2H), 3.13 (br s, 2H).

¹⁹F NMR (300 MHz, CD₃CN) δ −76.9 (s).

MS m/z: [M+H]⁺=315, 317.

### C. N-(2-Chloro-5-piperidin-4-yl-benzyl)-2,2,2-trifluoro-acetamide hydrochloride

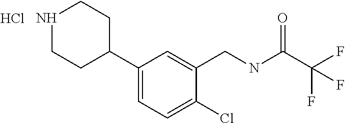

By proceeding in a similar manner to the method described in Example 112I using N-(2-chloro-5-pyridin-4-yl-benzyl)-2,2,2-trifluoro-acetamide as the starting material, the titled product is prepared as a yellowish solid.

¹H NMR (300 MHz, DMSO-d6) δ 10.05 (t, 1H), 9.13 (br s, 1H), 8.99 (br s, 1H), 7.44 (m, 1H), 7.22-7.19 (m, 2H), 4.47 (d, 2H), 3.36-3.32 (m, 2H), 3.06-2.73 (m, 3H), 1.94-1.80 (m, 4H).

¹⁹F NMR (300 MHz, DMSO-d6) δ −74.6 (s).

MS m/z: [M+H]⁺=321, 323.

### D. N-(2-Chloro-5-{1-[1-(2-methoxy-ethyl)-7-methyl-1H-indole-3-carbonyl]-piperidin-4-yl}-benzyl)-2,2,2-trifluoro-acetamide

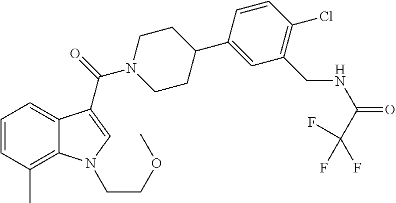

By proceeding in a similar manner to the method described in Example 2I using 7-methyl-1-(2-methoxy-ethyl)-1H-indole-3-carboxylic acid and N-(2-chloro-5-piperidin-4-yl-benzyl)-2,2,2-trifluoro-acetamide hydrochloride as starting materials, the titled product is prepared as a white solid.

¹H NMR (300 MHz, DMSO-d6) δ 9.95 (t, 1H), 7.61 (s, 1H), 7.52 (d, 1H), 7.41 (d, 1H), 7.29-7.26 (m, 2H), 7.02-6.91 (m, 2H), 4.56 (t, 2H), 4.46 (d, 2H), 4.39 (br d, 2H), 3.66 (t, 2H), 3.22 (s, 3H), 3.03 (t, 2H), 2.88-2.80 (m, 1H), 2.67 (s, 3H), 1.82-1.78 (m, 2H), 1.62-1.51 (m, 2H).

¹⁹F NMR (300 MHz, DMSO-d6) δ −74.6 (s).

MS m/z: [M+H]⁺=336, 338.

### E. [4-(3-Aminomethyl-4-chloro-phenyl)-piperidin-1-yl]-[1-(2-methoxy-ethyl)-7-methyl-1H-indol-3-yl]-methanone hydrochloride

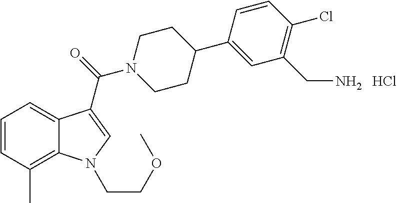

By proceeding in a similar manner to the method described in Example 3B using N-(2-chloro-5-{1-[1-(2-methoxy-ethyl)-7-methyl-1H-indole-3-carbonyl]-piperidin-4-yl}-benzyl)-2,2,2-trifluoro-acetamide as the starting material, the titled compound is prepared as a pale yellow powder.

¹H NMR (300 MHz, DMSO-d6) δ 8.39 (br s, 2H), 7.62 (s, 1H), 7.56-7.52 (m, 2H), 7.46 (d, 1H), 7.35 (dd, 1H), 7.00 (t, 1H), 6.93 (d, 1H), 4.57 (t, 2H), 4.41 (br d, 2H), 4.12 (dd, 2H), 3.68-3.65 (m, 4H), 3.05 (br t, 2H), 2.90-2.82 (m, 1H), 2.67 (s, 3H), 1.84-1.80 (m, 2H), 1.68-1.57 (m, 2H).

MS m/z: [M+H]⁺=440, 442.

### Example 119

### (4-Aminomethyl-3′,4′,5′,6′-tetrahydro-2′H-[2,4′]bipyridinyl-1′-yl)-[1-(2-methoxy-ethyl)-7-methyl-1H-indol-3-yl]-methanone hydrochloride

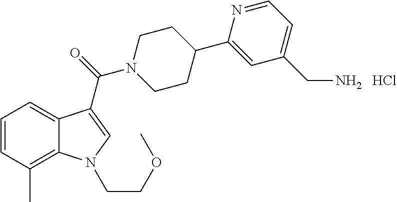

### A. 4-Aminomethyl-3′,6′-dihydro-2′H-[2,4′]bipyridinyl-1′-carboxylic acid tert-butyl ester

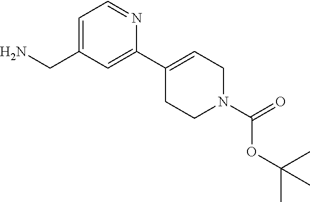

The title compound is prepared according to the procedure by Eastwood, P. R., Tet. Lett., 2000, vol. 41, pp. 3705-3708 & WO 2007/092435 (p 140-145) using C-(2-bromopyridin-4-yl)methylamine and 4-(4,4,5,5-tetramethyl[1,3,2]dioxaborolan-2-yl)-3,6-dihydro-2H-pyridine-1-carboxylic acid tert-butyl ester as starting materials to provide the titled compound as a viscous brown oil.

¹H NMR (300 MHz, CDCl₃) δ 8.50 (d, 1H), 7.35 (s, 1H), 7.13 (d, 1H), 6.61 (br s, 1H), 4.16-4.11 (m, 2H), 3.91 (s, 2H), 3.65 (t, 2H), 2.66 (br s, 2H), 1.86 (br s, 2H), 1.24 (s, 9H)

MS m/z: [M+H]⁺=290.

### B. 4-[(2,2,2-Trifluoro-acetylamino)-methyl]-3′,6′-dihydro-2′H-[2,4′]bipyridinyl-1′-carboxylic acid tert-butyl ester

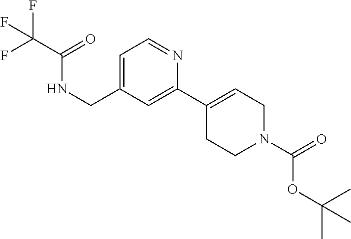

By proceeding in a similar manner to the method described in Example 1F using 4-aminomethyl-3′,6′-dihydro-2′H-[2,4′]bipyridinyl-1′-carboxylic acid tert-butyl ester as the starting material, the titled product is prepared as a viscous brown oil.

¹H NMR (300 MHz, CDCl₃) δ 8.95 (t, 1H), 8.69 (d, 1H), 7.62 (s, 1H), 7.53 (d, 1H), 6.80 (br s, 1H), 4.67 (d, 2H), 4.19 (m, 2H), 3.65 (t, 2H), 2.60 (br s, 2H), 1.48 (s, 9H). ¹⁹F NMR (300 MHz, CDCl₃) δ −76.1 (s).

MS m/z: [M+H]⁺=386.

### C. 4-[(2,2,2-Trifluoro-acetylamino)-methyl]-3′,4′,5′,6′-tetrahydro-2′H-[2,4]bipyridinyl-1′-carboxylic acid tert-butyl ester

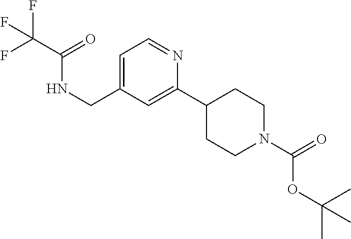

By proceeding in a similar manner to the method described in Example 114C using 4-[(2,2,2-trifluoro-acetylamino)-methyl]-3′,6′-dihydro-2′H-[2,4′]bipyridinyl-1′-carboxylic acid tert-butyl ester as the starting material, the titled product is prepared as a brown solid.

¹H NMR (300 MHz, CDCl₃) δ 8.80 (br s, 1H), 8.64 (br s, 1H), 7.53-7.48 (m, 2H), 4.67 (br s, 2H), 4.25 (br d, 2H), 3.20 (m, 1H), 2.85 (br s, 2H), 1.92 (m, 2H), 1.68 (m, 2H), 1.47 (s, 9H).

¹⁹F NMR (300 MHz, CDCl₃) δ −76.0 (s).

MS m/z: [M+H]⁺=388.

### D. 2,2,2-Trifluoro-N-(1′,2′,3′,4′,5′,6′-hexahydro-[2,4]bipyridinyl-4-ylmethyl)-acetamide dihydrochloride

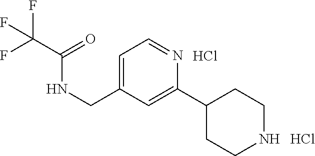

By proceeding in a similar manner to the method described in Example 111I using 4-[(2,2,2-trifluoro-acetylamino)-methyl]-3′,4′,5′,6′-tetrahydro-2′H-[2,4]bipyridinyl-t-carboxylic acid tert-butyl ester as the starting material, the titled product is prepared as a brown solid.

¹H NMR (300 MHz, DMSO-d6) δ 10.34 (t, 1H), 9.21 (br s, 1H), 9.10 (br s, 1H), 8.70 (d, 1H), 7.61-7.58 (m, 2H), 4.62 (d, 2H), 3.32 (m, 2H), 3.07-2.96 (m, 2H), 2.15-2.10 (m, 2H), 2.06-1.93 (m, 2H).

¹⁹F NMR (300 MHz, DMSO-d6) δ −74.7 (s).

MS m/z: [M+H]⁺=288.

### E. 2,2,2-Trifluoro-N-{1′-[1-(2-methoxy-ethyl)-7-methyl-1H-indole-3-carbonyl]-1′,2′,3′,4′,5′,6′-hexahydro-[2,4]bipyridinyl-4-ylmethyl}-acetamide

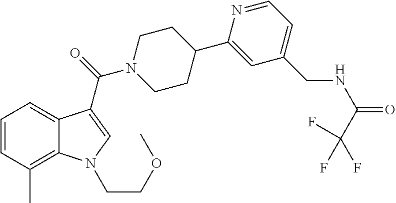

By proceeding in a similar manner to the method described in Example 2I using 7-methyl-1-(2-methoxy-ethyl)-1H-indole-3-carboxylic acid and 2,2,2-trifluoro-N-(1′,2′,3′,4′,5′,6′-hexahydro-[2,4]bipyridinyl-4-ylmethyl)-acetamide dihydrochloride as starting materials, the titled product is prepared as a white solid.

¹H NMR (300 MHz, DMSO-d6) δ 10.11 (t, 1H), 8.57 (d, 1H), 7.62 (s, 1H), 7.52 (d, 1H), 7.44 (br s, 1H), 7.31 (m, 1H), 7.0-6.92 (m, 2H), 4.57 (t, 2H), 4.50 (d, 2H), 4.40 (br d, 2H), 3.66 (t, 2H), 3.22 (s, 3H), 3.15-2.98 (m, 3H), 2.67 (s, 3H), 1.92-1.88 (m, 2H), 1.79-1.67 (m, 2H).

¹⁹F NMR (300 MHz, DMSO-d6) δ −74.7 (s).

MS m/z: [M+H]⁺=503.

### F. (4-Aminomethyl-3′,4′,5′,6′-tetrahydro-2′H-[2,4]bipyridinyl-1′-yl)-[1′-(2-methoxy-ethyl)-7-methyl-1H-indol-3-yl]-methanone dihydrochloride

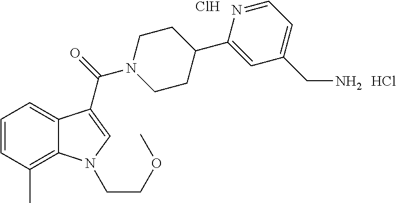

By proceeding in a similar manner to the method described in Example 3B using 2,2,2-trifluoro-N-{1′-[1-(2-methoxy-ethyl)-7-methyl-1H-indole-3-carbonyl]-1′,2′,3′,4′,5′,6′-hexahydro-[2,4]bipyridinyl-4-ylmethyl}-acetamide as the starting material, the titled product is prepared as an off-white solid.

¹H NMR (300 MHz, DMSO-d6) δ 8.74 (br d, 4H), 7.90 (br s, 1H), 7.72 (m, 1H), 7.64 (s, 1H), 7.55 (d, 1H), 7.01 (t, 1H), 6.93 (d, 1H), 4.57 (t, 2H), 4.42 (br d, 2H), 4.23 (dd, 2H), 3.67 (t, 2H), 3.22 (s, 3H), 3.10 (t, 2H), 2.68 (s, 3H), 1.99-1.95 (m, 2H), 1.83-1.70 (m, 2H).

MS m/z: [M+H]⁺=407.

### Example 120

### 2,3-Dihydro-benzofuran-7-carboxylic acid [3-[4-(5-aminomethyl-2-fluoro-phenyl)-piperidine-1-carbonyl]-1-(2-methoxy-ethyl)-1H-indol-4-yl]-amide hydrochloride

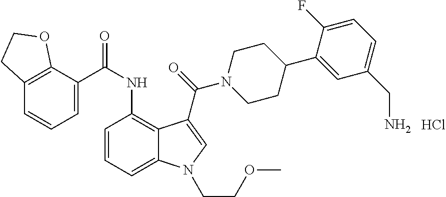

### A. 2,3-Dihydro-benzofuran-7-carboxylic acid [3-(4-{2-fluoro-5-[(2,2,2-trifluoro-acetylamino)-methyl]-phenyl}-piperidine-1-carbonyl)-1-(2-methoxy-ethyl)-1H-indol-4-yl]-amide

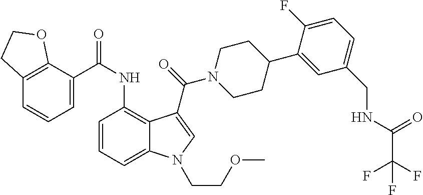

The title compound is prepared in a similar manner as described in Example 6E using 7-coumarancarboxylic acid and N-(3-{1-[4-amino-1-(2-methoxy-ethyl)-1H-indole-3-carbonyl]-piperidin-4-yl}-4-fluoro-benzyl)-2,2,2-trifluoro-acetamide as the starting materials.

¹H NMR (300 MHz, DMSO-d6) δ 10.61 (s, 1H), 9.94 (t, 1H), 8.01 (d, 1H), 7.71 (s, 1H), 7.55 (d, 1H), 7.40-7.34 (m, 2H), 7.22 (t, 1H), 7.16-7.11 (m, 3H), 6.90 (t, 1H), 4.67 (t, 2H), 4.45 (br s, 1H), 4.41-4.37 (m, 3H), 4.33 (d, 2H), 3.65 (t, 2H), 3.30-3.23 (m, 2H), 3.20 (s, 3H), 3.10-2.99 (m, 3H), 1.77-1.73 (m, 2H), 1.61-1.53 (m, 2H).

¹⁹F NMR (300 MHz, DMSO-d6) δ −74.8 (s), −121.8 (m).

MS m/z: [M+H]⁺=667.

### B. 2,3-Dihydro-benzofuran-7-carboxylic acid [3-[4-(5-aminomethyl-2-fluoro-phenyl)-piperidine-1-carbonyl]-1-(2-methoxy-ethyl)-1H-indol-4-yl]-amide hydrochloride

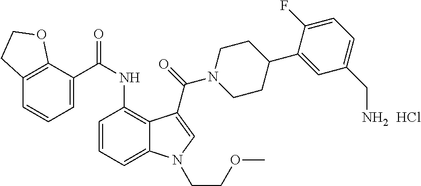

The title compound is prepared in a similar manner as described in Example 3B using 2,3-dihydro-benzofuran-7-carboxylic acid [3-(4-{2-fluoro-5-[(2,2,2-trifluoro-acetylamino)-methyl]-phenyl}-piperidine-1-carbonyl)-1-(2-methoxy-ethyl)-1H-indol-4-yl]-amide as the starting material.

¹H NMR (300 MHz, DMSO-d6) δ 10.63 (s, 1H), 8.20 (br s, 2H), 8.00 (d, 1H), 7.70 (s, 1H), 7.53 (d, 1H), 7.38-7.30 (m, 4H), 7.23-7.15 (m, 2H), 6.89 (t, 1H), 4.65 (t, 2H), 4.45-4.37 (m, 3H), 3.94 (br s, 2H), 3.64 (t, 2H), 3.28-3.21 (m, 3H), 3.18 (s, 3H), 3.10-3.00 (m, 3H), 1.76-1.72 (m, 2H), 1.59-1.55 (m, 2H).

¹⁹F NMR (300 MHz, DMSO-d6) δ −120.4 (br s).

MS m/z: [M+H]⁺=571.

### Example 121

### 7-Methyl-1H-indole-2-carboxylic acid [3-[4-(5-aminomethyl-2-fluoro-phenyl)-piperidine-1-carbonyl]-1-(2-methoxy-ethyl)-1H-indol-4-yl]-amide dihydrochloride

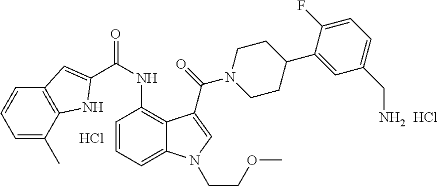

### A. 7-Methyl-1H-indole-2-carboxylic acid [3-(4-{2-fluoro-5-[(2,2,2-trifluoro-acetylamino)-methyl]-phenyl}-piperidine-1-carbonyl)-1-(2-methoxy-ethyl)-1H-indol-4-yl]-amide

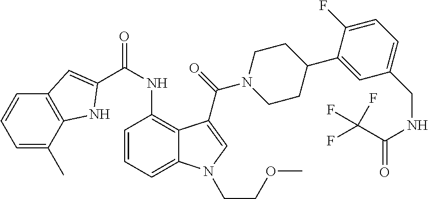

The title compound is prepared in a similar manner as described in Example 6E using 7-methylindole-2-carboxylic acid and N-(3-{1-[4-amino-1-(2-methoxy-ethyl)-1H-indole-3-carbonyl]-piperidin-4-yl}-4-fluoro-benzyl)-2,2,2-trifluoro-acetamide as the starting materials.

¹H NMR (300 MHz, DMSO-d6) δ 11.64 (s, 1H), 11.54 (s 1H), 9.87 (t, 1H), 8.24 (d, 1H), 7.86 (s, 1H), 7.49-7.47 (m, 2H), 7.37 (d, 1H), 7.28 (t, 1H), 7.19 (d, 1H), 7.12-7.11 (m, 1H), 7.09 (s, 1H), 7.01-6.94 (m, 2H), 4.71 (br d, 2H), 4.34 (t, 2H), 4.21 (d, 2H), 3.68 (t, 2H), 3.30-3.14 (m, 6H), 2.54 (s, 3H), 1.90-1.87 (m, 2H), 1.81-1.69 (m, 2H).

¹⁹F NMR (300 MHz, DMSO-d6) δ −74.9 (s), −121.9 (m).

MS m/z: [M+H]⁺=678.

### B. 7-Methyl-1H-indole-2-carboxylic acid [3-[4-(5-aminomethyl-2-fluoro-phenyl)-piperidine-1-carbonyl]-1-(2-methoxy-ethyl)-1H-indol-4-yl]-amide dihydrochloride

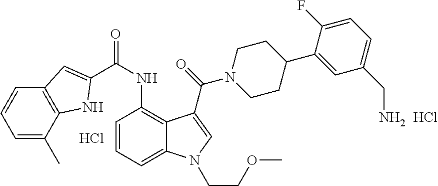

The title compound is prepared in a similar manner as described in Example 3B with 7-methyl-1H-indole-2-carboxylic acid [3-(4-{2-fluoro-5-[(2,2,2-trifluoro-acetylamino)-methyl]-phenyl}-piperidine-1-carbonyl)-1-(2-methoxy-ethyl)-1H-indol-4-yl]-amide as the starting material.

¹H NMR (300 MHz, DMSO-d6) δ 11.67 (s, 1H), 11.52 (s 1H), 8.28-8.21 (m, 3H), 7.86 (s, 1H), 7.46-7.44 (m, 3H), 7.37-7.23 (m, 3H), 7.16 (t, 1H), 6.99-6.92 (m, 2H), 4.70 (br d, 2H), 4.42 (t, 2H), 3.86-3.84 (m, 2H), 3.66 (m, 2H), 3.40-3.26 (m, 6H), 2.52 (s, 3H), 1.89-1.85 (m, 2H), 1.78-1.71 (m, 2H).

¹⁹F NMR (300 MHz, DMSO-d6) δ −74.9 (s), −121.9 (m).

MS m/z: [M+H]⁺=678.

### Example 122

### 1-Methyl-piperidine-3-carboxylic acid [3-[4-(5-aminomethyl-2-fluoro-phenyl)-piperidine-1-carbonyl]-1-(2-methoxy-ethyl)-1H-indol-4-yl]-amide hydrochloride

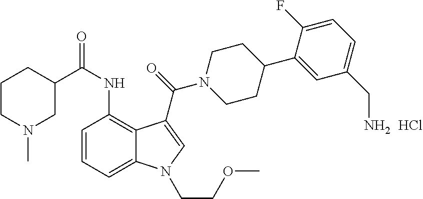

### A. 1-Methyl-piperidine-3-carboxylic acid [3-(4-{2-fluoro-5-[(2,2,2-trifluoro-acetylamino)-methyl]-phenyl}-piperidine-1-carbonyl)-1-(2-methoxy-ethyl)-1H-indol-4-yl]-amide

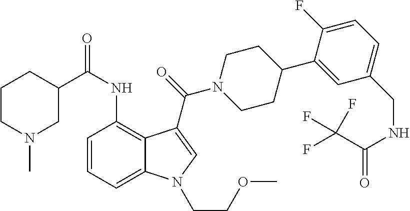

The title compound is prepared in a similar manner as described in Example 6E using 1-methylpiperidine-3-carboxylic acid hydrochloride and N-(3-{1-[4-amino-1-(2-methoxy-ethyl)-1H-indole-3-carbonyl]-piperidin-4-yl}-4-fluoro-benzyl)-2,2,2-trifluoro-acetamide as the starting materials.

¹H NMR (300 MHz, DMSO-d6) δ 10.80 (s, 1H), 9.92 (t, 1H), 7.90 (d, 1H), 7.78 (s, 1H), 7.31-7.24 (m, 2H), 7.19-7.13 (m, 3H), 4.59 (br d, 2H), 4.38 (t, 2H), 4.33 (d, 2H), 3.64 (t, 2H), 3.26-3.13 (m, 3H), 3.19 (s, 3H), 2.88 (br d, 1H), 2.66 (br d, 1H), 2.55-2.43 (m, 1H), 2.14 (s, 3H), 2.09-2.02 (m, 1H), 1.87-1.83 (m, 4H), 1.74-1.62 (m, 3H), 1.50-1.40 (m, 2H).

¹⁹F NMR (300 MHz, DMSO-d6) δ −74.8 (s), −121.6 (m).

MS m/z: [M+H]⁺=646.

### B. 1-Methyl-piperidine-3-carboxylic acid [3-[4-(5-aminomethyl-2-fluoro-phenyl)-piperidine-1-carbonyl]-1-(2-methoxy-ethyl)-1H-indol-4-yl]-amide hydrochloride

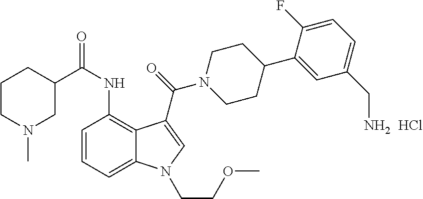

The title compound is prepared in a similar manner as described in Example 3B using 1-methyl-piperidine-4-carboxylic acid [3-(4-{2-fluoro-5-[(2,2,2-trifluoro-acetylamino)-methyl]-phenyl}-piperidine-1-carbonyl)-1-(2-methoxy-ethyl)-1H-indol-4-yl]-amide as the starting material.

¹H NMR (300 MHz, DMSO-d6) δ 10.95 (s, 1H), 8.50 (br s, 2H), 7.89 (d, 1H), 7.79 (s, 1H), 7.54 (d, 1H), 7.37-7.29 (m, 2H), 7.23-7.14 (m, 2H), 4.58 (br d, 2H), 4.38 (t, 2H), 3.96 (s, 2H), 3.63 (t, 2H), 3.31-3.10 (m, 3H), 3.18 (s, 3H), 2.92 (br d, 1H), 2.65 (br s, 1H), 2.39 (br s, 4H), 1.96-1.49 (m, 10H).

¹⁹F NMR (300 MHz, DMSO-d6) δ −119.4 (br s).

MS m/z: [M+H]⁺=550.

### Example 123

### 3-[4-(5-Aminomethyl-2-fluoro-phenyl)-piperidine-1-carbonyl]-1-(2-methoxy-ethyl)-1H-indole-4-carboxylic acid cyclopropylamide hydrochloride

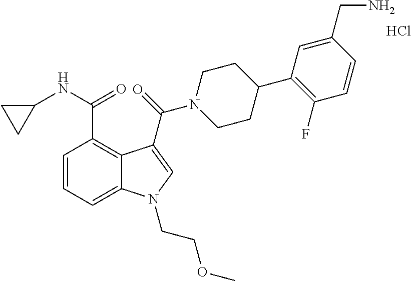

### A. 3-(4-{2-Fluoro-5-[(2,2,2-trifluoro-acetylamino)-methyl]-phenyl}-piperidine-1-carbonyl)-1-(2-methoxy-ethyl)-1H-indole-4-carboxylic acid cyclopropylamide

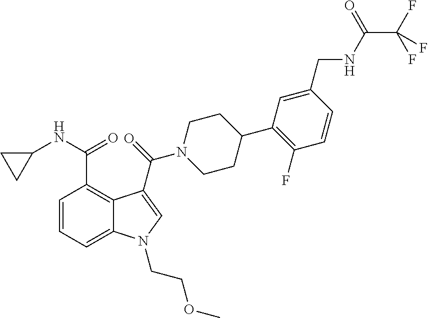

The title compound is prepared in a similar manner as described in Example 96A using 3-(4-{2-fluoro-5-[(2,2,2-trifluoro-acetylamino)-methyl]-phenyl}-piperidine-1-carbonyl)-1-(2-methoxy-ethyl)-1H-indole-4-carboxylic acid and cyclopropylamine as the starting materials.

¹H NMR (300 MHz, CDCl₃) δ 7.6 (bs, 1H), 7.5 (m, 3H), 7.4 (s, 1H), 7.3 (m, 1H), 6.95 (m, 1H), 6.4 (m, 1H), 4.9 (m, 1H), 4.5 (m, 2H), 4.3 (t, 2H), 3.7 (t, 2H), 3.3 (s, 3H), 3.2 (m, 2H), 3.0 (m, 2H), 1.9-1.70 (m, 4H), 1.6 (m, 2H), 0.8 (m, 4H).

MS m/z: [M+H]⁺=589.

### B. 3-[4-(5-Aminomethyl-2-fluoro-phenyl)-piperidine-1-carbonyl]-1-(2-methoxy-ethyl)-1H-indole-4-carboxylic acid cyclopropylamide hydrochloride

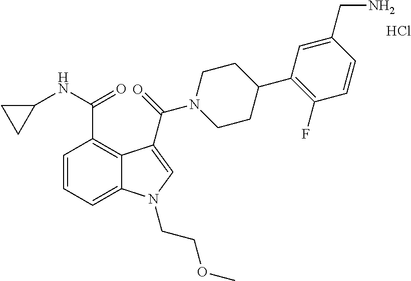

The title compound is prepared in a similar manner as described in Example 1K using 3-(4-{2-fluoro-5-[(2,2,2-trifluoro-acetylamino)-methyl]-phenyl}-piperidine-1-carbonyl)-1-(2-methoxy-ethyl)-1H-indole-4-carboxylic acid cyclopropylamide as the starting material.

¹H NMR (300 MHz, DMSO-d6) δ 8.4 (bs, 2H), 8.1 (m, 1H), 7.6 (m, 3H), 7.4 (m, 1H), 7.2 (m, 3H), 4.4 (t, 2H), 4.0 (m, 3H), 3.6 (t, 2H), 3.2 (s, 3H), 3.1-2.9 (m, 4H), 2.7 (m, 1H), 1.85 (m, 4H), 0.6 (m, 4H).

MS m/z: [M+H]⁺=493.

### Example 124

Oxazole-5-carboxylic acid [3-[4-(5-aminomethyl-2-fluoro-phenyl)-piperidine-1-carbonyl]-1-(2-methoxy-ethyl)-1H-indol-4-yl]-amide hydrochloride

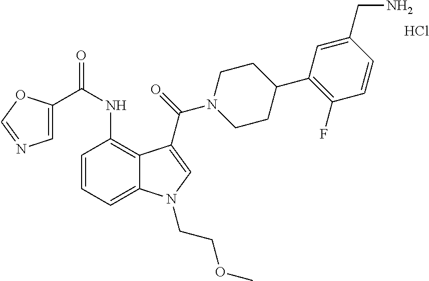

### A. Oxazole-5-carboxylic acid [3-(4-{2-fluoro-5-[(2,2,2-trifluoro-acetylamino)-methyl]-phenyl}-piperidine-1-carbonyl)-1-(2-methoxy-ethyl)-1H-indol-4-yl]-amide

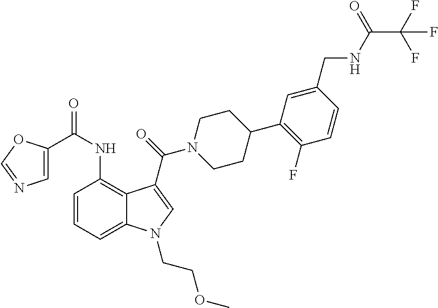

To a solution of oxazole-5-carboxylic acid (108 mg, 0.96 mmol) in DMF (10 mL) is added TBTU (0.37 g, 1.15 mmol), triethylamine (0.3 ml, 2.11 mmol), and N-(3-{1-[4-amino-1-(2-methoxy-ethyl)-1H-indole-3-carbonyl]-piperidin-4-yl}-4-fluoro-benzyl)-2,2,2-trifluoro-acetamide (0.5 g, 0.96 mmol). The resulting mixture is stirred at room temperature overnight. The mixture is diluted with EtOAc and washed with water, brine, dried over MgSO₄, filtered and concentrated in vacuo. Purification by flash chromatography on SiO₂ eluting with 70% ethyl acetate/heptanes affords the titled compound (0.52 g, 88%).

¹H NMR (300 MHz, CDCl₃) δ 11.5 (s, 1H), 8.2 (d, 1H), 8.0 (m, 2H), 7.4 (d, 1H), 7.3 (m, 1H), 7.2 (m, 3H), 7.0 (m, 2H), 4.8 (m, 2H), 4.5 (m, 2H), 4.3 (t, 2H), 3.7 (t, 2H), 3.3 (s, 3H), 3.2 (m, 3H), 1.9 (m, 2H), 1.7 (m, 2H).

MS m/z: [M+H]⁺=616.

### B. Oxazole-5-carboxylic acid [3-[4-(5-aminomethyl-2-fluoro-phenyl)-piperidine-1-carbonyl]-1-(2-methoxy-ethyl)-1H-indol-4-yl]-amide hydrochloride

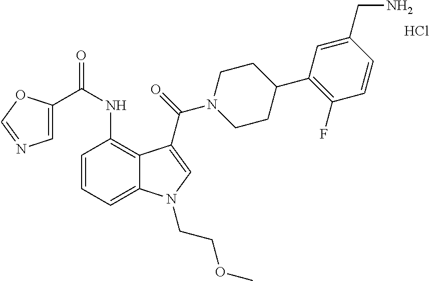

The title compound is prepared in a similar manner as described in Example 1K using oxazole-5-carboxylic acid [3-(4-{2-fluoro-5-[(2,2,2-trifluoro-acetylamino)-methyl]-phenyl}-piperidine-1-carbonyl)-1-(2-methoxy-ethyl)-1H-indol-4-yl]-amide as the starting material.

¹H NMR (300 MHz, DMSO-d6) δ 12.0 (s, 1H), 8.6 (s, 1H), 8.2 (m, 3H), 8.0 (s, 1H), 7.9 (s, 1H), 7.5-7.2 (m, 5H), 4.7 (m, 2H), 4.4 (m, 2H), 4.0 (m, 2H), 3.7 (m, 2H), 3.3 (m, 3H), 3.2 (s, 3H), 1.9 (m, 2H), 1.7 (m, 2H).

MS m/z: [M+H]⁺=520.

### Biological Activity

The properties of the compound of the present invention are demonstrated by: 1) its beta-Tryptase Inhibitory Potency (IC₅₀ and Kᵢ values).

### In Vitro Test Procedure

As all the actions of tryptase, as described in the background section, are dependent on its catalytic activity, then compounds that inhibit its catalytic activity will potentially inhibit the actions of tryptase. Inhibition of this catalytic activity may be measured by the in vitro enzyme assay and the cellular assay.

Tryptase inhibition activity is confirmed using either isolated human lung tryptase or recombinant human beta tryptase expressed in yeast cells. Essentially equivalent results are obtained using isolated native enzyme or the expressed enzyme. The assay procedure employs a 96 well microplate (Costar 3590) using L-pyroglutamyl-L-prolyl-L-arginine-para-nitroanilide (S2366: Quadratech) as substrate (essentially as described by McEuen et. al. Biochem Pharm, 1996, 52, pages 331-340). Assays are performed at room temperature using 0.5 mM substrate (2×Kₘ) and the microplate is read on a microplate reader (Beckman Biomek Plate reader) at 405 nm wavelength.

Materials and Methods for Tryptase primary screen (Chromogenic assay)

### Assay Buffer

50 mM Tris (pH 8.2), 100 mM NaCl, 0.05% Tween 20, 50 μg/mL heparin.

### Substrate

S2366 (Stock solutions of 2.5 mM).

### Enzyme

Purified recombinant beta Tryptase Stocks of 310 μg/mL.
Protocol (Single point determination)
Add 60 μL of diluted substrate (final concentration of 500 μM in assay buffer) to each well
Add compound in duplicates, final concentration of 20 volume 20
Add enzyme at a final concentration of 50 ng/mL in a volume of 20
Total volume for each well is 100
Agitate briefly to mix and incubate at room temp in the dark for 30 minutes
Read absorbencies at 405 nM
   Each plate has the following controls:
Totals: 60 μL of substrate, 20 μL of buffer (with 0.2% final concentration of DMSO),
   20 μL of enzyme
      Non-specific: 60 μL of substrate, 40 μL of buffer (with 0.2% DMSO)
      Totals: 60 μL of substrate, 20 μL of buffer (No DMSO), 20 μL of enzyme Non-specific: 60 μL of substrate, 40 μL of buffer (No DMSO)
      Protocol (IC₅₀ and Kᵢ determination)

The protocol is essentially the same as above except that the compound is added in duplicates at the following final concentrations: 0.01, 0.03, 0.1, 0.3, 1, 3, 10 μM (All dilutions carried out manually). For every assay, whether single point or IC₅₀ determination, a standard compound is used to derive IC₅₀ for comparison. From the IC₅₀ value, the Kᵢ can be calculated using the following formula: Kᵢ=IC₅₀/(1+[Substrate]/Kₘ).

The compounds of this invention display beta-Tryptase inhibition in the range of 1 μM to <1 nM.

### Biological Activity

The compounds of this invention display Ki values in the range of 1 μM to <1 nM as shown in Table 1 below.

TABLE 1

| EXAMPLE # | Tryptase Ki (nM) |
|---|---|
| 1 | 47 |
| 2 | 234 |
| 3 | 109 |
| 4 | 1303 |
| 5 | 96, 56 (8923:11) |
| 6 | 20 |
| 7 | 139 |
| 8 | 35 |
| 9 | 203 |
| 10 | 175 |
| 11 | 56 |
| 12 | 26 |
| 13 | 75 |
| 14 | 83 |
| 15 | 18 |
| 16 | 97 |
| 17 | 37 |
| 18 | 40 |
| 19 | 94 |
| 20 | 19 |
| 21 | 28 |
| 22 | 390 |
| 23 | 42 |
| 24 | 98 |
| 25 | 672 |
| 26 | 306 |
| 27 | 164 |
| 28 | 538 |
| 29 | 331 |
| 30 | 50 |
| 31 | 306 |
| 32 | 1280 |
| 33 | >10 uM |
| 34 | 1013 |
| 35 | 8.6 |
| 36 | 1150 |
| 37 | 87 |
| 38 | 66 |
| 39 | 10 |
| 40 | 25 |
| 41 | 62 |
| 42 | 123 |
| 43 | 850 |
| 44 | 51 |
| 45 | 753 |
| 46 | 94 |
| 47 | 56, 59 |
| 48 | 125 |
| 49 | 291 |
| 50 | 241 |
| 51 | 54 |
| 52 | 49 |
| 53 | 289 |
| 54 | 96 |
| 55 | 72 |
| 56 | 9 |
| 57 | 54 |
| 58 | 108 |
| 59 | 32 |
| 60 | 22 |
| 61 | 17 |
| 62 | 22 |
| 63 | 46 |
| 64 | 39 |
| 65 | 11 |
| 66 | 164 |
| 67 | 12 |
| 68 | 35 |
| 69 | 400 |
| 70 | 21 |
| 71 | 49 |
| 72 | 13 |
| 73 | 7 |
| 74 | 26 |
| 75 | 59 |
| 76 | 211 |
| 77 | 57 |
| 78 | 117 |
| 79 | 935 |
| 80 | 961 |
| 81 | 224 |
| 82 | 6 |
| 83 | 5 |
| 84 | 22 |
| 85 | 9 |
| 86 | 80 |
| 87 | 207 |
| 88 | 239 |
| 89 | 11 |
| 90 | 396 |
| 91 | 20 (719 54 nM; 8923 28 nM) |
| 92 | 90 |
| 93 | 160 |
| 94 | 21 |
| 95 | 44 (8923 15 nM, 719D 47 nM) |
| 96 | 18 |
| 97 | 28 |
| 98 | 147 |
| 99 | 25 |
| 100 | 17 |
| 101 | 11 |
| 102 | 22 |
| 103 | 41 |
| 104 | 204 |
| 105 | 100 |
| 106 | 149 |
| 107 | 315 |
| 108 | 15 |
| 109 | 142 |
| 110 | 1260 |
| 111 | 1017 |
| 112 | 998 |
| 113 | 656 |
| 114 | 526 |
| 115 | 390 |
| 116 | 746 |
| 117 | 756 |
| 118 | 832 |
| 119 | 180 |
| 120 | 3 |
| 121 | 99 |
| 122 | 44 |
| 123 | 50 |
| 124 | 8 |

Although the invention has been illustrated by certain of the preceding examples, it is not to be construed as being limited thereby; but rather, the invention encompasses the generic area as hereinbefore disclosed. Various modifications and embodiments can be made without departing from the spirit and scope thereof.

Material described in the specification, including examples, which is not within the ambit of the claims is not covered by the claimed invention. The scope of protection is as defined in the claims.

What is claimed is:

1. A compound of formula: 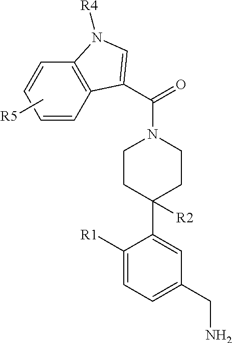
wherein
R1 is F, Cl, Br, OCH₂CO₂CH₃, OCH₂CONW1W2, CH₂OH or optionally substituted alkyl, haloalkyl, alkoxy or haloalkoxy; wherein
W1 and W2 are independently H or alkyl;
R2 is H, F, Cl, Br, OH, CH₂OH, alkyl or alkoxy;
provided R1 and R2 are not H at the same time;
R4 is alkyl optionally substituted by one or more groups selected from hydroxy, alkoxy, haloalkoxy, cycloalkyl, heterocycloalkyl, aryl, optionally substituted aryl and heteroaryl, or optionally substituted heteroaryl; and
R5 is H, halo, alkoxy, haloalkoxy, alkyl, amido, carboxyl, ureyl, sulfonyl amido, sulfonyl urea, alkyl optionally substituted by one or more groups selected from hydroxy, alkoxy, haloalkoxy, cycloalkyl, heterocycloalkyl, aryl, or heteroaryl; or
a salt thereof or an enantiomer or a diastereomer thereof.

2. The compound according to claim 1, wherein
R1 is selected from the group consisting of F, Cl, Br, OCH₂CO₂CH₃ and CH₂OH.

3. The compound according to claim 1, wherein R1 is F.

4. The compound according to claim 1, wherein
R2 is selected from the group consisting of H, F, Cl, Br, OH, CH₃, OCH₃ and CH₂OH.

5. The compound according to claim 1, wherein R2 is H.

6. The compound according to claim 1, wherein R1 is F and R2 is H.

7. A pharmaceutical composition comprising a compound as claimed in claim 1 in combination with at least one pharmaceutically acceptable excipient, diluent or a carrier.

8. A method of treating an inflammatory disease in a patient, comprising administering to said patient a therapeutically effective amount of a compound according to claim 1.

9. The method according to claim 8 wherein the inflammatory disease is COPD.
